SEQUENCE LISTING (83 sequences)

SEQ ID NO: 1 - DNA, 2056 bp, Artificial Sequence
  cDNA of TTM2
atgggtcaagacagcaatggaattgagtttcatcagaagagacatggtctcttgaaggatcaagtccaattggttaagagaagagactctattcggtatgaaattgtttctattcaagatcggttgtcatttgagaagggcttctttgcggttatccgtgcttgccaattgctttctcagaagaatgatgggatcatattggttggtgttgctggaccttctggtgctggaaagactgtattcactgagaagatactcaattttctgccaagtgttgctgtcatttcaatggacaattataatgattctagtcggattgttgatgggaactttgatgatccacggttaacggactatgacacattgctcaagaatcttgaagacttaaaggaaggaaagcaggttgaggttcctatttatgattttaagtccagctctcgtgttggatacaggacccttgatgtcccaccttctcggattgtgattattgaaggaatctatgctttgagtgaaaaactgcgacctttattggatcttcgtgtgtctgttactggtggagttcattttgaccttgttaaacgggttctccgtgatatacaacgtgcaggtcaacagccagaggagattatccatcagatatctgaaacagtatacccgatgtacaaagctttcattgagccagatctccagactgctcaaatcaaaatcattaataaattcaaccccttcactggttttcagagcccgacttacatcttgaagtcaagaaaggaggtatctgttgatcagatcaaggcggtcctttctgatggacatacagagactaaggaggagacctatgatatatatcttcttcctccgggtgaagatccagagtcgtgccaatcatatttgaggatgcggaataaagatggaaagtacagccttatgtttgaggaatgggttacggatactccttttgtcatatccccaaggattacatttgaagtcagtgttcgcctacttggtgggctcatggcattgggatacacaatagcaactatacttaaaaggaacagccatgtatttgctactgataaggtgtttgtgaaaatcgattggcttgagcaactgaatcgtcactacatgcaggtgcaaggtaaagatcggcaacttgtacagagtactgcagagcagctaggattggaaggatcgttcattccacgcacctatattgaacagatccaactcgaaaaactaataaatgaagtaatggccctaccagatgatctaaagaacaagcttagcttagatgaggatttggtgtctagttcaagccctaaggaagcactcttacgagcgtctgcagatagagtagccatgagaaataagaacctcaaaagaggcatgtcacactcatattcaacccaaagagataagaatctgtccaagcttgctggttattcttcaagcgataggaggtacgaagaaagaaatcacgactcgccagcgaacgaggggtttatgactctgctttcagaacaaatatcatctctcaacgagagaatggatgagttcacaagtcgaattgaagagctcaattcaaagttgagctgcaataaaaactctccaacacagcagagcttgtcaatccaaaccgaagtctgcaatgggtcagctcctacttcgtatttcatttctggtctggacaatggctgcttgacaaattccataatgccccattcatcatcctcctcccaactagccaaggattcacccttaatggaagagatatcgaccatatcacgaggacagcgtcaagttatgcatcagttggataatttgtgcaatctgatgagggaaagctcagcagaaaggtcacgcctagcaagaacagggagcagcaatagcggtaacagaggcagatcaagcaaaagctccttcttgtccaatgtggaatctaacaagctccctcttgtgttaaccgtggctatttgcagcataggtattatagtgatcaagagctacattaacaagcggcaataacatctattagccactatgggttttctcttct

SEQ ID NO: 2 - DNA, 3991 bp, Artificial Sequence
  genomic DNA sequence of TTM2
aatgttacctcctcgtgggtctgagatctttttccccagattctctacaaatcgctctccccgataaagaagaagctctcacaaaattcctctttctctctctctctctgattccccattattagtttctgtgttaaaattgaattgcgacataactctgccaaagtgataagccccgattcacactaattccgagagatttttctgtgtgagtgccatactaaactccgagaaatcggctcaagtttcgatttttgtttctgggttttaccttttcaaccaatctgtttgcgttttttcttttgttctgggtgttgttgttatagaacagtttgatcgtttcttctttgatggtttttgtttggattcgtttcgagctttcgcttgttttgtttcattgtatggctgcattttgatgataatttcatatccgctacttttggattagagtgctgcgttatctttagtctgcttgactcattcctccatgggtttaagagtaaatgtcactgttcctttaaaatgttccgtacaattcagtcttcactatgtgtgtttttggctctcttagcttttggtctctccatgtttcccagcttaagattatgtcttattaatgaaaatgtgttcttttttgcagattattgttcataatgggtcaagacagcaatggaattgagtttcatcagaagagacatggtctcttgaaggatcaagtccaattggttaagagaagagactctattcggtatgaaattgtttctattcaagatcggttgtcatttgagaagggcttctttgcggttatccgtgcttgccaattgctttctcagaagaatgatgggatcatattggttggtgttgctggaccttctggtgctggaaagactgtattcactgagaagatactcaattttctgccaagtgttgctgtcatttcaatggacaattataatgattctagtcggattgttgatgggaactttgatggtaagaattttcatcttgataggtcccatgaggaatgaagtcctatgacacattgttttgaaacttgaagtatcttgctgctgacaaaccttatgttttgaaacttagatccacggttaacggactatgacacattgctcaagaatcttgaagacttaaaggaaggaaagcaggttgaggttcctatttatgattttaagtccagctctcgtgttggatacaggtaatgcgtgacgtgattgtgcagtttccatttactgattcagtcatcattttgtactttatctaaacaaacaaccacttggtgtccattgtcacaaaagtttgatattacattcacatcagcatggtttctgtttattccactgaagcattgtttttaatgccatgatttaatttgctaggacccttgatgtcccaccttctcggattgtgattattgaaggaatctatgctttgagtgaaaaactgcgacctttattggatcttcgtgtgtctgttactggtggagttcattttgaccttgttaaacgggttctccgtgatatacaacgtgcaggtcaacagccagaggagattatccatcagatatctgaaacagtttgtcctcatttcttttatttcgtgtgactgtttggtttagtatatgagctgccaattgtttatattaacaactcactgtttatgtaggtatacccgatgtacaaagctttcattgagccagatctccagactgctcaaatcaaaatcattaataaattcaaccccttcactggttttcagagcccgacttacatcttgaaggtttgaaaagtgaccggatttctatccatcttatcatattaatcagtgctctgcaaactcagtattcaactattgacagcgtttggttaattgaagttcttttactattactttgttgtagtcaagaaaggaggtatctgttgatcagatcaaggcggtcctttctgatggacatacagagactaaggaggagacctatgatatatatcttcttcctccgggtgaagatccagagtcgtgccaatcatatttgaggatgcggaataaagatggaaagtacagccttatgtttgaggtttgttcagagtttattttccatgttctcatcaatatgactattcaatatctggaaaagctgacaatccctctgattctggtaagatgcttagtatctggtgaataactgtggttctggttttgacaaccaggaatgggttacggatactccttttgtcatatccccaaggattacatttgaagtcagtgttcgcctacttggtgggctcatggcattgggatacacaatagcaactatacttaaaaggaacagccatgtatttgctactgataaggtgtttgtgaaaatcgattggcttgagcaactgaatcgtcactacatgcaggtctgtctatctatactcattcaccatcatttgctagaaaattgattgttcatctggctttatgatgacagtactcttgttcccagttactatgaaatttctttatctccccaaaaaaatatgactacaatattcaaattttgttataaacaggtgcaaggtaaagatcggcaacttgtacagagtactgcagagcagctaggattggaaggatcgttcattccacgcacctatattgaacagatccaactcgaaaaactaataaatgaagtaatggtatgttttgctgttcgggttttgagttttgttttgactacattttatctggggtcctgactaaaaatcccatcacaggccctaccagatgatctaaagaacaagcttagcttagatgaggatttggtgtctagttcaagccctaaggaagcactcttacgagcgtctgcagatagagtagccatgagaaataagaacctcaaaaggtacacatcttttgaggagtgtgtgagaaagctttgttacttccaacccatgtgtccttagttatgccatttattatacacagaggcatgtcacactcatattcaacccaaagagataagaatctgtccaagcttgctggttattcttcaagcgataggaggtacgaagaaagaaatcacgactcgccagcgaacgaggttcaaatttgttctctttcattccctcttggcaactttgaagtcttccttttaacttaagggtgcacttcttctggttttcaactatttttaggggtttatgactctgctttcagaacaaatatcatctctcaacgagagaatggatgagttcacaagtcgaattgaagagctcaattcaaagttgagctgcaataaaaactctccaacacagcagagcttgtcaatccaaaccgaagtctgcaatgggtcagctcctacttcgtatttcatttctggtctggacaatggctgcttgacaaattccataatgccccattcatcatcctcctcccaactagccaaggattcacccttaatggaagaggtaagtaacctcacgcatctctcgtttatgaatttggattttattgcgttgctttgtaactttgagctgctctggtgcaacagatatcgaccatatcacgaggacagcgtcaagttatgcatcagttggataatttgtgcaatctgatgagggaaagctcagcagaaaggtcacgcctagcaagaacagggagcagcaatagcggtaacagaggcagatcaagcaaaagctccttcttgtccaatgtggaatctaacaagctccctcttgtgttaaccgtggctatttgcagcataggtattatagtgatcaagagctacattaacaagcggcaataacatctattagccactatgggttttctcttctttttttgttcttttgttttggtatttttctcactggaggcgttttgtgagcttccctggtttctctacgtagacaatgacgccagttctcttcccctaaattagtcgtttggaagacgttctcgattatttattcaataaagtttaggtttttagttt

SEQ ID NO: 3 - DNA, 2025 bp, Arabidopsis thaliana
atgggtcaagacagcaatggaattgagtttcatcagaagagacatggtctcttgaaggatcaagtccaattggttaagagaagagactctattcggtatgaaattgtttctattcaagatcggttgtcatttgagaagggcttctttgcggttatccgtgcttgccaattgctttctcagaagaatgatgggatcatattggttggtgttgctggaccttctggtgctggaaagactgtattcactgagaagatactcaattttctgccaagtgttgctgtcatttcaatggacaattataatgattctagtcggattgttgatgggaactttgatgatccacggttaacggactatgacacattgctcaagaatcttgaagacttaaaggaaggaaagcaggttgaggttcctatttatgattttaagtccagctctcgtgttggatacaggacccttgatgtcccaccttctcggattgtgattattgaaggaatctatgctttgagtgaaaaactgcgacctttattggatcttcgtgtgtctgttactggtggagttcattttgaccttgttaaacgggttctccgtgatatacaacgtgcaggtcaacagccagaggagattatccatcagatatctgaaacagtatacccgatgtacaaagctttcattgagccagatctccagactgctcaaatcaaaatcattaataaattcaaccccttcactggttttcagagcccgacttacatcttgaagtcaagaaaggaggtatctgttgatcagatcaaggcggtcctttctgatggacatacagagactaaggaggagacctatgatatatatcttcttcctccgggtgaagatccagagtcgtgccaatcatatttgaggatgcggaataaagatggaaagtacagccttatgtttgaggaatgggttacggatactccttttgtcatatccccaaggattacatttgaagtcagtgttcgcctacttggtgggctcatggcattgggatacacaatagcaactatacttaaaaggaacagccatgtatttgctactgataaggtgtttgtgaaaatcgattggcttgagcaactgaatcgtcactacatgcaggtgcaaggtaaagatcggcaacttgtacagagtactgcagagcagctaggattggaaggatcgttcattccacgcacctatattgaacagatccaactcgaaaaactaataaatgaagtaatggccctaccagatgatctaaagaacaagcttagcttagatgaggatttggtgtctagttcaagccctaaggaagcactcttacgagcgtctgcagatagagtagccatgagaaataagaacctcaaaagaggcatgtcacactcatattcaacccaaagagataagaatctgtccaagcttgctggttattcttcaagcgataggaggtacgaagaaagaaatcacgactcgccagcgaacgaggggtttatgactctgctttcagaacaaatatcatctctcaacgagagaatggatgagttcacaagtcgaattgaagagctcaattcaaagttgagctgcaataaaaactctccaacacagcagagcttgtcaatccaaaccgaagtctgcaatgggtcagctcctacttcgtatttcatttctggtctggacaatggctgcttgacaaattccataatgccccattcatcatcctcctcccaactagccaaggattcacccttaatggaagagatatcgaccatatcacgaggacagcgtcaagttatgcatcagttggataatttgtgcaatctgatgagggaaagctcagcagaaaggtcacgcctagcaagaacagggagcagcaatagcggtaacagaggcagatcaagcaaaagctccttcttgtccaatgtggaatctaacaagctccctcttgtgttaaccgtggctatttgcagcataggtattatagtgatcaagagctacattaacaagcggcaataa

SEQ ID NO: 4 - DNA, 1962 bp, Solanum lycopersicum
atgcctaaagatactagtaatggagaagctactcggagagctggtctcttaaaggaccaagttcaactagtcaagagaaagaactgtgatcgatatgaaatcatctcgatacctgataatttgtcatttgagaaaggtttctttgttgtaatccgtgcgtgccagttgttggttcaaaagaatgaaggattgataatacttggtgttgctggtccctccggagcaggaaagactgtgtttacggagaaaatactcagcttcatgccaagtgttgcagttatctcaatggataattacaatgatgctagtcgaatagttgatggaaactttgatgatccacgacttacagactacgatacattgctgaaaaacatcaatgatctaaaggccgggaagccggctgaggttcccatatatgatttcaaatctagctctcgcataggatacaggattcttgaagtacctagctcccgcattttgattattgagggtatctatgctttgagtgaaaagttgcggccttttctggatcttcgcgtatctgtgacaggtggggttcactttgatcttgtcaaaagggttttacgggatatacagcgtgccgggcaggaaccctcggagataatccatcaaatatctgaaacggtttatccaatgtacaaggcttttattgaacctgatctcaagactgcacatataaaaattatcaacaaatttaatccatttactggatttcagagtcctacatacattttaaagtcttttagggatgtgcaagtcgatcaaatcaagtctgcattatctgaagaacacactgaaagtacggagcaaacttacgacatataccttctgccacctggtgaagatccagagacatgccaatcatatctgaggatgagaaataaagatggaaagtacagtctcatgtttgaggagtgggtaactgattctccatttgtcatatcaccaagaatcagttttgaagttagtgtgaagcttcttggtggattgatggctttgggatacacaattgcgacaatactcaaaagaagtagtcatgtattttctgatgaaaaggtttgcgtgaaaattgattggcttgagcaactgaaccggcattatgttcaggtccaaggaagagatcgccttattgtaaaatctgttgctgatcagcttggactggagggttcatacactccgcgtacatatattgagcaaatacagctagagaagcttgtgaatgaagttatggcactaccagatgatttgaaaacgaagcttagcttagatgaagatctcgtctccagcccaaaagaagctctctctcgagcctcagcagagagagtagcatggagaaataagaatttaagaagtggattgtcccattcgtacgcaaatcacagagagaaaaatctgtccaagattgatactgatggtcagagatttgatgacagaaacactgattcagcaacattagcaaatcagggagctgttacacatttatcggaacaaatatctaccttgaatgatcggatggatgattttacatctaaaatggaagagcttaattccaagttaaccaaaaaaagaacttctcagagcacacaaagtctggctttgcaagccgaagcttgtaatggatccggcccaacttcttacttcatatctggtttaggaaatggttccttgacaggatccattatgccaaattcatcctcctcctctttgctaatcccaaaggagtccaatctgatggaagagctatctaacgtggcacgtggacaacgccaaattatgcatcagttagacagccttagcaatcttcttcgcgagagactgggagaacaatcacgacaagccagaacaagcaagagaagtgatctaaattcgatcagagtacctctcgttgtgaccttagcagtaagtggattggggttgtttctgtttaggagccgaaactga

SEQ ID NO: 5 - DNA, 1944 bp, Solanum lycopersicum
atggatatagatactgctaatgctgaatccattaatcagaaagctggtctcttaaaggatcaggttcgactaattaagagaaaggattgtgatagatacgagattgcctcaataccagataatttgtcatttgagaaaggattcttcattgtaatccgtgcatgccaagtgttggttcagaataatgaaggactgataatgataggagtcgctggtccctcgggtgctggaaagactgtatttacggataaaataatgaacttcatgccaagcattgcagttatatcaatggataactataatgatgctagtcgaattgttgatggcaattttgatgatccacgccttacagactatgatacgctgctgaaaaatatcaatgatctcaagactgggaaagcagcagagattccgatatatgatttcaaatctagttcccgaataggatacaggactgtcgaagtccctagctcccgcattgtggttattgagggcatctatgctctgaatgaaaagttgcggcctttccttgatcttcgcatatccgtaaatggtggagttcactttgaccttgttaaaagagttttgcgcgacatacaacgtgcagggcaggaaccatcagaaataatccatcaagtatctgaaacggtttatccaatgtacaaggcttacattgagcctgatctcaaaactgcacacataaaaattatcaacaaatttaatcctttttctggatttcagagtcctacgtacattctaaagtcgtcaaggaatctgaaggtagaacaaatcaagtctgtcttgtctgaagaccacactgaaagtacggaactgatctatgacatataccttctgccacctggtgaagatccagagacatgccaatcatatctgaggatgcggaataaagatgggaaatacaatctcatgtttgaggaatgggtcactgattctccatttgtcatatcaccaagaatcagttttgaagttagcgtgcgtcttcttggtggattgatggctttgggatacacaatggcagccatcctcaaaagaagcagccatgtattttctgatgaaagggtttgtgtgaaaattgattggcttgagcaactaaagcgccactatgttcaggtccaaggaagagatcgtgttgttgtaaaaggtattgctgagcagctgggtttggaaggttcatacactcctcgtacatatattgaacaaatacagctagagaagcttgtgaatgaggttatggcactaccagatgacttgaaaacaaagcttagcctagatgaagatattgtctcaagtcctaaagaagctctctctcaagcctcggcacagagggcgtggagaaataagaatatcagaagcggattgtcacattcatattcaactaacagggacaaaaatctcaatgttagttttgattatcagagaaatgacaggattgaggaatcagggacaagattagcaaaccagggagcgatcacacgtctattggagcaaatttctacgttgaatgaccggatggacaacttcacgtctataatggaagaacttaattctaagttgtgcagcagaatagcttctccgagaaacaaacaagcttcaccatgcatacaaaatctgacgttagattctgaagcatgcgttggatctgctcctactaactatttcatatctggtttggagaacggttcattgactgggtccatcatgcctaattcctcatcttttacttccactattggaaaggagtctgccttgatggaagagatatccaatattgcacgtggacagcgtcaacttatgcatcagctggacaacgttagcaatgttcttcgcgagggattaggagaacagtctcgacaagcaagagtgaataagaaaagtgatacaatcaaaaccatcagaaagcctctcattgtaacattagcagtt

SEQ ID NO: 6 - DNA, 2010 bp, Petunia hybrida
atggatgtagatactgcaaatgctgaattgtctaatcagagagcaggtctcttaaaggaacaggttcgactagttaagagaaaggattgtgatcgatacgagattgtctcgataccagataatttgtcatttgaaaaaggattcttcattgtaatccgtgcatgccaattgctggttcaaaagaatgacggactggtaatggtaggagtagctggtccatctggtgctggaaagactgtatttactgagaaaatacttaacttcatgccaagcgttgcagttatctcaatggataactacaatgatgctagtcgaattgttgacggaaactttgatgacccacgcataacagactatgatatgttgctgaataacatcaatgatctgaagactgggaagccagcagagcttccaatatatgatttcaaatctagttcccgaataggatacaggacccttgaagtccctagctctcgtattgtgattattgagggcatccatgctctgaatgaaaagttgcggcctttcctggatcttcgcgtatctgttaatggtggagtgcactttgatcttgttaaaagagttttacgcgacatacaacgtgctgggcaggaaccatcagagataatccatcaagtatctgaaacggtttatcccatgtacaaggcctatattgagcctgacctcaaaacggcacacataaaaattatcaacaaatttaatcctttttctggatttcagagtcctacttacattctaaaggcaagaccactaaacctccatatccagaatctggagatagaacaaatcaagtctgtcttgtctgaagaacacactgaatgtacggaacaggtctatgacatataccttctaccacctggtgaagatccagagacgtgccaatcatatctgaggatgcgaaataaagatgggaaatacaatctcatgtttgaggaatgggtcactgattctccatttgtcatatcaccaagaattagttttgaagttggtgtgcggcttcttggtggattgatggctttgggatacacaatagcggctatcctcaaaagaagtagccatgtattttctgatgaaaaggtctgtgtgaaaattgaccagctcgaacaattgaaccggcactatgtgcaggtccaaggaaaagatcgtgtcattgtaaaatgtattgctgatcagctgggtttggaaggttcatacactcctcgtacatatattgagcaaatacagctagagaagcttgtgaatgaggttatggcactaccagaggacttgaaaacaaagcttagcctagacgaagatattgtcgcaagccctaaagaagctctgtctcgagcctcagcagagagggtatcatggagaaataagaacatcagaagtggcttgtcacattcatattcaacgaacagggacaaaaatctacccaatcttacctctgattatcagagaaatgacgggagtgcggagtcagcaacaagattagcaaatgagggagcggtcaaacatttatcggagcaaatttctaccttgaacgaccggatggatgactttacatctagaatggaagagcttaattcccagttgagcagcagacgagcttctccgagaagcaaacgagcttctccaagcgcacacagtctggctttggaaactgaagtctgcaatggatctgccccaacttcctatttcatatctggtttggagaatggttccttgactgggtccatcatgccaaattcctcatcttttgcttcccttactgcaaaggagtctgcgtttatggaagagatatccaatattgcacgtggacagcgtcaagttatgcatcggttggacaatattagcaatgttctccgcgagagattagcagaactatctggacaggaaagaatgaacaagaaaagagatataactgatattcatcctattagaatccctatcatcttaacactagcagttggtggcctgggaatcctcttgtttaagagcctgcaa

SEQ ID NO: 7 - DNA, 1962 bp, Petunia hybrida
atggctaaagatattggtaatggtgaatcaactcatcggagagctggtctcttaaaggaccaggtccgactagttaagagaaaggataccagtcgatatgagattgtgtcgatacctgataatttgtcatttgagaaaggattctttgttgtaatccgtgcatgtcagttgttggttcaaaagaatgaaggattgacaataattggagtagctggtccttctggagctggaaagactgtatttactgagaaaatactcagcttcatgcccagtgttgcagttatctcaatggataattacaatgatgctagtcgaatagttgatggcaactttgatgatccacgactgacggactatgataaactgctggaaaacatcaatgatctaaaggctgggaagccggctgaggttccaatatatgatttcaaatctagctctcgcacaggatacaggactcttgaagtacctacctcccgcattttgattattgagggtatctatgctttgagtgaaaagttgcggcctttgctggatcttcgtgtatctgtgacaggtggagttcactttgatcttgttaaaagggttttacgggatatacaacgcgctgggcaggaaccttcagagataatccatcaaatatctgaaacggtttatccaatgtacaaggcttatattgaacctgatcttaagactgcgcatataaaaattatcaacaaatttaatccatttactggatttcagagtcccacatacatcctaaagtcttctaggaatgtgacagtagatcaaatcaagtctgccttatctgaagaacacaccgaaagtacggagcaaacttacgacatataccttctgccacctggtgaagatccagagacatgccaatcatatctgaggatgcgaaataaagatggaaaatacagtctcatgtttgaggagtgggtcactgattctccatttgtcatatcaccaagaatcagctttgaagttagtgtgaagcttcttggtggattgatggctttgggatacacaattgcggcgatactcaagagaagcagccacgtattttctgacgaaaaggtctgcgtgaaaattgattggctagaacaactgaaccgccactatgttcaggtccaaggaaaggatcgccttattgtaggatctgttgctgatcagctgggactggagggttcgtacactcctcgtacatatattgagcaaatccagctagagaagcttgtgaatgaagttatggcactgccagatgatttaaaaacaaagcttagcttagatgaagatctagtctccagccccaaagaagctctctctcgagcctcagcagacagagtagcatggagaaataagaatttaagaagtggattgtcacagtcatatgcaaatcacagggagaaaaatctatccaagattaatactgatagtcggaggttcgatgacagaaatggtgattcagcaacaacattagcaaaccagggatctgttacacatttatcggaacaaatatcgaccttgaatgatcggatggatgactttacttctcgaatggaagagcttaactccaagttaagcagcaaaagagcttctccgagcacacaaagtctggctttgcaagctgaagcctgtaacggatccggcccaacttcttacttcatatccagtttagaaaatggttccttgacgggatcaattatgccaaattcctcatcttcttcttccctaattccaaaagagtctacgctgatggaagagctatctaatgttgtacgtggacaacgccaaatcatgcatcagttggacaatcttagcaatcttcttcgcgacagattgggtgaacgatcacgacaagcaagaacaggcaagaggagagatattgttgatattgattcggtcaagctacctctcatcgtgactttagcagttggtggattaggattcttgatgttt

SEQ ID NO: 8 - DNA, 2169 bp, Cucumis sativus
atggctcaagatcattctggttctgaatctcatcagaaacgggctggtcttttgaaagatcaagtgagattgattaagagaaaggattctgatcgctatgaaattgtttcaatccaagatcctttgtcatttgagaaagggttcttcatagttattcgtgcctgccagttgcttgcacagaagaatgatggaataatattggttggtttagccggtccttctggggctggtaaaacagtttttacagaaaagataatgaattttatgccgagtattgccatcatatcaatggatgactataatgatgcaagtcgaattgttgatggcaactttgatgatccacgtttgactgactatgacaccttgctccagaatgtccaagatctaaaagcagggaagcaagttcaggtcccaatttatgatttcaaatcaagttccagaattggatacaggacagttgaggttcccagctcaaggatcgtgatcatcgagggaatttacgctttaagtgaaaggttgcggccactattggaccttcgagtatctgttagaggtggagtgcactttgatcttgtgaaacgggtgttacgtgacatccaacgtgctggccaagaaccagaagaaattattcatcaaatttctgaaacagtgtatccaatgtacaaggcttttatcgaaccagatctcgaaactgcacatataaaaatcatcaacaagtttaatccctttacaggatttcaaagtcctacctacattttgaagtcagcgagaaagataacagtggatcaaattaaggctgtgttagctgaagatcatacagaacataaggagcagacatatgacatatatctgttgccaccaggtgaagatccggagtcttgtcaatcatatctaaggatgcgcaataaagaaggaaaatacagtcttatgtttgaggagaactacgcagtgttgctcttctttaagtttgcttggattccatattacatttatggtttggttattgttgaatatccagggattatcatctacaagcctgaatgggttaccgataatccatttatcatttctccaagaataacttttgaggtcagtgtgcgccttctaggtggactgatggccttaggatacacaatagcaacgattcttaaaagaagcagccatgtattctcagatcacagagtgtgtgtgaaaattgattggctggaacaattaaaccgccagaatgttcaggtacaaggaaaagatcgtctagttgtaaaacatgtagcagagcagctgggcttggatggttcctacattcctcgtacctacattgaacagattcaactggaaaagcttgtaaatgaagtcatggccttgccagatgatttgaagtctaaactcagtcttgacgaggatctagtttcaagccccaaagaagcactctctagagcctctgctgatagagtttccttgagaaataggaatctcaaaagtggcatatctcagtcgtatacaacccaaagggagaaaaagctgtcaggatatggttccaacaaccaaagatttgttgacagaaataccgagtctcaagcaatgctcgcaaaccagggagccattactcagctttctgaacaaatttcatctctcaatgatcggatggatgagtttacagctcgaattgaggagttgaactcaaagttaagcttcaagaggaattctcccagccaacagaacataaatctccaatccgaaacgtgtaacggctctgtgccgacttcttacttcgtctctggattgggtaatggttccttgactggatccattatacccagttcttcatccttttcccagttggccaaggattctcctataatggacgagatatcaggaatctcgagggggcaacgacaagtcatgcaccaattagattgcctgagcaatcttctccgggaacgtgacaacgcaggggacagatcccgtcaagtaagaacaaagaagaaagctataatgcctgatcctgagcctcttaaacttcctctcttgttgactctagctgtgggtggtgtaggagttgtcttgtacaagagctttttaagtctttccacaaattgtgtcgcagtcatcactcttcgacgcgggaaagacgacaggattgatttaactgatgagtctggtagtcgttga

SEQ ID NO: 9 - DNA, 1710 bp, Capsicum annuum
atggatatagatactgctaatgctgaatcgattaatcagagagctggattcttaaaggatcaggttcgactagttaagagaaaggactgtgatcgatacgagattgcctcaataccagataatttgtcatttgagaaaggattcttcattgtaatccgtgcgtgccaattgttggttcagaagaatgatggactgataatgataggagttgctggtccctctggtgctgggaagactgtatttactgagaaaataatgaactttatgccaagcattgcagttatatcaatggataactacaatgatgctagtcgaattgttgatggaaactttgatgacccacgcctaacagactatgatacgttgctgaaaaacatcaatgatctgaagactgggaaagcagcagagcttccaatatatgatttcaaatctagttcccgaataggatacaggaccctcgaagtccctagctcctgcattgtgattattgagggaatctatgctctgaatgaaaagttgcggccgttcctggatcttcgcatatctgtaaatggtggagttcactttgaccttgttaaaagagttttacgcgacatgcaacgtgctgggcaggaaccatcagaaataattcaccaagtatctgaaacggaatgggtcactgattctccatttgtcatatcaccaagaatcagttttgaagttagtgtgcgccttcttggtggattgatggcattgggatacacaatggcggctatcctcaaaagaagcagccatgtattttctgatgaaagggtttgtgtgaaaattgatttgcttgaacaactgaagcgccactatgtgcaggtccaaggaagagatcgtgttgttgtaaaatctatagctgatcagctgggtttggacggttcatacacccctcgtacgtatatcgaacaaatacagctagagaagcttgtgaatgaggttatggcactaccagatgacttgaaaacaaagcttagcttagatgaagatattgtcgcaagccctaaagaagctttttctcgagcctcgggagagagggcatcatggagaaataagaaaatcagaagcggattttcgcattcatattcaactaacagggacaaaaatctatctgatgttagttctgattatcggagtaatggcaggattgaagaatcaggggctagattatcaaaccagggagcggtcacacatttattggagcaaatatctaccttgaatgaccggatggacgacttcacgtctaaaatggaagaacttaattctaagtggagcagcagaagagcttctccaagaatcaaacaagcttcaccgagcactcaaaatctggcgttagaaactgaagcatgcattggatcagctcctactaactatttcatatctggtttggagaatggttccttgactgggtccatcatgcctaattcctcatctttcacttccgttaatggaaaggagtttaccttgatggaagagatatcaaatattgcacgtggacagcgtcaagttatgcatcagctggacaacgttagcaatgttcttcgcgagagattaggagaagagtctcgacaagaaagaatgaacaagaaaagagatacaatggatcccatcagagtgcctctcatcgtagcattagcagttggtggcctgggaattctcttgtttaagagcctgcaacaccaaacctaa

SEQ ID NO: 10 - DNA, 1977 bp, Capsicum annuum
atggctaaagatactagtaatgctgaatctgctaatcggagagctggtctcttgaaggaccaggttcgactagtcaagagaaagaacagtgatcgatatgaaattgtctcgatacctgataatttgtcatttgagaaaggtttctttgttgtaatccgtgcttgccaattgttggttcaaaagaatgaaggattgatagtaattggtgttgctggcccctccggagcggggaaaactgtgtttactgagaaaatactcagcttcatgcctagtgttgcagttatctcaatggataattacaatgatgctagtcgaatagttgatggaaactttgatgatccacggcttacagactatgatacattgctaaaaaacatcaatgatttgaaggccgggaagccggctgaggttccaatatatgatttcaaatctagctctcgcataggattcaggactcttgaagtacctagctcccgcattctgattattgaaggtatctatgctttgagtgaaaagttgcggccttttctcgatcttcgcgtatctgtgactggtggagtgcactttgatcttgtaaaaagggttttacgggatatacaacgtgccgggcaggaaccctcggagataatccatcaaatatctgaaacggtttatccaatgtacaaggcttttattgaaccagatctcaagactgcacatataaaaattatcaacaaatttaatccatttactggatttcagagtcctacttacattctaaagtcttttagggacgtgaaagtcgatcaaatcaggtctgtcctatccgaagaacacactgaaagtacggagcaaacttacgacatataccttctgccacctggtgaagatccagagacatgccagtcatatctgaggatgagaaataaagatggaaaatacagtctcatgtttgaggagtgggtcactgatactccatttgtaatatcgccaagaatcagttttgaagttagtgtgaagcttcttggtggattgatggctctgggatacacaattgcgacaatactcaaaagaagcagtcacgtattttctgatgaaaaggtttgcgtgaaaattgattggctagagcaactgaatcggcattatgttcaggtagtccaaggaagagatcgccttgttgtaaaatcttttgctgatcagctgggactggaggggtcatatactccacgtacatatattgagcaaatacaactagagaagcttgtgaatgaagttatggcactaccagatgatttgaaaacaaagcttagcttagatgaagatctcgtctccagcccaaaagaagctctctctcgagcctcagcagatagagtagcatggagaaacaagaattcaagaggtggattgtcccattcgtattcaaatcacacagagagaaatctatccaagattgatactgatagtcgaaggtttgatgacagaaacagtgattcagcagcaacattagcaaaccagggagctgttacacatttatcggaacaaatttcttccttgaatgatcggatgaatgactttacatctagaatggaagaacttaattccaagttaagcagcaaaagagcttctccaagcgcacaaagtctggctttgcaagccgaagtttgtaatggatccggcccaacttcttacttcatatctggtttagaaaatggttccttgaccggatccatcatgccaaattcgtcatcctcttcttcgctaattccaaaggagtccactcttatggaagagctatctaatgttgcgcgtggacaacgccaaattatgcatcaattggacaaccttagcaatgttcttcgtgacagactgggagaacaatcacggcaagcaagaacaagcaagacaagagacgttaattcgatcagagtacctcttgttgtgaccttagcagctggtggattggggctgtttctgtttaagagcctacaaaaccagaag

SEQ ID NO: 11 - DNA, 1959 bp, Oryza sativa
atggaccgaagtgattccatggctgattcgccaaggaggcgtaatggccttctgcgggaccaggtccagttggtgaaaaggaaggacacaaatcgctacgaaatcgtccgctttcctgatccattgtcatttgagaaaggcttctttgttatgatccgtgcctgccagcttctggtgcagcacaatgaagggatgatttttgttggcgttgctggaccctctggggccgggaagactgtgtttactgagaaggtgctcaatttcatgcccggtgttgctgtgatatcaatggataactacaatgatgcaagtcgcatagttgatggcaattttgatgatccacgcctaacagactacgacacactgttggaaaatattcatggtctgaaggagggaagatcagttcaggttccaatatatgatttcaagatgagctgccggactggatacagaacagttgatgtccctagctctaggattgttatcattgaaggcatctatgcattgagtgaaaagttacgatctgtattggacctacgtgtttctgtcactggtggtgttcattttgacctggtgaagagggtcctgagggacatacagagagctggccaggagcctgaggaaataattcatcagatatctgaaacggtttatccgatgtataaggctttcattgagccagacttgcagacagcacatataaaaataatcaacaagttcaacccattctcagggttccaaaatcctatgtacattctaaagtctccacgaactataacacctactgacatcaaagttgctcttggtgaagatcatacagaaagcattgaagaaacttatgatatatatttgcttcctccaggcgaagatccagaatcatgccaatcttatttgagaatgaggaatagggaagggaaatataatctgatgtttgaggagtgggtgaccgataatccttttatcatatcaccaaggattacgtttgaagtcagtgttcgtcttcttggaggtttgatggcattgggatatactatagcagctatactgaagagaaagagtcgtgtgttttctgatggcaaggcaactgttaaaattgattggttggagcaacttaaccgaaattatatacaggtgcaaggaagagatagaaatcatgtcaaatttgtagcggagaagttaggtctggatggctcttatattccacgcacatatattgaacaaattcagctggagaaactgataaacgatgttatggcattaccagaagatctcaagacaaagctcagcattgatgatgagctggtttcaagtccaaaagaggccctttctcgggtttctgctgataagagaaacaaacacctgaaaagtggtctatctcattcatattcaactcatggagataaaaatattgtgaaattgagtaaattgacagaaaccaacagaaggtttggaagtgggcgagcccctgaacctcctgcaattaatcagggtgctatcactcagctatccgaacagatttcaacgttgaatgagaggatggacgagttcacctctcgagttgaagaactcaactgtaaatttgcaataaagaaatcatcgaccagtcagcaaaatttagctcttccaaatgaaacttgcaatggttctgcacctacaaatctctttgtttctcatttgggcaatggtaccctgatacctcattcgtcatcatcgaaccaacttttgaaggagtcccccattgtggatgagattaatgccatatcaagaggtcagcgccaagtgatacatcagcttgacaatctgaccagcttgcttcatgagcatttggccttgacccgccaaggaaatgcagtccgcagaaacgggatcctggaaatggacatgtccatctgcccactcattgccctgacaattggcggtttcggctatttggtgttcaagagcctcaaccggagctaa

SEQ ID NO: 12 - DNA, 1959 bp, Hordeum vulgare
atggagcgtagtgattccctgtccgagtcgccaaggaagcggaatggccttctgcgagaccaggtgcagctggtgaaaaggaaggacacaagccgctatgagatcgtcccctttccagagccactgtcttttgagaaaggcttctttgtcatgatccgcgccatccagctcctggtgcagaacaatgaagggataatatttgtgggagttgctggcccctctggggctggtaagacggtgtttaccgagaaggtcctcaatttcatgcccagtgttgctgtgatatcaatggacaactacaatgatgcaagtcgcatagttgatggcaattttgatgatccacgtctaacagactatgacacactgttggaaaatattcatggtctgaaggaaggaaggtccgttcaggttccaatatatgatttcaagacgagttgccggactggatacagaacagttgatgtccctagctccaggattgttattattgaaggtatatatgcactgagtgataagttacggccaatactggatctgcgtgtttctgtcactggtggtgttcattttgacctggtgaagagagtcctaagggacatacaacgagctggccaggagcctgaggaaataatccaccagatctcagaaacggtttatccaatgtacaaggctttcattgagccggatttgaagacagcgcacatacgaattatcaacaagttcaaccctttctcagggttccagaatcctatgtacatcctgaagtcaccacggtctctaacacctgatcaaatcaaagctgctcttggtgaagatcaaacagaaagcaatgaagaaacctatgacatatatttacttccaccgggtgaagatccagaagcatgccaatcttatttgagaatgcggaatagagaagggaaatataacctgatgtttgaggagtgggtgactgataatccttttatcatatcaccaaggattacttttgaagtcagtgttcgtcttcttggtggtttgatggcgttgggatatactatagcagctatactgaagagaagcagtcgtgtgttttctgatggcaaggctactgttaaaattgactggctggagcaacttagccgaaggtatatacaggtgcaaggtagagatagactttatgtcaaatttgtagcggagcagttaggtttggatggttcttatatcccgcgcacgtatattgaacaaattcaactggagaagctgatgaatgatgttatggcattaccagatgatttgaggacaaagctcagcattgatgatgagctggtttcaagtccaaaagaagctttttcccgggcctctgctgataggagaaacgaacttatgaaaagtgggctatctcattcgtattcaacacacggagataaaagtatggtgaaattgaataaactgacagaaagcaacagaaggtttggcagtcggcgaacccctgaacctcctgcgatcaaccagggtgcaatcaaccagctgtcagaacagatatcgacattgaatgagaggatggatgagtttacctgtcgggttgaagatcttaactcaaaatttacactgataaaatcttcaccaagtcagcaaaatttagctcctccaagtgatactcgtaatggttccgcacctaccaatctctttgtttctcagttgggtaatggcacccttatacctcactcatcatcatcaaaccaactttcaaaagagtctccattgacggaagagatcatggtcttatcaagaggtcagcgccaagtaatacatcagctcgacaacctcactaacctgcttcatgagcatttggtcttgacccgccaaggaaacaccacgagcaggaaccgtatccaggaaggaatcgacatggccatctgcccgttgataatcctgacaattggcagtgtcggttactttgtgttcaggaatctcaaccggaca

SEQ ID NO: 13 - DNA, 1962 bp, Zea mays
atggaccaacatgattccatggctgactcaccaaggaggcggcacaaccttttgcgtgacaaggtccaattggtgaaaaggaaagattcagatcgctatgagatcgtccgcttccatgatccactgtcttttgagaaaggcttctttgttgtgatccgtgcttgccagcttttggcacagcacaatgacgggataatatttgttggtgttgctggcccctctggtgctgggaagactgtgttcactgagaaggtggtcaatttcatgcctgatgttgctgtgatatcaatggacaactataatgacgcaactcgcatagtcgatggcaattttgatgatccacgtctgacagactatgacacactgctggaaaatatacatggtctgaaggaaggaaggtcagttcaggttccaatatatgatttcaagtcaagctgccggactggttatagaacagttgatgtccctagctcccgaattgttatcattgaaggtatttatgcactgagtgagaagttacggccagtgatagacttgcgtgtttctgtcactggtggtgttcattttgacctagtcaagagggttttaagggatatacagcgagctggccaggagcctgaagaaataattcatcagatctctgaaacggtttatccaatgtacaaggctttcatcgaaccagatttggagaccgcacatattaagatcatcaacaagttcaacccattttcagggttccaaaatcctatgtacattctaaagtcaccaaggtctctaggacctgagaaaatcaaggctgtgcttggtgatgatcatacggaaagcaacgaagaaacttatgatatatatctgcttccacctggtgaagacccagaagcatgccaatcttacttgagaatgcggaatagggaagggaaatataatctaatgtttgaggaatgggtgaccgacaattcttttatcatatcaccaaggatcacttttgaagtcagtgttcgtcttcttggtggtttgatggcattgggatatactatagcagctatactgaagagaagcactcgtgttttttctgatggcaaggcaactgttaaaattgattggctggaacaacttaacagacaatatatacaagtgcaaggaagagatcggctttatgttaaatctgtagcggatcagttaggtttggatggttcttacataccacgcacatatattgaacaaattcagctggagaaattgattaatgatgttatggcgttaccagaagatttgaagacaaagctcagcatcgacgatgagctggtctcaagtccaaaggaagccttttcccgtgtttcggctgatcggagaaacaaacttatgaagagtggcctatctcaatcatattcaacacacggagacaaaaatattgtgaaattgagtaaactaacagaaaccaataggaggtttggtggagggcgggcccttgaaccgcctgctatcaaccagggtgcaatcacccagctttcagaacagatatcaacattgaatgaaaggatggatgagtttacctctcgagttgaagaactcaactcgaaatttacagtgaaaaaacatttgcccagtcagcagaacttagctcttccaaatgatgcttgcaatggttccacgcctacaaatctctttgtctctcatttaggcaatggtactctgatacctcattcttcatcatcaaaccaactttcaaaggattccccgatgatagaagagattatgaacataacaagaggtcagcgccaggttatacatcagctcgacaatctgacaaatctgcttcacgagcatttggtcttaacacgccagactaataataccgccagcaggaaccgggtgctggatagcgacacactcatctgtccacttatttgcctgactgttgccagtatcggttacttcatgtttaaaggtctcagtaggggctga

SEQ ID NO: 14 - DNA, 1962 bp, Brachypodium distachyo
atggagcggagtgattccatgtccgaatcaccaaggaagcggaatggccttctgcgagaccaggttcagctggtgaaaaggaaggacgcgggccgctatgaaatcgtcccctttccagagccactgtcttttgagaaaggcttctttgttatgatccgtgccatccagcttctagtgcagcacaatgaagggataatattcgtgggagttgctggcccctctggagctgggaagacggtatttactgagaaggtgctcaatttcatgcctagtgttgctgtgatatcaatggacaactacaatgatgcaagtcgcatagttgatggaaattttgatgatccacgtctaacagactatgacacactgctggaaaatattcatggtctgaaggaaggaaggtccgttcaggttccaatatatgatttcaagatgagctgccggacaggatacagaacagttgatgttcctagctccaggattgttattattgaaggtatatatgcgttgagtgataagttacggcctatattggatctgcgtgtttctgtcactggtggtgttcattttgacctggtcaagagggtcctaagagacatacaacgagctggccaggagcctgaggaaataattcatcagatctcggaaacggtttatccaatgtacaaggctttcattgagccggatttgaagacagcacacataagaattatcaacaagttcaatcctttctctgggttccaaaatcctatgtatattctgaagtcaccgcggtctctaacacctgatgaaatcaaagctgctcttggtgaagatcaaacagaaagcaatgaagaaacatatgatatatatttacttccaccaggtgaagatccagaagcatgccaatcttatttgagaatgaggaatagggaagggaaatataatctcatgtttgaggagtgggtgacggataatccttttatcatatcaccgaggattacttttgaagtcagtgtacgtcttcttggtggcttgatggcgttgggatatactatagcagctatattgaagagaagcagtcgtgtgttttctgatggcaaggctactgttaaaattgactggctggagcaacttagccgaaggtatatacaggtgcaaggtagagatagactttatgtcaaatttgtagcagagcagttaggtttggatggttcttatatcccgcgcacatatattgaacaaattcagctggagaaactgatgaatgatgttatggcattaccagatgatttgaaaacaaagctcagcattgatgatgagctggtttcaagtccaaaggaagctttttcccgggcttctgctgataggagaaacaaactcatgaaaagtgggctatctcattcatattcaacacatggagataaaagtattgtgaaattgaataaactaaccgaaaccaacagaaggtttggcagtgggcgaacccctgaaccacctgcgattaaccagggtgcaatcaaccagctgtcagaacagatatcaacattgaatgagaggatggatgagtttacgtgtcgagttgaagagctcaactcaaaatttacactgatgaaaccttcatcgagtcagcaaaatttagctcttccaagtgaaactcgcaatggttctgcacctacaaatctctttgtttctcagttgggcaatggtacccttatacctcattcgtcatcatcaaaccaactttcaaaagagtccccaatgatggaagaggttatgaacttatcaagaggtcagcgccaagtaatacatcagctggacaatctgaccaacctgcttcatgagcatttggtcttgacccgccaaggaaattccatgagcaggaaccgtatcctggagggtttcgatatggccatctgtcctcttataatcctgacaatcggcagtgtcgggtattttgcgttcaagagtctcaaccggacctaa

SEQ ID NO: 15 - DNA, 1995 bp, Prunus persica
atggctcaagatatgtctggtgttgattcacaccaaagacggcaaggccttctaaaagatcaagttcgattggttaagaaaaaggattctcattatgagattgtgccaatccaaagtcccttgtcatttgagaagggtttctttatagtaatccgtgcatgccaattgttggcccaaaagaatgatgggataatattggttggagtcgcaggtccttctggggctggaaagactgtgttcacagaaaagatactcaactttatgcccagtgttgctgtcatatcaatggacaactacaatgatgccagtcggattgttgatggcaactttgatgatccacgattgacggactacgacacgttactccagaatgtaaatgacttaaaagcagggaaggaagttcaggttccagtttatgatttcaagtctagttcccgcacaggattcaggacagttgaagtcccaagctctcggattgtgatcattgaaggcatctatgctttgagtgaaaagttgcggcctttcctggatctacgagtatctgtcacaggcggtgttcactttgacctggtcaaaagggttttacgggacatacaacgagctggccaggaaccagaagaaataattcatcaaatatctgaaactgtatatccaatgtacaaggcctttattgagccagaccttcaaacagcacatataaaaattatcaacaagtttaacccatttactggatttcaaagtcccacttacattttgaagtcagcaaaggacttgtcagcggatcagattaaggctgtcttttctgaagatcacacagaagcaaaggaggagacctatgatatataccttctaccacctggtgaagatccagaatcttgccaatcatatctgaggatgcgggataaagatgggaaatatagtctaatgtttgaggaatgggtgacagataatccatttgttatatcaccaagaataacttttgaggtcagcgtgcgacttcttggtggactaatggccttggggtacacgattgcaactatccttaaaagaagcagccatgtattctccaatgatggggtgtgtgtaaaaattgattggctagagcaactaaatcgtcgatatattcaggtgcaaggaaaagatcgtgtacttgtaagatgtgttgcggagcagcttggcttggaaggttcatacattcctcgtacctatattgaacaaattcaactggaaaagcttgtaaatgaggtcatggccttgccagatgatttgaagacgaggcttagcctagatgaggatcttgtttcaagccccaaagaagcactttccagagcctccgcagatagagttgccatgagaaataagcatctcaaaagtggtatgtcgcagtcatatacaacccaaagggacaaaagtacatctaagcttacaggatatgcttccaacagccaaaggtttgatgagagaaattcagagtcatcagcaacactagcaagccagggagtagttactcagctatcagaacaaatgtcttcgctgaatgatagaatggatgagtttacaaatcgagttgaagagctaaattccaagttggctgtgaagaaaagttctcctagccaacaaaacatggctcttcaagctgaaacctgcaatggctctgttcccacttcttatttcatctctggcttaggcaatggttctttaaccgggtcaatattgccaaattcctcatcttcctcgcagctggctaaggaatcttcagtaatggaagagatgtcaagtattgcacgggggcaacgtcaaatcatgcatcagttagacaatctcagcaatcttctccgggaaaacatgggagagagaaatcgcccagtaagaactaacagcaggaaaagcaccattgctcaacctgatcagcccttgacagttcctcttgcaataacactggctgttggagttctaggactcatcatatacaagggtatctttactcgaaattga

SEQ ID NO: 16 - DNA, 1995 bp, Malus x domesetica
atggctcaagatatgtctggtgctgattcacaccaaagacggcaaggccttttaaaagatcaagttcgattggttaagagaaaggattcttctcatwacgagattgcaccaatccaaagtcctttgtcatttgagaagggtttctttatagtaatccgtgcatgccagttgttggctcaaaagaatgaaggaatagtattggtaggagttgctggtccttctggggctggaaagactatatttacagaaaagatagtcaactttatgcccagcgttgctgtcatatcaatggacaactacaatgatgccagtcggattgtcgatggcaactttgatgatccacgcttgacagactacgayacattgctccagaatgtcaatgatttaaaagcagggaagccggttgaggttccagtttatgacttcaagtctagttcccgcacaggatacaggacagttgaagtcccaagctcccgtattgtgatcattgagggcatctatgctttgagtgaaaagttgcgaccttatctggatctacgagtatcagtcacaggcggtgttcactttgaccttgtcaaaagggttttacgggacatacaaagagctggccaagaaccagaagaaataatacatcaaatatctgaaactgtatatccaatgtacaaggcatttattgagccagaccttcaaacagcacatataaaaattatcaacaaatttaacccatttactggattccagagtcccacttacattttgaagtcagcaaagaacttgtctgcggatcaaattaaggctgtgttttctgaagatcacacagaagcaaaggaggagacgtatgatatataccttctaccacctggtgaagatctcgaagcttgccaatcatatctgaggatgcgggataaagatgggaaatatagtctaatgtttgaggaatgggtgacagataatccatttgttatatcaccaagaataacttttgaggtcagcgtgcgtcttcttggtggactaatggccttgggttacaccattgcaactatccttaaaaggagcagccatgtattctccaacgatggagtgtgtgtaaaaattgattggttagagcaattaaatcgtcgatatattcaggtgcaaggaaaagatcgggtagttgtaagatgtgttgccgagcagcttggcttggaaggttcatacattcctcgtacgtacattgaacagattcaactggaaaagcttgtaaatgaggtcatggctttgccagatgatttgaagacgaggcttagcctagatgaggaccttgtttcaagccccaaagaagcactttccagagcctctgcagatagagttgccatgagaattaagaayctcaagagtggtatgtcgcagtcatatacaagccaacgggacaaaagtacatctaagcttaccgggtattcttccaacagtcaaaggtttgatgagagaaacacagagtcatcagcaacrctagaaagccagcacggagtagctactcagctttcagaacaaatgtcttcgctgaatgatagaatggatgactttacaaatcgaatcgaagagctgaattccaagctgaccatgaagaactctcctagccaacacaacatggctcttcaagctgaaaactgcaatggctccgttcccacttcttatttcatctctggcttaggcaacggttcgttaactggggcrataatgcckaattcctcctcttcctcacaggtgaataaggagtcttcagtggtggaagagatgtcaagcgttgcacgsggacaacgtcaaatcatgcatcagttagacaatctcagcaatcttctccgggatagcatgggagagagrcctcgtccggtaaaaactaacagcaggaagaacatcgtagcccaacccgaacccttgacggttcctcttgcagtaacgcttgcagttggcgttctaggggtgatcatatacaagggtatcctaactaggaactga

SEQ ID NO: 17 - DNA, 1914 bp, Sorghum bicolor
atggatcaacatgattccatggctgactcaccaaggaggcttcataatcttctgcgagacaaggtccagttggtgaaaaggaaggactcaaatcgctatgagattgtccgcttccgtgatccattgtcttttgagaaaggcttctttgttgtgatccgtgcctgccagcttttggcgcagcacaatggcgggataatatttgttggtgttgctggcccctctggtgctgggaagacggtgtttactgagaaggtggtcaatttcatgcccgatgttgctgttatatcaatggacaactataatgacgcaactcgcatagttgatggcaattttgatgatccacgtctaacagactatgacacactgctggaaaatattcatggtctgaaggaaggacgatcagttcaagttccaatatatgatttcaagtcgagctgccggattggttatagaacagttgatgtccctagctcccgaattgttatcattgaaggtatttatgcactgagtgagaagttacggcctgtgatggacttgcgtgtttctgtcactggtggtgttcattttgacctagtgaagagggttttaagggatatacaacgagctggccaggagcctgaagaaataattcatcagatctctgaaacagtttatccaatgtacaaggctttcatcgaaccagatttggagactgcacatattaaaatcgtcaacaagttcaacccattctcagggttccaaaatcctatgtacattctaaagtcaccaagatctctattacctgagaaaatcaagtccgtacttggtgatgatcatatggaaagcaacgaagaaacttatgatatatatctacttccacctggtgaagacccagaagcatgccaatcttatttgagaatgcggaatagggaaggaaaatataatctaatgtttgaggaatgggtgaccgacaattcatttatcatatcaccaaggatcacttttgaagttggtgtacgtcttcttggtggtttgatggcattgggatatactatagcagctatactgaagagaagcagtcgtgttttttctgatggcaaggcaactgttaaaattgattggctggaacaactcaaccgacactatatacaagtgcaaggaagagatcggctttatgttaaatttgtagcggatcagttaggtttggatggttcttatataccgcgcacatatattgaacaaattcagctggagaaattgattaatgatgttatggcattaccagaagatttgaagacaaagctcagcattgatgatgagctggtctcaagtccaaaggaagccttttcccgtgtttctgctgatcggagaaacaaacttatgaaaagtggcctatctcaatcatattcaacgcatagagacaaacgtgttgtgaaattgagtcaactgacagagaggaggtttggtggagggcaagcccttgaacctcctgctattgaccagggtggtatcatccagctttctgaacagatatcaacattgaatgaaaggatggatgaatttacctctcgagttgaagaactcaattcaaaattttcagtgaagaaacatttgcccagtcagcagaacttagctcttccaaatgatgcttgtagtggttccacacctacaaatctctttgtctctcagttaggcaatggtactttgatacctcattcttcatcgtcaaaccaactttcaaaggattccccaatgatagaagaggttatgaacatatcaagaggtcagcgccaggtcatacatcagctcgacaatctgaccaacctgcttcatgagcatttggtcttaacacgccaagcaaattcatgcccacttatttgcctgactgttgccagtattgggtacttcatgtttaaaggtctcaaccggagctga

SEQ ID NO: 18 - DNA, 1989 bp, Aquilegia coerulea
atggcaaaagacgcttccggtcctgactcaccccacagaaagcatggtctgttaagagatcaggttcagttggttaagaaaagggattctgatcgatacgagattgtccctatccaagatttactctcgtttgagaagggatttttcattgtaattcgtgcatgtcagttattggctcagaaaaatgagggaataatattggttggagtcgcaggtccctctggggccgggaagactgtgtttaccgagaaggtgcttaatttcatgccaagtatagcagtcatatcaatggataactacaatgacgccagtcgtattattgatggcaactttgatgacccacgtttgacagactatgatacactgctcgagaatattcatggcctaaaagaggggaagcctgttcaggtgcctgtatatgatttcaaatctagctctcgcacaggatacaggacaatcgaagtcccaagctcccgaattgtaattattgaaggcatctatgctttgagtgaaaaactgaggcctcttttagatcttcgagtatctgtcacgggtggtgttcactttgaccttgtaaaacgagtattacgcgacatacaacgtgctggccaagaacctgaagaaataatccatcagatctctgaaacggtgtatccgatgtacaaggcttttattgaaccagatctgcaaactgctcatataaaaattagaaacaagttcaatccatttgcaggattccaaaaccccacatatattttaaagtcgacgaggactgtaaccgtggatcaaatcaaggctgttatctctggggaatgtaaagatcatatggaagaaatatatgatatatttcttctaccacctggcgaggatccagaagcttgccaatcatatttgaggatgcgaaacagggatgggaaatacaatcttatgtttgaggaatgggttacagatagtccattcatgatatctccaagaatcacttttgaagtgagtgtacgtcttcttggtgggctgatggcattgggatatactatagcaactatcttgaaaagaagcagccatgtctttttcaatgatagggtgtgtgtgaaaattgattggttggaacaactgaatcgccaatatgttcaggtacaaggtagagatcgtttgtatgtgaagtttattgcggaggaactgggtttggaaggttcatatgttcctcgtacatatatccaacagattcagctggaaaagcttgtaaatgaagtaatggcactgccagatgatctaaagacaaagctcagcatagatgaggacctagtttcttcaagccccaaagaagcactttcccgtgcctcagctgataggatcgcaatgagaaataagcattccaagagtggcatgtcacaatcgtactcaacacatggcgacaagaatccttttaagcttactagactttctgtcaataatcagaagtttgatgggggaaaccttgagtcaccagcagccgtcaccaatcagggagtcattaatcaacttctggaacaaatttccacacttaatgagagaatagatgagttcacttctcggattgaagagttgaattcaaaatttaaaacgaggaatgtctctggtagccaacaaaacttgtctttgcaagctgagccatgtaatggatctggacagacatctttctttgtatctggtctagggaatggttctttaactggatcactactacccaattcgtcatcatcttctcaattagcaagggaagctccattaatggaagaggtagcagtcattgcgcggggacaacgtcagctgatgcatcaattagacaatcttagcaattttgttcatgaaaatatagaacagtcgcgtcatggaagaacagagggaagccgattaactaattttgaacacatcggaattccagttcttctgtccttggcaattggtggtgttggaatccttttgtttaagggtctgtcatcccaaaattga

SEQ ID NO: 19 - DNA, 1971 bp, Mimulus guttatus
atgtcgaaagacacttacaacgacgagtcgagtcaccgaaaaaccggtcttttgaaggatcaagttaaattggttaaaaagaaggactcgaaccggtacgagatttcccccattccgaacgaattgtcgtttgagaaaggcttctttgctacagtgcgtgcatgccagctgttgactcagaaaaacgagggtcttgtattgataggtgtagcggggccctctggtgccggaaagaccatatttaccgagaagatagtcaactttatgccgagcattgccgttatttcaatggacaattacaacgatgctagtcgaattatcgatggcaactttgacgatccaagattaacggattacgatactttgttgaaaaatattaacgacctcaaagaagggaagcaggttgatattccgatatacgacttcaaatccagctcacgcaccggatacaggacacttgaagtaccgagctcgcgaattgtgataatcgaaggaatctatgctttaagtgaaaagttacgtcctttacttgatctacgggtatccgtgacaggtggcgttcactttgatctcgttaaacgggttttacgtgacatcaatagagctgatcaagaaccagaagaaataatacatcaaatatcggaaacagtgtatccgatgtacaaggcttttatcgagcctgacctggacaccgcacatataaaaatcggcaacaaatttaacccgtttactggttttcagtcccctacttatattcttaagtcactgaagagtgtacccgtggagcaaatcaaatctgtcatgtctgaagaacacacagaatgtacagaagagacgtacgacatatttcttctaccgcccggtgaagatccagagacatgtcaatcttatcttaggatgcgtaataaagacgggaaatataatctcatgtttgaggaatgggtgaccgatactccgtttgtgatatctccgagaataactttcgaagttagtgtgcgtctactcggtggattaatggcattgggatacactatagcagccatacttaaaagaagtagccatattttcggcgacgaaaaagtctgcgtaaaaatcgactggctcgagcagttaaaccgtaattatattcaggttcaaggtagagataggcttgtagtaaaatccgttgccgagcaactgggattggacggttcatatgttccgagaacttatatcgaacaaatacagttggagaaaattgtgaatgatatgatggtaatgccagatgatttgaaaaacaagcttagcatagacgacgattttgcctcgagtccaaaggaagcgctttcccgagcctctgcattagccagaatcaagaaccttagaagtggcatgtcgcaatcgtatacaacaaaccgggacaaaaacctgtcgaagatgaacaggagattcgacgaccatacagcagattcacctgcaattttagcaaatcagggagctgtgacacagctttcggagcaaatttctacattaaacgagcgtatggatgattttacgtcacgcatcgaagagctaaattccaagttaacgagcaaaaacggttctcctaattcacaaagcataggattgcaagctgaagcctgtaacgggtcggccccaacttcttacttcatatccggattaggaaacggatccttaaccgggtcgatgctgcctaattcatcgtcttcttcccaattagcaaaggattcatctttgaccgaagagctgtcgagtatatcgcgagggcagcgccaagttatgcaccagctggacaatctgaacaatctcttacgagaaaatatgggggataaatcggttcgtgacagaagaaagaaacgaagtgggattgctgaaaacgatacgaatagaaatgcggtgattttattagcatcgttagcagttggtgggttgggaatttttttgttcaagggtgtattgtcccgaaattga

SEQ ID NO: 20 - DNA, 1962 bp, Solanum tuberosum
atgcctaaagatactagtaatggtgaatctactcagagagctggtctcttaaaggaccaggttcgactagtcaagagaaagaactgtgatcgatatgaaatcatctcgatacctgataatttgtcatttgagaaaggtttctttgttgtaatccgtgcgtgccagttgttggttcaaaagaatgaaggattgataatacttggtgttgctggtccctccggagcaggaaagactgtgtttacagagaaaatactcagcttcatgccaagtgttgcagttatatcaatggataattacaatgatgctagtcgagtagttgatggaaactttgatgatccacgacttacagactatgatacattgctgaaaaacatcaatgatctaaaggccgggaagccggctgaggttcccatatatgatttcaaatctagctctcgcataggatacaggactcttgaagtacctagctcccgcattctgattattgagggtatctatgctttgagtgaaaagttgcggccttttctggatcttcgcatatctgtgacaggtggagttcactttgatcttgtcaaaagggttttacgggatatacagcgtgccgggcaggaaccctcggagataatccatcaaatatctgaaacggtttatccaatgtacaaggcttttattgaacctgatctcaagactgcacatataaaaatcatcaacaaatttaatccgtttactggatttcagagtcctacatacattctaaagtcttctagggatgtgcaagtcgatcaaatcaagtctgcattatctgaagaacacattgaaagtacggagcaaacttacgacatataccttctgccacctggtgaagatccagagacatgccaatcatatctgaggatgagaaacaaagatggaaaatacagtctcatgtttgaggagtgggtcactgattctccctttgtcatatcgccaagaatcagttttgaagttagtgtgaagcttcttggtggattgatggccttgggatacacgattgcaacaatactcaaaagaagtagtcatgtattttctgatgaaaaggtttgcgtgaaaattgattggctagagcaactgaaccggcattatgttcaggtccaaggaagagatcgcctaattgtaaaatcggttgctgatcagctgggactggagggttcatacactccgcgtacatatattgagcaaatacaactagagaagcttgtgaatgaagttatggcactaccagatgatttgaaaacgaagcttagcttagatgaagatctcgtctccagcccaaaagaagctctctctcgagcctcagcagagagagtagcatggagaaataagaatttaagaagtggattgtcccattcctacgcaaatcacagagagaaaaatctatccaagattgatactgatagtcagaggtttgatgacagaaacactgattcagcaacattagcaaaccagggagctgttacacatttatcggaacaaatatctaccttgaatgatcggatggatgattttacatctaaaatggaagaacttaattccaagttaaccaaaaaaagagcttctccgagcacacaaagtctggctttgcaagccgaagcttgtaatggatccggcccaacttcttacttcatatctggtttagaaaatggttccttgacaggatccattatgccaaattcatcatcctcttctttgctaattccaaaggagtccaatctgatggaagagctatctaatgttgcacgtggacaacgccaaattatgcatcagttagacagccttagtaatcttcttcgcgacagactgggagaacaatcacggcaagccagaacaagcaagagaagagatcttaattcgatcagagtacctctcattgtgaccttagcagttagtggattggggttctttctgtttaagagccgaaactga

SEQ ID NO: 21 - DNA, 1992 bp, Vitis vinifera
atggctcaagatacttctggtgccgaatcacctcagccgaggccaggtctcttaaaagatcaagttcgattggttaggcgaaagaactctgatcgctatgagattgtcccaatccaagatcgtctgtcatttgagaagggttttttcatagttatccgtgcatgccaattgctagcccaaaataatgatggaataatattggtaggtttagcaggtccctctggggctgggaagactgtttttactgaaaagatactcaactttatgcccagcattgctgtcatatcaatggacaactacaatgatgctagccgaattgttgatggcaactttgatgatccccgtttgacagactatgatacattgctccagaatgtccatgatctgaaggctggaaagcaggttcagattccaatttatgatttcaagtctagctcccgcacaggatacaggacacttgaagtcccaagctcccgtattgtgatcattgagggtatccatgctttgagtgaaaagatgcgacctttattggacctccgggtatctgtaacaggtggaattcactttgatcttgtcaaacgggttttgagggacatacaacgtgctgggcaagaaccagaagaaatcattcaacaaatatctgaaacggtatatccaatgtacaaggcttttattgagccagatctcggaacagcacatataaaaatcaccaataagttcaacccattcactggatttcagagtcccacttatattttgaagtctgcgaggaatgtgacagtggatcaaatcaaggctgtcatcactgaagaacatgcagaaactatggaggaagtctatgatatttatcttctaccccctggtgaagatcctgaatcttgccaatcatatttaaggatgcggaataaagatggaaaatacagtctcatgtttgaggaatgggttactgataatccatttgttatatcaccaagaataacttttgaagtcagtgtgcggcttcttggtgggctaatggccttggggtacacaatagcaactatcctgaaaagaaacagccatgcattttctgatgacagggtgtgtgtgaaaattgattggttggaacagctaaaccgtcaatatattcaggtgcaaggaaaagaccgtttggtggtaaaatgtgttggggagcagctccagttggaaggttcatatagtcctcgtacttacatagaacaaattcagctcgaaaagcttgtaaatgaagttatggcattgccagatgatttgaagacaaagcttagtatagatgaggatatggtttccagccccaaagaggctctttctcgattctctgcagacagtgttgctatgagaaataagcacttcaagagtggtatgtcgcactcatattcaacccatcgagacaagaatctgtctaatcatactggattttccgtcagcaaccgaaagtttgatgacagacccacagaattaccagcaatgctagcaaacaagggagccattactcagctgtcagaacaaatttcatcactaaatgataggatggatgaatttacaagtcagattgaagagctgaattccaagttaaccatgaagaaagtttctgcaagccaacaaaacttggcttttcaggctgaagcaaacaatggctctgctcctactttcttcatccctggcataggcaatggttccttgactggaaccatgatgcacaaatcctcatcttcctcccagttggctaaggagcttcctttaatggaagagatactaggaattgcacggagtcaacggcaagtgatgcatcagttggacaatattagcagtcttgtgcgggaggacataggaggagggagatcagagggaggaagaagagagaggaaaagcataatgcttgacattgaacccactagagttcctctgatattggcactggcagttggtggcttgggtatattcttgtttaaagcatttctacccagaaactga

SEQ ID NO: 22 - DNA, 1986 bp, Vitus vinifera
atggctcaggatacatcttctggtgctgattcacctaggcggaagtctggtctattaagggatcaagttcaattggtcaaaaggaaggattctactcgttacgagatagttccaatccaaaattctctgtcatttgaaaaaggtttctttatagtgatacgtgcatgccagttgctggctcaaaacaatgatgggataatattagtaggagtagcaggtccctctggggctgggaagactgtgcttactgagaaggtgctcaactttatgcccagcattgctgtcatttccatggacaactacaatgattccagccgtatcattgatggcaactttgatgacccacgcttgacagattatggcacattgcttgagaatatccatggtttaaaagcagggaagcccgttcaagttcctatttacgattttaaatctagctctcgcataggttacaggacagttgatgttcccagttcccgcattgtcattattgaaggcatatatgccttgagtgaaagattgcgccctttgctagatcttcgagtatctgttactggtggagtacactttgaccttgtcaaaagggttttacgggacattcaacgtgctggccaagagcctgaagaaataatccatcaaatctctgaaacggtatatcctatgtacaaggcttttattgagccagatctacaaacagcacatataaaaatcatcaacaaattcaatccattcactggatttcagaaccccacttatattttgaagtcaacgagggatctttcagtggatcaaatcaaagctgttctttctgaagaacacactgaaactacagaggaaacttatgatatatatcttctaccgcctggtgaagatcctgaagcatgccaatcatatctgaggatgaggaacagggatggcaaatacaatctcatgtttgaggaatgggttacagatagtccgttcatcatttcaccaagaataacttttgaagttagtgtccgccttcttggtggcctgatggccttggggtacacaattgcagctatcctgaaaagaaccagccattgcttctttgatgataaagtgtgtattaaaactgattggctagaacaacttaatcgtcggtatgttcaggtgcaaggaaaagaccgtttgtatgttaaaaatgtagcagagcaactgggtttggatggttcatatgttcctcgcacatatatagaacacattcagctggagaagcttgtgaatgatgttatggcgttaccagatgatttgaagacaaagctcagtattgatgatgatttggcttcaagccctaaagaagcactatcccgggcctctgcagataggagaatgaaatatctcaaccgtgatataccacattcatattcaacacaaagggataagaatttgcctaagctgaccagacttgcagttaacagtagaaggtttgatgtaagaaccccagattctcctgcaacagttccaaatcagggagtcatcactcagctctcagaacaaatttccacactgaatgagagaatggatgagttcacatctcgcattgaagagttaaattccaagttctctatcaggaaagtttctgctagccaacaaaacttggctgtgcaggctgatgcctgcaatggctctgctcccacttctcttttcatggctggcttaggaaatggttctttgactgggtctatactgcctaattcttcctcttcttctcaattggctaaagactcccctctgatggatgagatattagttgttgtgcgtgggcaacgtcagataatgcatcaattagacaatctgaacagtcttctgcatgagtactggggagagaggtcacgagaaggaagaacagatagggcaaacaggatgattgacattgaatccatgggcattccaattatcttatccctggcaattggtggtttaggtgtcttctttttcaaaagtttgtcttcccaaaagtga

SEQ ID NO: 23 - DNA, 1989 bp, Eucalyptus grandis
atgactcaagatggttctagtgttgaatcaaaccaacgaaagccaggactattgaaagatcaggtcagattagtgaagaggaaggacagtggtcgccatgagatagtttcaatccaagatcctttgtcatttgagaaagggttcttcatagtaattcgtgcatgccaattactggctcagaagaatgaaggaatcatattggtgggcttggctggtccctctggtgctggaaagactgtatttacagaaaagattgtgaactttatgcctagcatatctgttatatcattggacaactacaatgactctagtcgaattgttgatggcaactttgatgatccacgcataacagactatgagactctgctcaggaatgtcgaggagttaaaagcagggaaaccagtccaagtacctatttatgacttcaagactagctcccgcacaggatacaggactcttgaagttccaagttccagaattgttattattgagggaatctatgcactgaatgaaaaattgagacctttgctggatcttcgcatatcagtcactggtggagttcattttgatcttgtcaaacgggttttaagggatatacagcgggctggtcaagaaccagaagagataatccaccaaatatctgaaacggtatatccaatgtacaaagccttcattgagcctgaccttcaaacagcacatattaaaataatcaacaagttcaacccatttacgggattccagaatcccacttacatattgaagtcatcgaggaatgtagcggtcgatcaaatcaaggctgtattttctgaggaacatgttgaaacttccgagcagacatacgatatctatcttctacccccaggggaagatccagaatcttgccaatcgtatttgaggatgcggaataaagatggaaagtacagtcttatgtttgaggaatgggtgactgacaacccttttgtgatatcacctagaataacgtttgaagtgagtgtccgcctcctaggtgggttgatggccttgggatacacaatagcaacaattctcaagagaagcagccacgtgttctccgatgatagggtgactgtgaagattgattggctagaacaattgaaccgtcagtatcttcaggtgcaaggaagagatcgcctggttgtgcaaggtgttgccgagcagcttgggttggaaggctcatatgttgcccgcacgtatattgaacaaatacaactggaaaagcttgttaatgaggtcatggcactgccagaagacttaaagactaaacttagcctagacgaggatcttgttgcaagtccaaaagaagccctttctcgagcatctgctgatagggtagctatgagaagtaagcatttgaagagtggtatgtcacactcatattcaacccaaagggacaagaatctgtcaaaggttgctggctttgctttcagcaatccgagatatgatgatagaatctcgggtgcatcgttaacacttcctaaccagggtgtcatttctcagctttcagatcaaatttcctccctgcatgacagaatggatgattttaccgcccgtattgaagagttgaattccaagttaacaataaagaagaattccttgagccaacaaaatatggcccttaatcctgaggcttgcaatggatcagctccaacttcatattttatttctggtttgggaaatggttctttgactggatccataatgcccaattcctcgtcatcctcccaacttgctagggagtctccgttgatggaagagatatcaacagttgcacgtgggcagagacaaatcatgcatcagcttgacaacctcagtaatctactccgggagacattaggggagagatcccgccatctaaccacaagcaggaaaagtgtgcttgacccagaccgtgtgaaaattcctcttatttgcaccctggctttcggcggtctaggaatcctcgtgttcaagggtctttcaaacagaaattga

SEQ ID NO: 24 - DNA, 1866 bp, Citrus sinensis
atggctcaagatagttctaatgctgaatcacaccacaaaaaatcagtcctcttgaaagaccaagtccggttggttaagagaaaggactctgatcggcatgagatagttcccatccaagatcctttgtcatttgagaagggtttctttgtagttatccgtgcatgtcaattgctagcacagaagaatgatggcataatattgataggtttagcaggtccttctggggctggtaagaccattttcactgaaaagattctcaactttatgcccagcattgctgtcatatctatggacaactacaacgattccagtcgagttgttgatggcaactttgatgatccacgcttaacagactatgacacattactcgaaaatgtccgtgatttaagagaaggaaagccagtacaggttccaatctacgatttcgagtctagctcccgcacaggatacaggaccgttgaagtcccaagctctcgtattgtgattattgagggcatctatgctttgagtgagaagctgcgaccattattggatctgcgcgtatctgtcacagggggagttcattttgacctagttaaacgggttttacgtgacatacaacgtgttggtcaagaaccagaagaaataatccaacaaatatcggaaacgtcagcaaagaatttgtcagtggatcagattaaggctgtttatcctgaaggacatacagaaacaatggagcagacttatgatatttatctgctaccacctggtgaagatccagaatcttgccaatcatatctgaggatgcggaataaagatggaaaatacagtctcatgtttgaggagtgggtgacagatattccatttgtgatatctccaagaattacttttgaagtcagtgtgcgccttcttggtgggctaatggccctggggtacacgattgcaaccatacttaaaagaagcagccatgtattctgtgatgataggggggtgtgcgtgaaaattgattggctagaacaactaaatcgccaatatattcaggtgcaaggaaaagatcgcttgattgtaaaaaatgttgcagagcagctgggtttggaaggttcgtacgttccacgcacctatatagaacagattcaactggaaaaactagtcaatgaggttatggccttgccagaagatttaaagacaaagctcagcctagatgaggatctcgtatcgagccctaaagaagcactttcacgagcctctgctagcagggttgccatgagaaataagaatctccgaagtggtatgtcacattcgtattcaactcagggggacaaaaatctgtctaagcttactggatatgttatgaatgaccgaaggtttgatggtagaagtgtggagtcgtcagcaacactagccagtcatggtgtcatcactcacctttcagaacagatctcctcacttactgatagaatggatgattttacaactcatattgaggagctaaatgcgaagttgaccaagaaaaattcttcaagtcaacaaaatatggctcttccagctgaagcatgcaatggctctgctccgacttctttcttcatctcaagtctaggcaatgggtccttaatgcctaattcctcatcttcatcccagttggctaaggagtcacctttaatggatgagatatcagggattgtgcgaggacaacgtcaagtcatgcatcagttggacaatcttagcaatcttcttcatgagagcatgggggagaggtctcaccagggaagaaagaccaagaaaagtaattggccagatgccgaaccctttaaagttcctctaattataaccttggcagctggtggcgtaggaatcttcttgttcaagaaattcttaacccgaaattga

SEQ ID NO: 25 - DNA, 1977 bp, Theobroma cacao
atggctcaagatgcttctagttttgaatcacaccataagaaaccaggtctcttaaaagatcaagtacggctggtgaagcgaaagggctgtgatcgccatgagattgttccaatccaagatcctttgtcttttgagaaggggttctttattgttatccgtgcatgccaactgttagcacagaaaaatgatggaataatattagtaggattagcaggtccttctggagcagggaagactgtatttacagaaaagatactcaacttcatgcccagcattgctattatatcaatggacaattataatgattctagtcgaatagttgatggaaactttgatgatccacgcttgacagattatgatacattgctccagaatgttcatgatttaaagcaagggaaggaagtccaggttccaatctatgatttcaagactagttcccgaacaggatacaggacccttgaagttccaagctcccgcattgtaattattgagggcatttatgctttgagtgaaaagttacgacctatgatggatcttcgagtatctgttacagggggagttcactttgaccttgttaaacgagtcttacgagacatacagcgtgctggccaagaacctgaagaaataatccatcaaatatctgaaactgtatatccaatgtacaaggcctttattgagccagatctccaaacagcacatataaaaatcattaacaagttcaatccatttacaggatttcaaagtcccacttatattttaaagtcagcaaggaagtttacagtggatcaaattaagagtgttgtatctgaagaacatgcagaaacagaggagcagacttatgatatatatcttctacctcctggtgaagatccagaatcttgccaatcatatctgcggatgcggaataaagatggaaaatacagtctcatgtttgaggaatgggtgacggatagtccatttgtcatatctccaaggattacatttgaagttagtgtgcgtcttcttggtggactaatggccttgggttatacaatagctgccatccttaaaagaaacagccatgtattctctgatgatagagtttgtgtgaaaattgattggctagaacagctaaatcgtcaatatcttcaggtgcaaggaagagatcgcttagttgtaaaatgtgttgcagagcagcttggtttggaaggttcatacattccacgtacctatattgaacaaatccaattggaaaagcttgtaaatgaggttatggccctgccggaggatttgaagacaaagctcagcctagatgaggatctggtgtcaagcccaaaagaagctctcttaagagcctctgcagatagggttgccttgagaaataagcacctcaaaagtggtatgtcacattcatattcaactcaaagggagaagaatataccccactttgctggatatagtgtgaacaaccggaggtttgatgagagaaattcagactcagctctagcacaccagggagtcattactcagctttcagatcagatatcctctctgaatgataggatggatgagtttacaacccgggtcgaagagttgaattccaagttaaccatcaagagaagtagttccagccaacaaaacttggcttttcaagcggatccttgcaatggctctgctccaacttcttacttcatcaatggtttaggcaatggttccataatgcccaattcctcatcttcctcccagctggctaaggattcacctttaatcgaagagatatccattgttgcaaggggacaacgccaaatcatgcaccagttagacaaccttagtaatctccttcatgagagacttggagagaggtctcagcaagcaagtacaagaaggaaaaacatggtggctgatgcagagcctatcaaagttcctctagttctaacaacattggtaattggcggtctaggaatcttcttatacaggggctttttaacccgagactga

SEQ ID NO: 26 - DNA, 1989 bp, Gossypium raimondii
atgccagaggtagctgtcatattttcaatggctcaagacgcttctggtattgaaacatgccataagaaaccaggtcttttaaaagatcaagttccactggttaagcgaaaggatattgatcgccatgagattgtttcaatcgaagatcctttgtcttttgagcaggggttctttattgttatccgtgcatgccaatcattagcacaaaaaaatgatggaatagtattggtaggattagcaggtccttctggggcagggaagactgtatttaccgaaaagatgctcaacttcatgcccaacattgctattataacaatggacaactataatgattctagtcggatagttgatggcaactttgatgatccacgcttgacagattatgatatgctgctccagaatgttcatgatttaaaggaagggaaggatgtccaggttcccctctatgatttcaagactagttcccgaacaggatacaggactcttgaagttccaatctcccgaattgtaattattgaaggcatctatgctttgagtgaaaagttgagacctatgctggatcttcgagtatctgtcactggaggagttcactttgacctcgttaaacgagttctacgagacatacagcgtgctggccaagaacctgaagaaataatccaccaaatttctgaaactgtatatccaatgtacaaggcctttattgagccggatctccaaacagcacatataaaaatcattaacaagttcaacccgtttaccggatttcaaagccccacttatattttaaagtcagcaaggaagttcacagttgatcaaattaagagtgttgtatccaaagcacatatggaaacagaggagcagacttatgatatataccttctacctcctggtgaagatccagattcttgccaatcatatctgcggatgcagaataaagatggaaaatacagtctcatgtttgaggaatgggtaacagatattccatttgtcatatctccaagaattacatttgaagttagtgttcgccttcttggtgggctaatggccttgggttatacaatatcagccatccttaaaagaaacagccatgtattctccgatgataatgtttgcgtgaaaatcgattggctggagcagctaaatcgtcaatatcttcaggtacaaggaagagataggtcagttgtaaaagatgttgcagagaggcttggtttggaaggttcttacattccacgtacctatattgaacaaattcgattggaaaagcttgtaaatgaggttatggctctgcctgaggatttgaaaacaaagctcagcctagatgaggatttggtgtcaagccccaaagaagcactcttgggagcctccatggatatggttgctttgagaaatatgcatctcaggagagagaagtatatatcaaactttgctggatatagtgtgaacaaccagaggtttggtgagagaaactcggagtcagctctagcaaacaagggagtcattaatcagctttcggagcagatatcctctctaaatgatagaatggatgaatttaaaacccgtgtcgaagagttgaattccaagttaaccatcaagaggaggacttccagccaacaaaacctggcttttcgagctgaatcttgcaagggatctgcttgtacttcttactttataaatggtttaggcaatggttccataataaccaattcttcatcttcctcccaactggctaaggattcacctttaatggaagagatatctactgttgcagagggacaacgtcgaatcatgcatcagttagacagccttagtaatctcctccatgagagacttggagagaggtctgagcaagcaaatacaaaaaggaaatacatggtggctggtgcagaacctattaaagttcctctaattttaacaacattgaccattggtggtctgggaatctttctatttaggggctttctttgtaactcttcctctcaggttttctag

SEQ ID NO: 27 - DNA, 1977 bp, Gossypium raimondii
atgcctcaagatgcttctggtcttgaagcacagcagaaaagagcaggtctcttaaaagatcaggttcggctggttaagcgaaaggattgtgatcgccatgagattgttccaatccaaggacctttgtcctttgagaagggtttttttattgtcatccgtgcgtgccaattgttggcacagaaaaacgatgggataatattggtaggattagcgggtccttctggagctgggaagactgtatttacagaaaagatactcaacttcatgcccagcattgctaccatatcaatggacaactataatgattctagtagaattgttgatggcaactttgatgatcctcgattgacagattatgatactttgctccagaaccttcatgatttaaaggaagggaaagaagttcaggtccccatatatgatttcaaggctagttcccgaataggatacaggacgcttgaagttccaagctcccgcattgtaattattgagggcatctatgccttgagtgaaaaattgcgacccatgcttgatctacgagtatctgtcacagggggagttcactttgaccttgttaaacgagtcctacgagacatacagcgtgctggccaagaacctgaagaaataatccatcaaatatctgaaactgtatatccaatgtacaaggccttcattgagccagatctccaaacggcacaaataaaaatcattaacaagttcaacccatttactggatttcagagtcccacttatattttaaagtcagcaagagagttaacagttgagcaaattaagagtgccatatctgatgaacatatagaaacacaggagcagacttatgatatataccttctacctcctggtgaagatcctgagtcttgccaatcatatctgcggatgcggaataaagatggaaaatacagtctcatgtttgaggaatgggtaacggatattccatttgttatttctccaagaattacatttgaagtcagtgtgcgccttcttggtgggctaatggccttgggttatacaatagcaaccatcctcaaaagaaacagccatgtcttctctgatgataaagtttgtgttaaaattgattggctagagcagctaaatcgtcaatactttcaggtgcaaggaagggaccggtcagttgtaaaatgtgttgcagagaagcttggtttggaaggttcatatatttcacgtacctatattgaacaaattcaattggaaaagcttgtaaatgaggttatggccctgccagaggatttgaagacaaagctcagcctagatgaggatctggtgtcaagccccaaagatgcactcttgcgggcctctgtggatagggttgccttgagaaataggcatctcaaaagtggcatttcacattcatattctactcaaagggagaagaatatgtcgaactttgctggatataatgtgaacaaccggcggtttggtgagagaaattcagagtcggctctagcaaatgagggagtcataactcagctttcagaacagatatcctctctgaatgatagaatggatgagtttacgacccgtattgaagagttgaattccaagttaaccatcaagagatatacttctagccaacaaaacttggctttccaagctgaatcatgcaatggctctgctccaacttctcacttcatcaatggtttaggaaatggttccataatgcccaattcctcatcatcctcccagctggctaaggattcacctataatggaagagatatccagtgtggcacgcggacaacggcaaatcatgcatcagttagacaaccttagtaatctcctgcgtgagggaattggagaaaggtcacaagcagctagtacaagaaagaaaaacatgatggctggtggtgaagattctataaaagttcctgtaatattaacattggcaattggcggtctgggaatcttcttatataggggcattttaacccgacactga

SEQ ID NO: 28 - DNA, 1992 bp, Carica papaya
atggcacaagatacgaacggtgctgacttgcatcagaagaagccagttctactaaaagatcaggttcgtttggtcaagagaaaggactctgatcgatatgaggttgttccaatccaagatcctttatcatttgaaaagggtttcttcgtagtcatccgtgcatgccaattgttagctcagaagaatgatggaataatactgataggcttggcaggcccttccggtgctgggaagaccgtattttctgaaaagatactcaatttcatgcccagtataggtttaatttcaatggacaactacaatgattctagtcgaattgtggatggcaattttgacgacccacgcttgacagactatgatacgttactgaacaacctgcgtgatataaagtctggaaagcaggccgagattcctatatacgatttcaagtccagctctcgcataggatacaggacacttgaagttccaagttctcgaattgtcattattgagggcatctatgccttgagtgaaaagttgcgacctcttttggatcttcgagtatctgttacaggaggagtccattttgatcttgtcaaacgggttttacgggatatacagcgtgctggacaagaaccagaagagataatccatcaaatatctgaaacagtatacccaatgtataaagccttcatagagccagatctcaagacagcacagataaaaataatcaacaaatttaatcctttcactggttttcagagtcccacttatatcctaaagtcggcacgaaatgtgtcagtggatcaaatcaaggctgttttctgtgaagaacattccgaaacagatgagcagacttatgatatttatcttcttcctcctggtgaagatccagaatcatgccaatcatatttgaggatgcggaataaagaagggaaatacagtctcatgtttgaggaatgggtgacagataatccttttgtcatatcgccaagaattacttttgaagtcagtgtgcgccttcttggggggctaatggctttgggatacactatagcaaccattcttaaaagaaacagtcatgtttgctcagatgatagggtgtgtgtaaaaattgattggcttgaacaattgaatcggcaatatattcaggtccaaggaaaagacaggtcagttgtacaacatgttgcagagcagctggctctagaaggatcatacattccacgaacttatattgagcagatccaactagagaagctcgtaaatgaagttatggccttgccagatgatttgaagacaaagctcagtctggatgaagatctcgtgtcaagccccaaagaagcactttctcgagcttctgctaggagagttgctttgagaaataagaatctcagaagtggtatgtcacattcatattcaactgaaagggataataatctgtcaaagcctactggatttgcattgaaaaatcggagatttgatgagagtaatccggagtctcaaacaaatgtagcaaaccagggagctatcactcaactttctgaacaaatttcctcactaaatgataggatggatgagtttacgactcggatggaagagctaaattcaaaattaagcttcaagaaaaattctcccagtcaacagaatattgccctccaagccgaagcctgcaatggctctgctcccacttcttacttcatctctggtttaggcaatggttcactgactggatcaatcatgcctaactcttcctcatcatcccagttggttaaggaatcaccgttgatggaggagatatcaggaattgcgagaggacaacgtcaaatcatgcatcagttggacaacctgagcaattttcttcgcgaaagcatgggacagaagtctcgacaagaaagacagggcaggaaaagcataatttctgatgttgaacccattaaagttcctctgatgataacactggccataggtggtataagcatagtcttattcaggggctttttaacccgaagttga

SEQ ID NO: 29 - DNA, 2025 bp, Thellungiella halophila
atgggtcaagacaccaatgggatcgagtttcatcagaagagacatggtttgttgaaggatcaagtccaattggttaagagaagggactcggttcggtacgaaatagttccaattcaagatcggttgtcatttgagaagggcttctttgctgtcatccgtgcttgccaattgctttctcagaagaatgatgggataatattggttggtgttgctggtccttctggtgccggaaagacggtttttactgagaagatactcaactttctgcctagcgttgctgtcatatcaatggacaactataatgacgccagtcggattgttgatggaaactttgatgatccacggttaacggattatgacacattgctcaagaatcttgaagatttaaaggaagggaagcaggttgaagttcctatttatgattttaagtccagctctcgtgttggatacaggacccttgatgtcccagcttctaggattgtgattattgaaggaatctatgctttgagtgaaagattgcgacccttattggatcttcgtgtgtctgttactggtggagttcactttgaccttgttaaacgggttcttcgtgatatacaacgtgcaggccaacagccagaggagataatccatcagatttctgaaacagtgtatccgatgtacaaagctttcatagagccagatctccagactgctcagattaaaatcattaataaattcaatcccttcactggttttcagagcccaacatacatcttgaagtcaagaaaggatgtatccgttgatcagatcaaggcggtcctttctgaaggacatacagagaccaaggaggagacctatgatatctatctccttcctccgggtgaagatccagagtcgtgccaatcatatttgaggatgcggaataaagatggaaagtacagcctcatgtttgaggaatgggttacggatactccttttgtcatatccccaaggattacttttgaagtgagtgttcgcttacttggtgggcttatggcattgggatacacaatcgcaaccatacttaaaaggaacagccatgtatttgctactgataaggtgtttgtgaagatagattggcttgagcaactgaatcgtcactacatgcaggtgcaaggtaaagatcggcaacttgtacagagtactgcagagcagctaggattggaaggatcgttcattccacgaacctatattgaacagatccaactggaaaaactgataaatgaagtaatggcgctaccagaagatttgaagaacaagcttagcttagatgaggatttggtgtccagttcaagcccaaaggaagcactcttacgagcgtccgcagatagagtagccatgagaaacaagaacctcaaaagaggcatgtcacactcgtattcaacacaaagagataagaatttgtccaagcttgctgattattcttcaagcgataggaggtacgaagaaagaaatcatgactcaccagcaaacgaggggtttatgactcagctttcggaacaaatatcgtctctcaatgagagaatggatgagttcacaagccggatcgaagagctaaattcaaagttgagctgcaacaaaaactctccaacacagcagagcatgacactccaagctgaagtctgcaatgggtcagctcctacttcgtatttcatttctggtctggacaatggctgtttgacgaattccataatgccccattcatcatcatcctcccaattagccaaggattcgccattaatggaagagatatcgaccatttcacgtggacagcgtcaagtgatgcatcagttggataatttgtgcaatctgatgagagaaagctcagcagaaaggacacgcctagcaagagcagggagcagcaatagcagcagcagaggcagatcaagcaaaagcttctttttgtccaatgcggaatctaagagcatccctctcgtgttaaccttggctttttgcagcataggcattgtagtgatcaagagctacattaacaaacgtcaataa

SEQ ID NO: 30 - DNA, 2028 bp, Brassica rapa
atgggtcaagacaccaatgggatcgagtttcatcagaagaggcacggtcttttgaaggatcaagtccaattggttaagagaagagactctgttcggtacgagatagttccgattcaggatcggttgtcgtttgagaagggcttctttgctgttatccgtgcttgccagttgctttctcagaagaacgatgggatcatattggttggggttgctggtccttctggtgcggggaagacggtgtttaccgagaagatactcaactttctgcctagtgttgctgtcatatcgatggataactataatgacgctagtcggattgttgatggcaactttgacgatccacggttaacggactatgacacattgctcaagaatcttgaagatttgaaggagggaaaacaagttgaggttcctatatatgattttaagtccagctctcgtgttggatataggacacttgatgtcccagcttctaggattgtgatcattgaaggaatctatgctttgagtgaaaaattgagacctttgttggatcttcgtgtgtctgttactggtggagttcactttgatcttgttaaaagggttcttcgtgatatacaacgtgcaggccaacagcctgaggagatcatccatcagatatctgaaacagtgtatccgatgtacaaagctttcatagaaccagatcttcagactgctcagattaaaatcattaataaattcaaccccttcactggttttcagagcccaacatacatcttgaagtcaagaaaagatgtatctgttgatcagatcaaggctgttctttctgaaggatacacagagaataaggaggaaacctatgacatatatcttcttccaccgggtgaagatccagagtcgtgccaatcacatttgaggatgcggaataaagatggaaagtacagcctcatgtttgaggaatgggtcacggatactccttttgtcatatccccaaggattacttttgaagtgagcgttcgcttacttggtgggctcatggcactgggatacacaatagccaccatacttaaaaggaacagccatgtatttgctaccgaaaaggtttgtgtgaaaatcgattggcttgagcagctgaatcgtcactacatgcaggtgcaaggtaaagataggcaacttgtacagagcactgcagagcagctaggactggaaggatcattcattccgcgcacctatattgaacagatccaacttgaaaaactgataaatgaagtaatggctctaccagatgatttgaagcacaagcttagcttagatgaggatttggtgtctagttcaagcccaaaggaagcgctcttgagagcctctgcagatagagtagccatgagaaataagaacctcaaaagaggcatgtcacactcgtattcaacccaaagagataagaatctttccaagcttgctggttattcttcaagcgataggaggtacgaagagagaaaccatgactctccagccaacgaggggtttatgactcagctttcggaacaaatatcatcactcaatgagagaatggatgagttcacaaaccggatcgaagagctaaactcaaagctgagctgcaacaagaactctccaacacagcagagcatgacagtccaagccgaagtttgcaatggctcagctccaacttcgtatttcatttcaagtctggacaatggctgtttgacaaattccataatgccacattcatcttcatcttcccagttagccaaggattccccacttatggaagagatatcaaccctctcacgtgggcagcgtcaagtgatgcatcaactggataacctgtgcactctgatgagagaaagctcatcagcagaaaggtcacggctagcaagaacaggaagcaacaagagaagcggatcaagcaaaagcttcttcttgtcaaatgcggaatctaatagcctcctccctctcaagttaacagccttggcggctctttgcagcgtagggattgtagtgatcaagagttacattaacaagcggcaataa

SEQ ID NO: 31 - DNA, 1908 bp, Brassica rapa
atgacgggtcaagacatcaatgggatcgagtttcatcagcagagacacggtcttttgaaggaccaagtccaactggttaagagaagagactcggttcggtacgagatagttcctattcaagatcggttatcgtttgagaagggcttctttgctgttatccgtgcgtgccagttgctctctcagaagaacgatgggatcgtattggttggtgtggctggtccatctggcgctggaaagactgtgttcaccgagaagatactcaactttttgcctagtgttgctgtcatatcgatggataactataatgacgctagtagaattgttgatgggaactttgacgatccacggttaacggactatgacacattgctcaagaatcttgaggatttgaaggaagggaaacaagttgaggttcctgtatatgattttaagtccagctctcgtgttggatacaggacacttgatgttccagcttctaggattgtgattattgaaggaatctatgcgttgagtgaaaaattgagacctttgttggaccttcgtgtgtctgttactggtggagttcactttgatcttgtgaaacgggttcttcgtgatatacagcgtgcagggcaacagccggaggagatcatccatcagatatctgaaacggtgtatccgatgtacaaagctttcattgagccagatctccagactgctcaaatcaaaatcattaataagttcaatcccttcactggttttcagagcccaacatacatcttgaagtcaagaaaggatgtatctgttgatcagatcaaggcggtcctttctgaaggacatacagagaccaaggaggagacctatgatatatatcttcttcctcctggtgaagatccagagtcttgccaatcgtatttgagaatgcggaataaagatggaaagtacagcctcatgtttgaggaatgggttacggatactccttttgtcatatccccaaggattacttttgaagtgagcgttcgtttgcttggtgggcttatggcattgggatacacaatagcaaccatacttaaaaggaacagccatgtatttgctaccgataaggtgattgtcaaaatcgattggcttgagcaactgaatcgtcactacttgcaggtgcaaggtaaagatcggcaagttgtacagagcactgcagagcagctaggattggaaggatcgttcattccacgcacctatattgaacagatccaacttgagaagctgataaatgaagtaatggcactaccagatgatttgaagaacaagcttagcttagatgaggatttggtgtctagttcaagtcccaaggaagcactcttacgagcgtctgcagatagagtatccatgagaaataagaacctaagaggcatgtcacagtcatattcaacccaaagagataagaatatctccaagcttgctggttattcttcaagcgataggaggcacgaagagagaaaccacgactcaccagcgaacgaggggtttatgactcagctttcggaacaaatatcatctctcaatgagagaatggatgagttcacaaaccttatcgaagagctgaattcaaagttgagctgcaacaagaaccctccaacacagcagagcatagaagtctgcaatggctcagctccaacttcgtatttcatatctggtctggacaatggctgtttgacaaatgccataatgcctcattcatcttcatcttcccaggaagactcagccgaaaggtcacgcctagcaagaacaggaagcagcaatagcagcagatcaagcaaaagcttcttcttatccagtgtggaatctagtagcctccctctcgtgttaacaaccttggctctttgcagcgtaggagtagtagtgatcaagagctacattaacaaccggcaataa

SEQ ID NO: 32 - DNA, 2024 bp, Capsella rubella
atgggtcaagacagcaatggtatcgagtttcatcagaagagacatggtctcttgaaggatcaagttcaactggttaagaggagggattctgttcggtatgaaatagttcctattcaagatcgcttgtcatttgagaagggcttctttgcggttatacgtgcgtgccaattgctttctcagaagaatgatgggattatcttggttggggttgctggaccttctggtgccggaaagactgtctttaccgagaagatactcaactttctgccaagtgttgctgtcatatcaatggacaattacaatgattctagtcggattgttgataaaaacttcgatgatcctcggttaacggactatgatacattgctcaagaatcttgaagatttaaaggaaggaaagcaggttgaggttcccatttatgattttaagtccagctctcgtgttggatacaggacccttgatgtcccagcttcacgtattgtgattattgaaggaatctatgctttgagtgaaaaactgcgacctttattggatcttcgtgtgtctgttactggcggagttcatttcgaccttgttaaacgggttctccgtgatatacaacgtgcaggccaacagccagaggagattattcatcagatatctgaaacagtatacccgatgtataaggctttcatagagccagatctccagactgctcaaattaaaatcatcaataagtttaaccctttcactggtttccagagcccgacttacatcttgaagtcaagaaaggatgtatctattgatcagatcaaggaggtcctttctgatggacatacggagactaaggaggagacctatgatatatatcttctccctccaggtgaagatccagagtcttgccaatcatatttgaggatgcggaataaagatggaaagtacagccttatgtttgaggaatgggttacagatactccttttgtcatatccccaaggattacatttgaagtcagtgttcgtctacttggcgggcttatggcattgggatacacaatagcaactatacttaaaagaaacagccatgtatttgctactgataaggtgtttgtgaaaatcgattggcttgagcaactgaatcgtcactacatgcaggtgcaaggtaaagatcggcaacttgtacagagtactgcacagcagctaggattggaaggatcgttcattccacgcacctatattgaacagatccaactggaaaaattaataaatgaagtaatggccctaccagatgatctaaagcacaagcttagcttagatgaggatttggtgtctagttcaagcccaaaggaagcactcttgcgagcctctgcagatagagtagccatgagaaataagaacctcaaaagaggcatgtcacactcatattcaacccaaagagataagaatatgtccaagcttgctggttattcttcaagcgataggaggtacgaagaaagaaatcacgactcgccagcgaacgaggggtttatgactcagctttctgaacaaatatcatctctcaacgagagaatggatgagttcacaagtcggatcgaagagcttaattcaaagttgagctgcaacaaaaactctccaacacagcagagcttgtcaatccaaactgaagtctgcaatggctcagctcctacttcgtatttcatttctggtctggacaatggctgcttgacaaattccataatgccccattcatcatcatcctctcagttagccaaggaatcaccattaatggaagagatatcgaccatatcacgtggacagcgccaagtgatgcatcagttggataatttgtgcaatctgatgagggaaagctcagcggaaaggtcacgcctagcaagaacagggagcagcaatagcagtaacagaggagatcaagcaaaagcttcttcttgtccaatgtggaatctaagaggctccctctcgtattaaccttggctttttgcagcgtaggctttatagtgatcaagagctacattaacaagcggcaataa

SEQ ID NO: 33 - DNA, 1995 bp, Glycine max
atggcaaaagattctgccgatgctgattcccatcacaggcgtcttggtcttttgaaagatcaagttcacttggttaaaaggaaaggctctgatcgctatgagattgctccaatccaagaccaactatcatttgagaaggggtttttcatagtgatccgtgcatgccaattgttatcccaaaagaatgatggaataatattggtaggggtggcaggtccttcaggggctgggaagactatatttactgaaaagatactcaacttcatgcccagcattgcaatcatttcaatggataattacaatgatgctagtcgaattgttgatgggaattttgatgatccacgcttaacagactatgacacattactccagaatctgcacaatttaaaggaaggaaagccagttcaagttcccatatatgatttcaagtccagttcacgtacaggatacaggacagtcgaagccccgagttctcgaattgtgattatagagggcatctatgccttgagtgagaagttgcgacccctgatggaccttcgagtctctgttacaggaggagttcaccttgaccttgtaaaacgagttatacgtgatatacaacgtgctggtcaagaacctgaagaaattattcatcaaatatcagagacggtgtatccaatgtacaaggcatttatcgagccagatctccagacagcacatataaaaatcatcaacaaattcaatccatttactggattccagagtcctacttatatattaaagtcaggaaggaatgtagaagttgataaaattaaagctgttctcgctgaggattttaaggaaacaacagaacaaacttatgacatatatcttctaccacccggtgaagatccagaaacttgtcaatcatatttacggatgcgaaataaggatgggaaatacagtctcatgtttgaggaatgggtgacagataatccatttgtcatatcaccaagaattacttttgaggtcagtgtgcgccttcttggtggactaatggctttgggatacacaatagccactatccttaagagaaacagccatgtcttttctgatgatagggtttgtgtaaaactagattggcttgagcaacttaatagacattatgttcaggtacaaggcagagatcgtttagttctaaaatacataggagaacagctgggtatagaaggttcctacattcctcgtacctacattgagcaaattcaaattgaaaagcttgtgaatgaggttatggctctaccagatgatttgaagacaaggctcagcctagatgaggatttggtgtcaagcccgaaagaagcactatctagagcttctgccggcagagttgccatgaggaataaacatctgaggagtggaatatcacagtcgtataccaatcagagggacaaaaatctagctaaggttactggatatggtgccaacaatagaaggtttggtgaaagcaattcagattccaccgcaatgccagtaaatcagggaaccatcaatcagctttcagaccaaatttctgccctcaatgatagaatggacgagtttacaaatcgcattgaagagctgaattccaagttaaccatcaagaaaaattctccaagccaacaaaacatgtcacttcaagctgagacatgcaatggttctgctcccacctcttacttcatcactagtttgggcagtggttcccttactggatctaaaatgacaaattcctcttcgtcctcgcagttagctaaagattctcctttgatggatgagatatcaagcatcgcacgcagtcagcgtcaggtcatgcatcaattggacaatctcaataatcttctcagggggagcttgggggagaagtctcgcccaacaagaaccaacagcgggaaaagcatcaccatgtcatcggattccatgggagcttgtgttatggcggtggtcacagttggttgtttggggatcttcttgatgaagggttttttgaacaaaaagtga

SEQ ID NO: 34 - DNA, 1995 bp, Glycine max
atggcaaaagattctgctgatgctgattcccatcacagacgtcttggtcttttgaaagatcaagttcacttggttaaaaggaaaggctttgatcgctatgagattgctccaatccaagaccaactagcatttgagaagggttttttcatagtgattcgtgcatgccaattgttatcccaaaagaatgaaggaataatattggttggggtggcaggtccttcaggggcggggaagactgtatttactgaaaagatactcaacttcatgcccagcattgcagtcatttcaatggataattacaatgatgctagtcgaattgttgatgggaattttgatgatccacgcttaacagactatgacacattactccagaatctgcacgatttaaaggaaggaaagccagttcaagttccgatatatgatttcaagtccagttcacgtacaggatacaggacagtagaagtcccgagttctcgaattgtgattatagagggcatctatgccttgagtgagaagttgcgacctctgctggaccttcgagtctctgttacaggaggagttcaccttgaccttgtaaaacgagttatacgtgatatacaacgagctggtcaagaacctgaagaaattattcatcaaatatcagagacggtgtatccaatgtacaaggcatttatcgagccagatctccagacagcacacataaaaatcatcaacaaattcaatccatttactggattccagagtcctacttacatattaaagtcaggaaggaatgtagaagttgataaaattaaagctgttctcgctgaggattttaaggaaacaacagagcaaacttatgacatatatcttctaccacccggtgaagatccagaaacttgccaatcatatttacggatgcgaaataaagatgggaaatacagtctcatgtttgaggaatgggtgacagataacccatttgtcatatcaccaagaattacttttgaggtcagtgtgcgccttcttggtggactaatggctttgggatacacaatagctactatccttaagagaaacagccatgtcttttctgatgatagggtttgtgtgaaactagattggcttgagcaacttaatagacattatgttcaggtacaaggaagagatcgtttagttctaaaatatataggagaacagctgggtctagaaggttcctacattcctcgtacctacattgagcaaattcaaattgaaaagcttgtgaatgaggttatggccctaccagatgatttgaagacaaagctcagcctagatgaggatttggtgtcaagccctaaagaagcactatctagagcctctgccgacagagttgccatgagaaataaacatctgaggagtggaatatcacagtcgtataccaatcagagggacaaaaatctagctaaggttactggatatggtgccaacaatggaaggtttggtgaaaaaaatttagattccaccgcaatgccagtaaatcagggagcgatcaatcagctttcggaccaaatttctgccctcaatgatagaatggatgagtttacaaatcgcattgaagagctgaattccaagttaaccataaagaaaagttctccaagtcaacaaaacatgtcacttcaagctgagacatgcaatggatctgctcccacctctcacttcatcactagtttgggcaatggttccctaactggatctaaaatgacaaattcctcttcgtcgtcgcagttagctaaagagtctcctttgatggatgagatatcaagcattggacgcagtcagcgtcagatcatgcatcaattggacaatctcaataatcttctcagggggagcttgggggagaagtctcacccaacaagaaccaacagcaggaaaagcattgacatgtcatcggattccgtgggagctcgtgttatggtggtggcggcagttggttgtttggggatcttcttgatgaaggttttgttgaacaaaaagtga

SEQ ID NO: 35 - DNA, 1989 bp, Phaseolus vulgaris
atgggtaaaaacacttccgaatctgattcccatcacaaacgtcttggtcttttaaaagatcaagttcacttggttaaaaggaaagactctgatcgccatgagattgctccaatccaagaccaactatcatttgagaagggtttcttcattgtaattcgtgcatgtcaattgttagcccaaaagaatgatggaatagtattagtaggggtggcgggtccttccggagcaggcaagactgtatttactgaaaagatactcaacttcatgcccagcattgctgtcatttcaatggataattacaatgattctagtcggattgttgatggaaactttgatgatccacgcttaacagactacgacacattactccagaatttaggtgatttaaaggaaggaaagcctgttcaagttcccatatatgatttcaaatccagtacacgaacaggatacaggacagtagaagccccaacttctcgtattgtgtttatagagggcatctatgcattgagtgaaaagttgcgacctctactggaccttcgagtctctgttacaggaggagttcaccttgaccttgtaaaacgagttatacgtgatattcaacgtgctggtcaagaacctgaagaaattattcatcaaatatctgagacggtgtatccaatgtacaaggcctttattgagccagatctccagacagcacatataaaaatcatcaacaaattcaatccatttactggattccagagcccaacttacatattaaagtcagcaaagaatctagcagtggatcaaatcaaagctgttctctctgaggattttaaggaaacaacagagcaaacttatgacatatatcttctaccacccggtgaagatccagaaacttgccagtcatatttgcggatgcgaaataaggacgggaaatacagtctcatgttcgaggaatgggtgacagataatccatttgtcatatcaccaagaattacttttgaggtcagtgtgcgccttcttggtggactaatggccttgggatacacaatagctactatccttaagagaaacagccatgttttttctgatgatagggtttgtgtgaaactcgattggcttgagcaactcaatagacattatgttcaggtacaaggaagagatcgcttggttttaaaatacataggacagcagctaggactagatggttcctacattcctcgtacgtacattgagcaaattcaaattgaaaagcttgtggatgaggttatggccctaccagatgatttgaagacaaaactcagcctggatgaggatttggtgtcaagccccaaagaagcactttctagagcctctgctgacagagttgccatgagaaataaacatctgaggagtgggatatcacagtcatataccaatcaaagggacaaaaaccttgccaaggttggatttgatggcaacaatagaaggtttggtgaaagaaattcagagtccagtacaatgtcagttaatcagggagccatcaatcagctttcagaccaaatttctgccctcaatgatagaatggatgagtttacaaatcgcattgaagagctgaattccaagttaaacatcaagagaaattctccaacccaacaaaatatgtcacttcaagctgagacatgcaatggatcagctcccacctcatacttcatcactggtttgggcaatggttccctaactggatctaaaatggcaaattcctcttcgtcatcacaattaactaaggattttcctttaatggatgagatatcaagcattgcacgcagtcagcgtcagatcatgcatcaattggacaatctgaataatcttctcagagggaactcaggggagaagtctcagcaaacaagaactaaccggagaagcatagatacagcatcagattccatgggaacttctgttatggcagtggtggcagttggttgcttgggtatcttcttgatgaagggtttgttgaaccgaaattga

SEQ ID NO: 36 - DNA, 1980 bp, Populus trichocarpa
atggctaaagatacttctggtgccgaatcacacccaaaacggcagggactcttgaaagatcaagccagattaactaagaaaaaggactgtgatcgctttgagatagtcccaatccaaaatcctttgtcatttgagaaaggattctttattgttatccgtgcctgccaattgttagcacaaaataatgacggaatgatactggtaggtatagcaggcccttcaggagctgggaaaaccattttcactgaaaaaattctcaacttcatgcccagtgttgccaccatatcgatggacaactacaatgattcaagccgaattgttgatggcaattttgacgacccgcgcttgacggactatgacatgttgctcaagaatgttcttgacttaaaggatgggaaaccagttgaggttccaatctatgatttcaagtcgagcacccgaacaggatacaggacactcgaagtaccatcttctcgtatagtgattattgaaggaatctatgcactgagtgaaaagttgcgacctttgttagacctacgagtatctgtaactgggggagttcattttgatcttgtaaaacgggttttaagggacatccaacgagctggccaagaaccagaggaaataatccagcagatatctgaaacggtatatccgatgtacaaggcctttattgagccagatctcaaaacagcccatataaaaattacaaacaagttcaaccccttctctggatttcaaagtcctacttatatattaaagtcagcaaggaaggtaacagtggaccagatcaagcctgttctctctgaagattataaagagacaatggagcagatttatgacatatatcttttaccacctggtgaagatccagaatcatgccaatcatatttgaggatgcgaaataaagatggaaaatacaatctcatgtttgaggaatgggttgcagatgctccatttatcatatcaccaaggatcacttttgaagtcactgttaaactcctcagtgggctgatggccttgggatacacaatagctgctatccttcaaagaagtagccattcattctctgatgatagggtgtgtgtgaaaattgattggctaggacaactaaatcgtcaatatgttcaggtgcaaggaagagatcgcttggttgtaaaatacattgcagagcagctaggtttggaaggctcatacactccacgtacctatatagaacagattcaactagaaaggcttgtaaatgaggtcatggccttgccagatgacttgaagacaaagctcagcttagatgaggatctggtttcaagccccaaagaagcacttttgcaagcctctgctgatagggttgccaggagatttaagaatggcaaaagcggtatgtcacactcatattcctctcaaagggacaagaacttatctaagcttactgggcctgctgcaacctctaataggtttgatgatagcaatctagagtcaccagctgcgctagccaatcagggagccatgactcaactttcagaacagatttcttcactaaatgatagaatggatgagttcacaacctgtattgaagagttaaattccaagttaatcatcaataagaaccctcctagccaacaaaatatggctctccaagctgaagtgcacaatggttctgctccaacttcttacttcgtatctggtttaggcaacggttccttgactggatccagaatgtccaattcctcatcttcatctctgttggctaaggagtcacctttaatggaggagatatcaggaattgcccgggcacagcgtcaagtcacgcttcagttggatacgttgagcaatcttcttcatgatagcttgggagagaggtttcagggagtaagaaaaaacaggaatatcttggctgtccgtgatggccaagctccccttattgtagcattggcgattggttgtgtcggactatgttggtttattcgagcccagaattga

SEQ ID NO: 37 - DNA, 1986 bp, Populus trichocarpa
atggctaaagatacttctgaagctgaatcacacccaaaacggctgggactcttgaaagatcaagtcagatcaactaagaaaaaggactctgatcgctttgagatagttccaatccaaaatcctctgtcatttgagaagggattttttgttgttatccgtgcctgccaattgttagcacaaaagaatgatggaataatattggtaggtatagcaggcccttcaggagctgggaagaccattttcactgaaaaaattctaaacttcttgcccagcgttgctgtcatatcaatggacaactacaatgattcaagccgaattgttgatggcaattttgatgacccacgcttgacggactatgacacgttgctcaagaatgttcatgatttaaaggcagggaaaccagttgaggttccaatctatgatttcaagtctagcacacgaacaggatacaggacactcgaagtaccatcttctcgtatagtgattattgaaggaatctatgctctaagtgcaaatctgcgacctttgttagacctccgagtatctgtgactgggggagttcactttgaccttgtaaaacgggttttaagggacatccaacgtgctggccaagaaccagaggaaataattcaacagatatctgaaacggtatatccaatgtacaaagcctttattgagccagatctccaaacagcgcatataaaaattataaataagttcaaccccttctctggattccaaagtccaacttatatattaaagtcagcaagaaaggtaacggtggaccagatcaaagctgttctctctgaagaccaaaaagagacaatggagcagatttatgacatatatcttttaccacccggtgaagatccagaaacatgccaatcgtatctgaggatgcgaaataaagatgggaaatacaatcttatgtttgaggaatgggttgcagatgttccatttatcatatcaccaaggatcacttttgaagtcactgttaaactccttagtgggctgatggccttgggatacacaatagctgctatccttcaaagaagtagccatatattctctgatgatagagtgtgtgtgaaaattgattggttaggacaacttaatcgtcaatatgttcaggtgcaaggaagagatcggttggttgtaaaattcattgcggagcagctaggtttggaaggttcatacactccacgtacctatatagaacagattcaactagaaaggcttgtaaatgaggtcatggctttgccagatgacttgaagacaaagctcagcttagatgaggatctggtttcaagccccaaagaagcacttttgcgagcctctgctgatagagttgccaggagacttaagaatggcaaaagtggcatgtcacattcatattcctctcaaagggacaagaacttgtccaagcttactgggcttgctgcaaccagtaaaaggtttgatgatagaaatctggagtcaccagctgcactagccaaccagggagccataactcaactttcagaacagatttcttcactaaatgacagaatggatgagttcacaacatgtattgaagagttaaattccaagctaaccatcaagaagaattctcctagccaacaaaacatggctctccaagctgaagtgtgcaatggttctgctccagcttcttacttcgtatctggtttaggcaatgcttccttgaatggatccagaatgttcaattcctcatcttcatcccagttggctaaggagtcacctttaatggaggagctatcaggaattgcccgagtgcaacgtcaagtcatgcttcggttagatacgttgagcaatcttgttcgtgatagcttgggagagaggtctcaggaagtgagaaaaaacaggaatagattgattgtccgtgatggccaagcacctcttattgcagcattggcggttgggtgcgttggactgtgttggtttgttcgagctcgaaatggactctag

SEQ ID NO: 38 - DNA, 2091 bp, Linum usitatissimum
atgggagaagatgcttcgggttctgaatcgcacccaaagaagcagggacttttgaaagaccaggttaggttggcgaagaggaaggattctgaccgatacaagattgttccaattcaagagtcactgtcattcgagaagggcttctttgtagtcatacgtgcttgccaattactggcccaaaagaatgatgggatcattttggtaggtttggctggtccttctggggctggtaagaccatgttcactgaaaagataatcaatttcatgcccagcgttgctgtcatatctatggacaattacaacgatgccagccggattgttgatggaaattttgatgatccccgattgactgactatgacacattgcttaaaaatgttcaagatctaaaagctggaaaatctgccgaggtaccagtctatgatttcaagtctagctcccgcacgggatacagaacagttgaagttccagaatctcgcattgtgatcatagaagggatatatgccctaagtgaaaagttgcgccctttgctggacttgcgaatatctgttagaggggggattcattttgatctcgttaagcgtgttttacgagatattcaacgtgctggacaggaaccggaagaaataatccaccagatatctgagacggtatacccaatgtataaagccttcatagagccagatcttcaaactgctcacataaaaattataaacaagttcaacccatttaccggattccagaatccgacttacatcttgaaggttggtcttgttggcgatatcattttatttggatctctgtatccctttgcaatcctgaccttgttgatgctgctttggcttttagatgatttctttgattggccctcagcaagaaacgtggctgtagataaaatcaaggctgtcttgtctgaaggacacaccgagacggaggagcaaacttatgacatatatcttctgccacctggcgaagatccagagtcttgccaatcatatctgaggatgcgcaacaaagaagggaaatacaatctcatgtttgaggaatgggtgactgatgcaccattcgttatttctccgaggatcacgttcgaagttagcgtccgtcttcttggtgggttgatggctttggggtatacgattgcaaccatccttaaaaggagtagccatgtgttcttcaatgacaaagtatgtgtcaaagttgattggctagaacaactgaatcgtcagtatattcaggtgcaaggaagagatcgactggctgtgagatgtgttgcagagcaactaggtttggaagggtcatatattccccgcacatatatcgaacaaatccaactggagaagcttgtggatgaagtgatggctttgcctgatgatttgaagaataagcttagcctagatgaggacttagtgtctagccccagagaagcacttcttcgagcatctgccgatagggttgcaatgagaaacaagaacctcagtggcatgtcacattcatattcgactcaaggagacaagagctactccaagctcgctgcattctcgaatggaaaccggaggtacgaggaccgaaattcagagcctcaagcaatgctggcaaaccagggagtgataactcagctttcggaacagatgacatcactaaacgatagaatggatgattttacatcccagctagaagaactgaacaccaagctaaccgtgatgaacaattctccacgtcagcagaacatcggcgcgcaaggagagtcgagcaacggctctgctcctacatcgtacttcgtgtccggcttaagcaacggttccctgaccggatcaaaacaacaactgcctcattcgtcctcgttttcccagttagccaaggattcacctctactggaagagatcgtacgaggacaacgccaaatcatgcacaagctggacagtttcaccagcctcctgcatgaggcgacagcagagaggtccaagcaagctaggaaaggaaacaagctaacggtggctgacatgaacaccattggagttcctctggttttgtccatggcgattggtgggttagggctgttgctgtttaggagctttcgtaacagcagataa

SEQ ID NO: 39 - DNA, 1986 bp, Linum usitatissimum
atgggagaagatgcttcgggttctgaatcacacccaaagaagcagggacttttgaaggaccaggttaggttggcgaagaggaaggattctgaccgatacaagattgttccgattcaagagtcattgtcattcgagaagggcttctttgtagtgatacgtgcatgccaattactggcccaaaagaatgatgggatcattttggtaggtctggctggtccttctggggctggtaagaccatgttcactgaaaagataatcaatttcatgcccagcgttgctgtcatatctatggacaattacaacgacgccagccggattgttgatggaaattttgatgatccacgattgactgactatgacacattgcttaaaaatgttcaagatctaaaagctggaaaatctgcagaggtaccagtctatgatttcaagtctagctcccgcacgggatacagaacagttgaagttccagaatcacgcattgtgatcatagaagggatatatgccctaagtgaaaagttacgccctttgctggacttgcgaatatccgttagaggggggattcattttgatctcgttaagcgtgttttacgagatattcaacgtgctggacaggaaccggaagaaataatccaccagatatctgaaacggtatacccgatgtataaagccttcatagagccagatcttcaaactgctcacataaaaattataaacaagttcaacccattcactggattccagaatccgacttacatcttgaagtcagcaagaaacgtggctgtagataaaatcaaggctgtcttgtccgaaggacacaccgagacggaggagcaaacatatgacatatatcttctgccacctggtgaagatccggagtcatgccaatcatatctaaggatgcgcaacaaagaaggaaaatacaatcttatgtttgaggaatgggtgactgatgcgccgtttgtcatttctccgaggatcacgttcgaagttagtgtccgtcttcttggtgggttgatggctttggggtatacgattgcaaccatccttaaaaggagtagccatgtgttcttcaacgacaaagtatgtgtcaaagttgattggctagaacaactgaatcgtcagtatattcaggtgcaaggaagagatcgactggccgtgaggtatgttgcagagcaactaggtttggaaggttcatatattccacgtacatatatcgaacaaatccaactggagaagcttgtagatgaagtgatggctttgccagatgatttgaagaataagcttagcctagatgatgacttagtttctagccccagagaagcacttcttcgagcatctgccgatcgggttgcaatgagaaacaagcacctcagtggaatgtcacattcatattcaactcaaggtgacaagagctactccaagctcgctgcattctcaaatggaaaccgtaggtatgaggatagaaattcggagccccaagcaatgctggcaaaccagggagtgatgactcagctttcggaacagatgacatcactaaatgatagaatggatgattttacatctcaactagaagaactgaacaccaagctaaccgtgatgaacaattctcctcgtcagcaaaacatcggcgggcagggcgagtcaagcaacggctctgctcctacatcttacttcatgtccggcttaagcaactgttcgctgaccggttcaaagcagcaactgcctcattcgtcctcgttttcccagttagccaaggattcgcctctactggaagagatcgttcgaggacaacgccaaatcatgcacaagttggacagtttcaccagcctcctgcacgaggcgacagcagagaggtccaagcaagctaggaaaggaaacaagctaacagtggctgacatgaacaccattggagttcctctggttttatccatggcaattggtgggttagggctgttgctgtttaggagctttcataacagcggataa

SEQ ID NO: 40 - DNA, 1974 bp, Ricinus communis
atggcccaaggtatgtctggcattgaattgcaccagaaaaagcagggcctcttaaaagaccaagtgagattggttaagagaaaggactgtgaccgctatgagattgttccaatccaacagacttacacctttgagaaaggcttttttttatttattcgtgcatgccaattgttggcccaaaagaatgatgggattatcctggtaggtttagcaggtccttcaggggctgggaagactgttttcacagagaaggtactccatttcatgcccagtgttgctgttatttcaatggacaactacaatgattctagcagaattgtagacggcaactttgacgatccacgcttgactgactatgacacattgctgaagaatgtccaagatttaaagtcaggaaaagcagttgaggttccaatctatgatttcaaatctagctcacgcattggatataggacacttgaagtcccaacttctcgcatagtgattattgagggtatctatgctttaagtgaaaagctgcggcctatgctagacctacgagtatcagtaacaggaggagttcattttgatcttgtgaaacgagttttacgggacatccaacgtgctggccaagcaccagaggaaattatccagcagatatctgaaacggtgtatccaatgtataaagcttttattgagccggatcttcaaacagcacatataaaaatcataaacaaattcaaccctttctctggattccagagtcctacttacatattaaagtcagcaaagaaagtgaaagtggatcagatcaaggctgttctttctgaagagcatacagaggcaacagagcagacttatgatatatatcttttaccacctggtgaagatccagaatcttgccagtcatatctgagaatgaggaataaagatggaaaatacaatcttatgtttgaggaatgggtcactgataatccatttgttatatcgccaaggatcacttttgaagtcagcgtgcggcttctcggtgggttgatggccctaggatatacaatagcaaccatccttaaaagaagcagccatgtattctttaatgatagagtgtgtgtaaaaattgattggttggaacaactaaatcgtcaatatatccaggtgcaaggaagagatcggttggttgtgagatgtgttgcggagcagctaggtttagagggctcatatgttccacgtacctatatagaacagattcaactggaaaagcttgtaaatgaagttatggctttaccagatgatttaaagacaaaactcagcctggatgaggatttagtttcaagccccaaagaagcacttttgcgagcctctgctgacagggtagcaatgagaaacaagaacctcaaaagtggtatgtcgcattcatattcaagtcaaagggacaagaacttgtccaagcttgcaggacttgctgcaagcgacagaagatataatgaaagaaactcagattcatcagcagtgcaagcaaacgagggaatgctcactcaactatcagaacagatctcctcgcttaatgatagaatggatgagttcacgaaccggatggaggagctaaattccaaattaaacaacaaaagctctccaagccaacagaacttggctcttcaagctgaagcatgtaacggcactgctcccacttcttacttcctctccggtttgagcaacggttccttgactggaccaaaattatctaattcctcgtcttccacccaattggccaaggaatcacctttgctggaggagataactggcattatgaggggacaacgacaagtgatgcatcagttggatacgttaaataatctccttcgcgaaaatgcaggagagaggtctcggcaagtgagaacaaacaggagaagcatgattgctgattctgatatcacaaaaatcgctctagttttgtctattggtgttataggatttagcatgttccggcgaattttctag

SEQ ID NO: 41 - DNA, 1980 bp, Manihot esculenta
atggctcaagataattctagtgctgaattacagaagaaaaggcatggcctgttgaaagaccaagtgagattgtctaagagaaaggactgtggtcgctatgagattgttccaattcaacagacatatacatttgagaaaggattctttttatttattcgtgcgtgccaattgttagcccaaaacaatgatggaataatactggtaggcttagcaggtccttcaggggcagggaagactgttttcacagagaaggtactcaacttcatgcccagtgtcgctgtcatttcaatggacaactacaatgattctagccgaattattgacggcaattttgatgatccacgcttgacagattatgacacgcttctcaaaaatatccatgatttaaaggcaggaaaatcagctgagactccaatctatgatttcaagtctagttctcgtgttggatacaggatggttgaagtcccaacttctcgcatagtgattattgaaggaatttatgccttgagcacaaagctgcgacctatgctagaccttcaagtatcagtgactgggggagttcattttgatctggtaaaacgggtcctacgtgacatccaacgagctggccaagcgccagaggaaattatccagcagatatctgagacggtgtatccaatgtacaaagcttttattgagccagatctccaaacagcacatataaaaattataaacaaattcaacccattctctggtttccagaatcctacttatgtattgaagtcagcgaagaaggtgatggtggatcagatcaaggctgttctctctgaggaacatacagagatgacagagcagacttatgatatttttcttttaccacctggtgaagatcctgaatcttgtcagtcatatctgagaatgaggaataaagatggaaagtataatcttatgtttgaggaatgggtaacagatgctccttttgttatatcaccaaggatctcttttgaagtgagtgtgcgacttcttggtgggctaatggccttggggtatacaatagcaactatccttaaaagaagcagccacgtgttccttaatgacaatgtttgcgttaaaattgattggcttgaacaactaaaccgtcaatatgttcaggtgcaaggaaaagatcgcttggttgtgagatatgtcgcagagcagctaggtttggaaggctcatatgttccgcgtacctatatagaacagattcaactggaaaagcttgtagatgatgttatggctttaccagatgatttgaagacaaagctcagcctagatgaggatttggtttcaagccccaaagaagcacttctgcgagcctctgccgatagggttgctatgagaaacaagaatctcaaaagtggtatgtcacattcatattcaaaccaaagggagagcaacttttccaaacttgctgtactcgctgcaagtaacggaggctatactgagagaaacacagtgtcaatggcagtgctagcaaatcagggcatcctcactcaactttcggaacagatatcctctctgaacgatagaatggatgagtttgcagcccgtattgaagatctaaattccaaattaaacatcaccagcagttcttccagccaacaaaacttggttctccaagctgatgcatgtaatggctctgctcccacgtctcacttcctctctggtttaagcaatggttccttgaccggatgcaagttgcgtaattcctcatctttctctcagttggccaaggagtcaccattaatggaagaggtatctggcattgctcgggggcaacgacaggttatgcatcagttagatactttaagtaatatacttcgtgagagcttaggacagagatctcagcaagtaagaacaaacagaagaagaagtatgattgctgatctcgaaattacaaaacttgctcttattttgtcagttggtgttataggattcagtacgttaagaaggattttctag

SEQ ID NO: 42 - protein, 674 aa, Artificial Sequence
  TTM2 amino acid sequence
MGQDSNGIEFHQKRHGLLKDQVQLVKRRDSIRYEIVSIQDRLSFEKGFFAVIRACQLLSQKNDGIILVGVAGPSGAGKTVFTEKILNFLPSVAVISMDNYNDSSRIVDGNFDDPRLTDYDTLLKNLEDLKEGKQVEVPIYDFKSSSRVGYRTLDVPPSRIVIIEGIYALSEKLRPLLDLRVSVTGGVHFDLVKRVLRDIQRAGQQPEEIIHQISETVYPMYKAFIEPDLQTAQIKIINKFNPFTGFQSPTYILKSRKEVSVDQIKAVLSDGHTETKEETYDIYLLPPGEDPESCQSYLRMRNKDGKYSLMFEEWVTDTPFVISPRITFEVSVRLLGGLMALGYTIATILKRNSHVFATDKVFVKIDWLEQLNRHYMQVQGKDRQLVQSTAEQLGLEGSFIPRTYIEQIQLEKLINEVMALPDDLKNKLSLDEDLVSSSSPKEALLRASADRVAMRNKNLKRGMSHSYSTQRDKNLSKLAGYSSSDRRYEERNHDSPANEGFMTLLSEQISSLNERMDEFTSRIEELNSKLSCNKNSPTQQSLSIQTEVCNGSAPTSYFISGLDNGCLTNSIMPHSSSSSQLAKDSPLMEEISTISRGQRQVMHQLDNLCNLMRESSAERSRLARTGSSNSGNRGRSSKSSFLSNVESNKLPLVLTVAICSIGIIVIKSYINKRQ

SEQ ID NO: 43 - DNA, 1980 bp, Manihot esculenta
atggaccgagataattctagtgctgaactacaccagaaaaggcatgggctcttgaaagaccaagtcagattggttaagagaaaggattgtgatcgctacgagattgttccaattcaacagacttatacatttgagaaaggattttttctatttatccgtgcatgccagttgttggcccaaaacaatgatggaataatactgataggtttagcaggtccttcaggggctgggaagactgttttcacagagaaggtactcaacttcatgcccagtgttgctgtcatttcaatggacaactacaatgattctagccgaattgttgacggcaattttgatgatccacgcttgacagattatgacacattgctcaagaatgtccatgatttgaaggcaggaaaatcagcggaggttccaatatatgatttcaagtctagctcccgcattggatacaggacggttgaggtcccaacttctcgtatagtgattatcgaggggatatatgccttgagtgaaaagctgcgacctctgctagaccttcgagtatcagtgactgggggagttcattttgaccttgtgaaacgggtcctaagggacatccatcgtgctggccaagcaccagaggaaattatccagcagatatctgaaacggtatatccaatgtacaaagcttttattgagccagatcttcaaacagcacacataaaaatcataaacaaattcaatcctttctctggattccagagtcctacttacatattgaagtcagcaaaaaaggtgaaggtggatcaaatcaaggctgctctctctgaagaccatacggagacaacagaggagacttatgatatatatcttctaccacctggtgaagatcctgaatcttgccagtcctatctgcgaatgaggaacaaagatggaaaatacaatcttatgttcgaggaatgggtaacagatgctccttttgttatctcgccgaggatcacttttgaagtcagtgtgcggcttcttggtggtctaatggctttgggatacacaatagcaaccatccttaaaagaagcagccatgtattcatcaacgatagagtgtgtgtgaaaattgattggctggaacaattaaatcgtcagtatgttcaggtgcaaggaagagatcgcttggttgtgagatgtgttgcagagcagctaggtctggaaggctcatatgttccgcggacctatatagaacagattcagctggaaaagcttgttaatgaagttatggctttaccagatgatttgaagtcaaaactcagtctagatgaggatttggtgttcagccccaaagatgctcttttgccagcttctgctgatagggttgcaatgagaaacaagaatctcaaaagcggtatggcacattcatattcaagtcaaagggacaagaacttatccagtcttgctggacttgctgcaaataaccgggggtatagtgagagaaaccgggagtcgaaggcattactagcaaaccagggaattctcactcaactttcagaacagatttcctcactaaatgatagaatggacgagttcaccacccgtattgaagagctaaattccaaattaaacattaacgaaaactcttccggccaacaaaaactgggtctccagcctgaagcgtgcaatggatatgcttcttccatgtcttacttcacctctggtttaagcaatggctccttgactggatccaaaatgcacaattcctcatcttcctctcagttggctaaggagtcacaattaatggaagagatatctggcattgtgcggggacaacggcaagtgatgcaccagttagatactttaagcaatctacttcgagagagtttaggacagagatctgagcaagtaagaaggggcaggagaagcatgattcctgatattgaaattacgaaaattgctgttattttgtctgttggtgttttgggattcagcatgttgagaaggattttctag

SEQ ID NO: 44 - protein, 674 aa, Arabidopsis thaliana; identical to SEQ ID NO: 42

SEQ ID NO: 45 - protein, 653 aa, Solanum lycopersicum
MPKDTSNGEATRRAGLLKDQVQLVKRKNCDRYEIISIPDNLSFEKGFFVVIRACQLLVQKNEGLIILGVAGPSGAGKTVFTEKILSFMPSVAVISMDNYNDASRIVDGNFDDPRLTDYDTLLKNINDLKAGKPAEVPIYDFKSSSRIGYRILEVPSSRILIIEGIYALSEKLRPFLDLRVSVTGGVHFDLVKRVLRDIQRAGQEPSEIIHQISETVYPMYKAFIEPDLKTAHIKIINKFNPFTGFQSPTYILKSFRDVQVDQIKSALSEEHTESTEQTYDIYLLPPGEDPETCQSYLRMRNKDGKYSLMFEEWVTDSPFVISPRISFEVSVKLLGGLMALGYTIATILKRSSHVFSDEKVCVKIDWLEQLNRHYVQVQGRDRLIVKSVADQLGLEGSYTPRTYIEQIQLEKLVNEVMALPDDLKTKLSLDEDLVSSPKEALSRASAERVAWRNKNLRSGLSHSYANHREKNLSKIDTDGQRFDDRNTDSATLANQGAVTHLSEQISTLNDRMDDFTSKMEELNSKLTKKRTSQSTQSLALQAEACNGSGPTSYFISGLGNGSLTGSIMPNSSSSSLLIPKESNLMEELSNVARGQRQIMHQLDSLSNLLRERLGEQSRQARTSKRSDLNSIRVPLVVTLAVSGLGLFLFRSRN

SEQ ID NO: 46 - protein, 648 aa, Solanum lycopersicum
MDIDTANAESINQKAGLLKDQVRLIKRKDCDRYEIASIPDNLSFEKGFFIVIRACQVLVQNNEGLIMIGVAGPSGAGKTVFTDKIMNFMPSIAVISMDNYNDASRIVDGNFDDPRLTDYDTLLKNINDLKTGKAAEIPIYDFKSSSRIGYRTVEVPSSRIVVIEGIYALNEKLRPFLDLRISVNGGVHFDLVKRVLRDIQRAGQEPSEIIHQVSETVYPMYKAYIEPDLKTAHIKIINKFNPFSGFQSPTYILKSSRNLKVEQIKSVLSEDHTESTELIYDIYLLPPGEDPETCQSYLRMRNKDGKYNLMFEEWVTDSPFVISPRISFEVSVRLLGGLMALGYTMAAILKRSSHVFSDERVCVKIDWLEQLKRHYVQVQGRDRVVVKGIAEQLGLEGSYTPRTYIEQIQLEKLVNEVMALPDDLKTKLSLDEDIVSSPKEALSQASAQRAWRNKNIRSGLSHSYSTNRDKNLNVSFDYQRNDRIEESGTRLANQGAITRLLEQISTLNDRMDNFTSIMEELNSKLCSRIASPRNKQASPCIQNLTLDSEACVGSAPTNYFISGLENGSLTGSIMPNSSSFTSTIGKESALMEEISNIARGQRQLMHQLDNVSNVLREGLGEQSRQARVNKKSDTIKTIRKPLIVTLAV

SEQ ID NO: 47 - protein, 670 aa, Petunia hybrida
MDVDTANAELSNQRAGLLKEQVRLVKRKDCDRYEIVSIPDNLSFEKGFFIVIRACQLLVQKNDGLVMVGVAGPSGAGKTVFTEKILNFMPSVAVISMDNYNDASRIVDGNFDDPRITDYDMLLNNINDLKTGKPAELPIYDFKSSSRIGYRTLEVPSSRIVIIEGIHALNEKLRPFLDLRVSVNGGVHFDLVKRVLRDIQRAGQEPSEIIHQVSETVYPMYKAYIEPDLKTAHIKIINKFNPFSGFQSPTYILKARPLNLHIQNLEIEQIKSVLSEEHTECTEQVYDIYLLPPGEDPETCQSYLRMRNKDGKYNLMFEEWVTDSPFVISPRISFEVGVRLLGGLMALGYTIAAILKRSSHVFSDEKVCVKIDQLEQLNRHYVQVQGKDRVIVKCIADQLGLEGSYTPRTYIEQIQLEKLVNEVMALPEDLKTKLSLDEDIVASPKEALSRASAERVSWRNKNIRSGLSHSYSTNRDKNLPNLTSDYQRNDGSAESATRLANEGAVKHLSEQISTLNDRMDDFTSRMEELNSQLSSRRASPRSKRASPSAHSLALETEVCNGSAPTSYFISGLENGSLTGSIMPNSSSFASLTAKESAFMEEISNIARGQRQVMHRLDNISNVLRERLAELSGQERMNKKRDITDIHPIRIPIILTLAVGGLGILLFKSLQ

SEQ ID NO: 48 - protein, 654 aa, Petunia hybrida
MAKDIGNGESTHRRAGLLKDQVRLVKRKDTSRYEIVSIPDNLSFEKGFFVVIRACQLLVQKNEGLTIIGVAGPSGAGKTVFTEKILSFMPSVAVISMDNYNDASRIVDGNFDDPRLTDYDKLLENINDLKAGKPAEVPIYDFKSSSRTGYRTLEVPTSRILIIEGIYALSEKLRPLLDLRVSVTGGVHFDLVKRVLRDIQRAGQEPSEIIHQISETVYPMYKAYIEPDLKTAHIKIINKFNPFTGFQSPTYILKSSRNVTVDQIKSALSEEHTESTEQTYDIYLLPPGEDPETCQSYLRMRNKDGKYSLMFEEWVTDSPFVISPRISFEVSVKLLGGLMALGYTIAAILKRSSHVFSDEKVCVKIDWLEQLNRHYVQVQGKDRLIVGSVADQLGLEGSYTPRTYIEQIQLEKLVNEVMALPDDLKTKLSLDEDLVSSPKEALSRASADRVAWRNKNLRSGLSQSYANHREKNLSKINTDSRRFDDRNGDSATTLANQGSVTHLSEQISTLNDRMDDFTSRMEELNSKLSSKRASPSTQSLALQAEACNGSGPTSYFISSLENGSLTGSIMPNSSSSSSLIPKESTLMEELSNVVRGQRQIMHQLDNLSNLLRDRLGERSRQARTGKRRDIVDIDSVKLPLIVTLAVGGLGFLMF

SEQ ID NO: 49 - protein, 722 aa, Cucumis sativus
MAQDHSGSESHQKRAGLLKDQVRLIKRKDSDRYEIVSIQDPLSFEKGFFIVIRACQLLAQKNDGIILVGLAGPSGAGKTVFTEKIMNFMPSIAIISMDDYNDASRIVDGNFDDPRLTDYDTLLQNVQDLKAGKQVQVPIYDFKSSSRIGYRTVEVPSSRIVIIEGIYALSERLRPLLDLRVSVRGGVHFDLVKRVLRDIQRAGQEPEEIIHQISETVYPMYKAFIEPDLETAHIKIINKFNPFTGFQSPTYILKSARKITVDQIKAVLAEDHTEHKEQTYDIYLLPPGEDPESCQSYLRMRNKEGKYSLMFEENYAVLLFFKFAWIPYYIYGLVIVEYPGIIIYKPEWVTDNPFIISPRITFEVSVRLLGGLMALGYTIATILKRSSHVFSDHRVCVKIDWLEQLNRQNVQVQGKDRLVVKHVAEQLGLDGSYIPRTYIEQIQLEKLVNEVMALPDDLKSKLSLDEDLVSSPKEALSRASADRVSLRNRNLKSGISQSYTTQREKKLSGYGSNNQRFVDRNTESQAMLANQGAITQLSEQISSLNDRMDEFTARIEELNSKLSFKRNSPSQQNINLQSETCNGSVPTSYFVSGLGNGSLTGSIIPSSSSFSQLAKDSPIMDEISGISRGQRQVMHQLDCLSNLLRERDNAGDRSRQVRTKKKAIMPDPEPLKLPLLLTLAVGGVGVVLYKSFLSLSTNCVAVITLRRGKDDRIDLTDESGSR

SEQ ID NO: 50 - protein, 569 aa, Capsicum annuum
MDIDTANAESINQRAGFLKDQVRLVKRKDCDRYEIASIPDNLSFEKGFFIVIRACQLLVQKNDGLIMIGVAGPSGAGKTVFTEKIMNFMPSIAVISMDNYNDASRIVDGNFDDPRLTDYDTLLKNINDLKTGKAAELPIYDFKSSSRIGYRTLEVPSSCIVIIEGIYALNEKLRPFLDLRISVNGGVHFDLVKRVLRDMQRAGQEPSEIIHQVSETEWVTDSPFVISPRISFEVSVRLLGGLMALGYTMAAILKRSSHVFSDERVCVKIDLLEQLKRHYVQVQGRDRVVVKSIADQLGLDGSYTPRTYIEQIQLEKLVNEVMALPDDLKTKLSLDEDIVASPKEAFSRASGERASWRNKKIRSGFSHSYSTNRDKNLSDVSSDYRSNGRIEESGARLSNQGAVTHLLEQISTLNDRMDDFTSKMEELNSKWSSRRASPRIKQASPSTQNLALETEACIGSAPTNYFISGLENGSLTGSIMPNSSSFTSVNGKEFTLMEEISNIARGQRQVMHQLDNVSNVLRERLGEESRQERMNKKRDTMDPIRVPLIVALAVGGLGILLFKSLQHQT

SEQ ID NO: 51 - protein, 659 aa, Capsicum annuum
MAKDTSNAESANRRAGLLKDQVRLVKRKNSDRYEIVSIPDNLSFEKGFFVVIRACQLLVQKNEGLIVIGVAGPSGAGKTVFTEKILSFMPSVAVISMDNYNDASRIVDGNFDDPRLTDYDTLLKNINDLKAGKPAEVPIYDFKSSSRIGFRTLEVPSSRILIIEGIYALSEKLRPFLDLRVSVTGGVHFDLVKRVLRDIQRAGQEPSEIIHQISETVYPMYKAFIEPDLKTAHIKIINKFNPFTGFQSPTYILKSFRDVKVDQIRSVLSEEHTESTEQTYDIYLLPPGEDPETCQSYLRMRNKDGKYSLMFEEWVTDTPFVISPRISFEVSVKLLGGLMALGYTIATILKRSSHVFSDEKVCVKIDWLEQLNRHYVQVVQGRDRLVVKSFADQLGLEGSYTPRTYIEQIQLEKLVNEVMALPDDLKTKLSLDEDLVSSPKEALSRASADRVAWRNKNSRGGLSHSYSNHTERNLSKIDTDSRRFDDRNSDSAATLANQGAVTHLSEQISSLNDRMNDFTSRMEELNSKLSSKRASPSAQSLALQAEVCNGSGPTSYFISGLENGSLTGSIMPNSSSSSSLIPKESTLMEELSNVARGQRQIMHQLDNLSNVLRDRLGEQSRQARTSKTRDVNSIRVPLVVTLAAGGLGLFLFKSLQNQK

SEQ ID NO: 52 - protein, 652 aa, Oryza sativa
MDRSDSMADSPRRRNGLLRDQVQLVKRKDTNRYEIVRFPDPLSFEKGFFVMIRACQLLVQHNEGMIFVGVAGPSGAGKTVFTEKVLNFMPGVAVISMDNYNDASRIVDGNFDDPRLTDYDTLLENIHGLKEGRSVQVPIYDFKMSCRTGYRTVDVPSSRIVIIEGIYALSEKLRSVLDLRVSVTGGVHFDLVKRVLRDIQRAGQEPEEIIHQISETVYPMYKAFIEPDLQTAHIKIINKFNPFSGFQNPMYILKSPRTITPTDIKVALGEDHTESIEETYDIYLLPPGEDPESCQSYLRMRNREGKYNLMFEEWVTDNPFIISPRITFEVSVRLLGGLMALGYTIAAILKRKSRVFSDGKATVKIDWLEQLNRNYIQVQGRDRNHVKFVAEKLGLDGSYIPRTYIEQIQLEKLINDVMALPEDLKTKLSIDDELVSSPKEALSRVSADKRNKHLKSGLSHSYSTHGDKNIVKLSKLTETNRRFGSGRAPEPPAINQGAITQLSEQISTLNERMDEFTSRVEELNCKFAIKKSSTSQQNLALPNETCNGSAPTNLFVSHLGNGTLIPHSSSSNQLLKESPIVDEINAISRGQRQVIHQLDNLTSLLHEHLALTRQGNAVRRNGILEMDMSICPLIALTIGGFGYLVFKSLNRS

SEQ ID NO: 53 - protein, 653 aa, Hordeum vulgare
MERSDSLSESPRKRNGLLRDQVQLVKRKDTSRYEIVPFPEPLSFEKGFFVMIRAIQLLVQNNEGIIFVGVAGPSGAGKTVFTEKVLNFMPSVAVISMDNYNDASRIVDGNFDDPRLTDYDTLLENIHGLKEGRSVQVPIYDFKTSCRTGYRTVDVPSSRIVIIEGIYALSDKLRPILDLRVSVTGGVHFDLVKRVLRDIQRAGQEPEEIIHQISETVYPMYKAFIEPDLKTAHIRIINKFNPFSGFQNPMYILKSPRSLTPDQIKAALGEDQTESNEETYDIYLLPPGEDPEACQSYLRMRNREGKYNLMFEEWVTDNPFIISPRITFEVSVRLLGGLMALGYTIAAILKRSSRVFSDGKATVKIDWLEQLSRRYIQVQGRDRLYVKFVAEQLGLDGSYIPRTYIEQIQLEKLMNDVMALPDDLRTKLSIDDELVSSPKEAFSRASADRRNELMKSGLSHSYSTHGDKSMVKLNKLTESNRRFGSRRTPEPPAINQGAINQLSEQISTLNERMDEFTCRVEDLNSKFTLIKSSPSQQNLAPPSDTRNGSAPTNLFVSQLGNGTLIPHSSSSNQLSKESPLTEEIMVLSRGQRQVIHQLDNLTNLLHEHLVLTRQGNTTSRNRIQEGIDMAICPLIILTIGSVGYFVFRNLNRT

SEQ ID NO: 54 - protein, 653 aa, Zea mays
MDQHDSMADSPRRRHNLLRDKVQLVKRKDSDRYEIVRFHDPLSFEKGFFVVIRACQLLAQHNDGIIFVGVAGPSGAGKTVFTEKVVNFMPDVAVISMDNYNDATRIVDGNFDDPRLTDYDTLLENIHGLKEGRSVQVPIYDFKSSCRTGYRTVDVPSSRIVIIEGIYALSEKLRPVIDLRVSVTGGVHFDLVKRVLRDIQRAGQEPEEIIHQISETVYPMYKAFIEPDLETAHIKIINKFNPFSGFQNPMYILKSPRSLGPEKIKAVLGDDHTESNEETYDIYLLPPGEDPEACQSYLRMRNREGKYNLMFEEWVTDNSFIISPRITFEVSVRLLGGLMALGYTIAAILKRSTRVFSDGKATVKIDWLEQLNRQYIQVQGRDRLYVKSVADQLGLDGSYIPRTYIEQIQLEKLINDVMALPEDLKTKLSIDDELVSSPKEAFSRVSADRRNKLMKSGLSQSYSTHGDKNIVKLSKLTETNRRFGGGRALEPPAINQGAITQLSEQISTLNERMDEFTSRVEELNSKFTVKKHLPSQQNLALPNDACNGSTPTNLFVSHLGNGTLIPHSSSSNQLSKDSPMIEEIMNITRGQRQVIHQLDNLTNLLHEHLVLTRQTNNTASRNRVLDSDTLICPLICLTVASIGYFMFKGLSRG

SEQ ID NO: 55 - protein, 653 aa, Brachypodium distachyo
MERSDSMSESPRKRNGLLRDQVQLVKRKDAGRYEIVPFPEPLSFEKGFFVMIRAIQLLVQHNEGIIFVGVAGPSGAGKTVFTEKVLNFMPSVAVISMDNYNDASRIVDGNFDDPRLTDYDTLLENIHGLKEGRSVQVPIYDFKMSCRTGYRTVDVPSSRIVIIEGIYALSDKLRPILDLRVSVTGGVHFDLVKRVLRDIQRAGQEPEEIIHQISETVYPMYKAFIEPDLKTAHIRIINKFNPFSGFQNPMYILKSPRSLTPDEIKAALGEDQTESNEETYDIYLLPPGEDPEACQSYLRMRNREGKYNLMFEEWVTDNPFIISPRITFEVSVRLLGGLMALGYTIAAILKRSSRVFSDGKATVKIDWLEQLSRRYIQVQGRDRLYVKFVAEQLGLDGSYIPRTYIEQIQLEKLMNDVMALPDDLKTKLSIDDELVSSPKEAFSRASADRRNKLMKSGLSHSYSTHGDKSIVKLNKLTETNRRFGSGRTPEPPAINQGAINQLSEQISTLNERMDEFTCRVEELNSKFTLMKPSSSQQNLALPSETRNGSAPTNLFVSQLGNGTLIPHSSSSNQLSKESPMMEEVMNLSRGQRQVIHQLDNLTNLLHEHLVLTRQGNSMSRNRILEGFDMAICPLIILTIGSVGYFAFKSLNRT

SEQ ID NO: 56 - protein, 664 aa, Prunus persica
MAQDMSGVDSHQRRQGLLKDQVRLVKKKDSHYEIVPIQSPLSFEKGFFIVIRACQLLAQKNDGIILVGVAGPSGAGKTVFTEKILNFMPSVAVISMDNYNDASRIVDGNFDDPRLTDYDTLLQNVNDLKAGKEVQVPVYDFKSSSRTGFRTVEVPSSRIVIIEGIYALSEKLRPFLDLRVSVTGGVHFDLVKRVLRDIQRAGQEPEEIIHQISETVYPMYKAFIEPDLQTAHIKIINKFNPFTGFQSPTYILKSAKDLSADQIKAVFSEDHTEAKEETYDIYLLPPGEDPESCQSYLRMRDKDGKYSLMFEEWVTDNPFVISPRITFEVSVRLLGGLMALGYTIATILKRSSHVFSNDGVCVKIDWLEQLNRRYIQVQGKDRVLVRCVAEQLGLEGSYIPRTYIEQIQLEKLVNEVMALPDDLKTRLSLDEDLVSSPKEALSRASADRVAMRNKHLKSGMSQSYTTQRDKSTSKLTGYASNSQRFDERNSESSATLASQGVVTQLSEQMSSLNDRMDEFTNRVEELNSKLAVKKSSPSQQNMALQAETCNGSVPTSYFISGLGNGSLTGSILPNSSSSSQLAKESSVMEEMSSIARGQRQIMHQLDNLSNLLRENMGERNRPVRTNSRKSTIAQPDQPLTVPLAITLAVGVLGLIIYKGIFTRN

SEQ ID NO: 57 - protein, 664 aa, Malus x domesetica
  misc_feature (33)..(33): Xaa can be any naturally occurring amino acid
MAQDMSGADSHQRRQGLLKDQVRLVKRKDSSHXEIAPIQSPLSFEKGFFIVIRACQLLAQKNEGIVLVGVAGPSGAGKTIFTEKIVNFMPSVAVISMDNYNDASRIVDGNFDDPRLTDYDTLLQNVNDLKAGKPVEVPVYDFKSSSRTGYRTVEVPSSRIVIIEGIYALSEKLRPYLDLRVSVTGGVHFDLVKRVLRDIQRAGQEPEEIIHQISETVYPMYKAFIEPDLQTAHIKIINKFNPFTGFQSPTYILKSAKNLSADQIKAVFSEDHTEAKEETYDIYLLPPGEDLEACQSYLRMRDKDGKYSLMFEEWVTDNPFVISPRITFEVSVRLLGGLMALGYTIATILKRSSHVFSNDGVCVKIDWLEQLNRRYIQVQGKDRVVVRCVAEQLGLEGSYIPRTYIEQIQLEKLVNEVMALPDDLKTRLSLDEDLVSSPKEALSRASADRVAMRIKNLKSGMSQSYTSQRDKSTSKLTGYSSNSQRFDERNTESSATLESQHGVATQLSEQMSSLNDRMDDFTNRIEELNSKLTMKNSPSQHNMALQAENCNGSVPTSYFISGLGNGSLTGAIMPNSSSSSQVNKESSVVEEMSSVARGQRQIMHQLDNLSNLLRDSMGERPRPVKTNSRKNIVAQPEPLTVPLAVTLAVGVLGVIIYKGILTRN

SEQ ID NO: 58 - protein, 637 aa, Sorghum bicolor
MDQHDSMADSPRRLHNLLRDKVQLVKRKDSNRYEIVRFRDPLSFEKGFFVVIRACQLLAQHNGGIIFVGVAGPSGAGKTVFTEKVVNFMPDVAVISMDNYNDATRIVDGNFDDPRLTDYDTLLENIHGLKEGRSVQVPIYDFKSSCRIGYRTVDVPSSRIVIIEGIYALSEKLRPVMDLRVSVTGGVHFDLVKRVLRDIQRAGQEPEEIIHQISETVYPMYKAFIEPDLETAHIKIVNKFNPFSGFQNPMYILKSPRSLLPEKIKSVLGDDHMESNEETYDIYLLPPGEDPEACQSYLRMRNREGKYNLMFEEWVTDNSFIISPRITFEVGVRLLGGLMALGYTIAAILKRSSRVFSDGKATVKIDWLEQLNRHYIQVQGRDRLYVKFVADQLGLDGSYIPRTYIEQIQLEKLINDVMALPEDLKTKLSIDDELVSSPKEAFSRVSADRRNKLMKSGLSQSYSTHRDKRVVKLSQLTERRFGGGQALEPPAIDQGGIIQLSEQISTLNERMDEFTSRVEELNSKFSVKKHLPSQQNLALPNDACSGSTPTNLFVSQLGNGTLIPHSSSSNQLSKDSPMIEEVMNISRGQRQVIHQLDNLTNLLHEHLVLTRQANSCPLICLTVASIGYFMFKGLNRS

SEQ ID NO: 59 - protein, 661 aa, Aquilegia coerulea
MAKDASGPDSPHRKHGLLRDQVQLVKKRDSDRYEIVPIQDLLSFEKGFFIVIRACQLLAQKNEGIILVGVAGPSGAGKTVFTEKVLNFMPSIAVISMDNYNDASRIIDGNFDDPRLTDYDTLLENIHGLKEGKPVQVPVYDFKSSSRTGYRTIEVPSSRIVIIEGIYALSEKLRPLLDLRVSVTGGVHFLVKRVLRDIQRAGQEPEEIIHQISETVYPMYKAFIEPDLQTAHIKIRNKFNPFAGFQNPTYILKSTRTVTVDQIKAVISGECKDHMEEIYDIFLLPPGEDPEACQSYLRMRNRDGKYNLMFEEWVTDSPFMISPRITFEVSVRLLGGLMALGYTIATILKRSSHVFFNDRVCVKIDWLEQLNRQYVQVQGRDRLYVKFIAEELGLEGSYVPRTYIQQIQLEKLVNEVMALPDDLKTKLSIDEDLVSSSPKEALSRASADRIAMRNKHSKSGMSQSYSTHGDKNPFKLTRLSVNNQKFDGGNLESPAAVTNQGVINQLLEQISTLNERIDEFTSRIEELNSKFKTRNVSGSQQNLSLQAEPCNGSGQTSFFVSGLGNGSLTGSLLPNSSSSSQLAREAPLMEEVAVIARGQRQLMHQLDNLSNFVHENIEQSRHGRTEGSRLTNFEHIGIPVLLSLAIGGVGILLFKGLSSQN

SEQ ID NO: 60 - protein, 656 aa, Mimulus guttatus
MSKDTYNDESSHRKTGLLKDQVKLVKKKDSNRYEISPIPNELSFEKGFFATVRACQLLTQKNEGLVLIGVAGPSGAGKTIFTEKIVNFMPSIAVISMDNYNDASRIIDGNFDDPRLTDYDTLLKNINDLKEGKQVDIPIYDFKSSSRTGYRTLEVPSSRIVIIEGIYALSEKLRPLLDLRVSVTGGVHFDLVKRVLRDINRADQEPEEIIHQISETVYPMYKAFIEPDLDTAHIKIGNKFNPFTGFQSPTYILKSLKSVPVEQIKSVMSEEHTECTEETYDIFLLPPGEDPETCQSYLRMRNKDGKYNLMFEEWVTDTPFVISPRITFEVSVRLLGGLMALGYTIAAILKRSSHIFGDEKVCVKIDWLEQLNRNYIQVQGRDRLVVKSVAEQLGLDGSYVPRTYIEQIQLEKIVNDMMVMPDDLKNKLSIDDDFASSPKEALSRASALARIKNLRSGMSQSYTTNRDKNLSKMNRRFDDHTADSPAILANQGAVTQLSEQISTLNERMDDFTSRIEELNSKLTSKNGSPNSQSIGLQAEACNGSAPTSYFISGLGNGSLTGSMLPNSSSSSQLAKDSSLTEELSSISRGQRQVMHQLDNLNNLLRENMGDKSVRDRRKKRSGIAENDTNRNAVILLASLAVGGLGIFLFKGVLSRN

SEQ ID NO: 61 - protein, 653 aa, Solanum tuberosum
MPKDTSNGESTQRAGLLKDQVRLVKRKNCDRYEIISIPDNLSFEKGFFVVIRACQLLVQKNEGLIILGVAGPSGAGKTVFTEKILSFMPSVAVISMDNYNDASRVVDGNFDDPRLTDYDTLLKNINDLKAGKPAEVPIYDFKSSSRIGYRTLEVPSSRILIIEGIYALSEKLRPFLDLRISVTGGVHFDLVKRVLRDIQRAGQEPSEIIHQISETVYPMYKAFIEPDLKTAHIKIINKFNPFTGFQSPTYILKSSRDVQVDQIKSALSEEHIESTEQTYDIYLLPPGEDPETCQSYLRMRNKDGKYSLMFEEWVTDSPFVISPRISFEVSVKLLGGLMALGYTIATILKRSSHVFSDEKVCVKIDWLEQLNRHYVQVQGRDRLIVKSVADQLGLEGSYTPRTYIEQIQLEKLVNEVMALPDDLKTKLSLDEDLVSSPKEALSRASAERVAWRNKNLRSGLSHSYANHREKNLSKIDTDSQRFDDRNTDSATLANQGAVTHLSEQISTLNDRMDDFTSKMEELNSKLTKKRASPSTQSLALQAEACNGSGPTSYFISGLENGSLTGSIMPNSSSSSLLIPKESNLMEELSNVARGQRQIMHQLDSLSNLLRDRLGEQSRQARTSKRRDLNSIRVPLIVTLAVSGLGFFLFKSRN

SEQ ID NO: 62 - protein, 663 aa, Vitis vinifera
MAQDTSGAESPQPRPGLLKDQVRLVRRKNSDRYEIVPIQDRLSFEKGFFIVIRACQLLAQNNDGIILVGLAGPSGAGKTVFTEKILNFMPSIAVISMDNYNDASRIVDGNFDDPRLTDYDTLLQNVHDLKAGKQVQIPIYDFKSSSRTGYRTLEVPSSRIVIIEGIHALSEKMRPLLDLRVSVTGGIHFDLVKRVLRDIQRAGQEPEEIIQQISETVYPMYKAFIEPDLGTAHIKITNKFNPFTGFQSPTYILKSARNVTVDQIKAVITEEHAETMEEVYDIYLLPPGEDPESCQSYLRMRNKDGKYSLMFEEWVTDNPFVISPRITFEVSVRLLGGLMALGYTIATILKRNSHAFSDDRVCVKIDWLEQLNRQYIQVQGKDRLVVKCVGEQLQLEGSYSPRTYIEQIQLEKLVNEVMALPDDLKTKLSIDEDMVSSPKEALSRFSADSVAMRNKHFKSGMSHSYSTHRDKNLSNHTGFSVSNRKFDDRPTELPAMLANKGAITQLSEQISSLNDRMDEFTSQIEELNSKLTMKKVSASQQNLAFQAEANNGSAPTFFIPGIGNGSLTGTMMHKSSSSSQLAKELPLMEEILGIARSQRQVMHQLDNISSLVREDIGGGRSEGGRRERKSIMLDIEPTRVPLILALAVGGLGIFLFKAFLPRN

SEQ ID NO: 63 - protein, 661 aa, Vitis vinifera
MAQDTSSGADSPRRKSGLLRDQVQLVKRKDSTRYEIVPIQNSLSFEKGFFIVIRACQLLAQNNDGIILVGVAGPSGAGKTVLTEKVLNFMPSIAVISMDNYNDSSRIIDGNFDDPRLTDYGTLLENIHGLKAGKPVQVPIYDFKSSSRIGYRTVDVPSSRIVIIEGIYALSERLRPLLDLRVSVTGGVHFDLVKRVLRDIQRAGQEPEEIIHQISETVYPMYKAFIEPDLQTAHIKIINKFNPFTGFQNPTYILKSTRDLSVDQIKAVLSEEHTETTEETYDIYLLPPGEDPEACQSYLRMRNRDGKYNLMFEEWVTDSPFIISPRITFEVSVRLLGGLMALGYTIAAILKRTSHCFFDDKVCIKTDWLEQLNRRYVQVQGKDRLYVKNVAEQLGLDGSYVPRTYIEHIQLEKLVNDVMALPDDLKTKLSIDDDLASSPKEALSRASADRRMKYLNRDIPHSYSTQRDKNLPKLTRLAVNSRRFDVRTPDSPATVPNQGVITQLSEQISTLNERMDEFTSRIEELNSKFSIRKVSASQQNLAVQADACNGSAPTSLFMAGLGNGSLTGSILPNSSSSSQLAKDSPLMDEILVVVRGQRQIMHQLDNLNSLLHEYWGERSREGRTDRANRMIDIESMGIPIILSLAIGGLGVFFFKSLSSQK

SEQ ID NO: 64 - protein, 662 aa, Eucalyptus grandis
MTQDGSSVESNQRKPGLLKDQVRLVKRKDSGRHEIVSIQDPLSFEKGFFIVIRACQLLAQKNEGIILVGLAGPSGAGKTVFTEKIVNFMPSISVISLDNYNDSSRIVDGNFDDPRITDYETLLRNVEELKAGKPVQVPIYDFKTSSRTGYRTLEVPSSRIVIIEGIYALNEKLRPLLDLRISVTGGVHFDLVKRVLRDIQRAGQEPEEIIHQISETVYPMYKAFIEPDLQTAHIKIINKFNPFTGFQNPTYILKSSRNVAVDQIKAVFSEEHVETSEQTYDIYLLPPGEDPESCQSYLRMRNKDGKYSLMFEEWVTDNPFVISPRITFEVSVRLLGGLMALGYTIATILKRSSHVFSDDRVTVKIDWLEQLNRQYLQVQGRDRLVVQGVAEQLGLEGSYVARTYIEQIQLEKLVNEVMALPEDLKTKLSLDEDLVASPKEALSRASADRVAMRSKHLKSGMSHSYSTQRDKNLSKVAGFAFSNPRYDDRISGASLTLPNQGVISQLSDQISSLHDRMDDFTARIEELNSKLTIKKNSLSQQNMALNPEACNGSAPTSYFISGLGNGSLTGSIMPNSSSSSQLARESPLMEEISTVARGQRQIMHQLDNLSNLLRETLGERSRHLTTSRKSVLDPDRVKIPLICTLAFGGLGILVFKGLSNRN

SEQ ID NO: 65 - protein, 621 aa, Citrus sinensis
MAQDSSNAESHHKKSVLLKDQVRLVKRKDSDRHEIVPIQDPLSFEKGFFVVIRACQLLAQKNDGIILIGLAGPSGAGKTIFTEKILNFMPSIAVISMDNYNDSSRVVDGNFDDPRLTDYDTLLENVRDLREGKPVQVPIYDFESSSRTGYRTVEVPSSRIVIIEGIYALSEKLRPLLDLRVSVTGGVHFDLVKRVLRDIQRVGQEPEEIIQQISETSAKNLSVDQIKAVYPEGHTETMEQTYDIYLLPPGEDPESCQSYLRMRNKDGKYSLMFEEWVTDIPFVISPRITFEVSVRLLGGLMALGYTIATILKRSSHVFCDDRGVCVKIDWLEQLNRQYIQVQGKDRLIVKNVAEQLGLEGSYVPRTYIEQIQLEKLVNEVMALPEDLKTKLSLDEDLVSSPKEALSRASASRVAMRNKNLRSGMSHSYSTQGDKNLSKLTGYVMNDRRFDGRSVESSATLASHGVITHLSEQISSLTDRMDDFTTHIEELNAKLTKKNSSSQQNMALPAEACNGSAPTSFFISSLGNGSLMPNSSSSSQLAKESPLMDEISGIVRGQRQVMHQLDNLSNLLHESMGERSHQGRKTKKSNWPDAEPFKVPLIITLAAGGVGIFLFKKFLTRN

SEQ ID NO: 66 - protein, 658 aa, Theobroma cacao
MAQDASSFESHHKKPGLLKDQVRLVKRKGCDRHEIVPIQDPLSFEKGFFIVIRACQLLAQKNDGIILVGLAGPSGAGKTVFTEKILNFMPSIAIISMDNYNDSSRIVDGNFDDPRLTDYDTLLQNVHDLKQGKEVQVPIYDFKTSSRTGYRTLEVPSSRIVIIEGIYALSEKLRPMMDLRVSVTGGVHFDLVKRVLRDIQRAGQEPEEIIHQISETVYPMYKAFIEPDLQTAHIKIINKFNPFTGFQSPTYILKSARKFTVDQIKSVVSEEHAETEEQTYDIYLLPPGEDPESCQSYLRMRNKDGKYSLMFEEWVTDSPFVISPRITFEVSVRLLGGLMALGYTIAAILKRNSHVFSDDRVCVKIDWLEQLNRQYLQVQGRDRLVVKCVAEQLGLEGSYIPRTYIEQIQLEKLVNEVMALPEDLKTKLSLDEDLVSSPKEALLRASADRVALRNKHLKSGMSHSYSTQREKNIPHFAGYSVNNRRFDERNSDSALAHQGVITQLSDQISSLNDRMDEFTTRVEELNSKLTIKRSSSSQQNLAFQADPCNGSAPTSYFINGLGNGSIMPNSSSSSQLAKDSPLIEEISIVARGQRQIMHQLDNLSNLLHERLGERSQQASTRRKNMVADAEPIKVPLVLTTLVIGGLGIFLYRGFLTRD

SEQ ID NO: 67 - protein, 662 aa, Gossypium raimondii
MPEVAVIFSMAQDASGIETCHKKPGLLKDQVPLVKRKDIDRHEIVSIEDPLSFEQGFFIVIRACQSLAQKNDGIVLVGLAGPSGAGKTVFTEKMLNFMPNIAIITMDNYNDSSRIVDGNFDDPRLTDYDMLLQNVHDLKEGKDVQVPLYDFKTSSRTGYRTLEVPISRIVIIEGIYALSEKLRPMLDLRVSVTGGVHFDLVKRVLRDIQRAGQEPEEIIHQISETVYPMYKAFIEPDLQTAHIKIINKFNPFTGFQSPTYILKSARKFTVDQIKSVVSKAHMETEEQTYDIYLLPPGEDPDSCQSYLRMQNKDGKYSLMFEEWVTDIPFVISPRITFEVSVRLLGGLMALGYTISAILKRNSHVFSDDNVCVKIDWLEQLNRQYLQVQGRDRSVVKDVAERLGLEGSYIPRTYIEQIRLEKLVNEVMALPEDLKTKLSLDEDLVSSPKEALLGASMDMVALRNMHLRREKYISNFAGYSVNNQRFGERNSESALANKGVINQLSEQISSLNDRMDEFKTRVEELNSKLTIKRRTSSQQNLAFRAESCKGSACTSYFINGLGNGSIITNSSSSSQLAKDSPLMEEISTVAEGQRRIMHQLDSLSNLLHERLGERSEQANTKRKYMVAGAEPIKVPLILTTLTIGGLGIFLFRGFLCNSSSQVF

SEQ ID NO: 68 - protein, 658 aa, Gossypium raimondii
MPQDASGLEAQQKRAGLLKDQVRLVKRKDCDRHEIVPIQGPLSFEKGFFIVIRACQLLAQKNDGIILVGLAGPSGAGKTVFTEKILNFMPSIATISMDNYNDSSRIVDGNFDDPRLTDYDTLLQNLHDLKEGKEVQVPIYDFKASSRIGYRTLEVPSSRIVIIEGIYALSEKLRPMLDLRVSVTGGVHFDLVKRVLRDIQRAGQEPEEIIHQISETVYPMYKAFIEPDLQTAQIKIINKFNPFTGFQSPTYILKSARELTVEQIKSAISDEHIETQEQTYDIYLLPPGEDPESCQSYLRMRNKDGKYSLMFEEWVTDIPFVISPRITFEVSVRLLGGLMALGYTIATILKRNSHVFSDDKVCVKIDWLEQLNRQYFQVQGRDRSVVKCVAEKLGLEGSYISRTYIEQIQLEKLVNEVMALPEDLKTKLSLDEDLVSSPKDALLRASVDRVALRNRHLKSGISHSYSTQREKNMSNFAGYNVNNRRFGERNSESALANEGVITQLSEQISSLNDRMDEFTTRIEELNSKLTIKRYTSSQQNLAFQAESCNGSAPTSHFINGLGNGSIMPNSSSSSQLAKDSPIMEEISSVARGQRQIMHQLDNLSNLLREGIGERSQAASTRKKNMMAGGEDSIKVPVILTLAIGGLGIFLYRGILTRH

SEQ ID NO: 69 - protein, 663 aa, Carica papaya
MAQDTNGADLHQKKPVLLKDQVRLVKRKDSDRYEVVPIQDPLSFEKGFFVVIRACQLLAQKNDGIILIGLAGPSGAGKTVFSEKILNFMPSIGLISMDNYNDSSRIVDGNFDDPRLTDYDTLLNNLRDIKSGKQAEIPIYDFKSSSRIGYRTLEVPSSRIVIIEGIYALSEKLRPLLDLRVSVTGGVHFDLVKRVLRDIQRAGQEPEEIIHQISETVYPMYKAFIEPDLKTAQIKIINKFNPFTGFQSPTYILKSARNVSVDQIKAVFCEEHSETDEQTYDIYLLPPGEDPESCQSYLRMRNKEGKYSLMFEEWVTDNPFVISPRITFEVSVRLLGGLMALGYTIATILKRNSHVCSDDRVCVKIDWLEQLNRQYIQVQGKDRSVVQHVAEQLALEGSYIPRTYIEQIQLEKLVNEVMALPDDLKTKLSLDEDLVSSPKEALSRASARRVALRNKNLRSGMSHSYSTERDNNLSKPTGFALKNRRFDESNPESQTNVANQGAITQLSEQISSLNDRMDEFTTRMEELNSKLSFKKNSPSQQNIALQAEACNGSAPTSYFISGLGNGSLTGSIMPNSSSSSQLVKESPLMEEISGIARGQRQIMHQLDNLSNFLRESMGQKSRQERQGRKSIISDVEPIKVPLMITLAIGGISIVLFRGFLTRS

SEQ ID NO: 70 - protein, 674 aa, Thellungiella halophila
MGQDTNGIEFHQKRHGLLKDQVQLVKRRDSVRYEIVPIQDRLSFEKGFFAVIRACQLLSQKNDGIILVGVAGPSGAGKTVFTEKILNFLPSVAVISMDNYNDASRIVDGNFDDPRLTDYDTLLKNLEDLKEGKQVEVPIYDFKSSSRVGYRTLDVPASRIVIIEGIYALSERLRPLLDLRVSVTGGVHFDLVKRVLRDIQRAGQQPEEIIHQISETVYPMYKAFIEPDLQTAQIKIINKFNPFTGFQSPTYILKSRKDVSVDQIKAVLSEGHTETKEETYDIYLLPPGEDPESCQSYLRMRNKDGKYSLMFEEWVTDTPFVISPRITFEVSVRLLGGLMALGYTIATILKRNSHVFATDKVFVKIDWLEQLNRHYMQVQGKDRQLVQSTAEQLGLEGSFIPRTYIEQIQLEKLINEVMALPEDLKNKLSLDEDLVSSSSPKEALLRASADRVAMRNKNLKRGMSHSYSTQRDKNLSKLADYSSSDRRYEERNHDSPANEGFMTQLSEQISSLNERMDEFTSRIEELNSKLSCNKNSPTQQSMTLQAEVCNGSAPTSYFISGLDNGCLTNSIMPHSSSSSQLAKDSPLMEEISTISRGQRQVMHQLDNLCNLMRESSAERTRLARAGSSNSSSRGRSSKSFFLSNAESKSIPLVLTLAFCSIGIVVIKSYINKRQ

SEQ ID NO: 71 - protein, 675 aa, Brassica rapa
MGQDTNGIEFHQKRHGLLKDQVQLVKRRDSVRYEIVPIQDRLSFEKGFFAVIRACQLLSQKNDGIILVGVAGPSGAGKTVFTEKILNFLPSVAVISMDNYNDASRIVDGNFDDPRLTDYDTLLKNLEDLKEGKQVEVPIYDFKSSSRVGYRTLDVPASRIVIIEGIYALSEKLRPLLDLRVSVTGGVHFDLVKRVLRDIQRAGQQPEEIIHQISETVYPMYKAFIEPDLQTAQIKIINKFNPFTGFQSPTYILKSRKDVSVDQIKAVLSEGYTENKEETYDIYLLPPGEDPESCQSHLRMRNKDGKYSLMFEEWVTDTPFVISPRITFEVSVRLLGGLMALGYTIATILKRNSHVFATEKVCVKIDWLEQLNRHYMQVQGKDRQLVQSTAEQLGLEGSFIPRTYIEQIQLEKLINEVMALPDDLKHKLSLDEDLVSSSSPKEALLRASADRVAMRNKNLKRGMSHSYSTQRDKNLSKLAGYSSSDRRYEERNHDSPANEGFMTQLSEQISSLNERMDEFTNRIEELNSKLSCNKNSPTQQSMTVQAEVCNGSAPTSYFISSLDNGCLTNSIMPHSSSSSQLAKDSPLMEEISTLSRGQRQVMHQLDNLCTLMRESSSAERSRLARTGSNKRSGSSKSFFLSNAESNSLLPLKLTALAALCSVGIVVIKSYINKRQ

SEQ ID NO: 72 - protein, 635 aa, Brassica rapa
MTGQDINGIEFHQQRHGLLKDQVQLVKRRDSVRYEIVPIQDRLSFEKGFFAVIRACQLLSQKNDGIVLVGVAGPSGAGKTVFTEKILNFLPSVAVISMDNYNDASRIVDGNFDDPRLTDYDTLLKNLEDLKEGKQVEVPVYDFKSSSRVGYRTLDVPASRIVIIEGIYALSEKLRPLLDLRVSVTGGVHFDLVKRVLRDIQRAGQQPEEIIHQISETVYPMYKAFIEPDLQTAQIKIINKFNPFTGFQSPTYILKSRKDVSVDQIKAVLSEGHTETKEETYDIYLLPPGEDPESCQSYLRMRNKDGKYSLMFEEWVTDTPFVISPRITFEVSVRLLGGLMALGYTIATILKRNSHVFATDKVIVKIDWLEQLNRHYLQVQGKDRQVVQSTAEQLGLEGSFIPRTYIEQIQLEKLINEVMALPDDLKNKLSLDEDLVSSSSPKEALLRASADRVSMRNKNLRGMSQSYSTQRDKNISKLAGYSSSDRRHEERNHDSPANEGFMTQLSEQISSLNERMDEFTNLIEELNSKLSCNKNPPTQQSIEVCNGSAPTSYFISGLDNGCLTNAIMPHSSSSSQEDSAERSRLARTGSSNSSRSSKSFFLSSVESSSLPLVLTTLALCSVGVVVIKSYINNRQ

SEQ ID NO: 73 - protein, 674 aa, Capsella rubella
MGQDSNGIEFHQKRHGLLKDQVQLVKRRDSVRYEIVPIQDRLSFEKGFFAVIRACQLLSQKNDGIILVGVAGPSGAGKTVFTEKILNFLPSVAVISMDNYNDSSRIVDKNFDDPRLTDYDTLLKNLEDLKEGKQVEVPIYDFKSSSRVGYRTLDVPASRIVIIEGIYALSEKLRPLLDLRVSVTGGVHFDLVKRVLRDIQRAGQQPEEIIHQISETVYPMYKAFIEPDLQTAQIKIINKFNPFTGFQSPTYILKSRKDVSIDQIKEVLSDGHTETKEETYDIYLLPPGEDPESCQSYLRMRNKDGKYSLMFEEWVTDTPFVISPRITFEVSVRLLGGLMALGYTIATILKRNSHVFATDKVFVKIDWLEQLNRHYMQVQGKDRQLVQSTAQQLGLEGSFIPRTYIEQIQLEKLINEVMALPDDLKHKLSLDEDLVSSSSPKEALLRASADRVAMRNKNLKRGMSHSYSTQRDKNMSKLAGYSSSDRRYEERNHDSPANEGFMTQLSEQISSLNERMDEFTSRIEELNSKLSCNKNSPTQQSLSIQTEVCNGSAPTSYFISGLDNGCLTNSIMPHSSSSSQLAKESPLMEEISTISRGQRQVMHQLDNLCNLMRESSAERSRLARTGSSNSSNRGRSSKSFFLSNVESKRLPLVLTLAFCSVGFIVIKSYINKRQ

SEQ ID NO: 74 - protein, 664 aa, Glycine max
MAKDSADADSHHRRLGLLKDQVHLVKRKGSDRYEIAPIQDQLSFEKGFFIVIRACQLLSQKNDGIILVGVAGPSGAGKTIFTEKILNFMPSIAIISMDNYNDASRIVDGNFDDPRLTDYDTLLQNLHNLKEGKPVQVPIYDFKSSSRTGYRTVEAPSSRIVIIEGIYALSEKLRPLMDLRVSVTGGVHLDLVKRVIRDIQRAGQEPEEIIHQISETVYPMYKAFIEPDLQTAHIKIINKFNPFTGFQSPTYILKSGRNVEVDKIKAVLAEDFKETTEQTYDIYLLPPGEDPETCQSYLRMRNKDGKYSLMFEEWVTDNPFVISPRITFEVSVRLLGGLMALGYTIATILKRNSHVFSDDRVCVKLDWLEQLNRHYVQVQGRDRLVLKYIGEQLGIEGSYIPRTYIEQIQIEKLVNEVMALPDDLKTRLSLDEDLVSSPKEALSRASAGRVAMRNKHLRSGISQSYTNQRDKNLAKVTGYGANNRRFGESNSDSTAMPVNQGTINQLSDQISALNDRMDEFTNRIEELNSKLTIKKNSPSQQNMSLQAETCNGSAPTSYFITSLGSGSLTGSKMTNSSSSSQLAKDSPLMDEISSIARSQRQVMHQLDNLNNLLRGSLGEKSRPTRTNSGKSITMSSDSMGACVMAVVTVGCLGIFLMKGFLNKK

SEQ ID NO: 75 - protein, 664 aa, Glycine max
MAKDSADADSHHRRLGLLKDQVHLVKRKGFDRYEIAPIQDQLAFEKGFFIVIRACQLLSQKNEGIILVGVAGPSGAGKTVFTEKILNFMPSIAVISMDNYNDASRIVDGNFDDPRLTDYDTLLQNLHDLKEGKPVQVPIYDFKSSSRTGYRTVEVPSSRIVIIEGIYALSEKLRPLLDLRVSVTGGVHLDLVKRVIRDIQRAGQEPEEIIHQISETVYPMYKAFIEPDLQTAHIKIINKFNPFTGFQSPTYILKSGRNVEVDKIKAVLAEDFKETTEQTYDIYLLPPGEDPETCQSYLRMRNKDGKYSLMFEEWVTDNPFVISPRITFEVSVRLLGGLMALGYTIATILKRNSHVFSDDRVCVKLDWLEQLNRHYVQVQGRDRLVLKYIGEQLGLEGSYIPRTYIEQIQIEKLVNEVMALPDDLKTKLSLDEDLVSSPKEALSRASADRVAMRNKHLRSGISQSYTNQRDKNLAKVTGYGANNGRFGEKNLDSTAMPVNQGAINQLSDQISALNDRMDEFTNRIEELNSKLTIKKSSPSQQNMSLQAETCNGSAPTSHFITSLGNGSLTGSKMTNSSSSSQLAKESPLMDEISSIGRSQRQIMHQLDNLNNLLRGSLGEKSHPTRTNSRKSIDMSSDSVGARVMVVAAVGCLGIFLMKVLLNKK

SEQ ID NO: 76 - protein, 662 aa, Phaseolus vulgaris
MGKNTSESDSHHKRLGLLKDQVHLVKRKDSDRHEIAPIQDQLSFEKGFFIVIRACQLLAQKNDGIVLVGVAGPSGAGKTVFTEKILNFMPSIAVISMDNYNDSSRIVDGNFDDPRLTDYDTLLQNLGDLKEGKPVQVPIYDFKSSTRTGYRTVEAPTSRIVFIEGIYALSEKLRPLLDLRVSVTGGVHLDLVKRVIRDIQRAGQEPEEIIHQISETVYPMYKAFIEPDLQTAHIKIINKFNPFTGFQSPTYILKSAKNLAVDQIKAVLSEDFKETTEQTYDIYLLPPGEDPETCQSYLRMRNKDGKYSLMFEEWVTDNPFVISPRITFEVSVRLLGGLMALGYTIATILKRNSHVFSDDRVCVKLDWLEQLNRHYVQVQGRDRLVLKYIGQQLGLDGSYIPRTYIEQIQIEKLVDEVMALPDDLKTKLSLDEDLVSSPKEALSRASADRVAMRNKHLRSGISQSYTNQRDKNLAKVGFDGNNRRFGERNSESSTMSVNQGAINQLSDQISALNDRMDEFTNRIEELNSKLNIKRNSPTQQNMSLQAETCNGSAPTSYFITGLGNGSLTGSKMANSSSSSQLTKDFPLMDEISSIARSQRQIMHQLDNLNNLLRGNSGEKSQQTRTNRRSIDTASDSMGTSVMAVVAVGCLGIFLMKGLLNRN

SEQ ID NO: 77 - protein, 659 aa, Populus trichocarpa
MAKDTSGAESHPKRQGLLKDQARLTKKKDCDRFEIVPIQNPLSFEKGFFIVIRACQLLAQNNDGMILVGIAGPSGAGKTIFTEKILNFMPSVATISMDNYNDSSRIVDGNFDDPRLTDYDMLLKNVLDLKDGKPVEVPIYDFKSSTRTGYRTLEVPSSRIVIIEGIYALSEKLRPLLDLRVSVTGGVHFDLVKRVLRDIQRAGQEPEEIIQQISETVYPMYKAFIEPDLKTAHIKITNKFNPFSGFQSPTYILKSARKVTVDQIKPVLSEDYKETMEQIYDIYLLPPGEDPESCQSYLRMRNKDGKYNLMFEEWVADAPFIISPRITFEVTVKLLSGLMALGYTIAAILQRSSHSFSDDRVCVKIDWLGQLNRQYVQVQGRDRLVVKYIAEQLGLEGSYTPRTYIEQIQLERLVNEVMALPDDLKTKLSLDEDLVSSPKEALLQASADRVARRFKNGKSGMSHSYSSQRDKNLSKLTGPAATSNRFDDSNLESPAALANQGAMTQLSEQISSLNDRMDEFTTCIEELNSKLIINKNPPSQQNMALQAEVHNGSAPTSYFVSGLGNGSLTGSRMSNSSSSSLLAKESPLMEEISGIARAQRQVTLQLDTLSNLLHDSLGERFQGVRKNRNILAVRDGQAPLIVALAIGCVGLCWFIRAQN

SEQ ID NO: 78 - protein, 661 aa, Populus trichocarpa
MAKDTSEAESHPKRLGLLKDQVRSTKKKDSDRFEIVPIQNPLSFEKGFFVVIRACQLLAQKNDGIILVGIAGPSGAGKTIFTEKILNFLPSVAVISMDNYNDSSRIVDGNFDDPRLTDYDTLLKNVHDLKAGKPVEVPIYDFKSSTRTGYRTLEVPSSRIVIIEGIYALSANLRPLLDLRVSVTGGVHFDLVKRVLRDIQRAGQEPEEIIQQISETVYPMYKAFIEPDLQTAHIKIINKFNPFSGFQSPTYILKSARKVTVDQIKAVLSEDQKETMEQIYDIYLLPPGEDPETCQSYLRMRNKDGKYNLMFEEWVADVPFIISPRITFEVTVKLLSGLMALGYTIAAILQRSSHIFSDDRVCVKIDWLGQLNRQYVQVQGRDRLVVKFIAEQLGLEGSYTPRTYIEQIQLERLVNEVMALPDDLKTKLSLDEDLVSSPKEALLRASADRVARRLKNGKSGMSHSYSSQRDKNLSKLTGLAATSKRFDDRNLESPAALANQGAITQLSEQISSLNDRMDEFTTCIEELNSKLTIKKNSPSQQNMALQAEVCNGSAPASYFVSGLGNASLNGSRMFNSSSSSQLAKESPLMEELSGIARVQRQVMLRLDTLSNLVRDSLGERSQEVRKNRNRLIVRDGQAPLIAALAVGCVGLCWFVRARNGL

SEQ ID NO: 79 - protein, 696 aa, Linum usitatissimum
MGEDASGSESHPKKQGLLKDQVRLAKRKDSDRYKIVPIQESLSFEKGFFVVIRACQLLAQKNDGIILVGLAGPSGAGKTMFTEKIINFMPSVAVISMDNYNDASRIVDGNFDDPRLTDYDTLLKNVQDLKAGKSAEVPVYDFKSSSRTGYRTVEVPESRIVIIEGIYALSEKLRPLLDLRISVRGGIHFDLVKRVLRDIQRAGQEPEEIIHQISETVYPMYKAFIEPDLQTAHIKIINKFNPFTGFQNPTYILKVGLVGDIILFGSLYPFAILTLLMLLWLLDDFFDWPSARNVAVDKIKAVLSEGHTETEEQTYDIYLLPPGEDPESCQSYLRMRNKEGKYNLMFEEWVTDAPFVISPRITFEVSVRLLGGLMALGYTIATILKRSSHVFFNDKVCVKVDWLEQLNRQYIQVQGRDRLAVRCVAEQLGLEGSYIPRTYIEQIQLEKLVDEVMALPDDLKNKLSLDEDLVSSPREALLRASADRVAMRNKNLSGMSHSYSTQGDKSYSKLAAFSNGNRRYEDRNSEPQAMLANQGVITQLSEQMTSLNDRMDDFTSQLEELNTKLTVMNNSPRQQNIGAQGESSNGSAPTSYFVSGLSNGSLTGSKQQLPHSSSFSQLAKDSPLLEEIVRGQRQIMHKLDSFTSLLHEATAERSKQARKGNKLTVADMNTIGVPLVLSMAIGGLGLLLFRSFRNSR

SEQ ID NO: 80 - protein, 661 aa, Linum usitatissimum
MGEDASGSESHPKKQGLLKDQVRLAKRKDSDRYKIVPIQESLSFEKGFFVVIRACQLLAQKNDGIILVGLAGPSGAGKTMFTEKIINFMPSVAVISMDNYNDASRIVDGNFDDPRLTDYDTLLKNVQDLKAGKSAEVPVYDFKSSSRTGYRTVEVPESRIVIIEGIYALSEKLRPLLDLRISVRGGIHFDLVKRVLRDIQRAGQEPEEIIHQISETVYPMYKAFIEPDLQTAHIKIINKFNPFTGFQNPTYILKSARNVAVDKIKAVLSEGHTETEEQTYDIYLLPPGEDPESCQSYLRMRNKEGKYNLMFEEWVTDAPFVISPRITFEVSVRLLGGLMALGYTIATILKRSSHVFFNDKVCVKVDWLEQLNRQYIQVQGRDRLAVRYVAEQLGLEGSYIPRTYIEQIQLEKLVDEVMALPDDLKNKLSLDDDLVSSPREALLRASADRVAMRNKHLSGMSHSYSTQGDKSYSKLAAFSNGNRRYEDRNSEPQAMLANQGVMTQLSEQMTSLNDRMDDFTSQLEELNTKLTVMNNSPRQQNIGGQGESSNGSAPTSYFMSGLSNCSLTGSKQQLPHSSSFSQLAKDSPLLEEIVRGQRQIMHKLDSFTSLLHEATAERSKQARKGNKLTVADMNTIGVPLVLSMAIGGLGLLLFRSFHNSG

SEQ ID NO: 81 - protein, 657 aa, Ricinus communis
MAQGMSGIELHQKKQGLLKDQVRLVKRKDCDRYEIVPIQQTYTFEKGFFLFIRACQLLAQKNDGIILVGLAGPSGAGKTVFTEKVLHFMPSVAVISMDNYNDSSRIVDGNFDDPRLTDYDTLLKNVQDLKSGKAVEVPIYDFKSSSRIGYRTLEVPTSRIVIIEGIYALSEKLRPMLDLRVSVTGGVHFDLVKRVLRDIQRAGQAPEEIIQQISETVYPMYKAFIEPDLQTAHIKIINKFNPFSGFQSPTYILKSAKKVKVDQIKAVLSEEHTEATEQTYDIYLLPPGEDPESCQSYLRMRNKDGKYNLMFEEWVTDNPFVISPRITFEVSVRLLGGLMALGYTIATILKRSSHVFFNDRVCVKIDWLEQLNRQYIQVQGRDRLVVRCVAEQLGLEGSYVPRTYIEQIQLEKLVNEVMALPDDLKTKLSLDEDLVSSPKEALLRASADRVAMRNKNLKSGMSHSYSSQRDKNLSKLAGLAASDRRYNERNSDSSAVQANEGMLTQLSEQISSLNDRMDEFTNRMEELNSKLNNKSSPSQQNLALQAEACNGTAPTSYFLSGLSNGSLTGPKLSNSSSSTQLAKESPLLEEITGIMRGQRQVMHQLDTLNNLLRENAGERSRQVRTNRRSMIADSDITKIALVLSIGVIGFSMFRRIF

SEQ ID NO: 82 - protein, 659 aa, Manihot esculenta
MAQDNSSAELQKKRHGLLKDQVRLSKRKDCGRYEIVPIQQTYTFEKGFFLFIRACQLLAQNNDGIILVGLAGPSGAGKTVFTEKVLNFMPSVAVISMDNYNDSSRIIDGNFDDPRLTDYDTLLKNIHDLKAGKSAETPIYDFKSSSRVGYRMVEVPTSRIVIIEGIYALSTKLRPMLDLQVSVTGGVHFDLVKRVLRDIQRAGQAPEEIIQQISETVYPMYKAFIEPDLQTAHIKIINKFNPFSGFQNPTYVLKSAKKVMVDQIKAVLSEEHTEMTEQTYDIFLLPPGEDPESCQSYLRMRNKDGKYNLMFEEWVTDAPFVISPRISFEVSVRLLGGLMALGYTIATILKRSSHVFLNDNVCVKIDWLEQLNRQYVQVQGKDRLVVRYVAEQLGLEGSYVPRTYIEQIQLEKLVDDVMALPDDLKTKLSLDEDLVSSPKEALLRASADRVAMRNKNLKSGMSHSYSNQRESNFSKLAVLAASNGGYTERNTVSMAVLANQGILTQLSEQISSLNDRMDEFAARIEDLNSKLNITSSSSSQQNLVLQADACNGSAPTSHFLSGLSNGSLTGCKLRNSSSFSQLAKESPLMEEVSGIARGQRQVMHQLDTLSNILRESLGQRSQQVRTNRRRSMIADLEITKLALILSVGVIGFSTLRRIF

SEQ ID NO: 83 - protein, 659 aa, Manihot esculenta
MDRDNSSAELHQKRHGLLKDQVRLVKRKDCDRYEIVPIQQTYTFEKGFFLFIRACQLLAQNNDGIILIGLAGPSGAGKTVFTEKVLNFMPSVAVISMDNYNDSSRIVDGNFDDPRLTDYDTLLKNVHDLKAGKSAEVPIYDFKSSSRIGYRTVEVPTSRIVIIEGIYALSEKLRPLLDLRVSVTGGVHFDLVKRVLRDIHRAGQAPEEIIQQISETVYPMYKAFIEPDLQTAHIKIINKFNPFSGFQSPTYILKSAKKVKVDQIKAALSEDHTETTEETYDIYLLPPGEDPESCQSYLRMRNKDGKYNLMFEEWVTDAPFVISPRITFEVSVRLLGGLMALGYTIATILKRSSHVFINDRVCVKIDWLEQLNRQYVQVQGRDRLVVRCVAEQLGLEGSYVPRTYIEQIQLEKLVNEVMALPDDLKSKLSLDEDLVFSPKDALLPASADRVAMRNKNLKSGMAHSYSSQRDKNLSSLAGLAANNRGYSERNRESKALLANQGILTQLSEQISSLNDRMDEFTTRIEELNSKLNINENSSGQQKLGLQPEACNGYASSMSYFTSGLSNGSLTGSKMHNSSSSSQLAKESQLMEEISGIVRGQRQVMHQLDTLSNLLRESLGQRSEQVRRGRRSMIPDIEITKIAVILSVGVLGFSMLRRIF

(19) United States
(12) Patent Application Publication
(10) Pub. No.: US 2017/0016019 A1
(43) Pub. Date: Jan. 19, 2017

(54) Plants Having Enhanced Pathogen Resistance and Methods of Modulating Pathogen Resistance in Plants

(71) Applicant: The Governing Council of the University of Toronto, Toronto (CA)

(72) Inventors: Keiko Yoshioka, Toronto (CA); Wolfgang Alexander Moeder, Toronto (CA); Chung Huoi Ung, Newmarket (CA)

(21) Appl. No.: 15/124,911

(22) PCT Filed: Mar. 12, 2015
(86) PCT No.: PCT/CA2015/050186

§ 371 Date: Sep. 9, 2016

Related U.S. Application Data
(60) Provisional application No. 61/951,577, filed on Mar. 12, 2014.

(51) Int. Cl. C12N 15/82; C12N 9/14

(52) U.S. Cl. CPC C12N 15/8279; C12N 9/14; C12Y 306/01

(57) ABSTRACT

The present invention relates to methods of modifying pathogen resistance in plants and plants having modified pathogen resistance. In particular, the present invention relates to modification of expression or activity of a negative regulator of plant immunity.

### FIELD OF THE INVENTION

The present invention pertains to the field of plant biology and pathogen resistance. In particular, the present invention relates to methods of modifying pathogen resistance in plants, plants having modified pathogen resistance and methods of modulating pathogen resistance and methods of screening for members of a plant population having modified pathogen resistance.

### BACKGROUND OF THE INVENTION

Plants have evolved a large number of defence systems to protect themselves against pathogen invasion. The first line of defence is basal immunity, which is triggered by the recognition of molecules that are conserved among many pathogens (pathogen-associated molecular pattern-PAMPs) and is thus referred to as PTI (PAMP-triggered immunity). One well studied PAMP is the flg22 peptide derived from the bacterial flagellin (Felix and Boller, 2003).

Pathogens, in turn, have evolved effector molecules that can block PTI (Jones and Dangl, 2006; Bent and Mackey, 2007). Plants have evolved a second, stronger response to pathogen infection, which is mediated by resistance (R) genes that can recognize either specific effectors from the pathogen directly or indirectly. This is also known as effector-triggered immunity (ETI; Bent and Mackey, 2007). The hypersensitive response (HR), which is characterized by apoptosis-like cell death at and around the site of pathogen entry is one common defence mechanism activated by R gene-mediated pathogen recognition (Hammond-Kosack and Jones, 1996; Heath, 2000). During HR development an increase in salicylic acid (SA) and the accumulation of pathogenesis-related (PR) proteins is observed (Vlot et al., 2008). Later, enhanced resistance with slightly elevated SA levels and PR gene expression can also be induced in uninfected leaves. This phenomenon is called systemic acquired resistance (SAR) and confers a long-lasting, broad-spectrum resistance to subsequent infection (Durrant and Dong, 2004; Vlot et al., 2008). SAR can also be triggered by exogenous treatment with SA or synthetic SA analogs, such as benzothiadiazole (BTH; Lawton et al., 1996).

Many components in the pathogen resistance signal transduction pathway have been identified through screens for mutants with altered susceptibility to pathogens. Isochorismate synthase1 (ICS1) is critical for the biosynthesis of pathogen-induced SA. sid2/ics1 mutants fail to produce elevated levels of SA after pathogen infection and are thus hypersusceptible to certain pathogens (Wildermuth et al., 2001). NPR1 (non expressor of PR genes1) is a key regulator of SA-mediated resistance and npr1 mutant plants fail to respond to exogenously supplied SA (Durrant and Dong, 2004). The lipase-like proteins, enhanced disease susceptibility1 (EDS1) and phytoalexin-deficient4 (PAD4) (Century et al., 1995; Glazebrook et al., 1996), participate in both basal and R protein-mediated defence responses (Falk et al., 1999; Jirage et al., 1999). EDS1 interacts with PAD4 and SAG101 (senescence associated gene101) and the combined activities of EDS1 and PAD4 are required for both HR formation and the restriction of pathogen growth (Feys et al., 2001; 2005). A second class of mutants exhibits heightened resistance, usually accompanied by elevated levels of SA and PR genes (Moeder and Yoshioka, 2008). These mutants frequently also spontaneously develop HR-like lesions and belong to autoimmune mutants (Hofius et al., 2009).

Given the economic impact of pathogen infection of agriculturally important crops, there is a need for plants having increased pathogen resistance, methods of enhancing a plant's immunity to pathogens and methods for screen populations of plants for plants exhibiting enhanced pathogen resistance.

### SUMMARY OF THE INVENTION

An object of the present invention is to provide plants having enhanced pathogen resistance and methods of modulating pathogen resistance in plants. In accordance with an aspect of the present invention, there is provided a nucleic acid encoding a negative regulator of plant immunity and comprising a sequence 50%, 60%, 65%, 70%, 75%, 80%, 85%, 90%, 95%, 98% or 100% identical to the sequence as set forth in as set forth in any one of SEQ ID NOs:1 to 41.

In accordance with another aspect of the present invention, there is provided a polypeptide which is a negative regulator of plant immunity and comprising a sequence 50%, 60%, 65%, 70%, 75%, 80%, 85%, 90%, 95%, 98% or 100% identical to the sequence as set forth in any one of SEQ ID NOs:42 to 83.

In accordance with another aspect of the present invention, there is provided a plant (and cells thereof) exhibiting enhanced pathogen resistance and having decreased expression or activity of TTM2, TTM2 homologs or TTM2 orthologs.

In accordance with another aspect of the present invention, there is provided a method of modulating pathogen resistance in a plant comprising modulating expression or activity of TTM2, TTM2 homologs or TTM2 orthologs.

In accordance with another aspect of the present invention, there is provided a method of enhancing pathogen resistance in a plant comprising inhibiting expression or activity of TTM2, TTM2 homologs or TTM2 orthologs.

### BRIEF DESCRIPTION OF THE FIGURES

FIG. 1 illustrates that AtTTM2 is down-regulated after pathogen infection. (A) Quantitative real-time PCR analysis of AtTTM2 expression in Hyaloperonospora arabidopsidis, isolate Emwa1-infected (Emwa1) or water-treated (H₂O) cotyledons of 10-day-old Col wild type plants 7 days after infection. (B) Quantitative real-time PCR analysis of AtTTM2 expression in uninfected true leaves of the same plants. Transcripts were normalized to AtEF1A. Each bar represents the mean of three independent experiments±SE. Each sample is a mix of 16 seedlings. Asterisks indicate statistical significance (Student's t-test, p<0.001 (**), p<0.05 (*)).

FIG. 2 illustrates that ttm2 exhibits enhanced resistance against Hyaloperonospora arabidopsidis (Hpa). (A) Infection phenotype of Col wild type (Col) and ttm2 mutant plants 10 days after infection with avirulent Hpa, isolate Emwa1. Shown is trypan blue staining of infected cotyledons (Cot) and uninfected true leaves (TL) revealing some hyphae in wild type (white arrows, Hy) and enhanced hypersensitive response (HR) cell death in the ttm2 mutant lines (red arrows). Uninfected true leaves (TL) also displayed enhanced HR-like cell death (red arrows). (B) Quantification of Hpa, isolate Emwa1, infection by quantitative real-time PCR of the oomycete marker, internal transcribed spacer2 (ITS2). Transcripts were normalized to AtEF1A. Each bar represents the mean of three technical replicates±SE. Each sample is a mix of 16 seedlings. Data from an independent experiment with the same result is shown in FIG. 12A. (C) Infection phenotype of Col wild type and ttm2 mutant plants 12 days after infection with virulent Hpa, isolate Emco5. Shown is trypan blue staining of infected cotyledons (Cot) and uninfected true leaves (TL) revealing hyphae (Hy) and oospores (Oo) in wild type (white arrows) and reduced hyphal growth in the ttm2 mutant lines. Uninfected true leaves (TL) of ttm2 mutants also displayed some HR-like cell death along veins (red arrow). (D) Quantification of Hpa, isolate Emco5, infection by quantitative real-time PCR of the oomycete marker, ITS2. Transcripts were normalized to AtEF1A. Each bar represents the mean of three technical replicates±SE. Each sample is a mix of 16 seedlings. Data from an independent experiment with the same result is shown in FIG. 12B. (E-F) Free salicylic acid (SA; E) and conjugated salicylic acid (SAG; F) levels in Hpa, isolate Emwa1-infected cotyledons 5 days after infection. Each bar represents the mean of three biological replicates±SE. Experiments were repeated three times with similar results. Bars=250 μm. Asterisks indicate a significant difference (Student's t-test, p<0.05). 10 day old seedlings were used for all infections.

FIG. 3 illustrates that ttm2 exhibits enhanced Systemic Acquired Resistance (SAR). (A) Primary infection of 10 day-old cotyledons of Col wild type and ttm2 mutant plants was performed with the avirulent Hpa isolate, Emwa1 (SAR +) or water (SAR −). After 7 days a challenge infection was performed on systemic true leaves with Hpa, Noco2 (virulent). Hyphal structures were visualized 10 days later by trypan blue staining. (B) Stained leaves were microscopically examined and assigned to different classes (see panels). Data shown is from two independent experiments and was taken from 50 plants each; Fisher Exact Probability Test indicates a significant difference between SAR+ ttm2 lines and Col (P<0.0001). The experiment was repeated three times with similar results. Data from an independent experiment with a similar result is shown in FIG. 14. Bars=250 μm

FIG. 4 illustrates that involvement of PAD4, NPR1, and SA in ttm2-mediated resistance.

Infection phenotype of Col wild type (Col), Ws wild type (Ws), pad4-1, sid2-1, npr1-1 and ttm2 mutants and corresponding double mutants 10 days after infection with avirulent Hpa, isolate Emwa1. Shown is trypan blue staining of infected cotyledons (Cot) and uninfected true leaves (TL). White arrows indicate hyphal growth, red arrows indicate HR cell death. Bars=250 μm. Hy=Hyphae, Oo=Oospores, HR=Hypersensitive Response. Experiments were repeated three times with similar results. 10-day-old seedlings were used for infection.

FIG. 5 illustrates that AtTTM2 expression is suppressed by SA and flg22 treatment. Quantitative real-time PCR analysis of Col wild type plants (A) 24h after treatment with 100 μM salicylic acid (SA) or water (H₂O). (B) 48h after treatment with 200 μM benzothiadiazole (BTH) or water. Shown is AtTTM2 and PR1 gene expression relative to AtEF1A. (C) Quantitative real-time PCR analysis of AtTTM2 in Col wild type (Col), sid2, pad4 and npr1 plants 4h after treatment with flg22 or water. Transcripts were normalized to AtEF1A. Each bar represents the mean of three technical replicates±SE. Each sample is a mix of 16 seedlings (A, B) or 4 leaves (C). Data from an independent experiment with the same result is shown in FIG. 16. For A and B 10-day old seedlings were used; for C 4-week old-plants were syringe-infiltrated.

FIG. 6 illustrates that overexpression of AtTTM2 causes enhanced susceptibility. (A) Quantitative real-time PCR analysis of AtTTM2 in Hpa-infected cotyledons 10 days after infection. Transcripts were normalized to AtEF1A. Each bar represents the mean of three technical replicates±SE. Each sample is a mix of 15 seedlings. Data from an independent experiment is shown in FIG. 18. (B) Trypan blue staining of Col wild type (Col), ttm2 and two independent 35S:AtTTM2 over-expressor lines (35S-2, -5) 13 days after infection with Hpa, Emco5. Bars=250 μm. (C) Quantitative assessment of infection. Stained leaves were microscopically examined and assigned to different classes (see panels). Data shown was taken from 15-16 plants; Fisher Exact Probability Test indicates a significant difference between over-expressor lines and Col (p<0.001), the experiment was repeated three times with similar results. (D) Quantitative real-time PCR analysis of ITS2 in Hpa-infected cotyledons 10 days after infection. Transcripts were normalized to AtEF1A. Each bar represents the mean of three technical replicates±SE. Each sample is a mix of 15 seedlings. Data from an independent experiment is shown in FIG. 18. The analysis of a third independent line is shown in FIG. 18B, C. 10 day old seedlings were used for all infections.

FIG. 7 illustrates that TTM2 function is conserved in crop species. Quantitative real-time PCR analysis of canola (Brassica napus var. Westar (A) and soybean (Glycine max var. Harasoy (B) plants treated with 200 μM BTH or water (H₂O) 48hrs after treatment. (A) Quantitative real-time PCR analysis of canola BnTTM2a, BnTTM2b and BnPR1. Transcripts were normalized to BnUBC21. (B) Quantitative real-time PCR analysis of soybean GmTTM2a/GmTTM2b and BnPR1. Transcripts were normalized to GmEF1B (Note: primers could not distinguish between the two soybean paralogues due to high sequence homology). Each bar represents the mean of three technical replicates±SE. Data from an independent experiment with the same result is shown in FIG. 19. 3-4 week old plants were used for treatments.

FIG. 8 illustrates that AtTTM2 displays pyrophosphatase activity. Substrate specificity of AtTTM2 was tested with 0.5 mM PPᵢ, ATP or PPᵢ. Reactions were performed at pH 9.0 in the presence of 2.5 mM Mg²⁺. 2 μg of protein was used. Black columns: GST-TTM2, white columns: GST. Each bar represents the mean of three replicates±SE. Experiments were repeated more than three times with similar results.

FIG. 9 illustrates a model showing that AtTTM2 is a negative regulator of the SA-mediated defence amplification loop. Recognition of pathogens suppresses the transcription of AtTTM2 to amplify defence responses. At a later time point, production of SA further leads to continuous transcriptional suppression of AtTTM2, further amplifying the feedback loop. The knockout mutants of AtTTM2, thus, behave like in a “primed” state and show enhanced resistance upon pathogen recognition. The mutant phenotype requires the known defence signalling components ICS1, PAD4 and NPR1.

FIG. 10 illustrates a visualization of the expression pattern of AtTTM2. Data is based on publicly available AtGenExpress data at the Botany Array Resource (Winter et al., 2007). Shown are relative gene expression values after treatment with PAMPs (flg22, HrpZ) or bacterial pathogens (virulent Pseudomonas syringae pv. tomato DC3000, avirulent Pseudomonas syringae pv. tomato DC3000 AvrRpm1, and Pseudomonas syringae pv. phaseolicola).

FIG. 11 illustrates T-DNA insertion line analysis. (A) T-DNA insertion position in ttm2-1 (SALK_145897) and ttm2-2 (SALK_114669). Number in the triangle indicates the exact location of the T-DNA insertion. Filled boxes represent exons, grey represents untranslated regions and lines represent introns. (B) RT-PCR analysis for AtTTM2 in Col wild type, ttm2-1 and ttm2-2, respectively. β-tubulin was used as a loading control. Primer sequences are listed in FIG. 22. (C) Morphological phenotype of Col wild type, ttm2-1 and ttm2-2. Photos show approximately 6 week-old plants. Scale bar=1 cm.

FIG. 12 illustrates that ttm2 exhibits enhanced pathogen resistance. (A) Quantification of Hpa, isolate Emwa1, infection by quantitative real-time PCR of the oomycete marker, internal transcribed spacer2 (ITS2). Transcripts were normalized to AtEF1A. Each bar represents the mean of three technical replicates±SE. Each sample is a mix of 16 seedlings. (B) Quantification of Hpa, isolate Emco5, infection by quantitative real-time PCR of the oomycete marker, ITS2. Transcripts were normalized to AtEF1A. Each bar represents the mean of three technical replicates±SE. Each sample is a mix of 16 seedlings. (C) Bacterial growth of Pseudomonas syringae DC3000 (AvrRps4). 4-week-old plants were infiltrated with 1×10⁵ CFU ml⁻¹ bacteria. Each bar represents the mean of three biological replicates±SE. Asterisks indicate statistical significance (Student's t-test, p<0.05).

FIG. 13 illustrates that ttm2 is not a lesion mimic mutant. (A) Trypan blue staining of untreated Col wild type (Col), ttm2-1 and ttm2-2 plants. (B) RT-PCR analysis of PR1 gene expression of untreated Col wild type, ttm2-1 and ttm2-2 plants and Col wild type plants treated with 100 μM salicylic acid (SA). β-tubulin served as a loading control. Cot=cotyledon, TL=first true leaf. Bar=250 μm. 4-week-old plants were used for the analysis.

FIG. 14 illustrates that ttm2 exhibits enhanced Systemic Acquired Resistance (SAR). (A) Primary infection of 10 day-old cotyledons of Col wild type and ttm2 mutant plants was performed with the avirulent Hpa isolate, Emwa1 (SAR +) or water (SAR −). After 7 days a challenge infection was performed on systemic true leaves with Hpa, Noco2 (virulent). Hyphal structures were visualized 10 days later by trypan blue staining. (B) Stained leaves were microscopically examined and assigned to different classes (see panels). Data shown is from two independent experiments and was taken from 50 plants each; Fisher Exact Probability Test indicates a significant difference between SAR+ ttm2 lines and Col (p<0.05). Bars=250 μm.

FIG. 15 illustrates epistatic analysis of ttm2. (A) HR index of cotyledons (Cot) of Col wild type, Ws wild type, pad4-1, sid2-1, npr1-1 ttm2 mutants and corresponding double mutants 10 days after infection with avirulent Hpa Emwa1. Stained leaves were microscopically examined and assigned to different classes (see panels). (B) HR index of uninfected true leaves (TL) of the same plants. Data was taken from 12 plants. The experiment was repeated three times with similar results.

FIG. 16 illustrates that AtTTM2 expression is suppressed by SA and flg22 treatment. Quantitative real-time PCR analysis of Col wild type plants (A) 24h after treatment with 100 μM salicylic acid (SA) or water (H₂O). (B) 48h after treatment with 200 μM benzothiadiazole (BTH) or water. Shown is AtTTM2 and PR1 gene expression relative to AtEF1A. (C) Quantitative real-time PCR analysis of AtTTM2 in Col wild type (Col), sid2, pad4 and npr1 plants 4h after treatment with flg22 or water. Transcripts were normalized to AtEF1A. Each bar represents the mean of three technical replicates±SE. Each sample is a mix of 16 seedlings (A,B) or 4 leaves (C). For A and B 10-day old seedlings were used; for C 4-week old-plants were syringe-infiltrated.

FIG. 17 illustrates that AtTTM2 down-regulation after Pseudomonas syringae infection does not require NPR1, ICS1 and PAD4. Shown is publicly available miroarray data from the Glazebrook lab (http://www.ncbi.nlm.nih.gov/geo/query/acc.cgi?acc=GSE11009). Samples were taken 24h after inoculation with MgCl₂ (Mock) or Pseudomonas syringae pv. maculicola ES4326 (Wang et al.,2008).

FIG. 18 illustrates that overexpression of AtTTM2 causes enhanced susceptibility. (A) Quantitative real-time PCR analysis of AtTTM2 and ITS2 in Hpa-infected cotyledons of Col wt and 35S lines #2 and #5 ten days after infection. Transcripts were normalized to AtEF1A. Each bar represents the mean of three technical replicates±SE. Each sample is a mix of 15 seedlings. (B) Quantitative real-time PCR analysis of AtTTM2 and ITS2 in Hpa-infected cotyledons of Col wt and 35S line #7 ten days after infection. Transcripts were normalized to AtEF1A. Each bar represents the mean of three replicates±SE. Each sample is a mix of 15 seedlings. 10 day old seedlings were used for infection. (C) Left: Trypan blue staining of Col wild type (Col) and 35S:AtTTM2 over-expressor line (35S #7) 13 days after infection with Hpa, Emco5. Bars=250 μm. Right: Quantitative assessment of infection. Stained leaves were microscopically examined and assigned to different classes (see panels). Data shown was taken from 15-16 plants; Fisher Exact Probability Test indicates a significant difference between the over-expressor line and Col (p<0.05).

FIG. 19 illustrates that AtTTM2 function is conserved in crop species. Quantitative real-time PCR analysis of canola (Brassica napus var. Westar (A) and soybean (Glycine max var. Harasoy (B) plants treated with 200 μM BTH or water (H₂O) 48hrs after treatment. (A) Quantitative real-time PCR analysis of canola BnTTM2a, BnTTM2b and BnPR1. Transcripts were normalized to BnUBC21.(B) Quantitative real-time PCR analysis of soybean GmTTM2a/GmTTM2b and BnPR1. Transcripts were normalized to GmEF1B (Note: primers could not distinguish between the two soybean paralogues due to high sequence homology). Each bar represents the mean of three technical replicates±SE. 3-4 week old plants were used for treatments.

FIG. 20 illustrates sequence alignment of TTM orthologues. (A) Amino acid sequence alignment of AtTTM2 and canola (BnTTM2a (Bra011014), BnTTM2b (Bra012464)) and soybean orthologues (GmTTM2a (Gm1g09660), GmTTM2b (Gm2g14110)). The Walker A motif is highlighted in yellow, the Walker B motif in green, the lid motif in magenta and the EXEXK motif in purple. Conserved catalytic residues are underlined. (B) Percent amino acid sequence identity of canola and soybean TTM2 orthologues to AtTTM2.

FIG. 21 illustrates that AtTTM2 is not an adenylate cyclase. cAMP detection by HPLC. Upper panel: standards of ADP, ATP and cAMP. Middle panel: no protein added. Bottom panel: Reaction products after 30 min at 3TC. mAU=milliAbsorbance Units.

FIG. 22 provides primer sequences.

FIG. 23 illustrates expression of SITTM2A and B in approximately 4-5 week old tomato (Solanum lycopersicum) 48 hours after BTH (200 μM) treatment.

FIG. 24a illustrates expression of CsTTM2 in approximately 4-5week old cucumber (Cucumis sativus) 48 hours after BTH (200 uM) treatment. FIG. 24b illustrates expression of CaTTM2 in approximately 4-5 week old pepper (Capsicum annuum) 48 hours after BTH (200 uM) treatment.

FIG. 25 illustrates expression of PhTTM2A and B in approximately 4-5week old Petunia (Petunia hybrida) 48 hours after BTH (200 uM) treatment.

FIG. 26 illustrates expression of OsTTM2 in 4 week old rice (Oryza sativa) plant and BdTTM2 in the model monocotyledonous plant Brachypodium distachyon 48 hours after BTH (200 uM) treatment.

FIG. 27 illustrates expression of SITTM2A and B in approximately 4 week old tomato (Solanum lycopersicum) 24 hours after infection with the bacterial pathogen, Pseudomonas syringae pv. Tomato, DC3000.

FIG. 28 illustrates bacterial titre for a family segregating for the loss of function in TTM2B.

FIG. 29 illustrates average disease severity of plants from a family segregating for the loss of function in TTM2B.

FIG. 30 provides protein identity/similarity and nucleic acid identity of AtTTM2 and TTM2 from various plants.

FIG. 31 provides the nucleic acid sequence of TTM2 from various plants.

FIG. 32 provides the amino acid sequence of TTM2 from various plants.

### DETAIL DESCRIPTION OF THE INVENTION

The present invention relates to methods of modifying pathogen resistance in plants, plants and plant cells exhibiting modified pathogen resistance and methods of screening for members of a plant (plant cell) population having modified pathogen resistance. More particularly, the invention relates to modulating plant immunity by modulating negative regulators of plant immunity. The present invention is based on the discovery that TTM2 acts as a negative regulator of plant immunity and TTM2 knockout mutants show enhanced resistance to pathogens, while TTM2 over-expressors display enhanced susceptibility to pathogens.

Accordingly, the present invention provides for regulators of plant immunity. In certain embodiments, the regulators are regulators of PAMP-triggered immunity. In other embodiments, the regulators are regulators of effector-triggered immunity. In other embodiments, the regulators are regulators of PAMP-triggered immunity and effector-triggered immunity. In some embodiments, the regulators are negative regulators of immunity. In other embodiments, the regulators are positive regulators of immunity. In certain embodiments, the regulator is TTM2.

Also provided are methods of modulating pathogen resistance in plants by modulating expression and/or activity of regulators of plant immunity and methods of screening a plant population for members with altered pathogen resistance by screening for members having one or more mutations in a gene encoding a regulator of plant immunity. In certain embodiments, there are provided methods of modulating pathogen resistance in plants by modulating expression and/or activity of TTM2 and methods of screening a plant population for members with altered pathogen resistance by screening for members having one or more mutations in TTM2. Also provided are plants and plant cells having altered pathogen resistance. In certain embodiments, the plants have modified expression and/or activity of TTM2. Plants and plant cells having either increased or decreased expression and/or activity of TTM2 are contemplated. A worker skilled in the art would readily appreciate that such regulators may not be pathogen-specific and, as such, in certain embodiments, modulation of pathogen resistance is not limited to a particular pathogen. In certain embodiments, the pathogen is any plant pathogen. In other embodiments, the pathogen is a plant pathogen that triggers the PAMP-triggered immunity. In other embodiments, the pathogen is a plant pathogen that triggers effector-triggered immunity. In other embodiments, the pathogen triggers both PAMP-triggered immunity and effector-triggered immunity. The plant pathogens include, for example fungi, oomycetes, bacteria, viruses, viroids, virus-like organisms, phytoplasmas, protozoa, nematodes and insects. Examples of fungal phytopathogens include but are not limited to Bremia sp. (including but not limited to Bremia lactucae), Botrytis cinerea, Oidium neolycopersici, Leveillula taurica, Didymella bryoniae, Erysiphe cichoracearum, Sphaerotheca fulignea, Ascomycota or Basidomycota. Specific examples of Ascomycetes include but are not limited to Fusarium spp.; Thielaviopsis spp.; Verticillium spp.; Magnaporthe grisea and Sclerotinia sclerotiorum. Specific examples of Basidiomycetes include but are Ustilago spp., Rhizoctonia spp., Phakospora pachyrhizi, Puccinia spp. and Armillaria spp.

Examples of oomycetes include but are not limited to members of the Phytophthora, Pythium, downy mildews and white blister rusts. In one embodiment, the pathogen is Hyaloperonospora arabidopsidis.

Examples of bacterial plant pathogens include but are not limited to Clavibacter michiganensis, Pseudomonas, Xanthomonas and Burkholderia.

Examples of plant viruses include but are not limited to pepino mosaic virus, Fulvia fulva, tomato mosaic virus, tomato spotted wilt virus, pepper mild mottle virus, tobacco mosaic virus, pepper mild mottle virus.

A worker skilled in the art would readily appreciate that certain pathogens may infect specific types of plants. For example, pathogens that infect tomatoes (Solanum lycopersicum) include but are not limited to gray mould (Botrytis cinerea), Pythium root rot (Pythium spp.), bacterial canker (Clavibacter michiganensis subsp. Michiganensis), powdery mildew (Oidium neolycopersici), pepino mosaic virus, fusarium crown and root rot (Fusarium oxysporum f. sp. radicis-lycopersici), late blight (Phytophthora infestans), leaf mould (Fulvia fulva), tomato mosaic virus, tomato spotted wilt virus. Pathogens that infect peppers (Capsicum annuum) include but are not limited to Pythium crown and root rot (Pythium spp), fusarium stem and fruit rot (Fusarium solani), gray mould (Botrytis cinerea), powdery mildew (Leveillula taurica), pepper mild mottle virus, tobacco mosaic virus, tomato spotted wilt virus, tomato mosaic virus, pepper mild mottle virus. Pathogens that infect cucumber (Cucumis sativus) include but are not limited to Pythium crown rot and root rot (Pythium aphanidermatum and other Pythium spp), fusarium root and stem rot (Fusarium oxysporium f. sp. radicic-cucumerinum), gummy stem blight (Didymella bryoniae), powdery mildew (Erysiphe cichoracearum, Sphaerotheca fulignea), botrytis grew mould (Botrytis cinerea).

TTM2 is highly conserved in a wide variety of plant species. Accordingly, the plant may be any plant species which expresses TTM2 or a TTM2-like regulator of immunity. The plants may be, for example, a grain crop, an oilseed crop, a fruit crop, a vegetable crop, a biofuel crop, an ornamental plant, a flowering plant, an annual plant or a perennial plant. Examples of plants include but are not limited to petunia, tomato (Solanum lycopersicum), pepper (Capsicum annuum), lettuce, potato, onion, carrot, broccoli, celery, pea, spinach, impatiens, melon, cucumber, rose, sweet potato, apple and other fruit trees (such as pear, peach, nectarine, plum), eggplant, okra, corn, soybean, canola, wheat, oat, rice, sorghum, cotton and barley.

In certain embodiments, the plant is selected from Petunia (Petunia hybrida), tomato (Solanum lycopersicum), pepper (Capsicum annuum), lettuce (Lactuca sativa), eggplant (Solanum melongena), potato (Solanum tuberosum), onions (Allium cepa), carrots (Daucus carota), cucumber (Cucumis sativus), rose (Rosa species), canola (Brassica napus, Brassica rapa), broccoli (Brassica oleracea), celery (Apium graveolens), peas (Pisum sativum), spinach (Spinacia oleracea), wheat (Triticum aestivum), barley (Hordeum vulgare), oat (Avena sativa), corn (Zea mays), soybean (Glycine max), rice (Oryza sativa), sorghum (Sorghum bicolour) and cotton (Gossypium species).

In some plant species, there is a duplication of the TTM2 gene. These duplicated genes are named TTM2A and TTM2B based on the order the genes were identified in the specific species. Non-limiting examples of plant species having a duplication of the TTM2 gene are Solanum lycopersicum, Petunia hybrid, Capsicum annuum, Vitis vinifera, Gossypium raimondii, Brassica rapa, Glycine max, Populus trichocarpa, Linum usitatissimum and Manihot esculenta. Both copies may respond to SAR induction through BTH treatment and may have overlapping function. Accordingly, in embodiments in which there is more than one TTM2 gene paralogue (including but not limited to a duplication of the TTM2 gene), there are provided methods of modulating pathogen resistance in plants by modulating expression and/or activity of one or more copies of the TTM2 gene and methods of screening a plant population for members with altered pathogen resistance by screening for members having one or more mutations in the one or more copies of the TTM2 gene. Also provided are plants and plant cells having altered pathogen resistance. In certain embodiments, the plants have modified expression and/or activity of one or more copies of TTM2. Plants and plant cells having either increased or decreased expression and/or activity of one or more copies of TTM2 are contemplated. In some embodiments (in plants having the duplication of the TTM2 gene), one copy of TTM2 is inactivated to provide enhanced resistance. In another embodiment, both copies have been inactivated to provide additive or synergistic enhanced resistance.

### TTM2 Nucleic Acids

The present invention provides for nucleic acids comprising nucleotide sequences encoding regulators of plant immunity. In certain embodiments, the nucleic acids encode regulators of PAMP-triggered immunity. In other embodiments, the nucleic acids encode regulators of effector-triggered immunity. In other embodiments, the nucleic acids encode regulators of PAMP-triggered immunity and effector-triggered immunity. In some embodiments, the regulators are negative regulators of immunity. In other embodiments the regulators are positive regulators of immunity. The nucleic acids include nucleic acids that encode TTM2 or TTM2-like nucleic acids, homologs, variants, mutants and fragments thereof. Nucleic acids include, but are not limited to, genomic DNA, cDNA, RNA, fragments and modified versions thereof.

In certain embodiments, the cDNA of TTM2 comprises the sequence as set forth in any one of SEQ ID NOs: 1 and 3 to 41.

In specific embodiments, the cDNA of TTM2 comprises the sequence as set forth below (SEQ ID NO:1).

ATGGGTCAAGACAGCAATGGAATTGAGTTTCATCAGAAGAGACATGGTCT
CTTGAAGGATCAAGTCCAATTGGTTAAGAGAAGAGACTCTATTCGGTATG
AAATTGTTTCTATTCAAGATCGGTTGTCATTTGAGAAGGGCTTCTTTGCG
GTTATCCGTGCTTGCCAATTGCTTTCTCAGAAGAATGATGGGATCATATT
GGTTGGTGTTGCTGGACCTTCTGGTGCTGGAAAGACTGTATTCACTGAGA
AGATACTCAATTTTCTGCCAAGTGTTGCTGTCATTTCAATGGACAATTAT
AATGATTCTAGTCGGATTGTTGATGGGAACTTTGATGATCCACGGTTAAC
GGACTATGACACATTGCTCAAGAATCTTGAAGACTTAAAGGAAGGAAAGC
AGGTTGAGGTTCCTATTTATGATTTTAAGTCCAGCTCTCGTGTTGGATAC
AGGACCCTTGATGTCCCACCTTCTCGGATTGTGATTATTGAAGGAATCTA
TGCTTTGAGTGAAAAACTGCGACCTTTATTGGATCTTCGTGTGTCTGTTA
CTGGTGGAGTTCATTTTGACCTTGTTAAACGGGTTCTCCGTGATATACAA
CGTGCAGGTCAACAGCCAGAGGAGATTATCCATCAGATATCTGAAACAGT
ATACCCGATGTACAAAGCTTTCATTGAGCCAGATCTCCAGACTGCTCAAA
TCAAAATCATTAATAAATTCAACCCCTTCACTGGTTTTCAGAGCCCGACT
TACATCTTGAAGTCAAGAAAGGAGGTATCTGTTGATCAGATCAAGGCGGT
CCTTTCTGATGGACATACAGAGACTAAGGAGGAGACCTATGATATATATC
TTCTTCCTCCGGGTGAAGATCCAGAGTCGTGCCAATCATATTTGAGGATG
CGGAATAAAGATGGAAAGTACAGCCTTATGTTTGAGGAATGGGTTACGGA
TACTCCTTTTGTCATATCCCCAAGGATTACATTTGAAGTCAGTGTTCGCC
TACTTGGTGGGCTCATGGCATTGGGATACACAATAGCAACTATACTTAAA
AGGAACAGCCATGTATTTGCTACTGATAAGGTGTTTGTGAAAATCGATTG
GCTTGAGCAACTGAATCGTCACTACATGCAGGTGCAAGGTAAAGATCGGC
AACTTGTACAGAGTACTGCAGAGCAGCTAGGATTGGAAGGATCGTTCATT
CCACGCACCTATATTGAACAGATCCAACTCGAAAAACTAATAAATGAAGT
AATGGCCCTACCAGATGATCTAAAGAACAAGCTTAGCTTAGATGAGGATT
TGGTGTCTAGTTCAAGCCCTAAGGAAGCACTCTTACGAGCGTCTGCAGAT
AGAGTAGCCATGAGAAATAAGAACCTCAAAAGAGGCATGTCACACTCATA
TTCAACCCAAAGAGATAAGAATCTGTCCAAGCTTGCTGGTTATTCTTCAA
GCGATAGGAGGTACGAAGAAAGAAATCACGACTCGCCAGCGAACGAGGGG
TTTATGACTCTGCTTTCAGAACAAATATCATCTCTCAACGAGAGAATGGA
TGAGTTCACAAGTCGAATTGAAGAGCTCAATTCAAAGTTGAGCTGCAATA
AAAACTCTCCAACACAGCAGAGCTTGTCAATCCAAACCGAAGTCTGCAAT
GGGTCAGCTCCTACTTCGTATTTCATTTCTGGTCTGGACAATGGCTGCTT
GACAAATTCCATAATGCCCCATTCATCATCCTCCTCCCAACTAGCCAAGG
ATTCACCCTTAATGGAAGAGATATCGACCATATCACGAGGACAGCGTCAA
GTTATGCATCAGTTGGATAATTTGTGCAATCTGATGAGGGAAAGCTCAGC
AGAAAGGTCACGCCTAGCAAGAACAGGGAGCAGCAATAGCGGTAACAGAG
GCAGATCAAGCAAAAGCTCCTTCTTGTCCAATGTGGAATCTAACAAGCTC
CCTCTTGTGTTAACCGTGGCTATTTGCAGCATAGGTATTATAGTGATCAA
GAGCTACATTAACAAGCGGCAATAACATCTATTAGCCACTATGGGTTTTC
TCTTCT

In certain embodiments, the nucleic acid molecule comprises the sequence as set forth in GenBank AY117297 or a variant or fragment thereof.

In certain embodiments, the nucleic acid comprises the genomic DNA sequence of TTM2 as set forth below (SEQ ID NO:2).

AATGTTACCTCCTCGTGGGTCTGAGATCTTTTTCCCCAGATTCTCTACA
AATCGCTCTCCCCGATAAAGAAGAAGCTCTCACAAAATTCCTCTTTCTC
TCTCTCTCTCTGATTCCCCATTATTAGTTTCTGTGTTAAAATTGAATTG
CGACATAACTCTGCCAAAGTGATAAGCCCCGATTCACACTAATTCCGAG
AGATTTTTCTGTGTGAGTGCCATACTAAACTCCGAGAAATCGGCTCAAG
TTTCGATTTTTGTTTCTGGGTTTTACCTTTTCAACCAATCTGTTTGCGT
TTTTTCTTTTGTTCTGGGTGTTGTTGTTATAGAACAGTTTGATCGTTTC
TTCTTTGATGGTTTTTGTTTGGATTCGTTTCGAGCTTTCGCTTGTTTTG
TTTCATTGTATGGCTGCATTTTGATGATAATTTCATATCCGCTACTTTT
GGATTAGAGTGCTGCGTTATCTTTAGTCTGCTTGACTCATTCCTCCATG
GGTTTAAGAGTAAATGTCACTGTTCCTTTAAAATGTTCCGTACAATTCA
GTCTTCACTATGTGTGTTTTTGGCTCTCTTAGCTTTTGGTCTCTCCATG
TTTCCCAGCTTAAGATTATGTCTTATTAATGAAAATGTGTTCTTTTTTG
CAGATTATTGTTCATAATGGGTCAAGACAGCAATGGAATTGAGTTTCAT
CAGAAGAGACATGGTCTCTTGAAGGATCAAGTCCAATTGGTTAAGAGAA
GAGACTCTATTCGGTATGAAATTGTTTCTATTCAAGATCGGTTGTCATT
TGAGAAGGGCTTCTTTGCGGTTATCCGTGCTTGCCAATTGCTTTCTCAG
AAGAATGATGGGATCATATTGGTTGGTGTTGCTGGACCTTCTGGTGCTG
GAAAGACTGTATTCACTGAGAAGATACTCAATTTTCTGCCAAGTGTTGC
TGTCATTTCAATGGACAATTATAATGATTCTAGTCGGATTGTTGATGGG
AACTTTGATGGTAAGAATTTTCATCTTGATAGGTCCCATGAGGAATGAA
GTCCTATGACACATTGTTTTGAAACTTGAAGTATCTTGCTGCTGACAAA
CCTTATGTTTTGAAACTTAGATCCACGGTTAACGGACTATGACACATTG
CTCAAGAATCTTGAAGACTTAAAGGAAGGAAAGCAGGTTGAGGTTCCTA
TTTATGATTTTAAGTCCAGCTCTCGTGTTGGATACAGGTAATGCGTGAC
GTGATTGTGCAGTTTCCATTTACTGATTCAGTCATCATTTTGTACTTTA
TCTAAACAAACAACCACTTGGTGTCCATTGTCACAAAAGTTTGATATTA
CATTCACATCAGCATGGTTTCTGTTTATTCCACTGAAGCATTGTTTTTA
ATGCCATGATTTAATTTGCTAGGACCCTTGATGTCCCACCTTCTCGGAT
TGTGATTATTGAAGGAATCTATGCTTTGAGTGAAAAACTGCGACCTTTA
TTGGATCTTCGTGTGTCTGTTACTGGTGGAGTTCATTTTGACCTTGTTA
AACGGGTTCTCCGTGATATACAACGTGCAGGTCAACAGCCAGAGGAGAT
TATCCATCAGATATCTGAAACAGTTTGTCCTCATTTCTTTTATTTCGTG
TGACTGTTTGGTTTAGTATATGAGCTGCCAATTGTTTATATTAACAACT
CACTGTTTATGTAGGTATACCCGATGTACAAAGCTTTCATTGAGCCAGA
TCTCCAGACTGCTCAAATCAAAATCATTAATAAATTCAACCCCTTCACT
GGTTTTCAGAGCCCGACTTACATCTTGAAGGTTTGAAAAGTGACCGGAT
TTCTATCCATCTTATCATATTAATCAGTGCTCTGCAAACTCAGTATTCA
ACTATTGACAGCGTTTGGTTAATTGAAGTTCTTTTACTATTACTTTGTT
GTAGTCAAGAAAGGAGGTATCTGTTGATCAGATCAAGGCGGTCCTTTCT
GATGGACATACAGAGACTAAGGAGGAGACCTATGATATATATCTTCTTC
CTCCGGGTGAAGATCCAGAGTCGTGCCAATCATATTTGAGGATGCGGAA
TAAAGATGGAAAGTACAGCCTTATGTTTGAGGTTTGTTCAGAGTTTATT
TTCCATGTTCTCATCAATATGACTATTCAATATCTGGAAAAGCTGACAA
TCCCTCTGATTCTGGTAAGATGCTTAGTATCTGGTGAATAACTGTGGTT
CTGGTTTTGACAACCAGGAATGGGTTACGGATACTCCTTTTGTCATATC
CCCAAGGATTACATTTGAAGTCAGTGTTCGCCTACTTGGTGGGCTCATG
GCATTGGGATACACAATAGCAACTATACTTAAAAGGAACAGCCATGTAT
TTGCTACTGATAAGGTGTTTGTGAAAATCGATTGGCTTGAGCAACTGAA
TCGTCACTACATGCAGGTCTGTCTATCTATACTCATTCACCATCATTTG
CTAGAAAATTGATTGTTCATCTGGCTTTATGATGACAGTACTCTTGTTC
CCAGTTACTATGAAATTTCTTTATCTCCCCAAAAAAATATGACTACAAT
ATTCAAATTTTGTTATAAACAGGTGCAAGGTAAAGATCGGCAACTTGTA
CAGAGTACTGCAGAGCAGCTAGGATTGGAAGGATCGTTCATTCCACGCA
CCTATATTGAACAGATCCAACTCGAAAAACTAATAAATGAAGTAATGGT
ATGTTTTGCTGTTCGGGTTTTGAGTTTTGTTTTGACTACATTTTATCTG
GGGTCCTGACTAAAAATCCCATCACAGGCCCTACCAGATGATCTAAAGA
ACAAGCTTAGCTTAGATGAGGATTTGGTGTCTAGTTCAAGCCCTAAGGA
AGCACTCTTACGAGCGTCTGCAGATAGAGTAGCCATGAGAAATAAGAAC
CTCAAAAGGTACACATCTTTTGAGGAGTGTGTGAGAAAGCTTTGTTACT
TCCAACCCATGTGTCCTTAGTTATGCCATTTATTATACACAGAGGCATG
TCACACTCATATTCAACCCAAAGAGATAAGAATCTGTCCAAGCTTGCTG
GTTATTCTTCAAGCGATAGGAGGTACGAAGAAAGAAATCACGACTCGCC
AGCGAACGAGGTTCAAATTTGTTCTCTTTCATTCCCTCTTGGCAACTTT
GAAGTCTTCCTTTTAACTTAAGGGTGCACTTCTTCTGGTTTTCAACTAT
TTTTAGGGGTTTATGACTCTGCTTTCAGAACAAATATCATCTCTCAACG
AGAGAATGGATGAGTTCACAAGTCGAATTGAAGAGCTCAATTCAAAGTT
GAGCTGCAATAAAAACTCTCCAACACAGCAGAGCTTGTCAATCCAAACC
GAAGTCTGCAATGGGTCAGCTCCTACTTCGTATTTCATTTCTGGTCTGG
ACAATGGCTGCTTGACAAATTCCATAATGCCCCATTCATCATCCTCCTC
CCAACTAGCCAAGGATTCACCCTTAATGGAAGAGGTAAGTAACCTCACG
CATCTCTCGTTTATGAATTTGGATTTTATTGCGTTGCTTTGTAACTTTG
AGCTGCTCTGGTGCAACAGATATCGACCATATCACGAGGACAGCGTCAA
GTTATGCATCAGTTGGATAATTTGTGCAATCTGATGAGGGAAAGCTCAG
CAGAAAGGTCACGCCTAGCAAGAACAGGGAGCAGCAATAGCGGTAACAG
AGGCAGATCAAGCAAAAGCTCCTTCTTGTCCAATGTGGAATCTAACAAG
CTCCCTCTTGTGTTAACCGTGGCTATTTGCAGCATAGGTATTATAGTGA
TCAAGAGCTACATTAACAAGCGGCAATAACATCTATTAGCCACTATGGG
TTTTCTCTTCTTTTTTTGTTCTTTTGTTTTGGTATTTTTCTCACTGGAG
GCGTTTTGTGAGCTTCCCTGGTTTCTCTACGTAGACAATGACGCCAGTT
CTCTTCCCCTAAATTAGTCGTTTGGAAGACGTTCTCGATTATTTATTCA
ATAAAGTTTAGGTTTTTAGTTT

In certain embodiments, the nucleic acid comprises the sequence of Gene ID At1g26190 or a variant or fragment thereof.

In certain embodiments of the present invention, there is provided a nucleic acid comprising a nucleotide sequence encoding a negative regulator of plant immunity, wherein the nucleotide sequence comprises the sequence as set forth in any one of SEQ ID NOs:1 to 41. In other embodiments, there is provided a nucleic acid comprising a sequence having at least 50%, 55%, 60%, 65%, 70%, 75%, 80%, 85%, 90%, 95%, 96%, 97%, 98%, 99% or 100% identity to any one of the sequences set forth in SEQ ID NOs:1 to 41 and fragments thereof or the complement thereof. In certain embodiments, fragments are at least 10, at least 20, at least 50 nucleotides in length. The fragments may be used, for example, as primers or probes.

In some embodiments of the present invention, there is provided a nucleic acid comprising the TTM2 nucleotide sequence comprising one or more substitutions, insertions and/or deletions. Such nucleotide sequences may or may not encode functional TTM2. For certain embodiments, the nucleic acid comprises a TTM2 nucleotide sequence which includes one or more T-DNA insertions. In other embodiments, the nucleic acid comprises a TTM2 nucleotide sequence which includes a selection marker cassette. In other embodiments, the nucleic acid comprises a TTM2 nucleotide sequence which includes one or more point mutations. In certain embodiments, the nucleic acid comprises a TTM2 nucleotide sequence includes a deletion. In certain embodiments, the nucleic acid comprises a TTM2 nucleotide sequence which includes rearrangement. In certain embodiments, the nucleic acid comprises a TTM2 nucleotide sequence which includes a frame shift.

In certain embodiments, there is provided a nucleic acid comprising a nucleotide sequence encoding the amino acid sequence set forth in any one of SEQ ID NOs:42 to 83. In specific embodiments, there is provided a nucleic acid comprising a nucleotide sequence encoding the amino acid sequence set forth below (SEQ ID NO:42).

MGQDSNGIEFHQKRHGLLKDQVQLVKRRDSIRYEIVSIQDRLSFEKGFFA
VIRACQLLSQKNDGIILVGVAGPSGAGKTVFTEKILNFLPSVAVISMDNY
NDSSRIVDGNFDDPRLTDYDTLLKNLEDLKEGKQVEVPIYDFKSSSRVGY
RTLDVPPSRIVIIEGIYALSEKLRPLLDLRVSVTGGVHFDLVKRVLRDIQ
RAGQQPEEIIHQISETVYPMYKAFIEPDLQTAQIKIINKFNPFTGFQSPT
YILKSRKEVSVDQIKAVLSDGHTETKEETYDIYLLPPGEDPESCQSYLRM
RNKDGKYSLMFEEWVTDTPFVISPRITFEVSVRLLGGLMALGYTIATILK
RNSHVFATDKVFVKIDWLEQLNRHYMQVQGKDRQLVQSTAEQLGLEGSFI
PRTYIEQIQLEKLINEVMALPDDLKNKLSLDEDLVSSSSPKEALLRASAD
RVAMRNKNLKRGMSHSYSTQRDKNLSKLAGYSSSDRRYEERNHDSPANEG
FMTLLSEQISSLNERMDEFTSRIEELNSKLSCNKNSPTQQSLSIQTEVCN
GSAPTSYFISGLDNGCLTNSIMPHSSSSSQLAKDSPLMEEISTISRGQRQ
VMHQLDNLCNLMRESSAERSRLARTGSSNSGNRGRSSKSSFLSNVESNKL
PLVLTVAICSIGIIVIKSYINKRQ

In certain embodiments, there is provided a nucleic acid comprising a sequence encoding the amino acid sequence as set forth in GenBank AAM51372.1 or a fragment or variant thereof.

In other embodiments, there is provided a nucleic acid encoding a polypeptide comprising a sequence at least 50%, 55%, 60%, 65%, 70%, 75%, 80%, 85%, 90%, 95%, 96%, 97%, 98%, 99% (or more) percent identity to any one of the sequences set forth in SEQ ID NOs:42 to 83 and fragments thereof.

Also provided are nucleic acids that hybridize to the nucleic acids of the present invention or the complement thereof. In certain embodiments, there is provided a nucleic acid that hybridizes to any one of the sequences as set forth in SEQ ID NOs:1 to 41 or the complement thereof under conditions of low, moderate or high stringency. A worker skilled in the art readily appreciates that hybridization and the strength of hybridization (i.e., the strength of the association between the nucleic acids) is impacted by such factors as the degree of complementary between the nucleic acids, stringency of the conditions involved, the ⁻I, of the formed hybrid, and the G:C ratio within the nucleic acids. Such a worker could readily determine appropriate stringent (see, for example, Sambrook, et al., Molecular Cloning: A Laboratory Manual, 2^{nd} ed., Cold Spring Harbor Laboratory Press, New York (1989) pp. 9.50-51, 11.48-49 and 11.2-11.3).

Typically under high stringency conditions only highly similar sequences will hybridize under these conditions (typically >95% identity). With moderate stringency conditions typically those sequence having greater than 80% identity will hybridize and with low stringency conditions those sequences having greater than 50% identity will hybridize.

A non-limiting example of “high stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed. A non-limiting example of “medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 500 nucleotides in length is employed. A non-limiting example “Low stringency conditions” when used in reference to nucleic acid hybridization comprise conditions equivalent to binding or hybridization at 42.degree. C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 5×SSPE, 0.1% SDS at 42° C. when a probe of about 500 nucleotides in length is employed.

The polynucleotides include the coding sequence TTM2 polypeptide, in isolation, in combination with additional coding sequences (e.g., a purification tag, a localization signal, as a fusion-protein, as a pre-protein, or the like), in combination with non-coding sequences (e.g., introns or inteins, regulatory elements such as promoters (including inducible promoters, tissue-specific promoters (such as root-specific or leaf specific promoters), enhancers, terminators, and the like), and/or in a vector or host environment in which the polynucleotide encoding a transcription factor or transcription factor homologue polypeptide is an endogenous or exogenous gene.

Appropriate additional coding sequences (e.g., a purification tag, a localization signal, as a fusion-protein, as a pre-protein, or the like), non-coding sequences (e.g., introns or inteins, regulatory elements such as promoters (including inducible promoters, tissue-specific promoters (such as root-specific or leaf specific promoters), enhancers, terminators, and the like), and vectors for use in plants/plant cells are known in the art.

### TTM2 Polypeptides

The present invention provides TTM2 or TTM2-like polypetides, homologs, variants, mutants and fragments thereof.

In embodiments of the present invention, there is provided a TTM2 comprising the sequence as set forth in any one of SEQ ID NOs:42 to 83. In other embodiments, there is provided a polypeptide comprising a sequence at least 50%, 55%, 60%, 65%, 70%, 75%, 80%, 85%, 90%, 95%, 96%, 97%, 98%, 99% (or more) percent identity to any one of the sequences set forth in SEQ ID NOs:42 to 83 and fragments thereof. In certain embodiments, fragments are at least 10, at least 20, at least 50 amino acids in length. In certain embodiments, the polypeptide sequences contain heterologous sequences.

A worker skilled in the art would readily appreciate the uses of the polynucleotides and/or polypeptides of the present invention. Non-limiting examples include use in methods for modifying a plant phenotype, genetic engineering and screening of populations.

### Production and Screening of Plants Having Modified Pathogen Resistance

The present invention provides for plants and plant cells having modified pathogen resistance as compared to wild-type plants (for example, original cultivars). In one embodiment, the plants have increased pathogen resistance. In an alternative embodiment, the plants have decreased pathogen resistance. The pathogen resistance may be associated with modified PAMP-triggered immunity and/or modified effector-triggered immunity. In certain embodiments, the plants exhibit enhanced systemic acquired resistance (SAR) and/or enhanced hypersensitive response. In certain embodiments, the plants have altered (increased or decreased) expression and/or activity of negative regulators of plant immunity as compared to wild type plants. In some embodiments, the plants have decreased expression and/or activity of TTM2 as compared to wild-type. In some embodiments, the plants have no expression and/or activity of TTM2. The plants may be homozygous or heterozygous for the modified TTM2 gene. In plant species having a multiplication of the TTM2 gene one or more copies of the gene may have modified (either increased or decreased) expression and/or activity.

For example, in plant species having a duplication of the TTM2 gene one or both of TTM2A and B may have modified (either increased or decreased) expression and/or activity.

A worker skilled in the art would readily appreciate that the plants could be engineered to have modified expression and/or activity of other proteins in addition to TTM2 or have mutations in other genes in addition to TTM2. For example, the plants may also include modified expression and/or activity of other molecules involved in plant immunity or pathogen/disease resistance. Likewise a worker skilled in the art would appreciate that the plants of the invention may be crossed with plants having specific phenotypes. Examples of specific phenotypes include but not limited to cold or heat tolerance, drought tolerance, high yield, variegation in morphology, and modification in life span.

The plants with modified pathogen resistance may be non-mutagenized, mutagenized or transgenic and the progeny thereof.

In certain embodiments, the plants exhibiting modified pathogen resistance are the result of spontaneous mutations.

In certain embodiments, the plants exhibiting modified pathogen resistance have been mutagenized by chemical or physical means. For example, a worker skilled in the art would readily appreciate that ethylmethane sulfonate (EMS) may be used as a mutagen or radiation, such as x-ray, γ-ray, and fast-neutron radiation may be used as a mutagen. In certain embodiments of the invention, the plant is mutagenized with EMS.

In certain embodiments, the mutagenized plant is selected from the group consisting of Petunia (Petunia hybrida), tomato (Solanum lycopersicum), pepper (Capsicum annuum), lettuce (Lactuca sativa), eggplant (Solanum melongena), potato (Solanum tuberosum), onions (Allium cepa), carrots (Daucus carota), cucumber (Cucumis sativus), rose (Rosa species), canola (Brassica napus, Brassica rapa), broccoli (Brassica oleracea), celery (Apium graveolens), peas (Pisum sativum), spinach (Spinacia oleracea), wheat (Triticum aestivum), barley (Hordeum vulgare), oat (Avena sativa), corn (Zea mays), soybean (Glycine max), rice (Oryza sativa), sorghum (Sorghum bicolour) and cotton (Gossypium species)

In certain embodiments, the plant mutagenized with EMS and screened for modified pathogen resistance is a Petunia×hybrid. In certain embodiments, the plant mutagenized with EMS and screened for modified pathogen resistance is a tomato. In certain embodiments, the plant mutagenized with EMS and screened for modified pathogen resistance is a cucumber.

In certain other embodiments, the plants exhibiting modified pathogen resistance have been genetically engineered.

In certain embodiments, antisense approaches may be used to down-regulate expression of a nucleic acid of the invention, e.g., as a further mechanism for modulating plant phenotype. That is, anti-sense sequences of the nucleic acids of the invention, or subsequences thereof, may be used to block expression of naturally occurring homologous nucleic acids. A variety of sense and anti-sense technologies are known in the art, e.g., as set forth in Lichtenstein and Nellen (1997) Antisense Technology: A Practical Approach IRL Press at Oxford University, Oxford, England. In general, sense or anti-sense sequences are introduced into a cell, where they are optionally amplified, e.g., by transcription. Such sequences include both simple oligonucleotide sequences and catalytic sequences such as ribozymes.

In one embodiment, a reduction or elimination of expression (i.e., a “knock-out”) of TTM2 or homologue in a transgenic plant can be obtained by insertion mutagenesis using the T-DNA of Agrobacterium tumefaciens or a selection marker cassette or any other non-sense DNA fragments. After generating the insertion mutants, the mutants can be screened to identify those containing the insertion in the TTM2 gene. Plants containing one or more transgene insertion events at the desired gene can be crossed to generate homozygous plants for the mutation (Koncz et al. (1992) Methods in Arabidopsis Research; World Scientific).

In another embodiment, a reduction or elimination of expression (i.e., a “knock-out” or “knock-down”) of TTM2 or homologue in a transgenic plant can be introducing an antisense construct corresponding TTM2 as a cDNA. For antisense suppression, the TTM2 cDNA is arranged in reverse orientation (with respect to the coding sequence) relative to the promoter sequence in the expression vector. The introduced sequence need not be the full length cDNA or gene, and need not be identical to the cDNA or gene found in the plant type to be transformed. Typically, the antisense sequence need only be capable of hybridizing to the target gene or RNA of interest. Thus, where the introduced sequence is of shorter length, a higher degree of homology to the endogenous transcription factor sequence will be needed for effective antisense suppression. While antisense sequences of various lengths can be utilized, preferably, the introduced antisense sequence in the vector will be at least 30 nucleotides in length, and improved antisense suppression will typically be observed as the length of the antisense sequence increases. Preferably, the length of the antisense sequence in the vector will be greater than 100 nucleotides. Transcription of an antisense construct as described results in the production of RNA molecules that are the reverse complement of mRNA molecules transcribed from the endogenous transcription factor gene in the plant cell.

Suppression of gene expression may also be achieved using a ribozyme. Ribozymes are RNA molecules that possess highly specific endoribonuclease activity. The production and use of ribozymes are disclosed in U.S. Pat. No. 4,987,071 and U.S. Pat. No. 5,543,508. Synthetic ribozyme sequences including antisense RNAs can be used to confer RNA cleaving activity on the antisense RNA, such that endogenous mRNA molecules that hybridize to the antisense RNA are cleaved, which in turn leads to an enhanced antisense inhibition of endogenous gene expression.

Vectors expressing an untranslatable form of the transcription factor mRNA, e.g., sequences comprising one or more stop codon, or nonsense mutation) may also be used to suppress expression of a gene, thereby reducing or eliminating it's activity and modifying one or more traits. Methods for producing such constructs are described in U.S. Pat. No. 5,583,021.

Preferably, such constructs are made by introducing a premature stop codon into the transcription factor gene. Alternatively, a plant trait can be modified by gene silencing using double-strand RNA (Sharp (1999) Genes and Development 13: 139-141).

Plant phenotype may also be altered by eliminating an endogenous gene, e.g., by homologous recombination (Kempin et al. (1997) Nature 389: 802).

A plant trait may also be modified by using the Cre-lox system (for example, as described in U.S. Pat. No. 5,658,772). A plant genome can be modified to include first and second lox sites that are then contacted with a Cre recombinase. If the lox sites are in the same orientation, the intervening DNA sequence between the two sites is excised. If the lox sites are in the opposite orientation, the intervening sequence is inverted.

In addition, silencing approach using small interfering RNA (siRNA), short hairpin RNA (shRNA) system, complementary mature CRISPR RNA (crRNA) by CRISPR/Cas system, virus inducing gene silencing (VIGS) system may also be used to make down regulated or knockout of TTM2 mutants. Dominant negative approaches and silencing by high copy expression of TTM2 may also be used to make down regulated or knockout of TTM2 mutants.

A worker skilled in the art would readily appreciate that other examples of site-directed mutagenesis include but are not limited to meganucleases and TALENs. A worker skilled in the art would also appreciate that post-translational gene silencing can also be used to down regulate gene expression.

Transgenic plants (or plant cells, or plant explants, or plant tissues) can be produced by a variety of well established techniques as described above. Following construction of a vector, most typically an expression cassette, including a polynucleotide, e.g., encoding a transcription factor or transcription factor homologue, of the invention, standard techniques can be used to introduce the polynucleotide into a plant, a plant cell, a plant explant or a plant tissue of interest. Optionally, the plant cell, explant or tissue can be regenerated to produce a transgenic plant.

The plant can be any higher plant. For example, the plants may be, for example, a commercial crop, produce crop, a biofuel crop, an ornamental plant, a flowering plant, an annual plant or a perennial plant. Examples of plants include but are not limited to petunia, tomato (Solanum lycopersicum), pepper (Capsicum annuum), impatiens, cucumber, rose, sweet potato, apple and other fruit trees (such as pear, peach, nectarine, plum), eggplant, okra,corn, soy, canola, wheat, rice and barley.

In certain embodiments, the plant is selected from the group consisting of Petunia (Petunia hybrida), tomato (Solanum lycopersicum), pepper (Capsicum annuum), lettuce (Lactuca sativa), eggplant (Solanum melongena), potato (Solanum tuberosum), onions (Allium cepa), carrots (Daucus carota), cucumber (Cucumis sativus), rose (Rosa species), canola (Brassica napus, Brassica rapa), broccoli (Brassica oleracea), celery (Apium graveolens), peas (Pisum sativum), spinach (Spinacia oleracea), wheat (Triticum aestivum), barley (Hordeum vulgare), oat (Avena sativa), corn (Zea mays), soybean (Glycine max), rice (Oryza sativa), sorghum (Sorghum bicolour) and cotton (Gossypium species).

In certain embodiments, the plant is selected from Solanum lycopersicum, Petunia hybrid, Cucumis sativus, Capsicum annuum, Oryza sativa, Hordeum vulgare, Zea mays, Brachypodium distachyo, Prunus persica, Malus×domesetica, Sorghum bicolor, Aquilegia coerulea, Mimulus guttatus, Solanum tuberosum, Vitis vinifera, Eucalyptus grandis, Citrus sinensis, Theobroma cacao, Gossypium raimondii, Carica papaya, Thellungiella halophila, Brassica rapa, Capsella rubella, Glycine max, Phaseolus vulgaris, Populus trichocarpa, Linum usitatissimum, Ricinus communis or Manihot esculenta.

Transformation and regeneration of plant cells is now routine, and the selection of the most appropriate transformation technique will be determined by the practitioner. The choice of method will vary with the type of plant to be transformed; those skilled in the art will recognize the suitability of particular methods for given plant types. Suitable methods can include, but are not limited to: electroporation of plant protoplasts; liposome-mediated transformation; polyethylene glycol (PEG) mediated transformation; transformation using viruses; micro-injection of plant cells; micro-projectile bombardment of plant cells; vacuum infiltration; and Agrobacterium tumeficiens mediated transformation. Transformation means introducing a nucleotide sequence into a plant in a manner to cause stable or transient expression of the sequence.

Successful examples of the modification of plant characteristics by transformation with cloned sequences which serve to illustrate the current knowledge in this field of technology, and which are herein incorporated by reference, include: U.S. Pat. Nos. 5,571,706; 5,677,175; 5,510,471; 5,750,386; 5,597,945; 5,589,615; 5,750,871; 5,268,526; 5,780,708; 5,538,880; 5,773,269; 5,736,369 and 5,610,042.

Following transformation, plants are preferably selected using a dominant selectable marker incorporated into the transformation vector. Typically, such a marker will confer antibiotic or herbicide resistance on the transformed plants, and selection of transformants can be accomplished by exposing the plants to appropriate concentrations of the antibiotic or herbicide.

After transformed plants are selected and grown to maturity, those plants showing a modified trait are identified. The modified trait can be any of those traits described above. Additionally, to confirm that the modified trait is due to changes in expression levels or activity of the polypeptide or polynucleotide of the invention can be determined by analyzing mRNA expression using Northern blots, RT-PCR, RNA seq or microarrays, or protein expression using immunoblots or Western blots or gel shift assays.

### Screening

The present invention also provides methods of screening plants for mutation(s) in the TTM2 gene and/or decreased expression and/or activity of TTM2. In plant species having a multiplication of the TTM2 gene, one or more copies of the gene may be screened. For example, in plant species having a duplication of the TTM2 gene, one or both of TTM2A and B genes may be screened.

In certain embodiments, the methods are high throughput. A worker skilled in the art would readily appreciate appropriate screening methods. For example, the methods include but are not limited to sequencing based methodologies, high resolution DNA melting methodologies, TILLING methodologies and hybridization methodologies. Also provided are methods for screening for expression and/or activity of TTM2. A worker skilled in the art would readily appreciate appropriate methodologies for screening for expression. For example, mRNA expression may be analyzed using Northern blots, slot-blots, dot-blots) RT-PCR, RNA sequence or microarrays, or protein expression may be analyzed using immunoblots or Western blots or gel shift assays.

Phenotypic evaluation of plants may be performed to determine if the mutations of interest have an effect on the performance of the plant under various conditions. Types of phenotypic analysis include, but are not limited to, evaluating drought stress responses, low temperature growth and/or disease susceptibility.

In certain embodiments, plant immunity is evaluated. In certain embodiments, pathogen resistance is evaluated. Methods of evaluating plant immunity and pathogen resistance are known in the art. For example, pathogen resistance may be assessed by inoculating test plants with the pathogen of interest and assessing disease progression at set time points. Activation of immunity may be tested by expression of marker genes and/or hormone measurement.

### Kits

Kits comprising one or more of reagents necessary for the methods set forth therein. For example, the kits may include any of one or more primers, probes, DNA polymerase and other reagents and instructions for use.

To gain a better understanding of the invention described herein, the following examples are set forth. It will be understood that these examples are intended to describe illustrative embodiments of the invention and are not intended to limit the scope of the invention in any way.

### EXAMPLES

### Example 1

### Arabidopsis Triphosphate Tunnel Metalloenzyme, AtTTM2, is a Negative Regulator of the Salicylic Acid-Mediated Feedback Amplification Loop for Defence Responses

### SUMMARY

The triphosphate tunnel metalloenzyme (TTM) superfamily represents a group of enzymes that are characterized by their ability to hydrolyze a range of tripolyphosphate substrates. Arabidopsis, encodes three TTM genes, AtTTM1, 2 and 3. Although AtTTM3 has previously been reported to have polytriphosphatase activity, recombinantly expressed AtTTM2 unexpectedly exhibited pyrophosphatase activity. AtTTM2 knockout (KO) mutant plants exhibit an enhanced hypersensitive response, elevated pathogen resistance against both virulent and avirulent pathogens, and elevated accumulation of salicylic acid (SA) upon infection. In addition, stronger systemic acquired resistance (SAR) compared to wild type plants was observed. These enhanced defence responses are dependent on SA, PAD4, and NPR1. Despite their enhanced pathogen resistance, ttm2 plants did not display constitutively active defence responses, suggesting that AtTTM2 is not a conventional negative regulator, but a negative regulator of the amplification of defence responses. The transcriptional suppression of AtTTM2 by pathogen infection or treatment with SA or the SAR activator, BTH, further supports this notion. Such transcriptional regulation is conserved among TTM2 orthologues in the crop plants, soybean and canola, suggesting that TTM2 is involved in immunity in a wide variety of plant species. This indicates the possible usage of TTM2 KO mutants for agricultural application to generate pathogen resistant crop plants.

### Introduction

The triphosphate tunnel metalloenzyme (TTM) superfamily comprises a group of enzymes that are characterized by their ability to hydrolyze a range of tripolyphosphate substrates. All members of this superfamily utilize triphosphate substrates and require a divalent cation cofactor for their activity, usually Mg²⁺ or Mn²⁺ (Bettendorff and Wins, 2013). This superfamily contains two previously characterized groups of proteins: RNA triphosphatases and CYTH domain proteins (Iyer and Aravind, 2002; Gong et al., 2006). The CYTH domain was named after its two founding members, the CyaB adenylate cyclase from Aeromonas hydrophila and the mammalian thiamine triphosphatase (Iyer and Aravind, 2002). Despite low overall amino acid sequence similarity, all TTM family members possess a tunnel structure composed of eight antiparallel β strands (β barrel) (Gong et al., 2006; Gallagher et al., 2006; Song et al., 2008; Moeder et al., 2013). The signature EXEXK motif (where X is any amino acid) located in the β barrel has been shown to be important for catalytic activity (Lima et al., 1999; Gallagher et al., 2006).

The enzymatic and biological function of most TTM family members is unknown. However, they appear to act on nucleotide and organophosphate substrates (Bettendorff and Wins, 2013) and acquired divergent biological functions in different taxonomic lineages (Iyer and Aravind, 2002). Known functions include adenylate cyclase for CyaB from Aeromonas hydrophila and YpAC-IV from Yersinia pestis (Sismeiro et al., 1998; Gallagher et al., 2006), thiamine triphosphatase in mammals (Lakaye et al., 2004) and RNA triphosphatase in fungi, protozoa, and some viruses (Shuman, 2002). In some instances, TTM proteins are fused to additional domains, such as a nucleotide kinase domain (Iyer and Aravind, 2002).

Plants possess two types of TTM proteins: one that comprises only the CYTH domain and another with a CYTH domain fused to a phosphate-binding (P-loop) kinase domain (Iyer and Aravind, 2002). Arabidopsis, as most other plant species, codes for three TTM genes, termed AtTTM (Triphosphate Tunnel Metalloenzyme) 1, 2 and 3. AtTTM3 possesses only a CYTH domain, while AtTTM1 and AtTTM2 encode a nucleotide/uridine kinase domain fused to the CYTH domain (Moeder et al., 2013). So far, the exact biological function of TTM proteins in plants is not clear. Previous analysis of AtTTM3 and found that it does not display adenylate cyclase activity despite its annotation, but acts on tripolyphosphate and with lower affinity, nucleotide triphosphates, releasing inorganic phosphate (Pᵢ), similar to the TTM proteins from Clostridium thermocellum (CthTTM) and Nitrosomonas europaea (NeuTTM) (Keppetipola et al., 2007; Delvaux et al., 2011; Moeder et al.; 2013; Bettendorff and Wins, 2013). Additionally, a T-DNA insertion knock out line of AtTTM3 displayed a delay in root growth as well as reduced length and number of lateral roots, suggesting a role for AtTTM3 in root development.

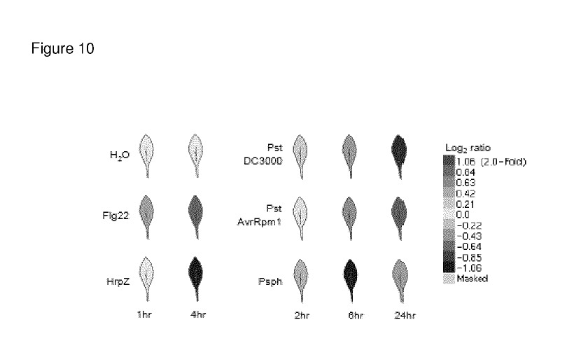

In order to gain insight into the biological function of AtTTM1 and AtTTM2 the Bio-Array Resource was surveyed (BAR; http://bar.utoronto.ca/efp/cgi-bin/efpWeb.cgi; Winter et al., 2007) for any publicly available expression analysis data that might provide clues for the biological role of these AtTTMs. The expression of AtTTM2 was suppressed almost 2-fold after treatment with flg22, the well-studied pathogen-associated molecular pattern (PAMP) peptide and after infection with various virulent and avirulent strains of Pseudomonas syringae (FIG. 10). This data suggests the possible involvement of AtTTM2 in pathogen defence responses in plants.

The plant defence system has been studied extensively in the last two decades and two levels of resistance responses have been reported. The first line of defence is basal immunity, which is triggered by the recognition of molecules that are conserved among many pathogens (above-mentioned PAMPs) and is thus referred to as PTI (PAMP-triggered immunity). Another line of defence is a stronger response to pathogen infection, which is mediated by resistance (R) genes that can recognize their cognate effectors from the pathogen either directly or indirectly. This is known as effector-triggered immunity (ETI; Bent and Mackey, 2007). The hypersensitive response (HR), which is characterized by apoptosis-like cell death at and around the site of pathogen entry is one common defence mechanism activated by R gene-mediated pathogen recognition (Hammond-Kosack and Jones, 1996; Heath, 2000). During HR development, an increase in salicylic acid (SA) and the accumulation of pathogenesis-related (PR) proteins are observed (Vlot et al., 2008). Later, resistance against virulent pathogens can also be seen in uninoculated systemic leaves. This phenomenon is called systemic acquired resistance (SAR) and confers a long-lasting, broad-range resistance to subsequent infection (Vlot et al., 2008; Shah and Zeier, 2013). Elevated SA levels and PR gene expression can also be detected in uninoculated leaves that exhibit SAR. Treatment with SA or synthetic SAR activators, such as benzothiadiazole (BTH), can also trigger SAR (Lawton et al., 1996; Vlot et al., 2008). Recently, a number of metabolites that are involved in long-distance signaling have been identified, such as methyl salicylate (MeSA), dehydroabietinal (DA), azelaic acid (AzA), glycerol-3-phosphate (G3P), and the lysine catabolite pipecolic acid (Pip) (Shah and Zeier, 2013).

Over the last two decades, significant effort has been made to identify components in the pathogen resistance signal transduction pathway. For instance, ISOCHORISMATE SYNTHASE1 (ICS1) has been revealed to play a critical role in the biosynthesis of pathogen-induced SA. sid2/ics1 mutants fail to produce elevated levels of SA after pathogen infection and are thus hypersensitive to pathogens (Wildermuth et al., 2001; Nawrath et al., 1999). NPR1 (NON EXPRESSOR OF PR GENES1) is a key regulator of SA-mediated resistance and npr1 mutant plants fail to respond to exogenously supplied SA (Cao et al., 1994). The lipase-like proteins, ENHANCED DISEASE SUSCEPTIBILITY1 (EDS1) and PHYTOALEXIN DEFICIENT4 (PAD4) (Parker et al., 1996; Glazebrook et al., 1996), participate in both basal and R protein-mediated defence responses (Falk et al., 1999; Jirage et al., 1999). EDS1 interacts with PAD4 and SAG101 (SENESCENCE ASSOCIATED GENE101) and both EDS1 and PAD4 are required for HR formation and the restriction of pathogen growth (Feys et al., 2001; 2005). A screen of mutants exhibiting constitutive activation of resistance responses also identified components in defence. They show heightened resistance, usually accompanied by elevated levels of SA and PR genes. These autoimmune mutants also frequently display spontaneous HR-like lesions, and thus are referred to as lesion mimic mutants (Moeder and Yoshioka, 2008; Hofius et al., 2009).

Here, we demonstrate that AtTTM2 acts as a negative regulator of plant immunity, likely at the positive amplification loop of defence responses. Knockout mutants for AtTTM2 show enhanced pathogen resistance, while over-expressors display enhanced susceptibility. The knockout mutants do not show constitutive activation of defence responses like most autoimmune mutants, but exhibit enhanced SAR upon treatments with pathogens, suggesting that they are in a primed state. Furthermore, the expression of TTM2 orthologues in canola and soybean display the same transcriptional down-regulation after BTH treatment, suggesting that the biological function of TTM2 in pathogen defence is conserved among agriculturally important crop plants.

### Results

### AtTTM2 is Down-Regulated After Pathogen Infection

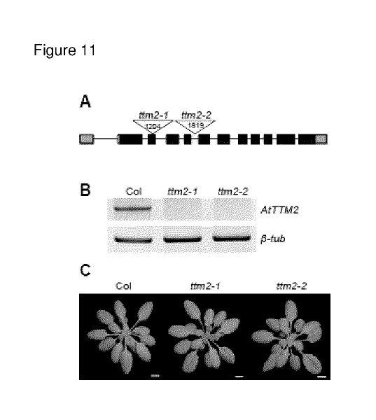

Three genes, At1g73980, At1g26190, and At2g11890, are annotated as CYTH domain proteins in the Arabidopsis thaliana genome, which have been named AtTTM1, 2, and 3 (triphosphate tunnel metalloenzyme; Moeder et al., 2013). Two allelic homozygous T-DNA insertion knockout (KO) lines were obtained for AtTTM2—Salk_145897 (ttm2-1) and Salk_114669 (ttm2-2). The T-DNA insertion positions were found to be located in exon 3 and intron 5 in ttm2-1 and ttm2-2, respectively (FIG. 11A). Reverse transcription (RT)-PCR analysis showed that both lines are indeed KO mutants (FIG. 11B). A morphological comparison showed no detectable difference in the size or shape of both ttm2 KO lines compared to wild type Columbia (Col) (FIG. 11C).

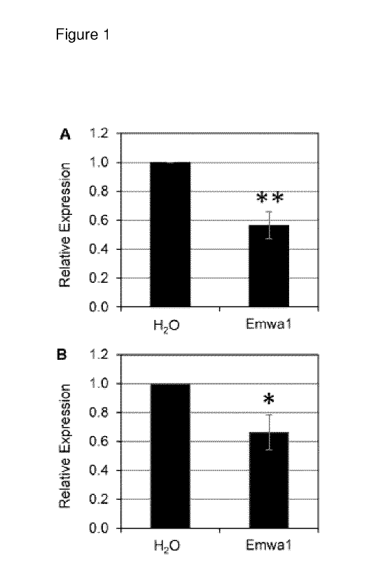

As mentioned, public microarray data revealed the down-regulation of AtTTM2 during pathogen infection (FIG. 10). To confirm these results, quantitative real-time PCR (qPCR) was conducted on Col wild type plants that were infected with the oomycete pathogen Hyaloperonospora arabidopsidis (Hpa), isolate Emwa1. We observed a 2-fold reduction in AtTTM2 transcript levels in infected cotyledons compared to mock treatment (FIG. 1A), indicating the involvement of AtTTM2 in pathogen defence. Interestingly, AtTTM2 was also down-regulated in uninfected systemic tissue of the same seedlings, indicating a role for AtTTM2 in SAR as well (FIG. 1 B).

ttm2 Exhibits Enhanced Resistance Against Hyaloperonospora arabidopsidis

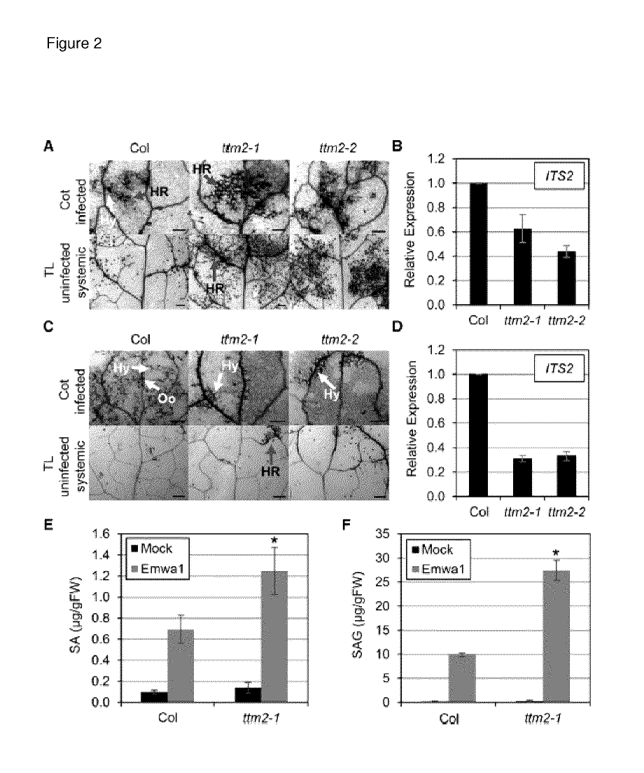

Since AtTTM2 is down-regulated after pathogen infection, we asked whether ttm2 mutants show alterations in defence related phenotypes. Cotyledons of 7 to 10 day-old seedlings were infected with the Hpa isolate, Emwa1, which is avirulent to the Col ecotype. It is notable that although the Emwa1 isolate is considered to have an incompatible interaction with the Col ecotype, the resistance in this ecotype is not perfect and initial layers of mesophyll cells may show the emergence of some hyphae (FIG. 2A, Cot). ttm2 lines, in addition to having fewer or no hyphae, also exhibited a greater manifestation of HR cell death on infected tissue compared to wild type suggesting enhanced resistance (FIG. 2A, Cot). qPCR analysis also showed approximately 2-fold less ITS2 (internal transcribed spacer2) transcript levels, a marker to quantify oomycete infection (Quentin et al., 2009; FIG. 2B, 12) indicating less growth of pathogens in ttm2 plants. We frequently observed the formation of micro-HR-like cell death in uninfected systemic leaves of wild type plants after avirulent infection on cotyledons (FIG. 2A, TL) similarly to the findings of Alvarez et al. (1998). Interestingly, ttm2 plants displayed significantly enhanced HR cell death on the uninfected systemic true leaves (FIG. 2A, TL).

To determine whether this enhanced resistance was specific to ETI or whether it also affected PTI, infection with the virulent Hpa isolate, Emco5, was conducted. Trypan blue analysis revealed little to no hyphae on infected tissue of ttm2 while in wild type plants, hyphal structures and oospore formation were clearly visible throughout the leaf (FIG. 2C, Cot). Consistent with this observation, ITS2 transcript levels in infected cotyledons of ttm2 seedlings were more than 2-fold lower compared to wild type (FIG. 2D, 12B). Interestingly, we also observed enhanced HR-like cell death along the veins of uninfected systemic leaves of ttm2 seedlings (FIG. 2C).

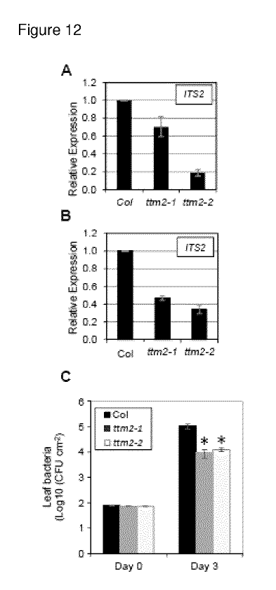

FIG. 12C shows that ttm2 plants also displayed enhanced resistance to the bacterial pathogen, Pseudomonas syringae DC3000 (AvrRps4). These data indicate that ttm2 plants exhibited enhanced resistance against both avirulent and virulent pathogens.

SA has been shown to be a critical signaling molecule in pathogen defence. In line with the resistance phenotype, a significant increase in free SA and its conjugated form, salicylic acid glucoside (SAG), was observed in ttm2 plants upon pathogen infection compared to wild type (FIG. 2E, F). Taken together, these data suggest that AtTTM2 is likely involved in SA-mediated defence signaling.

ttm2 is not a Lesion Mimic Mutant

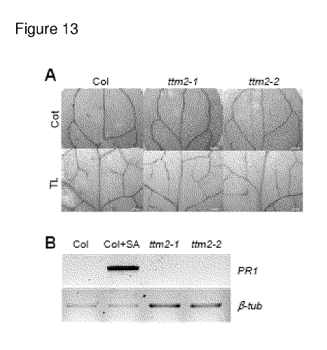

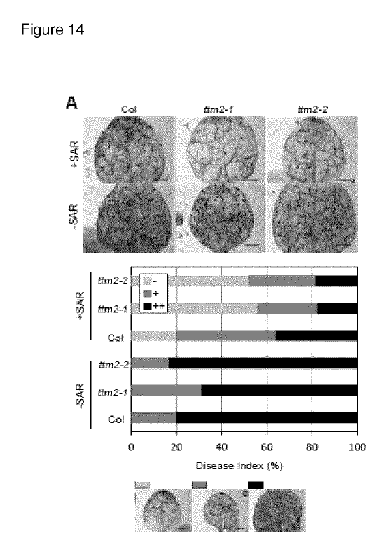

To date, various autoimmune mutants have been reported. They show enhanced resistance against various pathogens and often exhibit activation of resistance responses such as accumulation of SA and constitutive PR gene expression without pathogen infection. One well studied class of autoimmune mutants, called lesion mimic mutants, additionally exhibits spontaneous cell death formation without pathogen infection (Moeder and Yoshioka, 2008). To test whether resistance responses are activated without pathogen infection in ttm2, trypan blue analysis on uninfected ttm2 seedlings was conducted and revealed no spontaneous cell death formation (FIG. 13A). Additionally, no elevated expression of the defence marker gene, PR1 (Laird et al., 2004), was observed in ttm2 seedlings without pathogen infection (FIG. 13B). These data suggest that ttm2 is not a lesion mimic or conventional autoimmune mutant, but likely a priming mutant that exhibits enhanced resistance upon pathogen infection.

ttm2 Exhibits Enhanced SAR

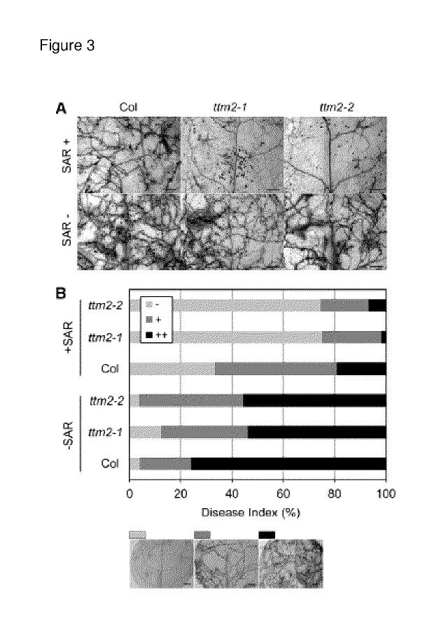

The observation that AtTTM2 was also down-regulated in uninfected systemic leaves (FIG. 1B) combined with the enhanced HR cell death in ttm2 seedlings (FIG. 2A) prompted us to investigate whether ttm2 is also affected in its SAR response. To assess SAR, we first treated cotyledons of wild type and ttm2 plants with either water (SAR −) or the avirulent Hpa isolate, Emwa1 (SAR +). We then performed challenge inoculation using the aggressive virulent Hpa isolate, Noco2, on the upper systemic leaves (FIG. 3A, 14A). We used very strong infection conditions, i.e. 1×10⁵ conidiospores, of the aggressive isolate, Noco2, in order to see a clear difference between SAR-induced and non-induced groups. Thus, both wild type and ttm2 plants displayed comparable hyphae growth in water-treated plants (SAR −, FIG. 3A, 14A lower panels). In contrast, Hpa-treated ttm2 plants (SAR +, FIG. 3A, 14A upper panels) revealed a stronger reduction in pathogen growth in systemic leaves compared to SAR+ wild type plants. Stained leaves were microscopically examined and assigned to different classes (FIG. 3B, 14B). Fisher Exact Probability Test indicated a significant difference between the ttm2 KO lines and Col wt (p<0.0001). These data suggest that ttm2 mutants exhibit enhanced SAR.

The Enhanced Resistance Phenotype of ttm2 Requires PAD4, ICS1, and NPR1

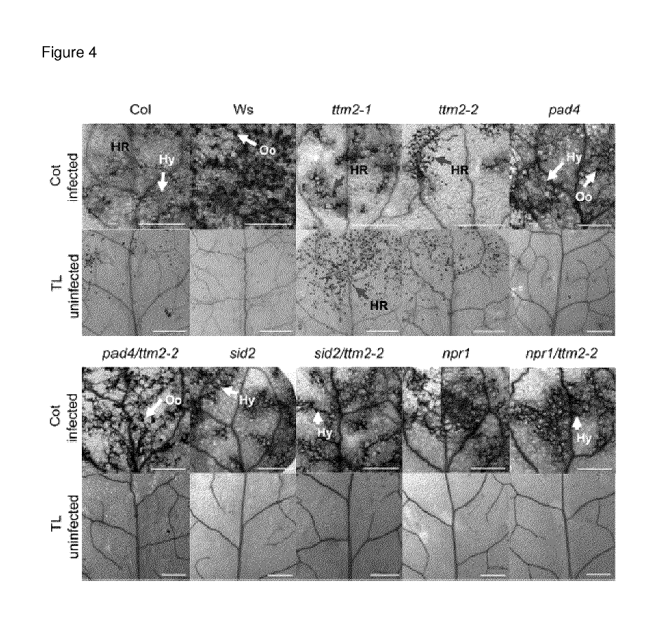

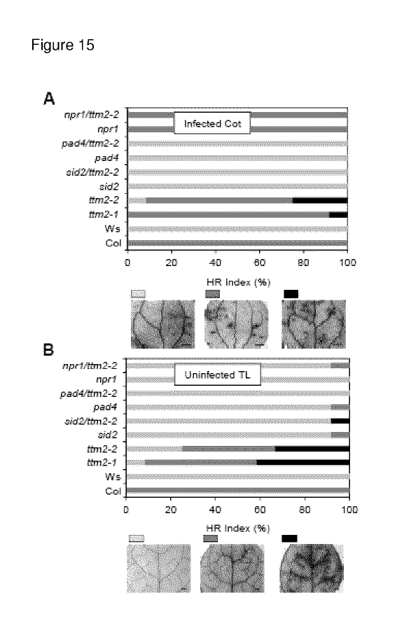

It has been shown that PAD4, SID2 (ICS1), and NPR1 play key roles in SA-dependent defence responses (Glazebrook et al., 1996; Jirage et al., 1999; Nawrath et al., 1999; Wildermuth et al., 2001; Cao et al., 1997). To investigate whether AtTTM2-mediated resistance requires these signaling components, we performed epistatic analyses using double mutants of ttm2-2 and pad4-1, sid2-1, or npr1-1. Col and Wassilewskija (Ws) ecotypes are resistant and susceptible, respectively, to the Hpa isolate, Emwa1 (FIG. 4). As expected, Col wild type exhibited resistance with some hyphae present on the infected tissue along with punctate areas of HR cell death in both infected tissue and uninfected systemic tissue, while Ws wild type exhibited susceptibility with massive hyphal growth and oospore formation in infected tissue and no visible signs of HR in the uninfected systemic leaves (FIG. 4, TL). pad4-1, sid2-1, and npr1-1 single mutants also exhibited susceptibility with little or no visible HR (FIGS. 4, 15), but a great presence of hyphae and in some cases, oospores (FIG. 4), as expected. All double mutants with ttm2 exhibited similar susceptibility as pad4-1, sid2-1, and npr1-1 single mutants (FIGS. 4, 15). These data indicate that PAD4, ICS1, and NPR1 are all required for the enhanced resistance phenotype of ttm2.

### AtTTM2 Expression is Negatively Regulated by SA and PAMP Treatment

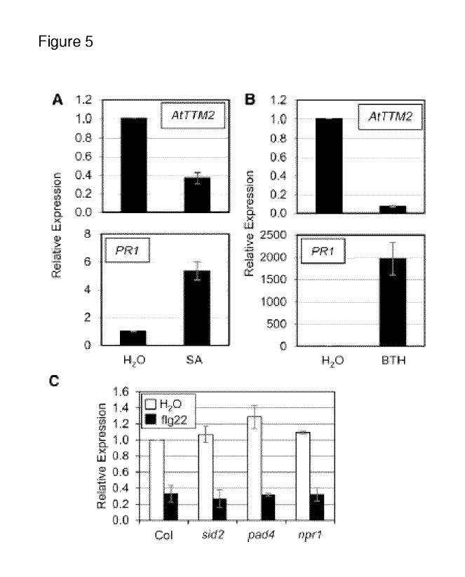

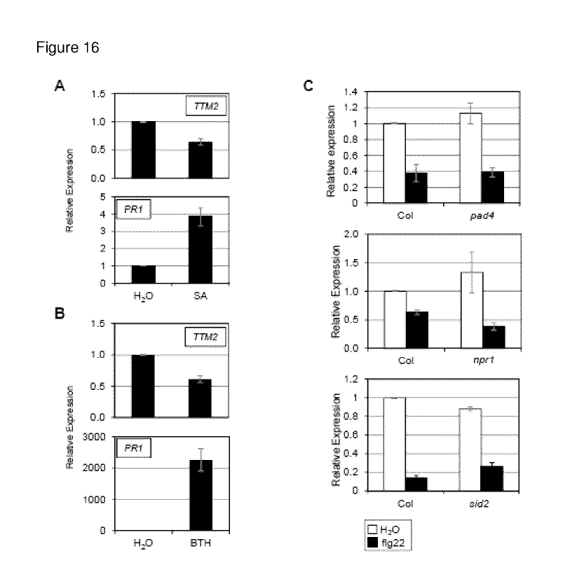

Since pathogen infection down-regulates the transcription of AtTTM2 (FIG. 1), the effect of SA on AtTTM2 expression was tested. Col wild type plants were sprayed with 100 μM SA and assessed 24h later for changes in expression levels. AtTTM2 was down-regulated by more than 2-fold after SA treatment (FIGS. 5A, 16A). This down-regulation was also observed after treatment with the SAR activator, BTH (200 μM) (FIGS. 5B, 16B). This was correlated with an increase in PR1 gene expression (FIGS. 5A, B and 16A, B bottom panels). Publicly available micro array data indicated that AtTTM2 is also down-regulated after treatment with the PAMP, flg22 (FIG. 10). Our qPCR confirmed that 4h after treatment with the flg22 peptide (5 μM), AtTTM2 was down-regulated by 70% (FIG. 5C, 16C).

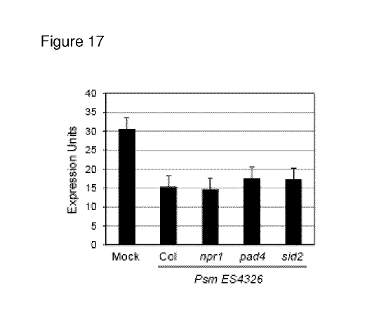

The fact that AtTTM2 gene expression was down-regulated upon pathogen infection (FIG. 1) as well as SA/BTH treatment and flg22 treatment (FIG. 5) made us assess the requirement of key components in SA-mediated resistance for the transcriptional regulation of AtTTM2. Interestingly, after treatment with flg22, sid2, pad4 and npr1 plants displayed the same level of AtTTM2 down-regulation as wild type plants (FIGS. 5C, 16C). A similar result was seen after infection with Pseudomonas syringae ES4326 (FIG. 17). Taken together these data suggest that SA, PAD4 and NPR1 are not required for the transcriptional down-regulation of AtTTM2, but are required for the resistance phenotype of the ttm2 mutants.

### Over-Expression of AtTTM2 Confers Enhanced Susceptibility to Pathogens

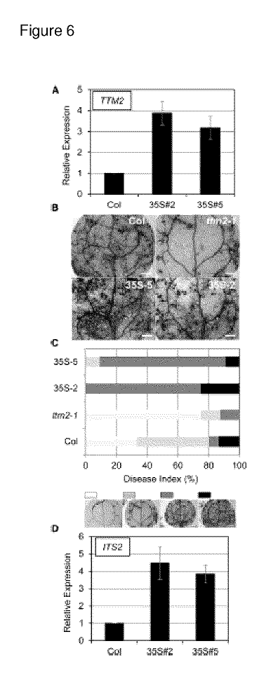

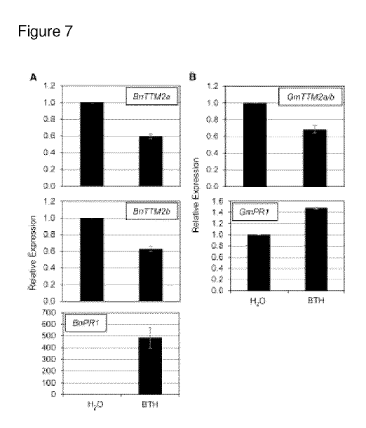

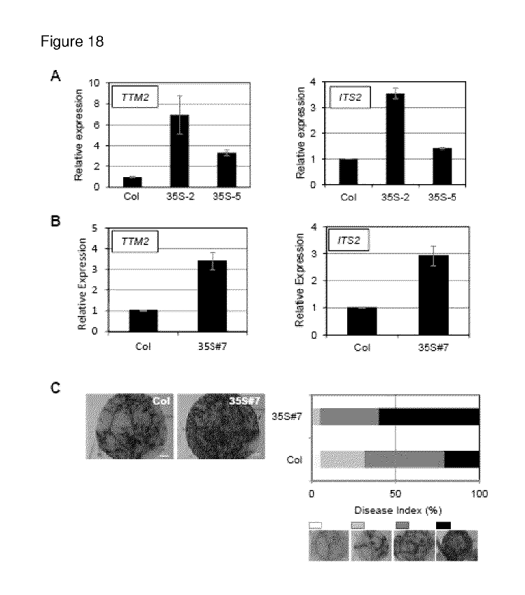

The observation that AtTTM2 is down-regulated upon pathogen infection and SA/flg22 treatment combined with the fact that ttm2 plants display enhanced disease resistance strongly suggests that AtTTM2 is a negative regulator of disease resistance. Therefore, constitutive expression of AtTTM2 may lead to enhanced disease susceptibility. Thus, we created AtTTM2 over-expressor lines, where AtTTM2 expression is driven by the strong CaMV 35S promoter. To detect differences in disease outcome, we used relatively moderate infection conditions with the virulent Hpa isolate, Emco5. We observed elevated expression of AtTTM2 in three independent transgenic lines even after pathogen infection (FIGS. 6A, 18). While only 60% of Col wild type plants and 30% of ttm2 plants exhibited heavy hyphal growth 10 days after infection, 100% of the plants of the three over-expression lines showed strong infection (FIGS. 6B, C, 18C). Fisher Exact Probability Test indicated a significant difference between the over-expressor lines and Col wt (p<0.001). This was also confirmed quantitatively by measuring the expression of the oomycete marker, ITS2 (FIGS. 6D, 18). This data strongly suggests that down-regulation of AtTTM2 is indeed required for normal levels of disease resistance.

### AtTTM2 Function is Likely Conserved Among Different Plant Species

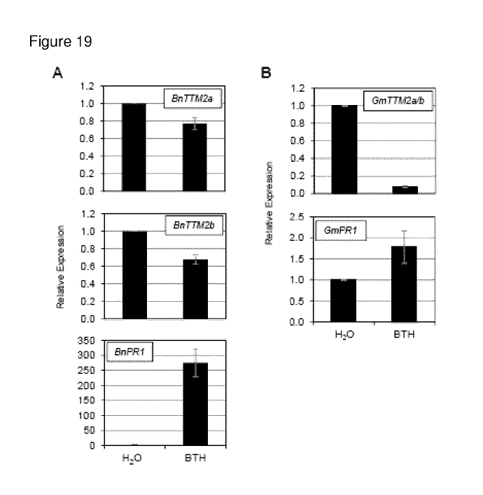

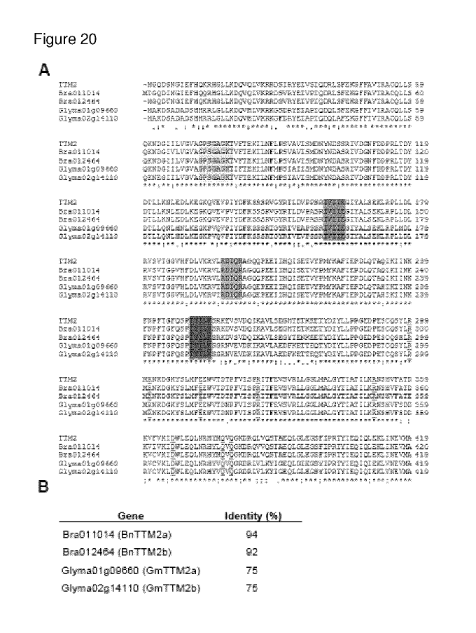

Data from Phytozome (www.phytozome.net) indicated that TTM2 is highly conserved in a wide variety of plant species. This may indicate that these orthologues are also involved in pathogen defence responses. Similarities in the transcriptional expression pattern of TTM2 orthologues can serve as an indication of functional conservation. Thus, the expression of AtTTM2 orthologues of soybean (Glycine max) and canola (Brassica napus) was analyzed by qPCR after treatment with BTH. Interestingly, the TTM2 orthologues in B. napus (BnTTM2a, BnTTM2b) (FIG. 7A, 19A) and in G. max (GmTTM2a/b; note that the two isoforms could not be distinguished due to high sequence identity) (FIGS. 7B, 19B) were similarly down-regulated in response to BTH as their Arabidopsis orthologues. This data combined with the high sequence identity (BnTTM2a, 94%; BnTTM2b, 92%; GmTTM2a, 75%; GmTTM2b, 75%; FIG. 20) suggests that the function of TTM2 as a negative regulator of defence responses is likely evolutionarily conserved in other plant species as well.

### AtTTM2 Displays Pyrophosphatase Activity

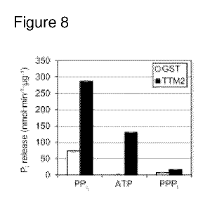

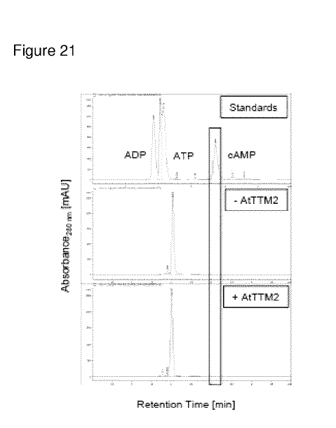

The three TTM genes in Arabidopsis are annotated as adenylate cyclases. However, we recently reported that AtTTM3 does not produce cyclic AMP (cAMP; Moeder et al., 2013). Similarly, recombinantly expressed AtTTM2 also was not able to produce cAMP (FIG. 21). Since AtTTM3 displayed strong tripolyphosphatase activity, we assessed the enzymatic properties of AtTTM2 on several organo-phosphate substrates. While AtTTM3 showed strong affinity for tripolyphosphate (PPPᵢ), weaker affinity for ATP and no affinity for pyrophosphate (PPᵢ) (Moeder et al., 2013), AtTTM2 surprisingly displayed strongest affinity for PPᵢ, weaker activity for ATP and almost none for PPPᵢ (FIG. 8). AtTTM2 was expressed as a GST-fusion protein. Protein extracted from E. coli expressing the GST tag alone confirmed that the observed activities are not due to contaminating bacterial proteins (FIG. 8). These data suggest divergent biological functions of the AtTTM genes, which is consistent with the different phenotypes observed in ttm2 and ttm3.

### Discussion

In order to understand the biological function of the triphosphate tunnel metalloenzyme, AtTTM2, we have characterized the AtTTM2 KO mutants, ttm2-1 and ttm2-2. Both lines displayed enhanced resistance against both virulent and avirulent pathogens, as they exhibited lower growth of both types of pathogens combined with an enhancement of HR cell death. In addition, SAR was also enhanced in these mutants. The enhanced resistance was dependent on the well-known defence signaling components, SA, PAD4 and NPR1, which indicates that AtTTM2 is involved in the bona fide defence signaling pathway and is likely a negative regulator. Transcriptional suppression of AtTTM2 after pathogen infection, PAMP recognition, or SA/BTH treatment further supports this notion. Interestingly, the enhanced pathogen resistance is only observed upon pathogen infection - no significant auto-activation of defence responses, such as spontaneous cell death formation and elevated levels of basal SA or PR1 gene expression were observed. This differentiates AtTTM2 mutants from the majority of conventional autoimmune mutants (Moeder et al., 2008; Hofius et al., 2009).

A similar phenomenon was reported in the Arabidopsis mutants enhanced disease resistance (edr) 1 and 2 (Frye and Innes 1998; Tang et al., 2005a). EDR1 and 2 encode a CTR1 family MAPKKK and an unknown protein with a PH, a START, and a DUF1336 domain, respectively (Frye et al., 2001; Tang et al., 2005a, 2005b; Vorwerk et al., 2007). Both mutants were identified in the same screen for decreased susceptibility against Pseudomonas syringae DC3000 without constitutive PR gene expression and also show enhanced resistance against other pathogens such as Erysiphe cichoracearum.

Interestingly, both mutants display stronger and faster defence responses upon pathogen infection; however, no obvious auto-activation of defence was observed, just like for ttm2. These phenotypes were suppressed in mutants with defects in the SA signal transduction pathway (e.g., sid2, pad4, npr1, eds1), but not by those with defects in the ethylene/jasmonate pathway, suggesting that they are hypersensitive to or have a lower threshold in activating the SA pathway (Frye et al., 2001; Tang et al., 2005; Vorwerk et al., 2007). The precise molecular mechanisms of these mutants are not yet clear; however the reported phenotypes are remarkably similar to those of ttm2. The only outstanding difference between ttm2 and edr2 is the enhanced SAR phenotype in ttm2. As shown, ttm2 displayed strong enhancement of SAR, including HR cell death, in uninfected systemic leaves, but edr2-mediated enhancement of resistance does not occur in uninfected systemic leaves. This indicates that although the mutant phenotypes are similar, the molecular mechanism behind the phenomena is fundamentally different.

In terms of SAR, AGD2-LIKE DEFENCE RESPONSE PROTEIN1 (ALD1) was shown to be involved in both local and systemic resistance (Song et al., 2004). ALD1 is transcriptionally induced by pathogen infection as well as BTH treatment in both inoculated and systemic tissues. ald1 mutant plants have increased susceptibility to avirulent pathogens and cannot activate SAR. The ALD1 aminotransferase is involved in the biosynthesis of the SAR regulator pipecolic acid, which accumulates in local and systemic tissue of SAR-induced plants (Návarová et al., 2012). Pipecolic acid has been shown to mediate signal amplification that enables systemic SA accumulation, SAR establishment and defence priming responses in SAR-induced plants. Considering that ttm2 also does not show constitutive activation of resistance and displays a SAR phenotype, AtTTM2 may act by fine-tuning the amplification of defence responses in both inoculated and uninoculated leaves. Indeed, an SA-mediated feedback amplification loop has been suggested for a long time (Shah, 2003). For instance, EDS1 and PAD4, which are important defence signaling components, are both regulators and effectors of SA signaling, strongly suggesting the existence of a SA-mediated feedback amplification loop (Dong, 2004). Likewise, ACCELERATED CELL DEATH6 (ACD6), which is believed to work upstream of SA biosynthesis, is transcriptionally induced by BTH (Lu et al., 2003).

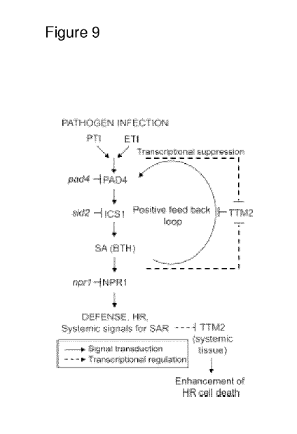

Thus, it can be hypothesized that recognition of pathogen infection suppresses the expression of AtTTM2, which acts as a negative regulator of the amplification loop, to facilitate a quick and strong resistance response. At a later time point, SA accumulation induced by pathogen infection further suppresses the expression of AtTTM2 to boost the positive feedback amplification loop of defence responses. Transcriptional down-regulation of AtTTM2 can already be seen 4h after treatment with flg22 and 24h after infection with Pseudomonas syringae (FIG. 5C, 17). Interestingly, AtTTM2 down-regulation was also observed in flg22-treated as well as Pseudomonas syringae-infected sid2, npr1 and pad4 mutant plants (FIGS. 5C, 16, 17), indicating that the down-regulation is triggered upstream of PAD4. SA/BTH treatment causes AtTTM2 down-regulation either through an additional mechanism or through feedback via the SA amplification loop (FIG. 9). In this scenario, AtTTM2 plays a role to prevent accidental activation of defence responses through the positive feedback amplification loop in the absence of pathogens. Thus, ttm2 exhibits a primed mutant phenotype: it can induce resistance responses stronger than wild type plants, but no constitutive activation of defence responses is observed. A model of this concept is presented in FIG. 9. While a SA-mediated feedback amplification loop has been discussed for quite some time (Shah, 2003), only a few studies have identified components of this feedback loop (Song et al., 2004; Raffaele et al., 2006; Roberts et al., 2013). Whether TTM2 negatively regulates defence amplification by attenuating pipecolic acid biosynthesis remains to be determined. The molecular mechanism of AtTTM2 will further our understanding of the SA-mediated feedback amplification loop.

All three Arabidopsis TTMs have been annotated as adenylate cyclases based on sequence similarity to CyaB from Aeromonas hydrophila Oyer and Aravind, 2002). However, in this and previous work, we have shown that recombinantly expressed AtTTM3 and AtTTM2 do not show adenylate cyclase activity (Moeder et al., 2013; FIG. 21). AtTTM3 rather exhibits strong tripolyphosphatase activity with a strong affinity for tripolyphosphate (PPPᵢ). On the other hand, AtTTM2 showed strongest affinity for PP, and only weak activities for ATP and PPPᵢ. Although the actual in vivo substrates are currently unknown, the difference in the in vitro substrate preference between AtTTM3 and 2 indicates distinct biological functions of these two TTM family members. Furthermore, in addition to a CYTH domain, both AtTTM1 and 2, but not AtTTM3, possess a P-loop kinase domain in their N-termini. It is annotated as a uridine/cytidine kinase and has conserved Walker A, Walker B, and lid module motifs (FIG. 20; Leipe et al., 2003). This indicates the possibility that AtTTM1 and 2 have dual enzymatic activities, both phosphatase and kinase. Alternatively, the CYTH domain may have lost its catalytic function in AtTTM1 and 2 and its function might be to bind and position their specific in vivo substrate for the kinase domain (Iyer and Aravind, 2002). This idea is supported by the fact that many of the conserved catalytic residues of TTM proteins are altered in AtTTM1 and 2. The stereotypical EXEXK motif of CYTH proteins (including AtTTM3) is altered to TYILK. Furthermore, the majority of the conserved basic and acidic residues in the β-barrel are not conserved in AtTTM1 and 2 (FIG. 20). These residue changes are conserved among the TTM2 orthologues in other plant species, indicating that they contribute to the unusual catalytic activity of AtTTM2. Unlike all other described TTM proteins, which act on triphosphate substrates, AtTTM2 prefers a diphosphate (pyrophosphate). In any case, the study of in vivo substrates for AtTTMs and the characterization of AtTTM1 will provide further insights into this group of proteins in plants and the possible role of this phosphatase/kinase in pathogen defence responses. The analysis of AtTTM1 is currently in progress.

Genomic sequence analyses indicated that all three TTM family members are conserved among most plant species, further indicating the distinct function of all three TTMs in plants. Interestingly, transcriptional suppression of TTM2 by BTH was observed in soybean and canola, as in Arabidopsis, strongly indicating that the orthologues of TTM2 in these crop plants likely also work as negative regulators of defence responses. This raises the possibility that KO crop mutants for TTM2 will also show enhanced resistance similar to Arabidopsis ttm2 plants, providing a useful tool in agricultural biotechnology to generate pathogen-resistant crop plants.

### Materials & Methods

### Plant Growth Conditions and Pathogen Assays

Arabidopsis (Arabidopsis thaliana accession Columbia), canola (Brassica napus var. Westar), and soybean (Glycine max var. Harasoy) plants were grown in Sunshine Mix at 22° C., 60% relative humidity (RH), and ˜140 μE m⁻² s⁻¹ with a 9h-photoperiod. 7-10 day-old Arabidopsis plants were infected with Hyaloperonospora arabidopsidis (Hpa). Spore counts of 1×10⁵ conidiospores ml⁻¹, 8×10⁵ cells ml⁻¹, and 2×10⁵ cells ml⁻¹ were used for Noco2, Emco5, and Emwa1 isolates, respectively. Seedlings were then infected via drop inoculation and left at 16° C., >90% RH for 7-10 days before disease assessment. 4-week-old plants were infiltrated with 1×10⁵ CFU ml⁻¹ of the bacterial pathogen, Pseudomonas syringae tomato DC3000 (AvrRps4) and bacterial growth was assessed at 0 and 3 days post infiltration.

### CaMV 35S Transgenic Lines

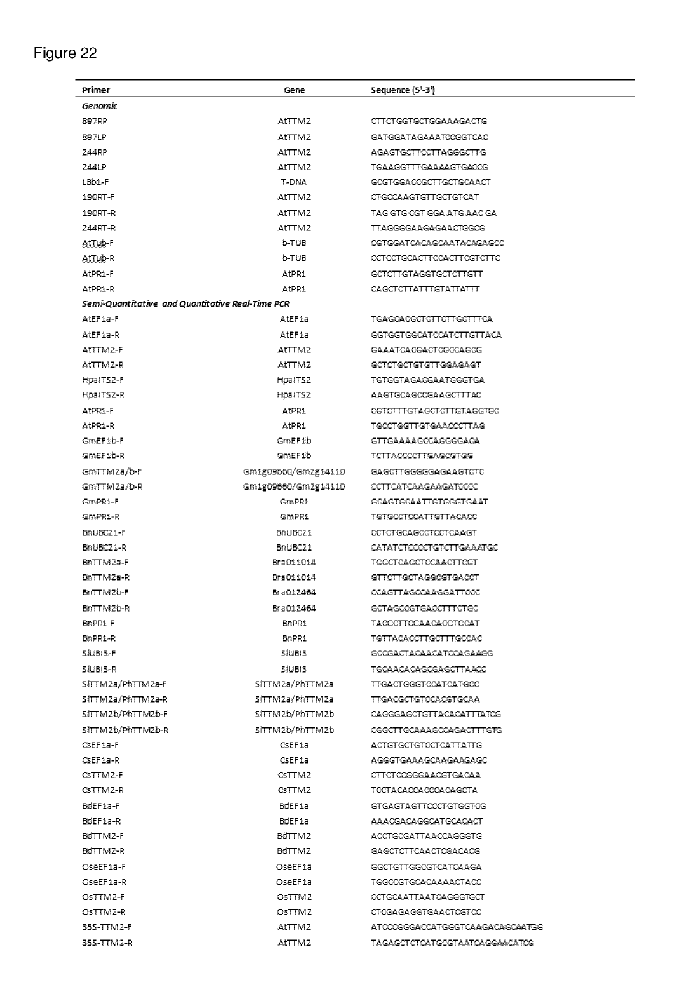

The coding sequence of AtTTM2 was amplified from Arabidopsis thaliana Columbia cDNA using the primers 35S-TTM2-F and 35S-TTM2-R (FIG. 22) and cloned into pBl121 (Clontech). The vector was transformed into Columbia wild type plants through Agrobacterium tumefaciens-mediated transformation using the floral dip method (Clough and Bent, 1998).

### RNA Extraction and RT-PCR

RNA extraction was carried out using the TRIzol reagent (Life Technologies, Carlsbad, Calif.), according to the manufacturer's instructions. Reverse transcriptase (RT)-PCR was performed using cDNA generated by SuperScript II Reverse Transcriptase (Life Technologies, Carlsbad, Calif.) according to the manufacturer's instructions. Expression of PR1 was visualized by gel electrophoresis of samples after RT-PCR with PR1 primers (AtPR1-F, AtPR1-R).

### Quantitative Real-Time PCR

Quantitative real-time PCR was performed using Fast SYBR Green Master Mix (Life Technologies, Carlsbad, Calif.). The expression of Arabidopsis genes were normalized to the expression of AtEF1A (elongation factor1-alpha) while the expression of soybean and canola genes were normalized to GmEF1B (elongation factor1-beta) and BnUBC21 (ubiquitin conjugating enzyme21), respectively. All primer sequences are listed in FIG. 22.

### Confirmation of T-DNA Insertion Knockout Lines

The SALK lines, SALK_145897 (ttm2-1) and SALK_114669 (ttm2-2), were obtained from the SALK Institute (Alonso et al., 2003). Homozygous plants were isolated using gene-specific primers for ttm2-1 (897RP, 897LP) and for ttm2-2 (244RP, 244LP) in combination with the T-DNA specific primer LBb1-F. RT-PCR was then performed on cDNA from both ttm2 lines to confirm the knockout status using the full-length TTM2 primers (190RT-F, 244RT-R). Expression was normalized to the expression of β-tubulin. Primer sequences are listed in FIG. 22.

### Epistatic Analysis

ttm2-2 was crossed with pad4-1 (Glazebrook et al., 1996; Jirage et al., 1999), ics1-1 (Wildermuth et al., 2001), and npr1-1 (Cao et al., 1997). Homozygous double mutants were isolated in the F2 generation.

SA, BTH, and flg22 Treatments

7- to 10-day old Arabidopsis seedlings were treated with 100 μM SA or 200 μM BTH. Treatments of canola and soybean plants were performed with the same concentrations, but on 3- to 4-week old plants, which were sprayed with the addition of 0.025% Silwet (v/v). Treatment with 5 μM flg22 was performed on 3- to 4-week old plants via syringe infiltration.

### SAR Experiments

Seedlings were grown for 7 to 10 days and drop-inoculated with either water or 2×10⁵ conidiospores ml⁻¹ of the avirulent Hpa isolate, Emwa1. Once true leaves emerged 7 days later, a secondary infection on upper systemic leaves with the virulent Hpa isolate, Noco2, was performed using 1×10⁵ conidiospores ml⁻¹ on all seedlings. Trypan blue analysis was then performed 7 to 10 days after.

### Trypan Blue Staining

Trypan blue staining was performed as previously described (Yoshioka et al., 2001).

### SA and SAG Measurements

Pooled tissue samples (n=18) were collected 5 days after infection with the avirulent Hpa isolate, Emwa1, and frozen in liquid nitrogen. Endogenous SA and SAG was extracted and analyzed as previously described (Mosher et al., 2010).

Protein Expression in E. coli

The coding region of AtTTM2 was cloned into pGEX-6P-1 from Arabidopsis Columbia ecotype cDNA using the primers, TTM2-TM-F and TTM2-TM-R, which excludes the annotated C terminal transmembrane domain starting from D648. Plasmids were introduced into E. coli BL21 (DE3) and grown overnight in LB medium at 37° C. The overnight culture was used to seed a larger volume of autoinduction medium containing 1×NPS solution (25 mM (NH₄)₂SO₄, 50 mM KH₂PO₄, and 50 mM Na₂HPO₄) and 1×5052 solution (0.05% glucose, 0.2% α-lactose, and 0.5% glycerol), which was grown at 37° C. for 3-4hrs until OD=0.4. The temperature was then lowered to 18° C. overnight before harvesting the cells by centrifugation at 4° C. (Studier, 2005).

### Protein Extraction

E. coli cultures were centrifuged and pellets were resuspended in 1×PBS pH 7.5 (137 mM NaCl, 2.7 mM KCl, 10 mM Na₂HPO₄, and 1.8 mM KH₂PO₄) containing 1 mM PMSF, 1 mM DTT, and 10 ug/ml DNasel. Cell suspensions were incubated on ice for 30 min before cell lysis by French press at 1000 psi. Soluble fractions were obtained by centrifugation and subjected to column purification using DE52 cellulose (Sigma) and GSH sepharose (Sigma). Purified protein samples were eluted using 10 mM reduced glutathione.

### Enzymatic Assays

Free phosphate released by AtTTM2 was measured with the Malachite Green assay (Bernal et al., 2005) as described in Moeder et al. (2013). The assay conditions were: 0.5 mM PPᵢ, ATP, or PPPᵢ, 2.5 mM Mg²⁺, pH 9.0 at 37° C. for 30 min. cAMP formation was assayed in 25 mM Tris pH 8, 1 mM ATP, 20 mM Mg²⁺ at 37° C. for 30 min. HPLC analysis was an isocratic run with 20% MeOH, 150 mM NaOAc, pH 5 on a Zorbax SB-C18 column (3.5 μm) (Agilent).

### Statistical Analysis

A two-tailed Student's T-test was performed for all comparisons between two sample groups. Fisher's exact test was performed for all comparisons between two samples with multiple groups. A p-value of less than 0.05 was used to denote significance.

### Accession Numbers:

Sequence data from this article can be found in the Arabidopsis Genome Initiative or GenBank/EMBL databases under the following accession numbers: AtTTM2 (At1g26190), Hpa-ITS2 (GU583836.1), PR1 (At2g14610), AtEF1A (At5g60390), β-tub (At5g23860), BnTTM2a (Bra011014), BnTTM2b (Bra012464), BnUBC21 (AC172883), BnPR1 (E F423806), GmTTM2a (Gm1g09660), GmTTM2b (Gm2g14110), GmEF1b (NM_001249608.1), GmPR1 (XM_003545723.1).

### Example 2

### Suppression of Expression of TTM2 in Tomato, Cucumber, Petunia and Pepper plants similar to Arabidopsis TTM2

### Plant Materials:

Tomato, Cucumber, Pepper and Petunia plants for BTH treatment were grown in Sunshine Mix at 22° C., 60% relative humidity, and approximately 140 uE m⁻² s⁻¹ with a 9-h photoperiod. Rice and Brachypodium distachyon plants for BTH treatment were grown in Rice Mix. tomato plants for Pseudomonas infection were grown in Sunshine Mix, at 22° C. 60% relative humidity, and natural light condition.

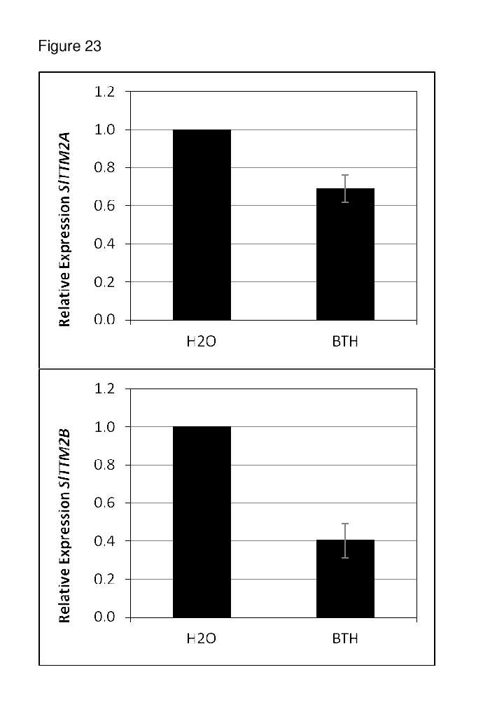

FIG. 23 illustrates expression of SITTM2A and B in approximately 4-5 week old tomato (Solanum lycopersicum) 48 hours after with and without BTH (200 μM) treatment. Solution was sprayed with the addition of 0.025% (v/v) Silwet. Expression of both genes was suppressed similar to Arabidopsis TTM2.

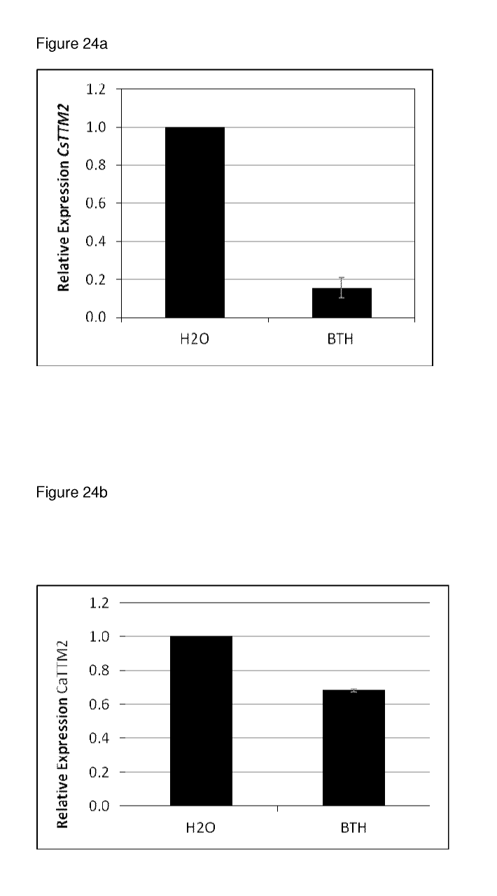

FIG. 24a illustrates expression of CsTTM2 in approximately 4-5 week old cucumber (Cucumis sativus) 48 hours after BTH (200 uM) treatment. Solution was sprayed with the addition of 0.025% (v/v) Silwet. Expression of the gene was suppressed similar to Arabidopsis TTM2.

FIG. 24b illustrates expression of CaTTM2 in approximately 4-5 week old pepper (Capsicum annuum) 48 hours after BTH (200 uM) treatment. Solution was sprayed with the addition of 0.025% (v/v) Silwet. Expression of the gene was suppressed similar to Arabidopsis TTM2.

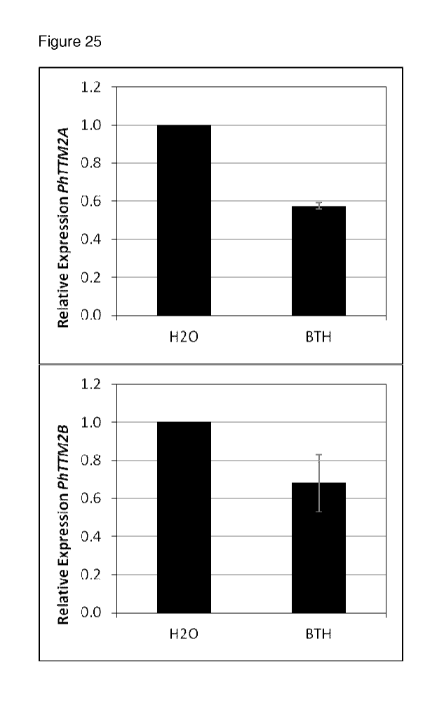

FIG. 25 illustrates expression of PhTTM2A and B in approximately 4-5 week old Petunia (Petunia hybrida) 48 hours after BTH (200 uM) treatment. Solution was sprayed with the addition of 0.025% (v/v) Silwet. Expression of both genes was suppressed similar to Arabidopsis TTM2.

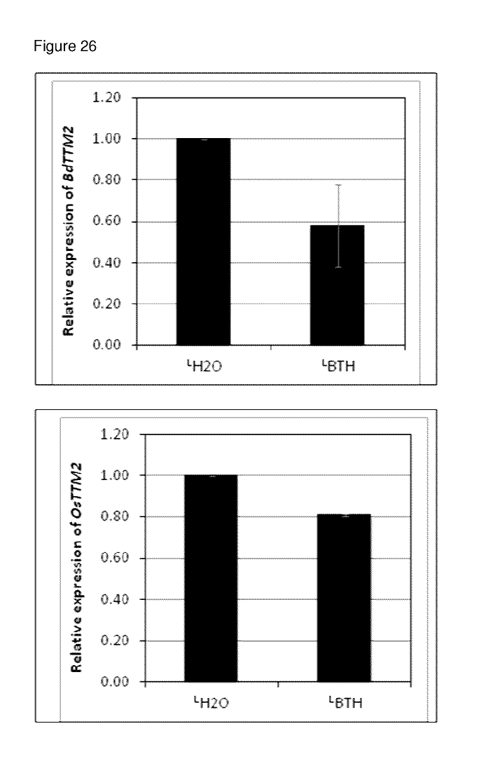

FIG. 26 illustrates expression of OsTTM2 in 4 week old rice (Oryza sativa) plant and BdTTM2 in the model monocotyledonous plant Brachypodium distachyon 48 hours after BTH (200 uM) treatment. Solution was sprayed with the addition of 0.025% (v/v) Silwet. Expression of both genes was suppressed similar to Arabidopsis TTM2.

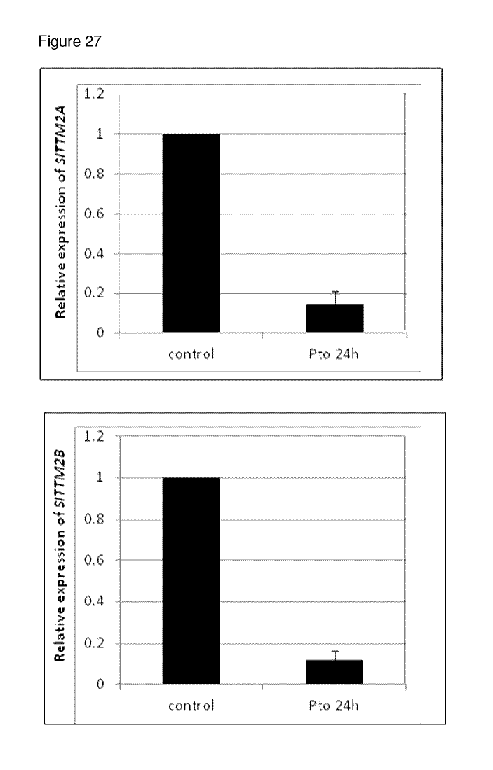

FIG. 27 illustrates expression of SITTM2A and B in approximately 4 week old tomato (Solanum lycopersicum) 24 hours after infection with the bacterial pathogen, Pseudomonas syringae pv. Tomato, DC3000. Infection was performed as described in Example 1. Expression of both genes was suppressed, similar to Arabidopsis AtTTM2.

### Example 3

### Bacterial Titre and Disease Severity for a Family of Tomato Plants Segregating for the Loss of Function in TTM2B

### Methods

### Plant Material

Tomato plants were grown in a greenhouse under 16 h day light, 23° C. day and night temperature. Seedlings were transplanted to 6″ pots at 3 weeks old, and inoculated at 4 weeks old.

### Inoculation

A 3 day old culture of Clavibacter michiganensis subsp. michiganensis (Cmm), grown on YDC agar at 28° C. was used for inoculation. Plants were inoculated using a sterilized 30G needle containing a tip-full of bacteria to pierce the stem at the first leaf adjacent to the petiole.

### Evaluation of Bacterial Titre

3 days post-inoculation, a 5 mm stem section, taken 1 cm above the point of inoculation was collected using a sterilized scalpel. Stem section was weighed, then ground in 0.5 mL of sterile 10 mM phosphate buffer, pH 7.4, using a sterile pellet pestle (Kimble Chase, Vineland, N.J.). Following grinding, 0.5 mL of 10 mM phosphate buffer, pH 7.4 was added for a total volume of 1 mL. Homogenized tissue was spun at 13,000 RPM for 3 min. Fifty μL of homogenized tissue diluted to 10⁻³ and 10⁻⁴ with 10 mM phosphate buffer, pH 7.4 was plated in duplicate onto NBY agar, and incubated at 28° C. for 3 days. Colonies were counted and expressed as CFU/mg tissue.

### Evaluation of Disease Severity

Bacterial canker disease severity was evaluated on plants through an assessment of wilt, at 4 time points following inoculation. Resistance to Cmm was evaluated using a 0-5 scale, where 0=healthy plant, no leaf wilt; 1=initial appearance of wilt, <10% of leaves collapsed; 2=10-25% of leaves showing wilt; 3=25-50% of leaves showing wilt; 4=75% of leaves showing wilt; 5=whole plant is wilted.

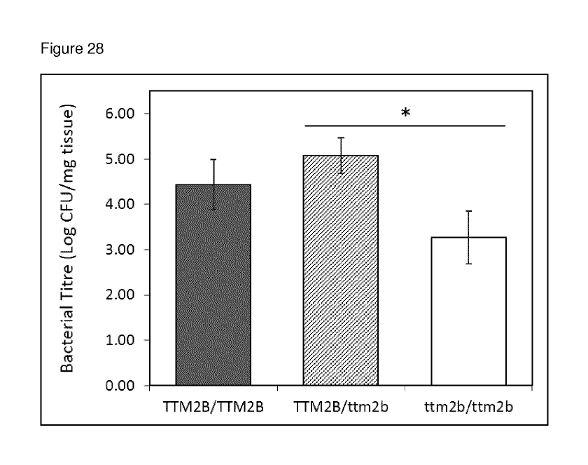

FIG. 28 illustrates bacterial titre for a family segregating for the loss of function in TTM2B. Plants were assessed for bacterial titre 3 days following inoculation with the bacterial pathogen, Clavibacter michiganensis subsp. michiganensis. A 5 mm stem section 1 cm above the site of inoculation was collected and bacterial titre was determined through a plating assay. TTM2=wild type allele; ttm2=loss of function allele. Asterisk represents significance at p<0.05. Capped lines=standard error of the mean.

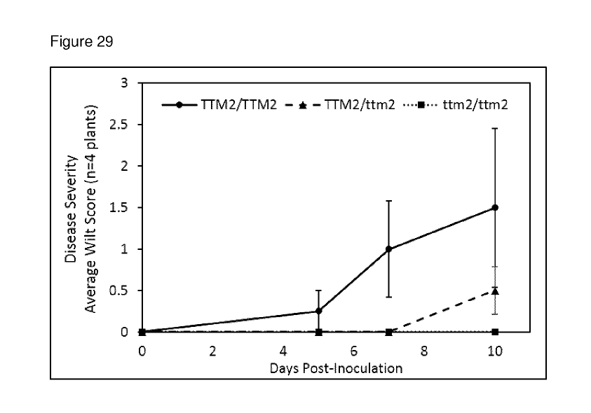

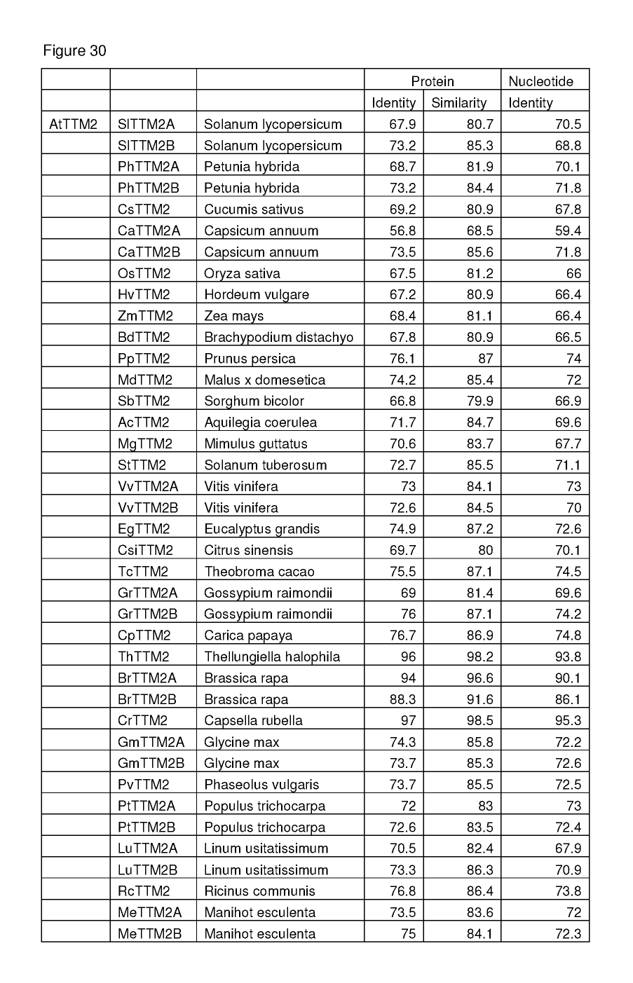

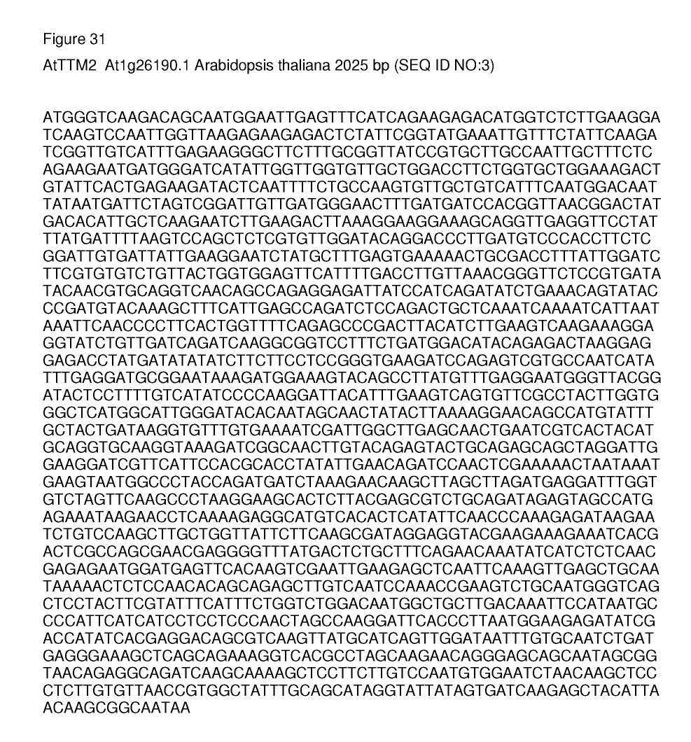

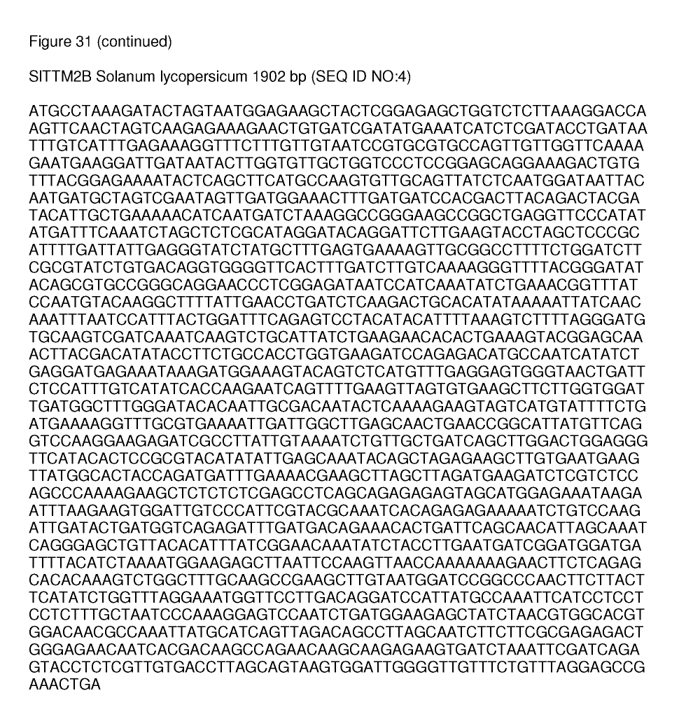

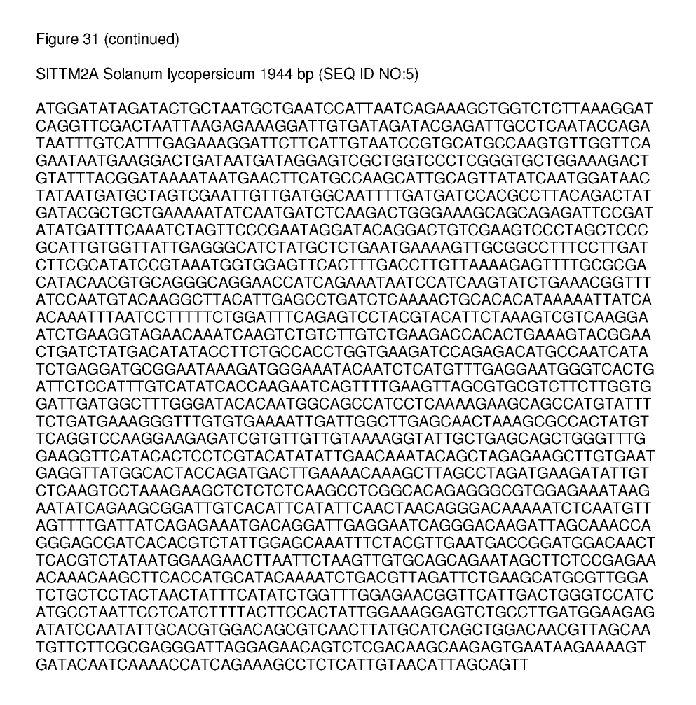

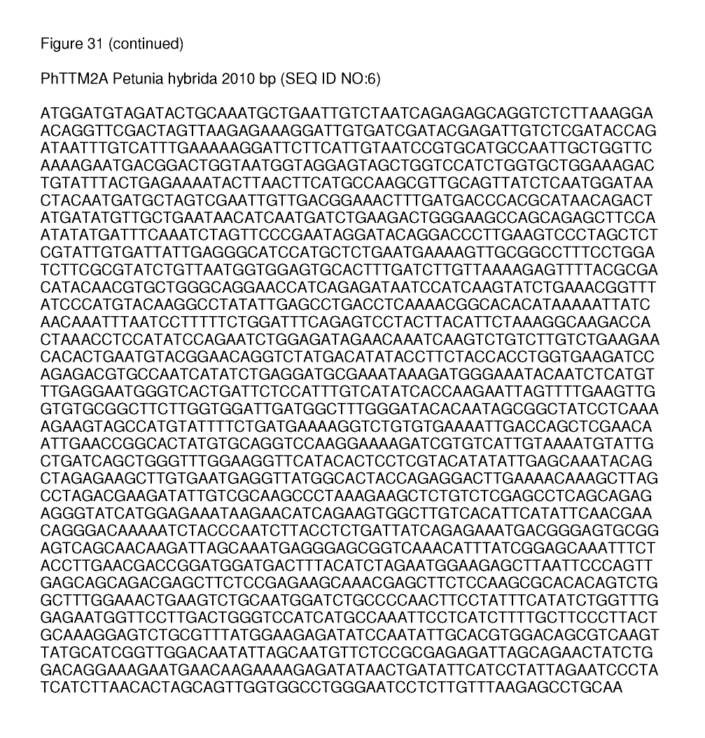

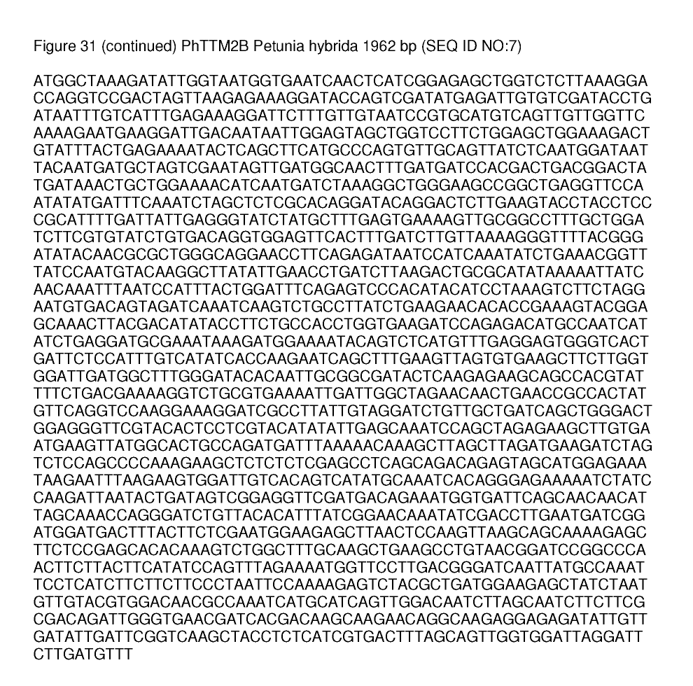

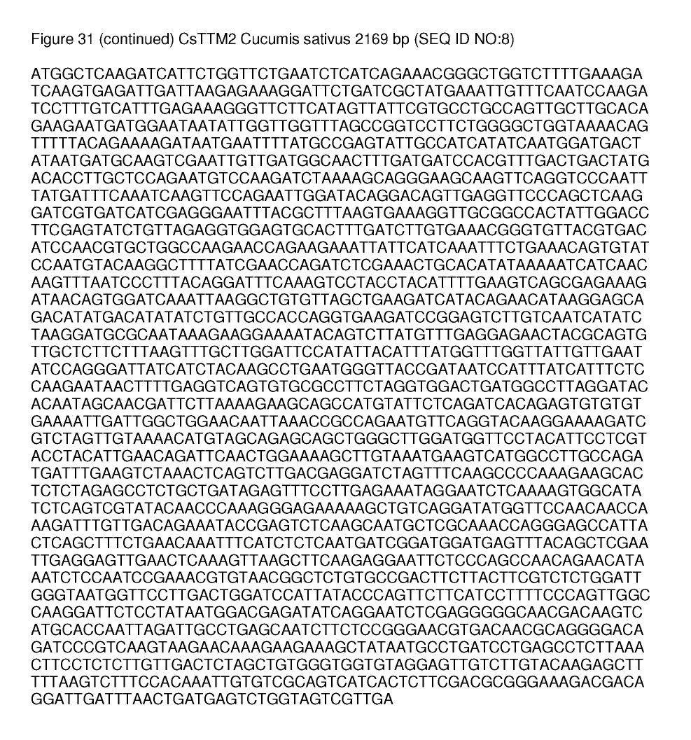

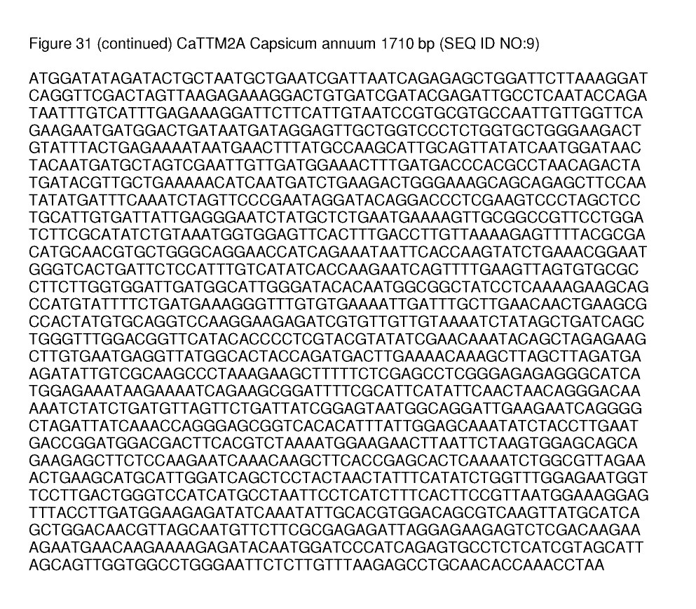

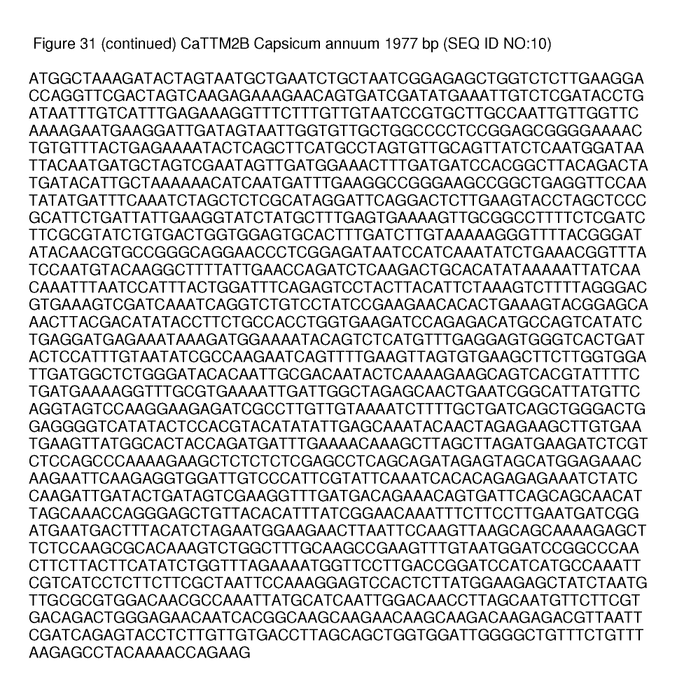

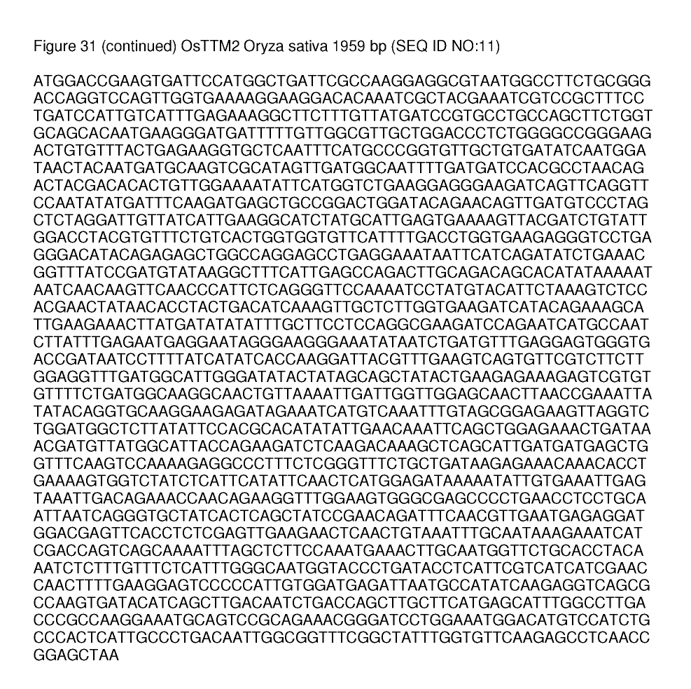

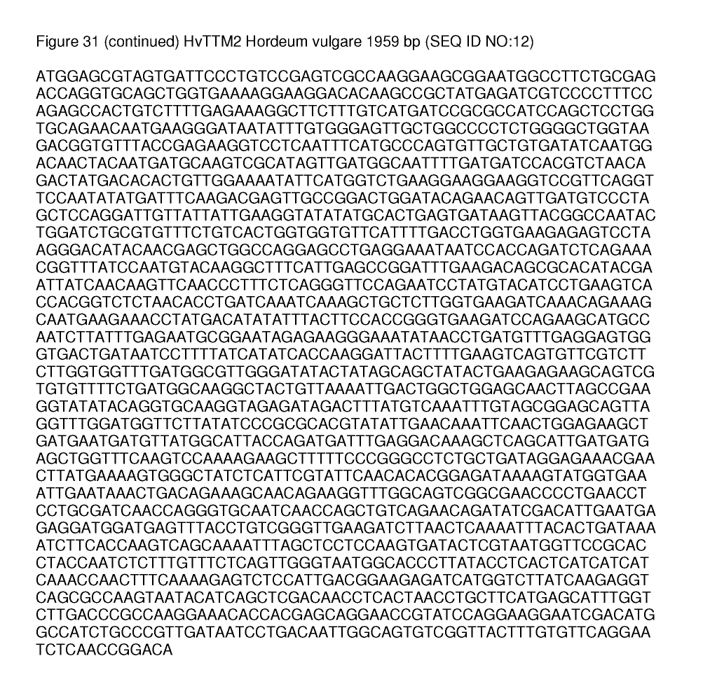

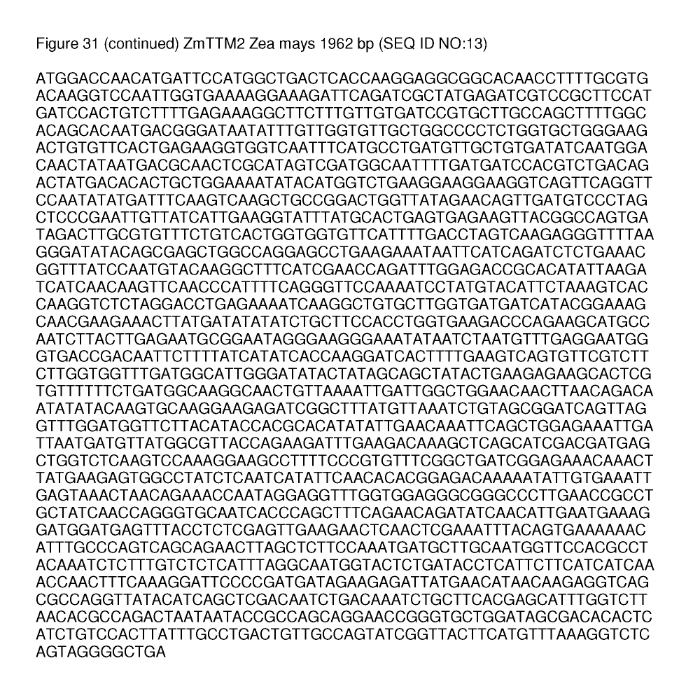

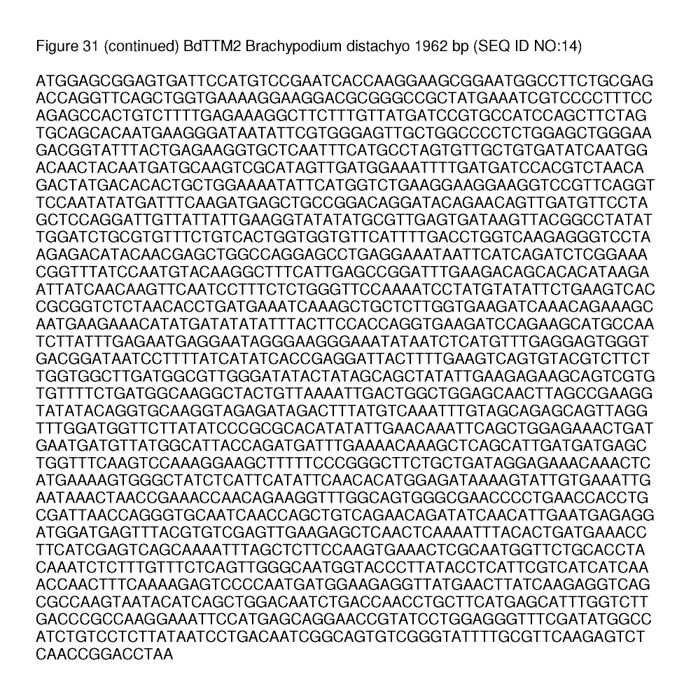

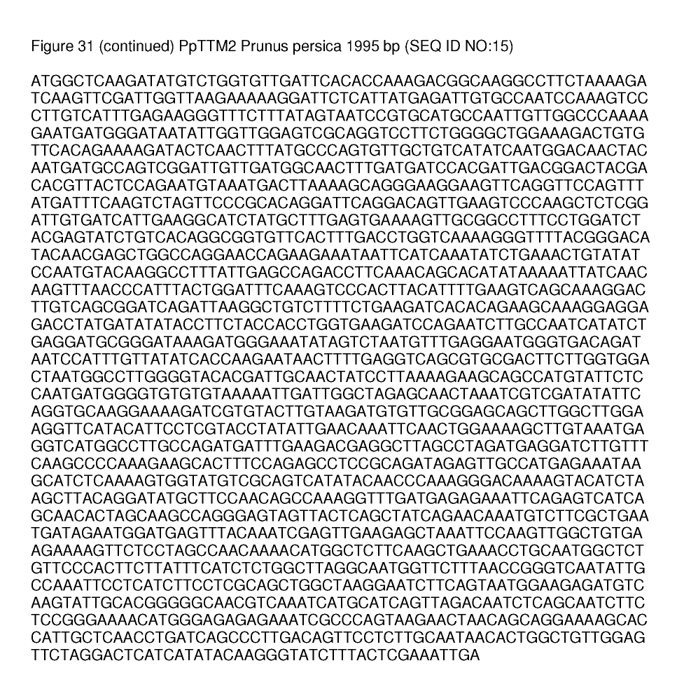

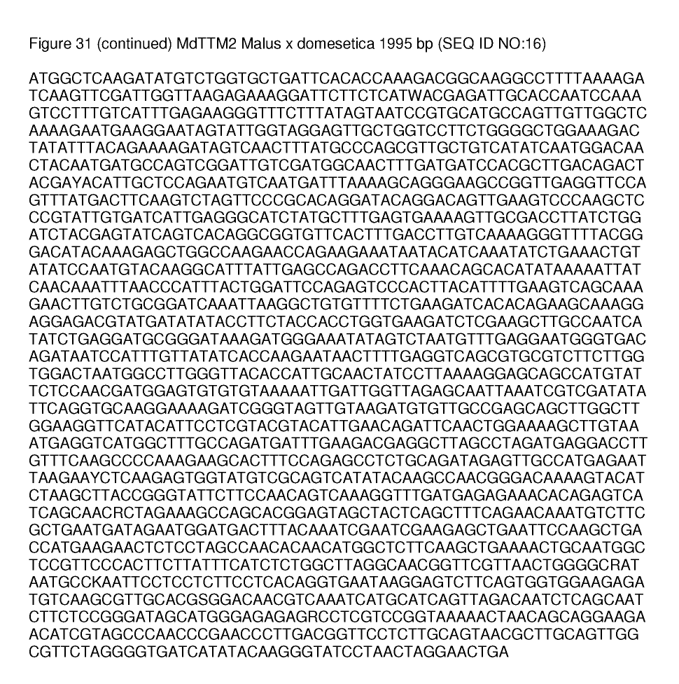

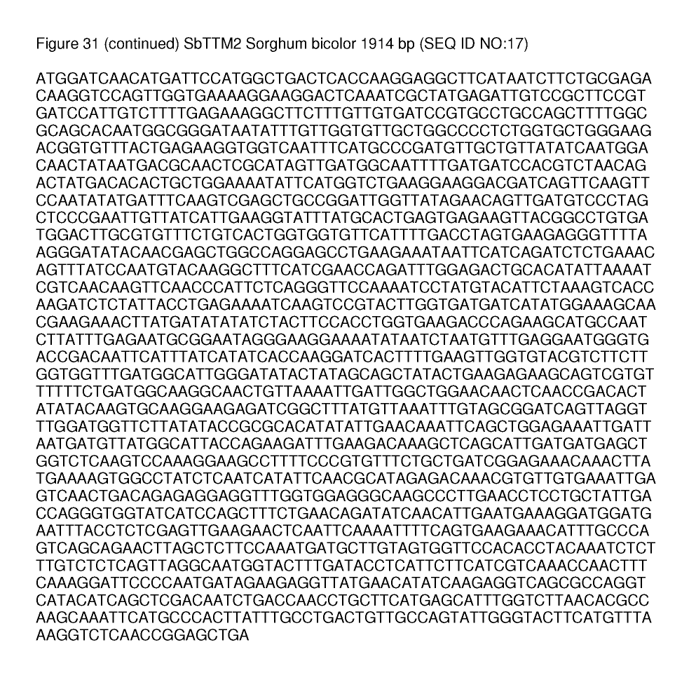

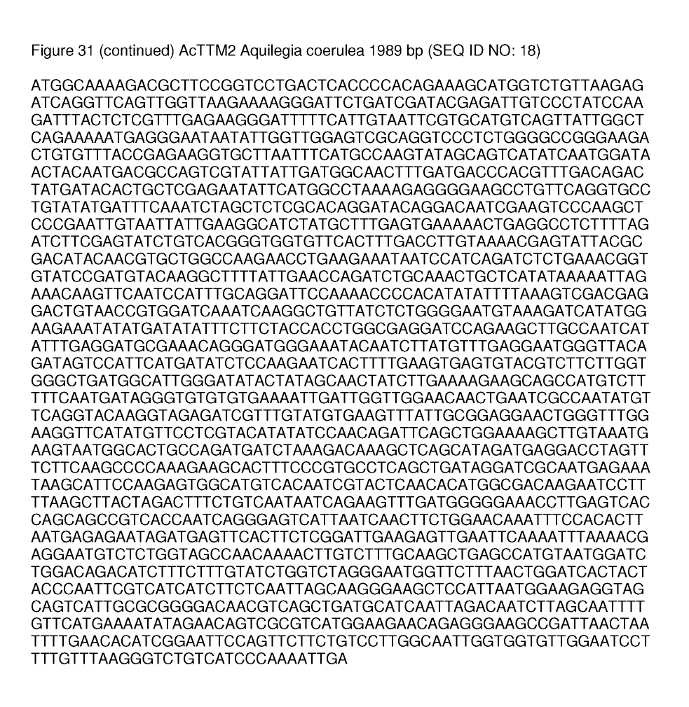

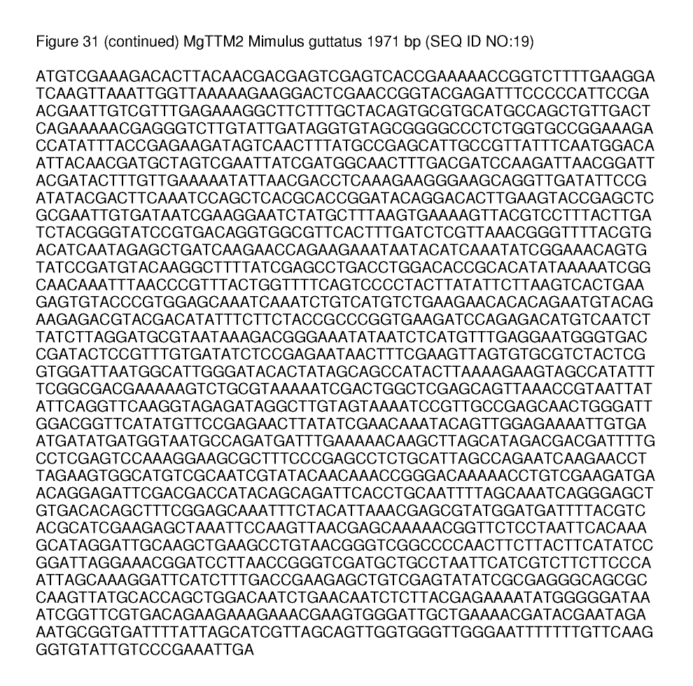

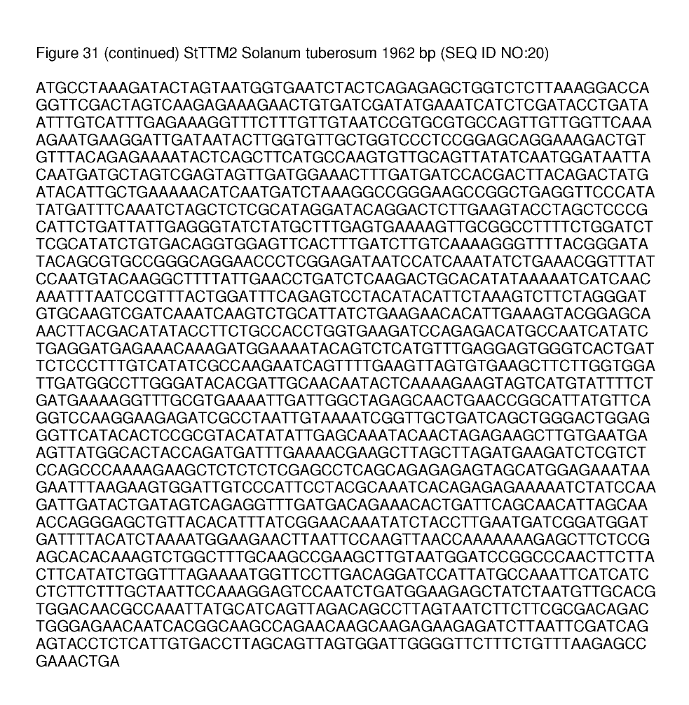

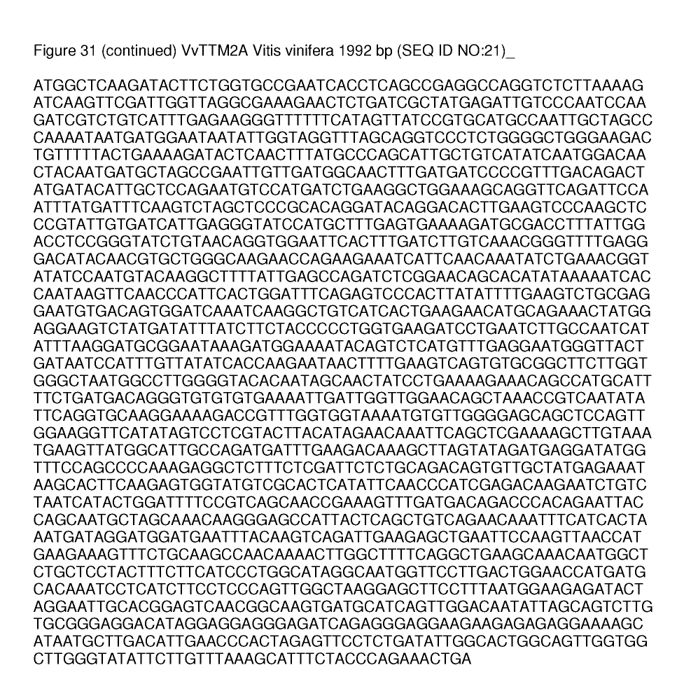

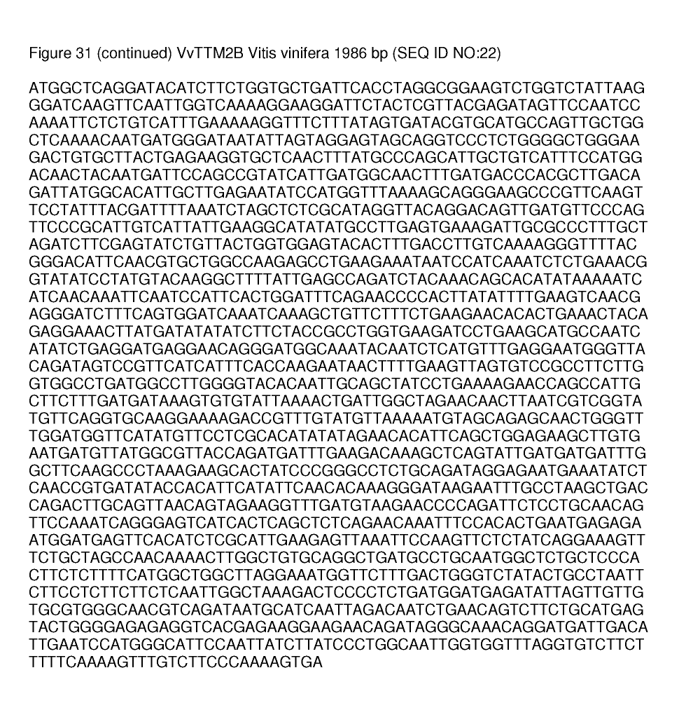

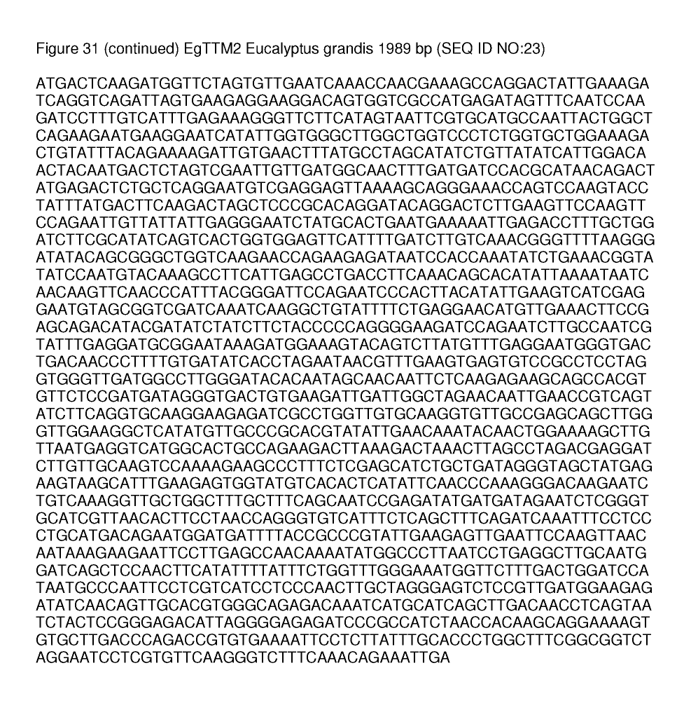

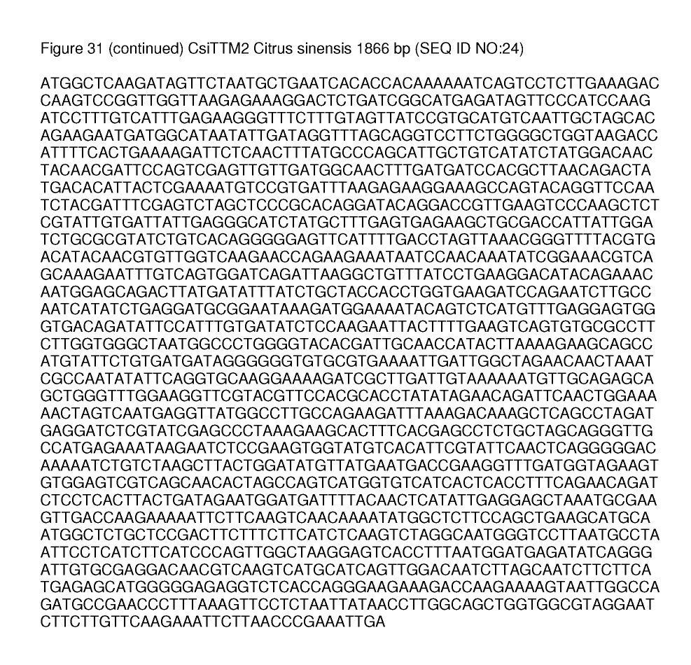

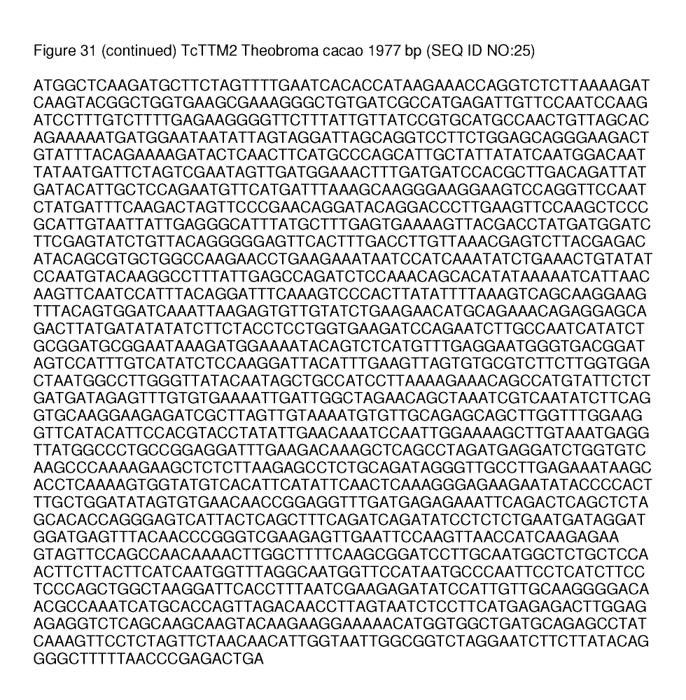

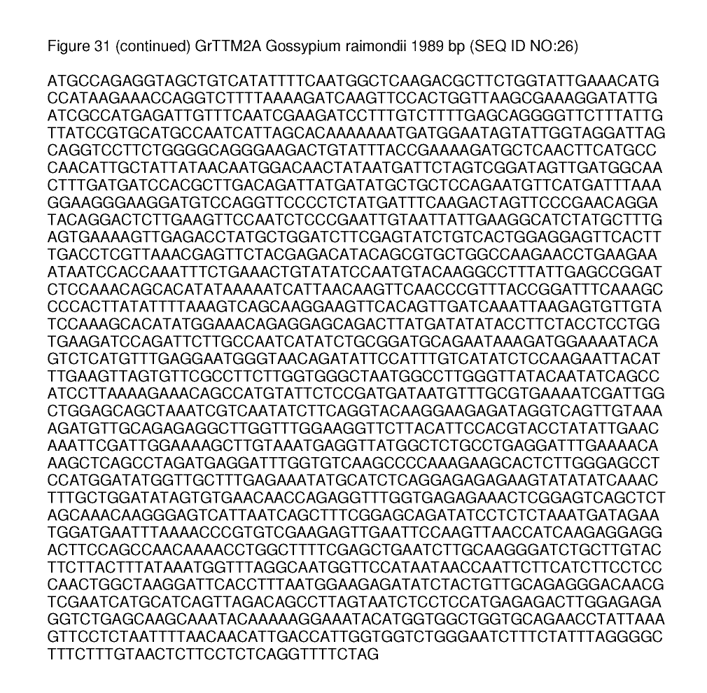

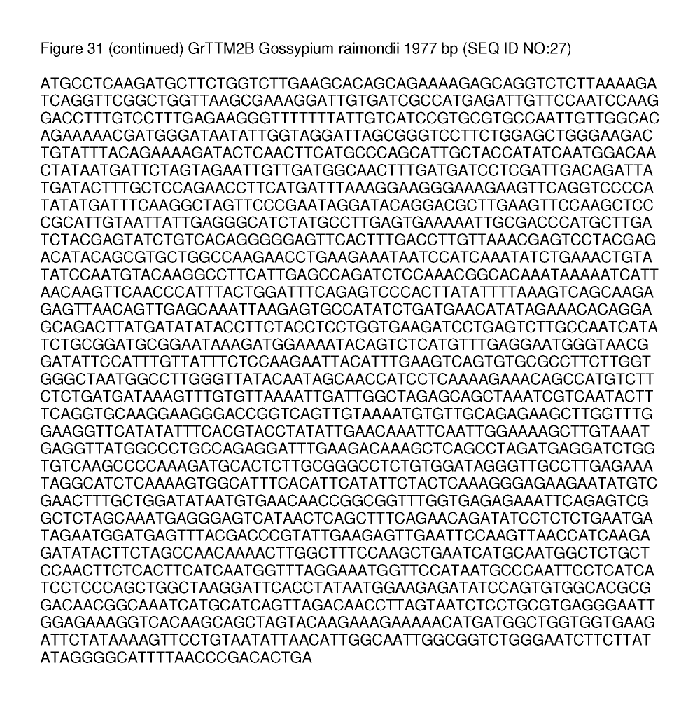

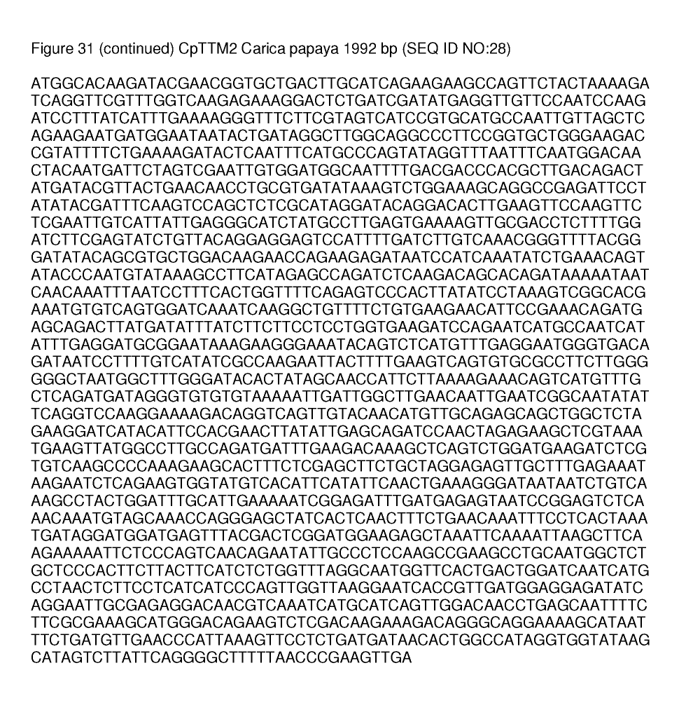

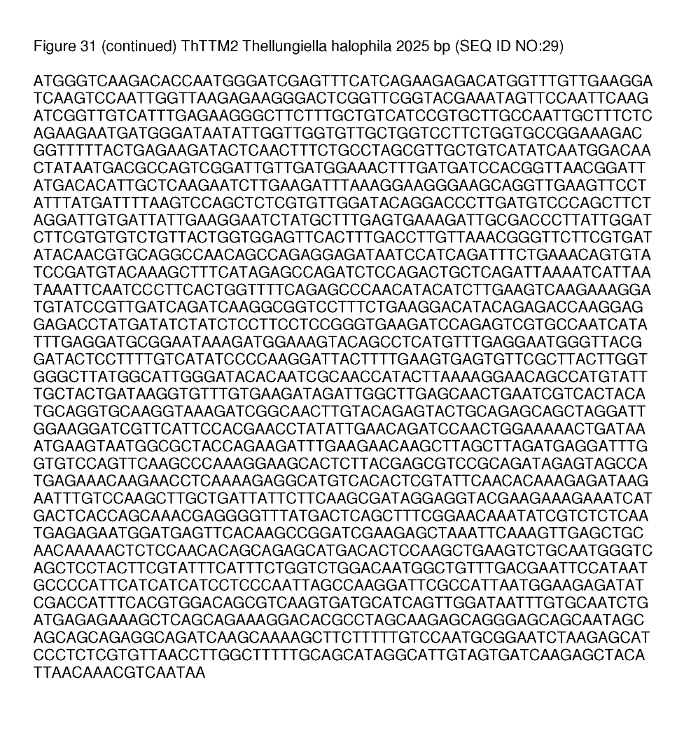

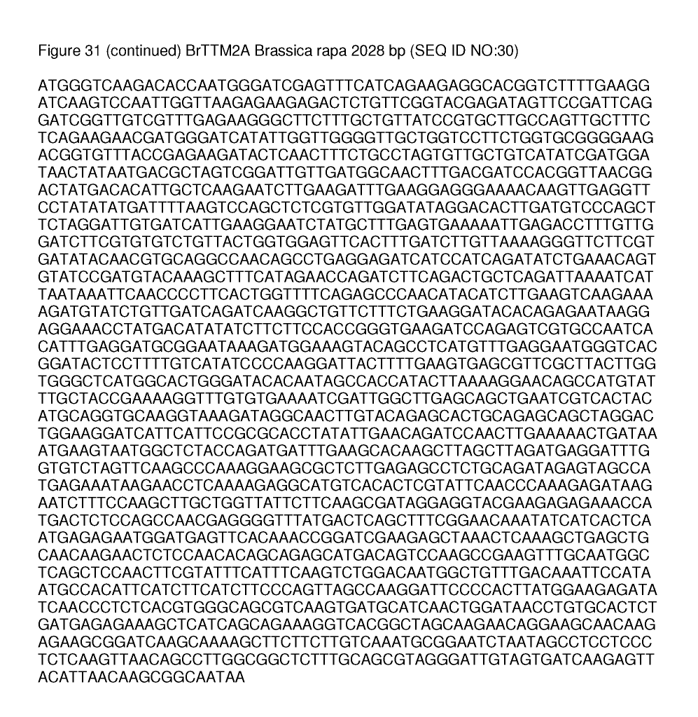

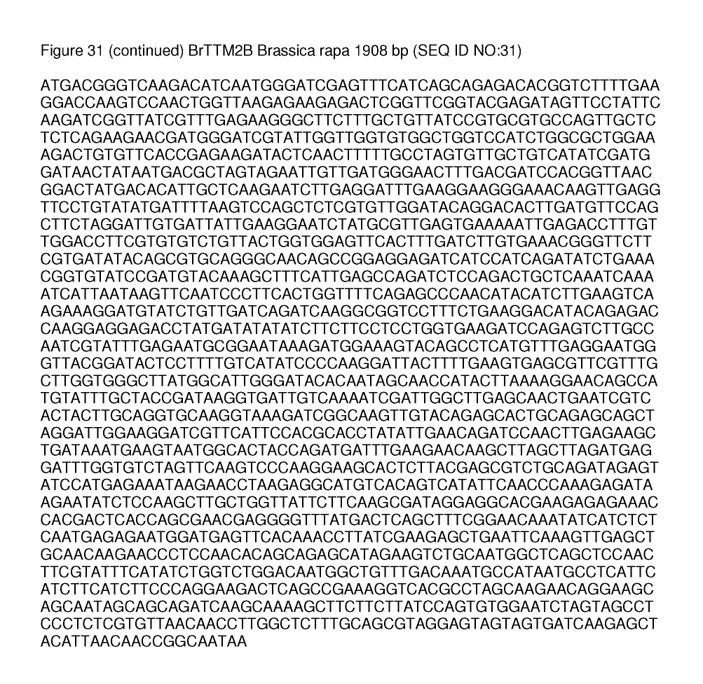

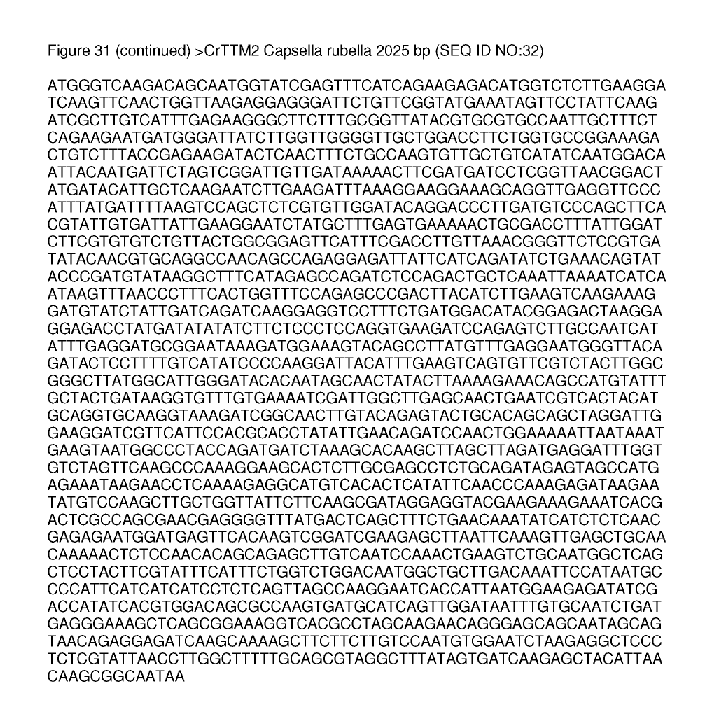

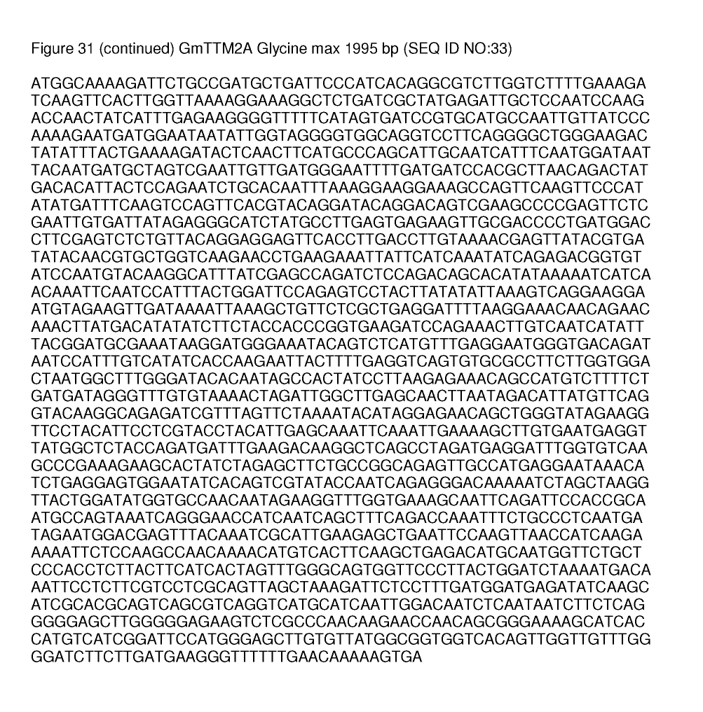

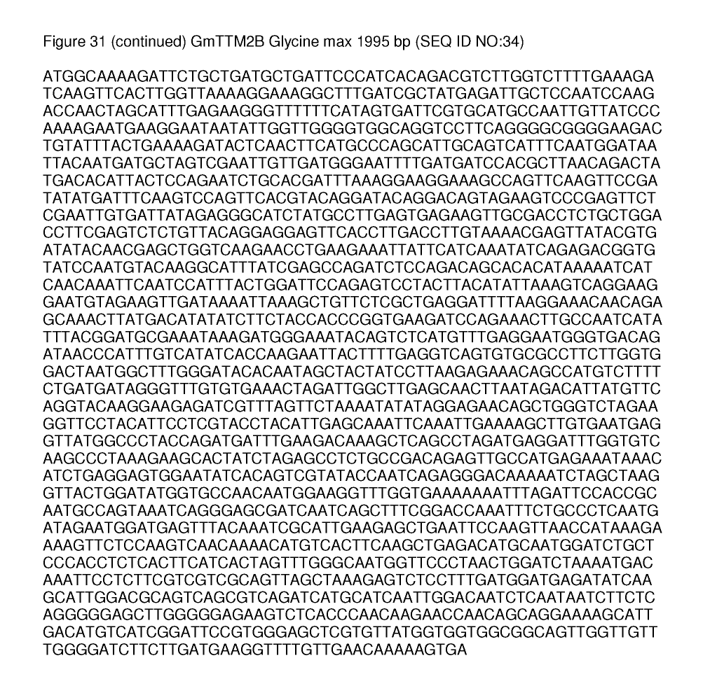

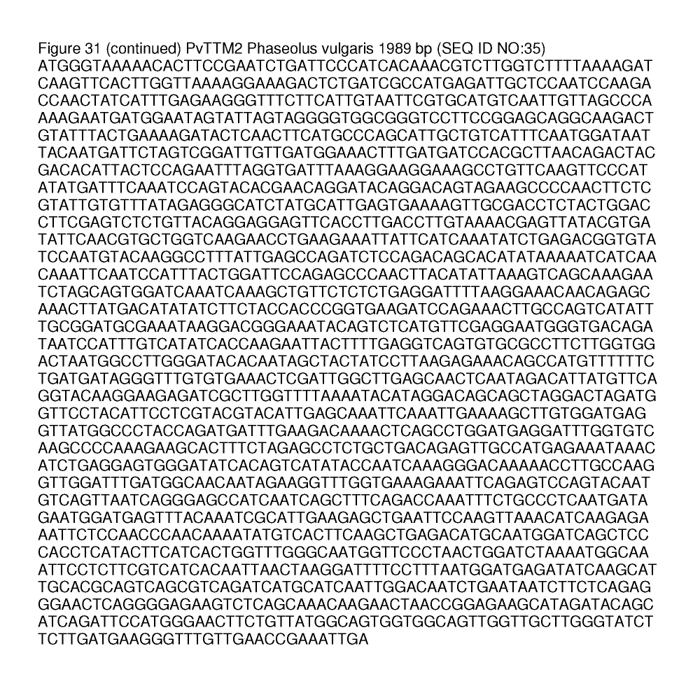

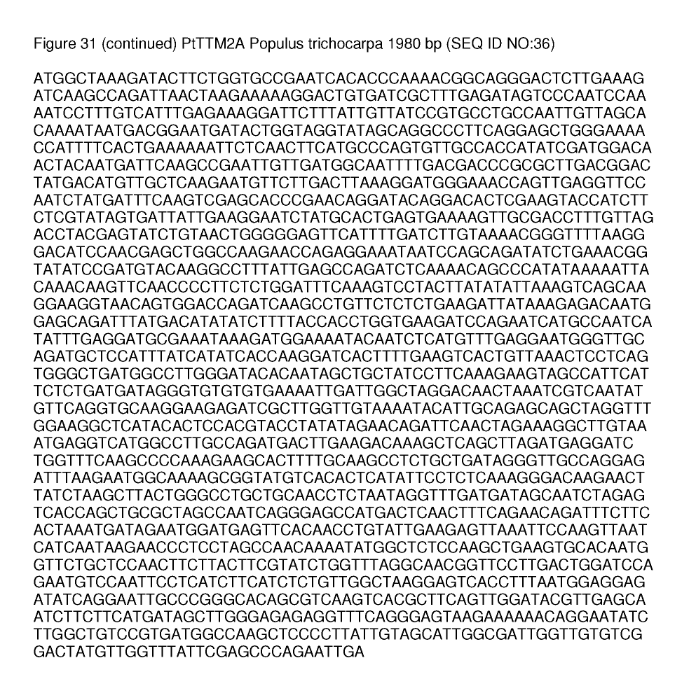

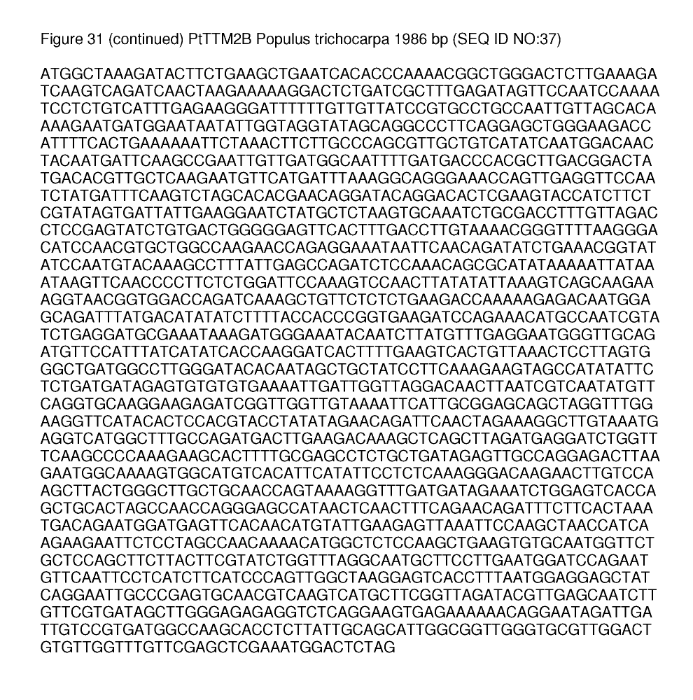

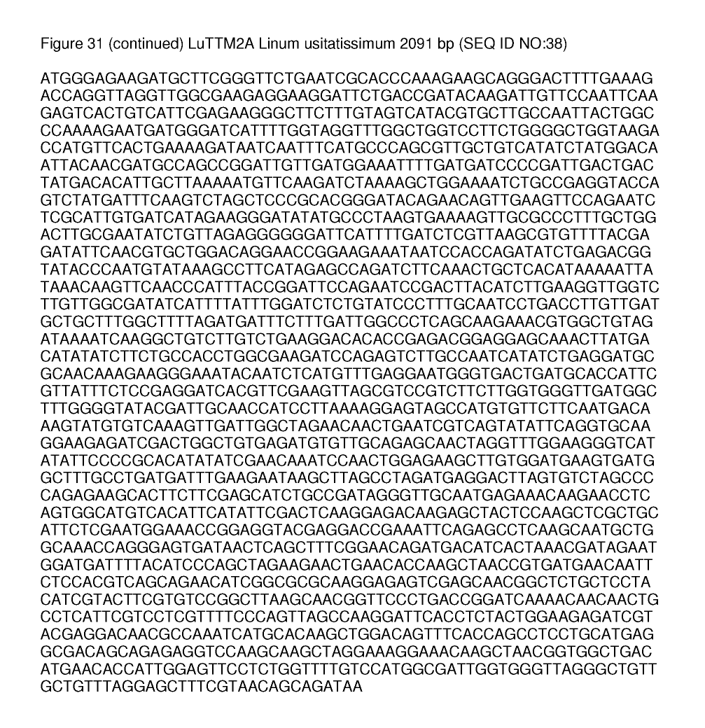

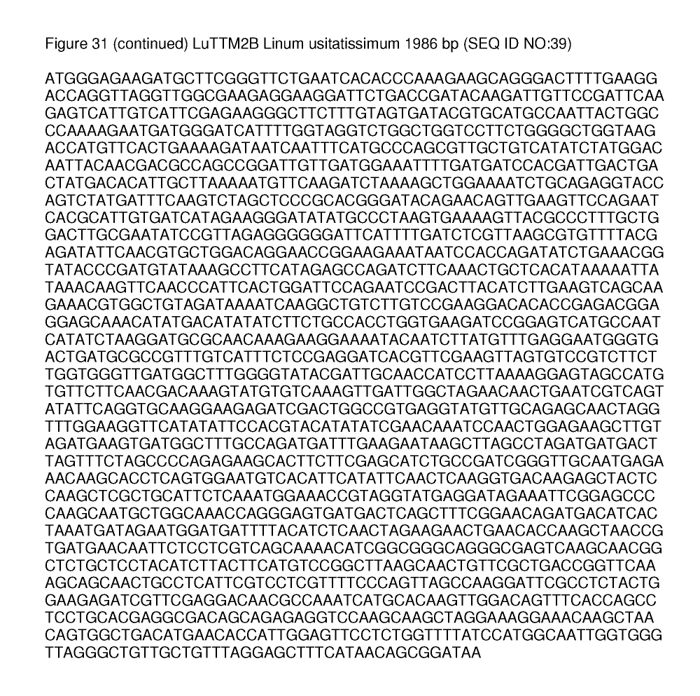

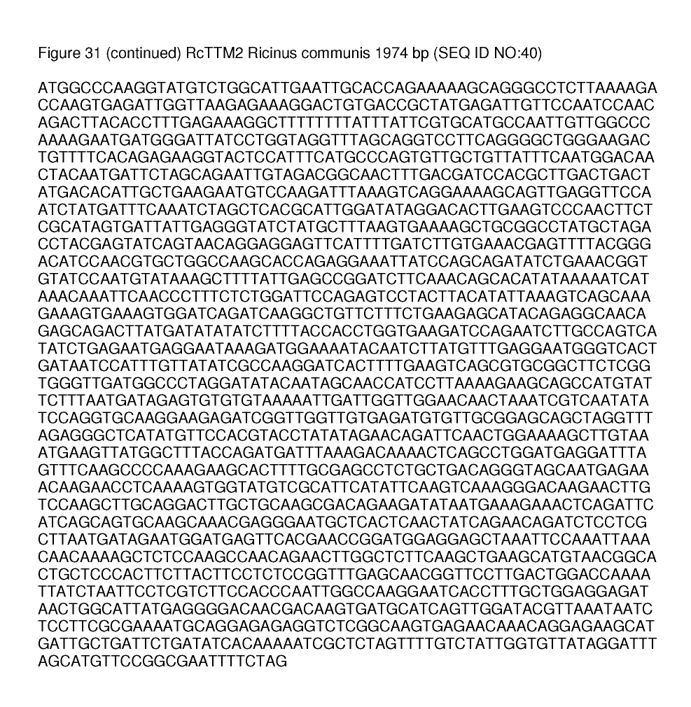

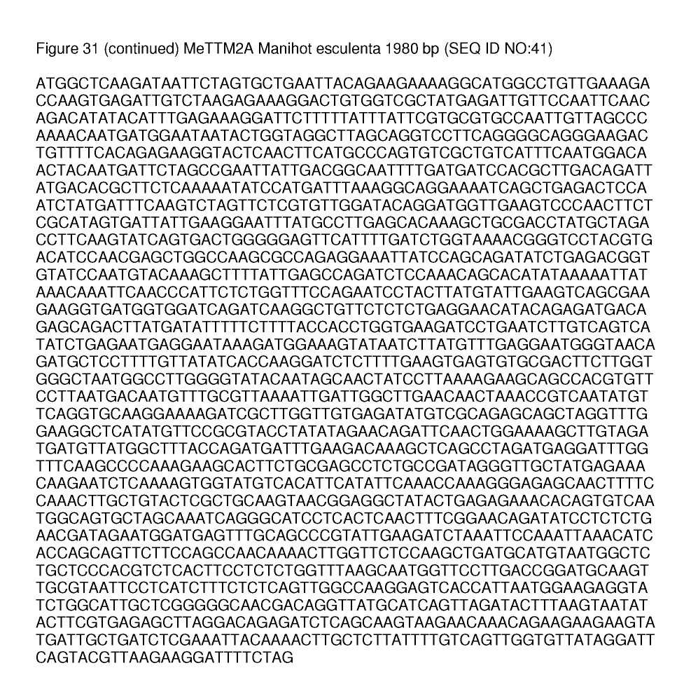

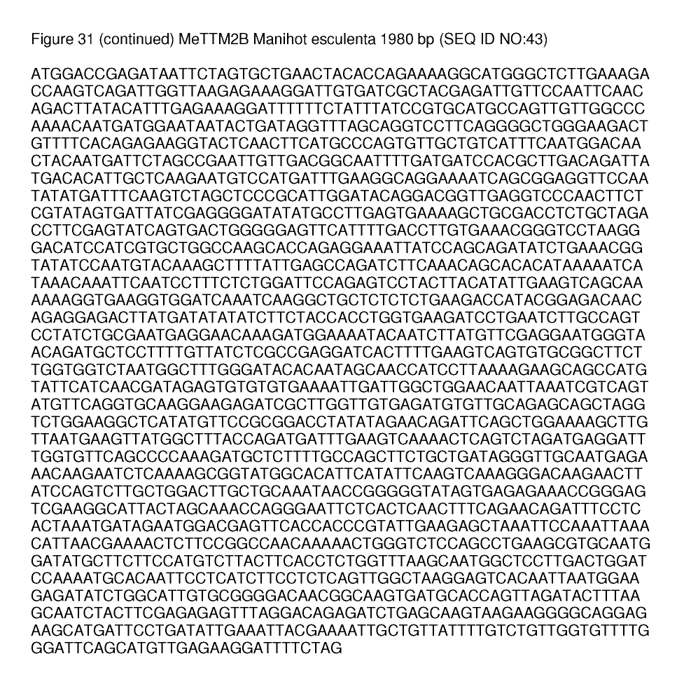

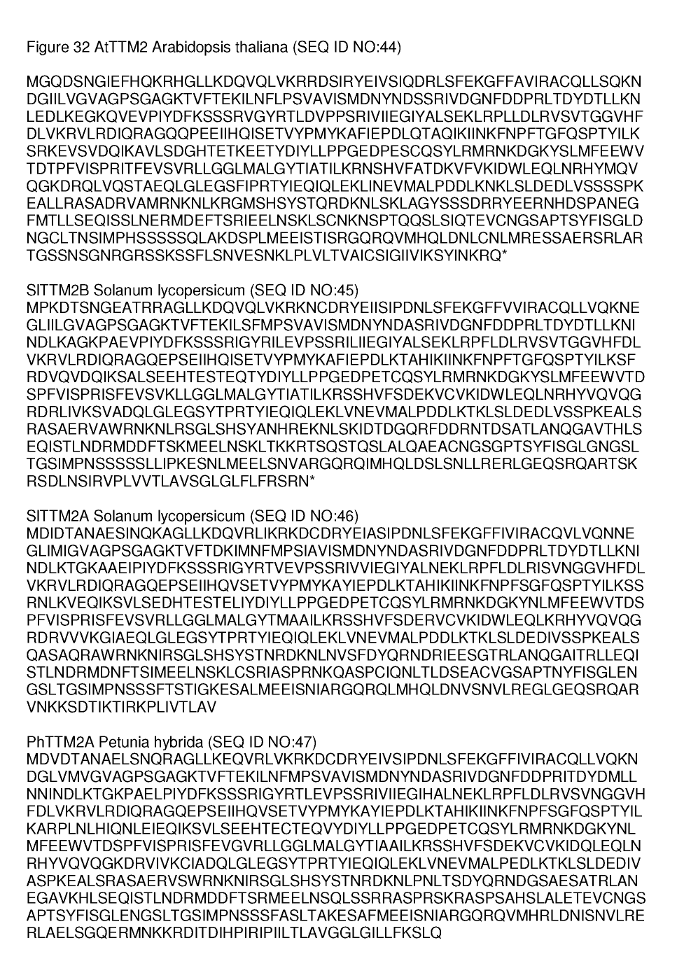

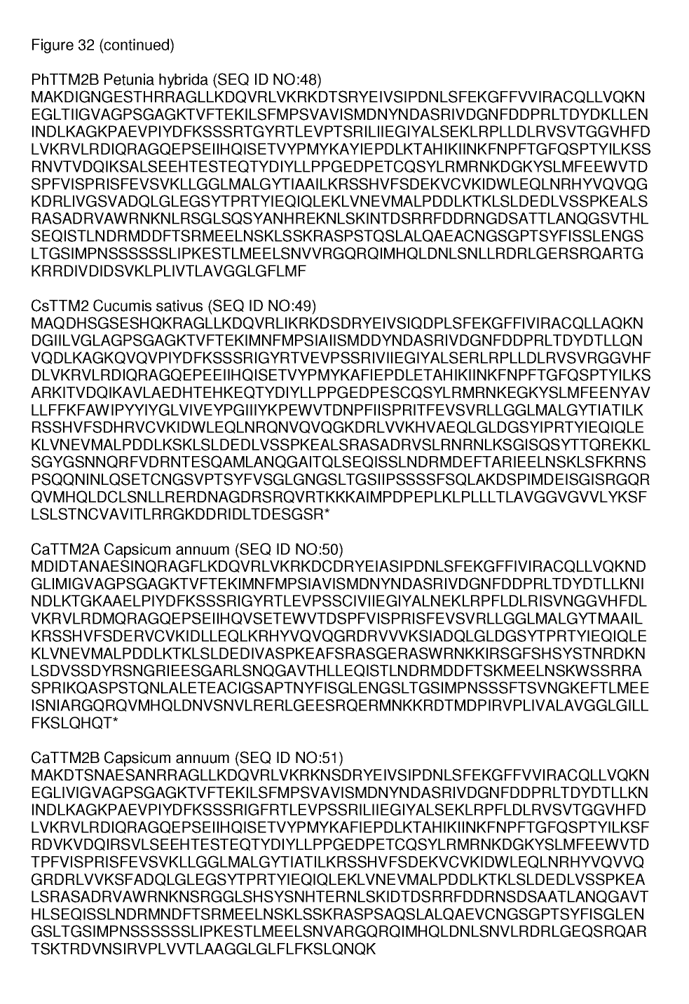

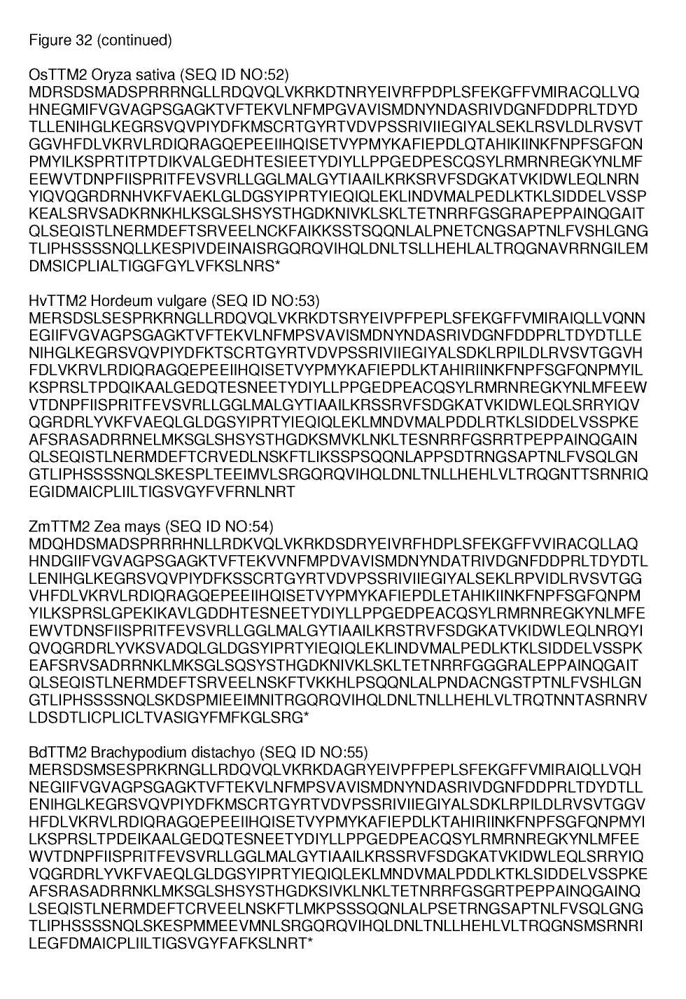

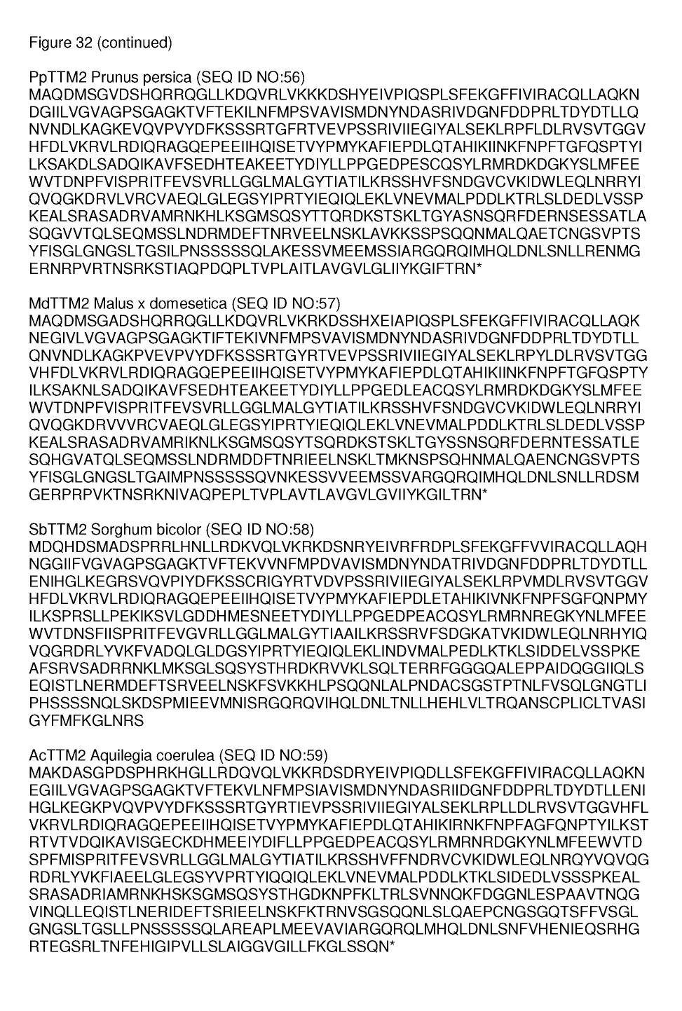

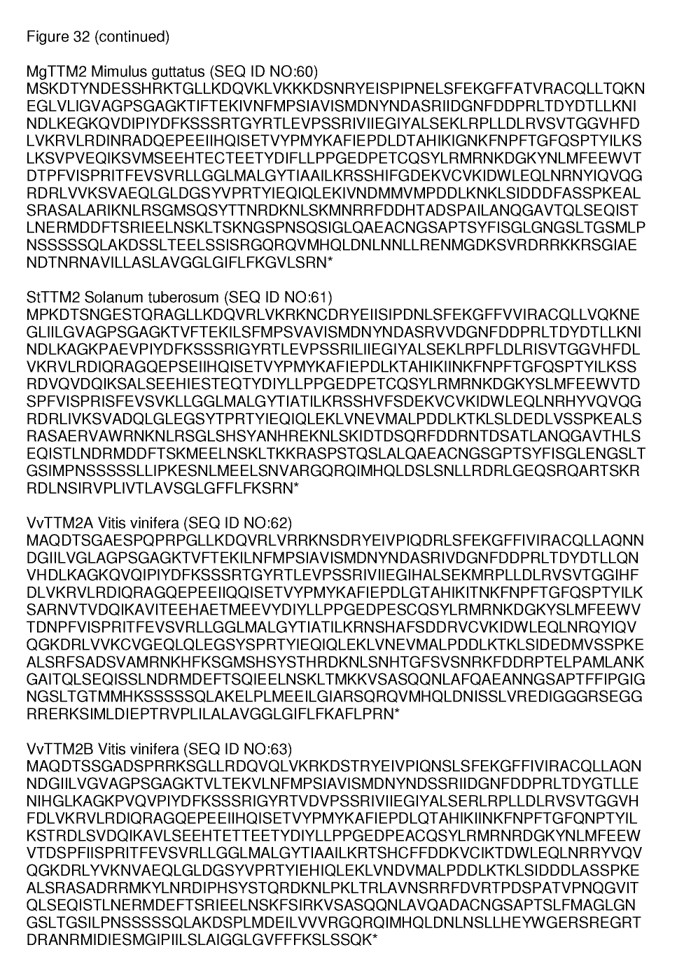

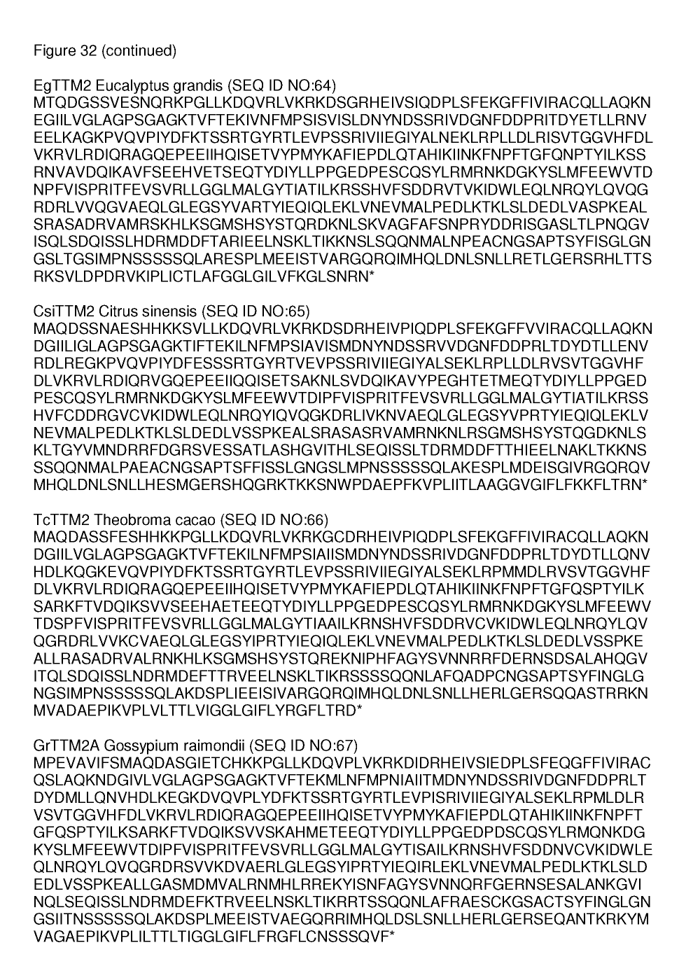

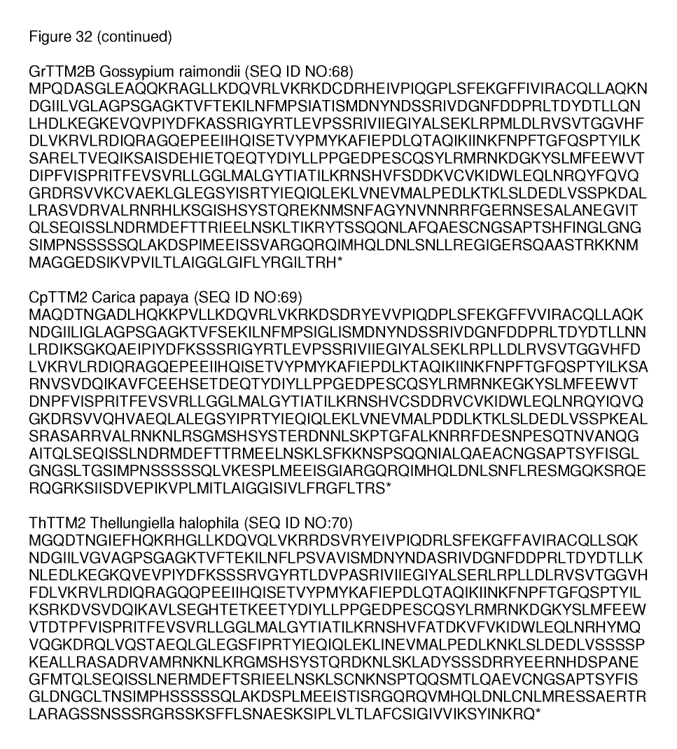

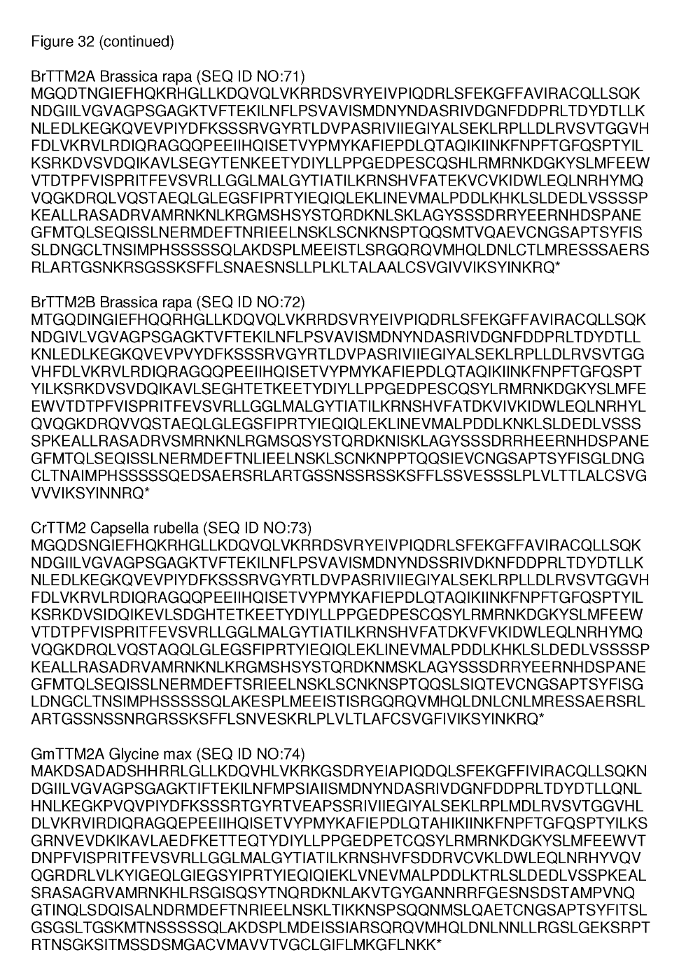

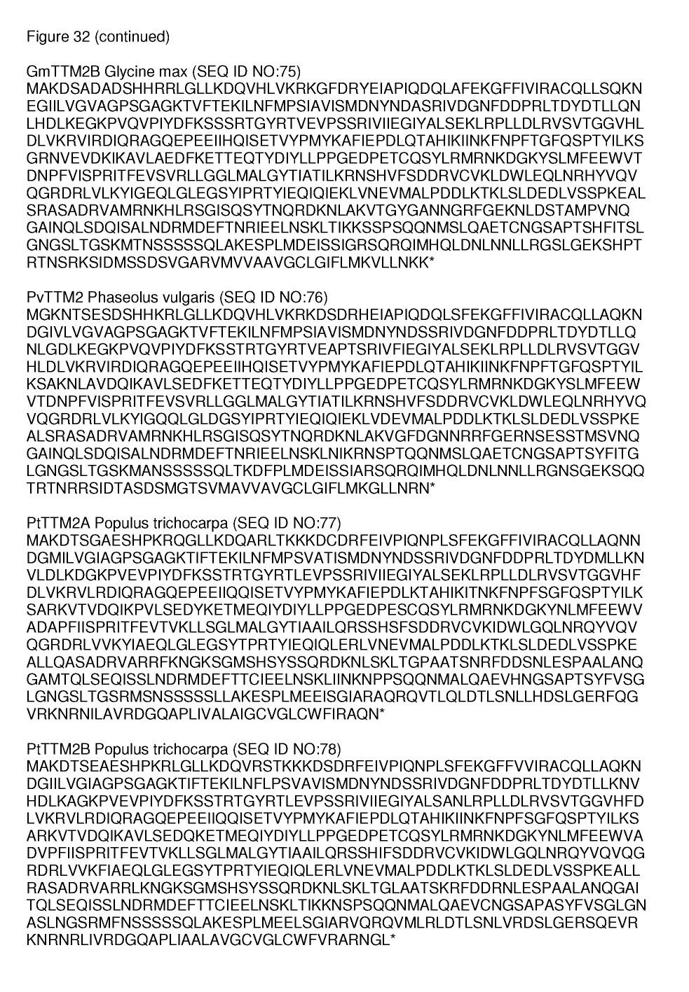

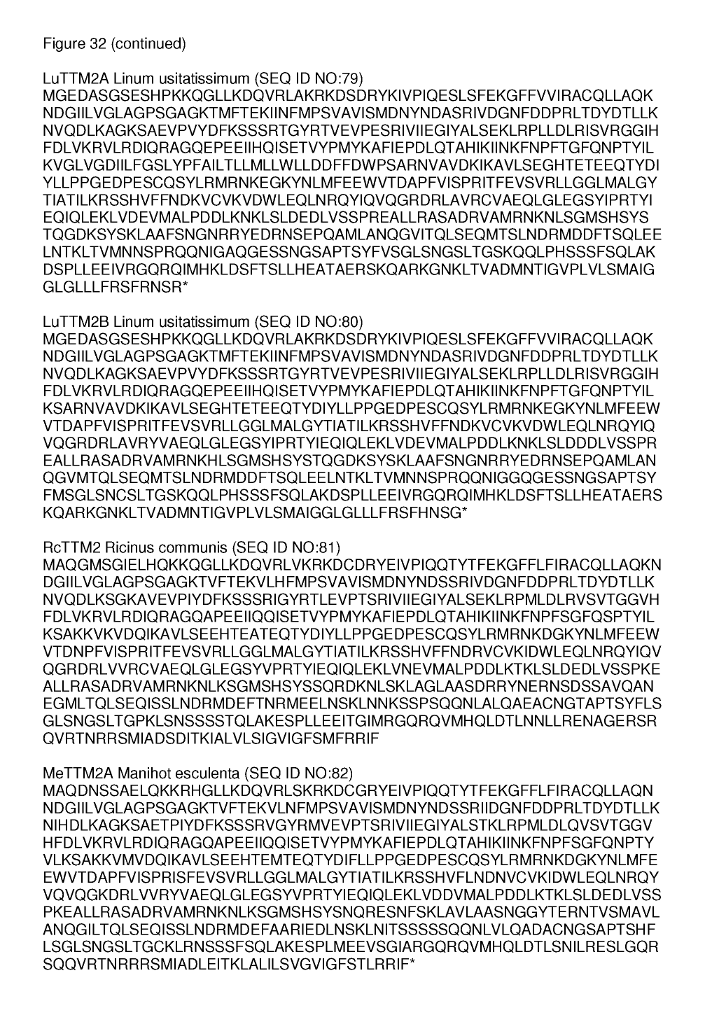

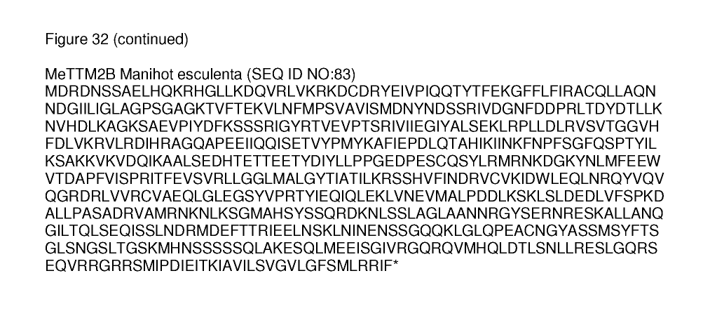

FIG. 29 shows average disease severity of plants from a family segregating for the loss of function in TTM2B. Plants were assessed at 4 time points following inoculation with the bacterial pathogen, Clavibacter michiganensis subsp. michiganensis. Wilting was evaluated using a scale of 0-5, where 0=healthy plant free of wilt; 1=initial appearance of wilt, <10% of leaves collapsed; 2=10-25% of leaves showing wilt; 3=25-50% of leaves showing wilt; 4=75% of leaves showing wilt; 5=100% leaves wilted. TTM2=wild type allele; ttm2=loss of function allele. Bars=standard error of the mean.

### LITERATURE CITED

### Alonso J M, Stepanova A N, Leisse T J et al. (2003) Science 301: 653-657

### Alvarez M E, Pennell R I, Meijer P J, Ishikawa A, Dixon R A, Lamb C (1998) Cell 92: 773-784

### Bent A F, Mackey D (2007) Annu Rev Phytopathol 45: 399-436

### Bernal C, Palacin C, Boronat A, Imperial S (2005) Anal Biochem 337: 55-61

### Bettendorff L, Wins P (2013) FEBS J 24: 6443-6455

### Cao H, Bowling S A, Gordon A S, Dong X (1994) Plant Cell 6: 1583-1592

### Cao H, Glazebrook J, Clarke J D, Volko S, Dong X (1997) Cell 88: 57-63

### Clough S J, Bent A F (1998) Plant J 16: 735-743

### Delvaux D, Murty MRVS, Gabelica V, Lakaye B, Lunin V V, Skarina T, Onopriyenko O, Kohn G, Wins P, De Pauw E, Bettendorff L (2011) J Biol Chem 286: 34023-34035

### Dong X (2004) Sci STKE: 221: pe6

### Falk A, Feys B J, Frost L N, Jones J D, Daniels M J, Parker J E (1999) Proc Natl Acad Sci USA 96: 3292-3297

### Feys B J, Moisan L J, Newman M-A, Parker J E (2001) EMBO J 20: 5400-5411

### Feys B J, Wiermer M, Bhat R A, Moisan L J, Medina-Escobar N, Neu C, Cabral A, Parker J E (2005) Plant Cell 17: 2601-2613

### Frye C A, Innes R W (1998) Plant Cell 10: 947-56

### Frye C A, Tang D, Innes R W (2001) Proc Natl Acad Sci USA 98: 373-378

### Gallagher D T, Smith N N, Kim S K, Heroux A, Robinson H, Reddy P T (2006) J Mol Biol 362: 114-122

### Glazebrook J, Rogers E E, Ausubel F M (1996). Genetics 143: 973-982

### Gong C, Smith P, Shuman S (2006) RNA 12: 1468-1474

### Hammond-Kosack K E, Jones J D G (1996) Plant Cell 8: 1773-1791

### Heath M C (2000) Plant Mol Biol 44: 321-334

### Hofius D, Mundy J, Petersen M (2009) Autophagy 5: 1206-1207

### Iyer L M, Aravind L (2002) BMC Genomics 3: 33

### Jirage D, Tootle T L, Reuber T L, Frost L N, Feys B J, Parker J E, Ausubel F M, Glazebrook J (1999) Proc Natl Acad Sci USA 96: 13583-13588

Keppetipola N, Jain R, Shuman S (2007) Novel triphosphate phosphohydrolase activity of Clostridium thermocellum J Biol Chem 282: 11941-11949

### Laird J, Armengaud P, Giuntini P, Laval V, Milner J J (2004) Planta 219: 1089-1092

### Lakaye B, Makarchikov A F, Wins P, Margineanu I, Roland S, Lins L, Aichour R, Lebeau L, E I Moualij B, Zorzi W, Coumans B, Grisar T, Bettendorff L (2004) Int J Biochem Cell Biol 36: 1348-1364

### Lawton K A, Friedrich L, Hunt M, Weymann K, Delaney T, Kessmann H, Staub T, Ryals J (1996) Plant J 10: 71-82

### Leipe D, Koonin E V, Aravind L (2003) J Mol Biol 333: 781-815

### Lima C D, Wang L K, Shuman S (1999) Cell 99: 533-543

### Lu H, Rate D N, Song J T, Greenberg J T (2003) Plant Cell 15: 2408-2420

### Moeder W, Garcia-Petit C, Ung H, Fucile G, Samuel M A, Christendat D, Yoshioka K (2013) Plant J 76: 615-626

Moeder W, Yoshioka K (2008) Lesion mimic mutants. Plant Signal Behav 3: 764-767

### Mosher S, Moeder W, Nishimura N, Jikumaru Y, Joo S H, Urquhart W, Klessig D F, Kim S K,

### Nambara E, Yoshioka K (2010) Plant Physiol 152: 1901-1913

### Návarová H, Bernsdorff F, Döring A C, Zeier J (2012) Plant Cell 24: 5123-5141

### Nawrath C and Métraux J P (1999) Plant Cell 11: 1393-1404

### Quentin M, Allasia V, Pegard A, Allais F, Ducrot P H, Favery B, Levis C, Martinet S, Masur C,

Ponchet M, Roby D, Schlaich N L, Jouanin L, Keller H (2009) PLoS Pathog 5: e1000264

### Parker J E, Holub E B, Frost L N, Falk A, Gunn N D Daniels M J (1996). Plant Cell 8: 2033-2046

### Raffaele S, Rivas S, Roby D (2006) FEBS Lett 580: 3498-504

Roberts M, Tang S, Stallmann A, Dangl J L, Bonardi V (2013) PLoS Genet 9: e1003465

### Shah J (2003) Curr Opin Plant Biol 6: 365-371

### Shah J, Zeier J (2013) Front Plant Sci 4: 30

### Shuman S (2002) Nat Rev Mol Cell Biol 3: 619-625

### Sismeiro O, Trotot P, Bivillem F, Vivares C, Danchin A (1998) J Bacteriol 180: 3339-3344

### Song J T, Lu H, McDowell J M, Greenberg J T (2004) Plant J 40: 200-212

### Song J, Bettendorff L, Tonelli M, Markley J L (2008) J Biol Chem 283: 10939-10948

### Studier F W (2005) Protein Expr Purif 41: 207-234

### Tang D, Ade J, Frye C A, Innes R W (2005a) Plant J 44: 245-257

### Tang D, Christiansen K M, Innes R W (2005b) Plant Physiol 138: 1018-1026

### Vlot A C, Klessig D F, Park S W (2008) Curr Opin Plant Biol: 11: 436-442

### Vorwerk S, Schiff C, Santamaria M, Koh S, Nishimura M, Vogel J, Somerville C, Somerville S (2007) BMC Plant Biol 7: 35

### Wang L, Mitra R M, Hasselmann K D, Sato M, Lenarz-Wyatt L, Cohen J D, Katagiri F, Glazebrook J (2008) Mol Plant Microbe Interact 21: 1408-1420

### Wildermuth M C, Dewdney J, Wu G, Ausubel F M (2001) Nature 414: 562-565

Winter D, Vinegar B, Nahal H, Ammar R, Wilson G V, Provart N J (2007) PLoS ONE 8: e718

### Yoshioka K, Kachroo P, Tsui F, Sharma S B, Shah J, Klessig D F (2001) Plant J 26: 447-459

1. A nucleic acid encoding a negative regulator of plant immunity and comprising a sequence 50%, 60%, 65%, 70%, 75%, 80%, 85%, 90%, 95%, 98% or 100% identical to the sequence as set forth in any one of SEQ ID NOs:1 to 41.

2. A polypeptide which is a negative regulator of plant immunity and comprising a sequence 50%, 60%, 65%, 70%, 75%, 80%, 85%, 90%, 95%, 98% or 100% identical to the sequence as set forth in any one of SEQ ID NOs:42 to 83.

3. A plant exhibiting enhanced pathogen resistance and having decreased expression or activity of TTM2, TTM2 homologs or TTM2 orthologs.

4. A cell of the plant of claim 3.

5. A method of modulating pathogen resistance in a plant comprising modulating expression or activity of TTM2, TTM2 homologs or TTM2 orthologs.

6. A method of enhancing pathogen resistance in a plant comprising inhibiting expression or activity of TTM2, TTM2 homologs or TTM2 orthologs.

7. The method of claim 5 or 6, wherein said TTM2, TTM2 homologs or TTM2 orthologs is encoded by a nucleic acid comprising a sequence 50%, 60%, 65%, 70%, 75%, 80%, 85%, 90%, 95%, 98% or 100% identical to the sequence as set forth in any one of SEQ ID NOs:1 to 41 or comprises a sequence 50%, 60%, 65%, 70%, 75%, 80%, 85%, 90%, 95%, 98% or 100% identical to the sequence as set forth in any one of SEQ ID NOs:42 to 83.

8. A plant produced by the method of claim 6 or 7.
